(19) United States
(12) Patent Application Publication
(10) Pub. No.: US 2011/0275673 A1
(43) Pub. Date: Nov. 10, 2011

(54) INHIBITORS OF SPHINGOSINE KINASE 1

(76) Inventors: Yibin Xiang, Acton, MA (US); Bradford Hirth, Littleton, MA (US); John L. Kane, Maynard, MA (US); Junkai Liao, Tewksbury, MA (US); Kevin Noson, San Francisco, CA (US); Christopher Yee, Needham, MA (US)

(21) Appl. No.: 13/119,386

(22) PCT Filed: Sep. 17, 2009
(86) PCT No.: PCT/US09/57318

§ 371 Date: Jun. 7, 2011

Related U.S. Application Data
(60) Provisional application No. 61/098,372, filed on Sep. 19, 2008.
(60) Provisional application No. 61/117,740, filed on Nov. 25, 2008.

(51) Int. Cl. A61K 31/454; A61K 31/4439; A61K 31/4245; A61K 31/427; A61K 31/4192; A61K 31/4155; A61K 31/404; A61K 31/4025; C07D 413/12; C07D 211/22; C07D 401/12; C07D 417/12; A61K 31/4196; C07D 403/12; C07D 405/12; A61P 35/00; A61P 3/10; A61P 31/18; A61P 31/12; A61K 31/45

(52) U.S. Cl. 514/326; 514/327; 514/343; 514/364; 514/365; 514/383; 514/406; 514/414; 514/422; 546/209; 546/221; 546/278.4; 548/131; 548/204; 548/255; 548/266.2; 548/364.1; 548/467; 548/525

(57) ABSTRACT

The invention relates to compounds of Formula (I). Compounds of the present invention are inhibitors of sphingosine kinase 3, and are useful in the treatment of various disorders and conditions, such as inflammatory disorders.

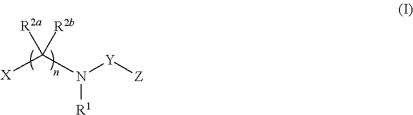

### PRIORITY CLAIMS AND RELATED APPLICATIONS

This application claims the benefit of U.S. provisional application Ser. Nos. 61/098,372 by Xiang, filed Sep. 19, 2008, and 61/117,740 by Xiang et al., filed Nov. 25, 2008, each of which is hereby incorporated herein by reference in its entirety.

### TECHNICAL FIELD

The invention relates to inhibitors of sphingosine kinase I (SKI). More particularly, the invention relates to inhibitors of SKI, methods for preparation thereof intermediates thereto, and pharmaceutical compositions and uses thereof in the treatment of various disorders and conditions, such as inflammatory and immune-mediated diseases, cancer, diabetes, and viral infections.

### BACKGROUND

The sphingolipid metabolic pathway is a highly regulated process that generates many biologically active metabolites, including sphingosine, ceramide, and sphingosine-1-phosphate (S1P). Balance of cellular levels of these bioactive lipids is increasingly recognized as a component to cell regulation and function (Taha et al., (2006) Journal of Biochemistry and Molecular Biology, 39(2)1113-131, hereinafter “Taha et al., 2006”). For example, it has been discovered that ceramide and sphingosine promote apoptosis and growth arrest phenotypes, while SIP mediates proliferation and angiogenic responses (Taha et al., 2006).

Sphingosine kinase 1 (SK1) is an important enzyme in the sphingolipid pathway. The enzyme is a component of a checkpoint that regulates relative levels of certain highly biologically active lipids. SK1 is involved in various disease states, such as immune-mediated diseases, cancer, and diabetes (Taha et al., 2006), as well as viral infections such as HIV and Hepatits C (Kaneider et al., (2004) The FASEB Journal, 18:1309-1311). Thus, inhibition of SK1 would be beneficial in the treatment of certain disease states.

Surprisingly, only a few reports appear in the literature of SK1 inhibitors (De Jonhe et al, 0999) Bioorg. Med. Chem Lett., 9:3175-3180; Kim et al. (2005) Bioorg. Med. Chem., 13;3475-3485; and French et al, (2003) Cancer Res., 63:5962-5969). A widely used SK1 inhibitor is dimethylsphingosine (Edsall et al, (1998) Biochem., 37:12892-12898). However, this molecule is a weak and non-specific inhibitor, and is also lipidic in nature, thus exhibiting unfavorable physical properties and poor compatibility with biological aqueous conditions.

There is an unmet need for inhibitors of SK1, and methods of treating various disorders and conditions mediated by SK1.

### SUMMARY

The invention is based in part on the unexpected discovery that novel inhibitors of SK1 can be made that have useful properties, such as pharmaceutical properties. An advantage of the compounds of the invention herein is the non-lipidic nature of these compounds. Thus, compounds of the invention are more compatible with biological aqueous conditions.

The invention herein provides in accordance with one aspect, compounds of Formula I;

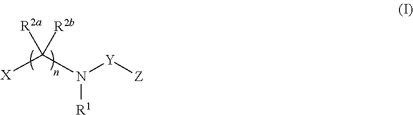

or a pharmaceutically acceptable salt, ester or prodrug thereof, wherein
X is a (C₆-C₁₀)aryl or (C₂-C₉)heteroaryl group, wherein the (C₆-C₁₀)aryl or (C₂-C₉)heteroaryl groups are optionally independently substituted by one or more groups selected from:
   (C₁-C₂₀)alkoxy, (C₁-C₂₀)alkoxy(C₁-C₂₀)alkyl,
   (C₁-C₂₀)alkyl, (C₁-C₂₀)alkyl((C₃-C₁₀)cycloalkyl, (C₁-C₂₀)alkyl(C₆-C₁₀)aryl, C₁-C₂₀)alkyl(C₆-C₁₀)aryl(C₂-C₉)heteroaryl, (C₁-C₂₀)alkyl(C₂-C₉)heteroaryl, (C₁-C₂₀)alkyloxo(C₁-C₂₀)alkyl, (C₁-C₂₀)alkylthio(C₁-C₂₀)alkyl, (C₁-C₂₀)alkyloxo(C₆-C₁₀)aryl, (C₁-C₂₀)alkylthio(C₆-C₁₀)aryl, (C₁-C₂₀)alkyl(C₂-C₉)heteroaryl(C₁-C₂₀)alkyl, (C₁-C₂₀)alkylsulfonyl(C₁-C₂₀)alkyl,
   (C₃-C₁₀)cycloalkyl, (C₃-C₁₀)cycloalkyl(C₁-C₂₀)alkyl, (C₃-C₁₀)cycloalkyl(C₁-C₂₀)alkyl(C₂-C₉)heteroaryl, (C₃-C₁₀)cycloalkyl(C₂-C₉)heteroaryl,
   (₆-C₁₀)aryl, (C₆-C₁₀)aryl(C₁-C₂₀)alkyl, (C₆-C₁₀)aryl(C₁-C₁₀)alkyl(C₂-C₉)heteroaryl, (C₆-C₁₀)aryl(C₃-C₁₀)cycloalkyl, (C₆-C₁₀)aryloxy(C₁-C₂₀)alkyl(C₆-C₁₀)aryl(C₆-C₁₀)aryl, (C₆-C₁₀)aryl(C₁-C₂₀)alkyl(C₆-C₁₀)aryl, (C₆-C₁₀)aryl(C₃-C₁₀)cycloalkyl(C₆-C₁₀)aryl, (C₆-C₁₀)aryl(C₁-C₂₀)alkyl(C₃-C₁₀)cycloalkyl,
   C₂-C₉)heteroaryl, (C₂-C₉)heteroaryl(C₁-C₂₀)alkyl, (C₂-C₉)heteroaryl(C₃-C₁₀)cycloalkyl, (C₂-C₉)heteroaryl(C₆-C₁₀)aryl, (C₂-C₉)heteroaryl(C₆-C₁₀)aryl(C₁-C₂₀)alkyl, (C₂-C₉)heteroaryl(C₁-C₂₀)alkyl(C₆-C₁₀)aryl, (C₂-C₉)heteroaryl(C₃-C₁₀)cycloalkyl(₆-C₁₀)aryl, (C₂-C₉)heteroaryl(C₁-C₂₀)alkyl(C₃-C₁₀)cycloalkyl,
   hydroxy,
   halogen,
   oxo(C₁-C₂₀)alkyl, and
   thio(C₁-C₂₀)alkyl;
   wherein the (C₁-C₂₀)alkyl, (C₃-C₁₀)cycloalkyl, (C₁-C₂₀)alkoxy, (C₆-C₁₀)aryl, (C₂-C₉)heteroaryl, oxo(C₁-C₂₀)alkyl, and thio(C₁-G₂₀)alkyl groups are optionally independently substituted by one or more groups selected from: (C₁-C₂₀)alkyl, amino, hydroxyl, carbonyl, and halogen;
n is 0, 1, 2, or 3;
Y is carbonyl or —CH₂—,
Z is (C₁-C₂₀)alkyl, (C₃-C₁₀)cycloalkyl, or (C₂-C₉)heterocycloalkyl, wherein the (C₁-C₂)alkyl or (C₂-C₉)heterocycloalkyl groups are optionally independently substituted by one or more groups selected from: amino, hydroxyl, carbonyl, —N[(C₁-C₂₀)alkyl]₂, —NH[(C₁-C₂₀)alkyl], —N[(C₃-C₁₀)cycloalkyl]₂, —NH[(C₃-C₁₀)cycloalkyl], —N(C₃-C₁₀cycloalkyl(C₁-C₂₀)alkyl]₂, —NH[(C₃C₁₀)cycloalkyl(C₁-C₂₀)alkyl], and halogen;
R¹ is hydrogen, (C₁-C₁₂)alkyl, (C₃-C₁₀)cycloalkyl, or (C₂-C₉)heterocycloalkyl; and
R^{2a}, R^{2b}
   are each independently hydrogen, halogen, hydroxyl, (C₁-C₁₂)alkyl, (C₃-C₁₀)cycloalkyl, or (C₂-C₉)heterocycloalkyl wherein each R^{2a} and R^{2b} is not halogen or hydroxyl at the position α to the nitrogen atom in Formula 1, and wherein optionally R^{2a} and R^{2b} may together form a 3- to 7-membered ring, and
      wherein optionally R¹ and one of R^{2a} and R^{2b} may together form a 3- to 7-membered ring.

In some embodiments of the compounds of Formula I, Y is carbonyl. In other related embodiments of the compounds of Formula I, n is 0, or 2. In yet other related embodiments of the compounds of Formula I, R¹ is hydrogen (C₁-C₆)alkyl or (C₃-C₆)cycloalkyl. In still other related embodiments of the compounds of Formula I. R¹ is hydrogen or (C₁-C₃)alkyl. In certain related embodiments of the compounds of Formula I, R¹ is hydrogen. In other related embodiments of the compounds of Formula I, X is (C₆)aryl, (C₄-C₅)heteroaryl, or (C₇-C₈)heteroaryl. In other related embodiments of the compounds of Formula I, X is (C₆)aryl and n is 0 or 1. In still other related embodiments of the compounds of Formula I, X is (C₄-C₅)heteroaryl and n is 0 or 1. In still other related embodiments of the compounds of Formula I, X is (C₇-C₈)heteroaryl and n is 0 or 1. In certain embodiments, the present disclosure provides a compound of formula II:

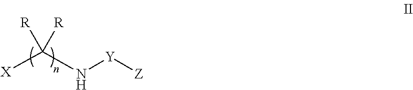

or a pharmaceutically acceptable salt, ester, or prodrug thereof, wherein

X is (C₆-C₁₀)aryl or (C₂-C₉)heteroaryl, wherein the (C₆-C₁₀)aryl or (C₂-C₉) heteroaryl groups are optionally substituted by one or more groups selected from the group consisting of: (C₁-C₁₂)alkoxy, (C₁-C₁₂)alkyloxy (C₁-C₁₂)alkyl, (C₁-C₁₂)alkyl, (C₁-C₁₂)alkyl(C₆-C₁₀)aryl, (C₁-C₁₂)alkyl(C₆-C₁₀)aryl(C₂-C₉)heteroaryl, (C₁-C₁₂)alkyl(C₂-C₉)heteroaryl, (C₁-C₁₂)alkylthio(C₁-C₁₂)alkyl, (C₁-C₂₀)alkylsulfonyl(C₁-C₂₀)alkyl, (C₆-C₁₀)aryl(C₁-C₁₂)alkyl(C₂-C₉)heteroaryl, (C₃-C₁₀)cycloalkyl, (C₃-C₁₀)cycloalkylalkyl, (C₃-C₁₀)cycloalkyl(C₁-C₁₂(C₂-C₉)heteroaryl, (C₃-C₁₀)cycloalkyl(C₂-C₉)heteroaryl, halo(C₆-C₁₀)aryl(C₁-C₁₂)alkyl(C₂-C₉)heteroaryl, halo(C₆-C₁₀)aryloxy(C₁-C₁₂)alkyl, halo(C₃-C₁₀)cycloalkyl(C₂-C₉)heteroaryl, hydroxyl, hydroxyl(C₁-C₁₂)alkyl, oxo(C₁-C₂)alkyl and halo;

each is independently hydrogen or (C₁-C₁₂)alkyl;

n is 0 1, or 2;

Y is carbonyl or —CH₂—; and

Z is (C₁-C₁₂)alkyl or (C₂-C₉)heterocycloalkyl, wherein the (C₁-C₁₂)alkyl or (C₂-C₉)heterocycloalkyl groups are optionally substituted by one or more amino or hydroxyl.

Those of skill in the art given the benefit of the present disclosure will appreciate that in embodiments in formula I:

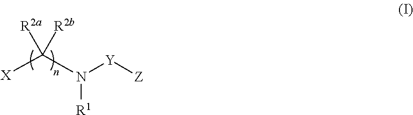

when n is 0, it is taken to mean that a direct single covalent bond will connect X and the N atom.

In certain embodiments of the compounds of Formula I, X is:

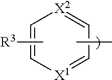

wherein:
X¹, X²
   are each independently N or —CH—, wherein at least one of X¹ and X² is nitrogen;
R³ is one or more groups selected from:
   (C₁-C₁₀)alkoxy, (C₁-C₂₀)alkoxyalkyl,
   (C₁-C₂₀)alkyl, (C₁-C₂₀)alkylcycloalkyl, (C₁-C₂₀)alkylaryl, (C₁-C₂₀)alkylarylheteroaryl, (C₁-C₂₀)alkylheteroaryl, (C₁-C₂₀)alkyloxoalkyl, (C₁-C₂₀)alkylthioalkyl, (C₁-C₂₀)alkyloxoaryl, (C₁-C₂₀))alkylthioaryl, (C₁-C₂₀)alkylheteroarylalkyl,
   (C₃-C₁₀)cycloalkyl, (C₃-C₁₀)cycloalkylalkyl, (C₃-C₁₀)cycloalkylalkylheteroaryl, (C₃-C₁₀)cycloalkylheteroaryl,
   (C₆-C₁₀)aryl, (C₆-C₁₀)arylalkyl, (C₆-C₁₀)arylcycloalkyl, (C₆-C₁₀)arylaryl, (C₆-C₁₀)arylalkylaryl, (C₆-C₁₀)arylcycloalkylaryl, (C₆-C₁₀)arylalkylcycloalkyl,
   (C₂-C₉)heteroaryl, (C₂-C₉)heteroarylalkyl, (C₂-C₉)heteroarylcycloalkyl, (C₂-C₉)heteroarylaryl, (C₂-C₉)heteroarylarylalkyl, (C₂-C₉)heteroarylalkylaryl, (C₂-C₉)heteroarylcycloalkylaryl, (C₂-C₉)heteroarylalkylcycloalkyl,
   hydroxyl,
   halogen,
   oxoalkyl, and
   thioalkyl;
   wherein the alkylcycloalkyl, alkoxy, aryl, heteroaryl, oxoalkyl, and thioalkyl groups are optionally independently substituted by one or more groups selected from: amino, hydroxyl, carbonyl, and halogen.

In certain other embodiments of the compounds of Formula I, X is:

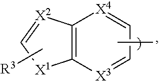

where
X¹ is N or O;
X², X³, X⁴
   are each N or —CH—, wherein at least one of X², X³, and X⁴ is nitrogen;
R³ is one or more groups selected from:
   (C₁-C₂₀)alkoxy, (C₁-C₂₀)alkoxyalkyl,
   (C₁-C₂₀)alkyl, (C₁-C₂₀)alkylcycloalkyl, (C₁-C₂₀)alkylaryl, (C₁-C₂₀)alkylarylheteroaryl, (C₁-C₂₀)alkylheteroaryl, (C₁-C₁₀)alkyloxoalkyl, (C₁-C₂₀)alkylthioalkyl, (C₁-C₂₀)alkyloxoaryl, (C₁-C₂₀)alkylthioaryl, (C₁-C₂₀)alkylheteroarylalkyl,
   (C₃-C₁₀)cycloalkyl, (C₃-C₁₀)cycloalkylalkyl, (C₃-C₁₀)cycloalkylalkylheteroaryl, (C₃-C₁₀)cycloalkylheteroaryl,
   (C₆-C₁₀)aryl, (C₆-C₁₀)arylalkyl, (C₆-C₁₀)arylcycloalkyl, (C₆-C₁₀)arylaryl, (C₆-C₁₀)arylalkylaryl, (C₆-C₁₀)arylcycloalkylaryl, (C₆-C₁₀))arylalkylcycloalkyl,
   (C₂-C₉)heteroaryl, (C₂-C₉)heteroarylalkyl, (C₂-C₉)heteroarylcycloalkyl, (C₂-C₉)heteroarylaryl, (C₂-C₉)heteroarylarylalkyl, (C₂-C₉)heteroarylalkylaryl, (C₂-C₉)heteroarylcycloalkylaryl, (C₂-C₉)heteroarylalkylcycloalkyl,
   hydroxyl,
   halogen,
   oxoalkyl, and
   thioalkyl;
   wherein the alkyl, cycloalkyl, alkoxy, aryl, heteroaryl, oxoalkyl, and thioalkyl groups are optionally independently substituted by one or More groups selected from: amino, hydroxyl, carbonyl, and halogen.

In related embodiments of the compounds of Formula I, X is substituted with a halogen, for example, F or Cl. In other related embodiments, X is an aryl or heteroaryl. In other related embodiments of the compounds of Formula I, X is substituted with

R⁵-A-R⁴,

wherein
A is (C₆)aryl or (C₂-C₅)heteroaryl;
R⁴ is a single bond or (C₁-C₄)alkylene or (C₁-C₃)heteroalkylene; and
R⁵ is a group selected, from:
   (C₂-C₂₀)alkyl, (C₂-C₂₀)alkylcycloalkyl, (C₂-C₂₀)alkylaryl,
   (C₁-C₁₉)heteroalkyl, and (C₁-C₁₉)heteroalkylcycloalkyl.

in related embodiments, R⁴ is a single bond. In other related embodiments, R⁵ is (C₂-C₁₂)alkyl or (C₁-C₁₁)heteroalkyl. In other related embodiments, A is a (C₂-C₃)heteroaryl. In still other related embodiments, A is selected from:

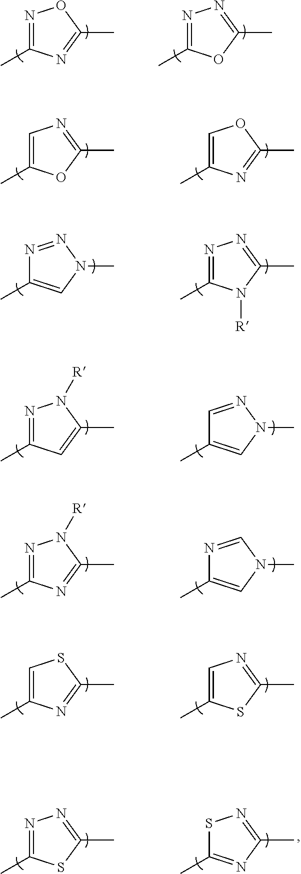

and all regioisomers thereof,
wherein
R′ is a group selected from the group of:
   (C₂-C₂₀)alkyl, (C₂-C₂₀)alkylcycloalkyl, (C₂-C₂₀)alkylaryl,
   (C₁-C₉)heteroalkyl, and (C₁-C₁₉)heteroalkylcycloalkyl,

In certain embodiments of the compounds of Formula I, Z is (C₁-C₁₂)alkyl, optionally substituted by one or more groups selected from: amino, hydroxyl, carbonyl, and halogen. In other embodiments of the compounds of Formula I, Z is (C₂-C₉)heterocycloalkyl, optionally substituted by one or more groups selected front amino, hydroxyl, carbonyl, and halogen, in other embodiments of the compounds of Formula I, Z is:

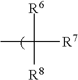

wherein,
R⁶, R⁷
   are each independently a hydrogen, halogen, hydroxyl,
   (C₁C₁₀)heteroalkyl, or
   (C₃-C₈)cycloalkyl or (C₂-C₇)heterocycloalkyl;
R⁸ is —B—(R^{8a})ₘ, wherein
   B is N, O, or S;
   m is I when B is O or S, or 2 when B is N;
   each R^{8a} is independently a hydrogen, (C₁-C₁₀)alkyl; (C₁-C₁₀)heteroalkyl, or (C₃-C₈)cycloalkyl or (C₂-C₇)heterocycloalkyl;
   wherein, when B is N, a R^{8a} may together form a ring with the other R^{8a}, R⁶, or R⁷; and
   when B is O or S, R^{8a} may together form a ring, with R⁶ or R⁷.

In related embodiments, B is N. In other related embodiments, R^{8a} together with R⁷ from a ring Q,

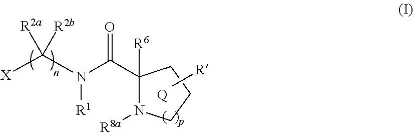

wherein
R′ is absent, one or more amino, halogen, hydroxyl, carbonyl, carbonyl, and (C₁-C₁₀)alkyl; (C₁-C₁₀)heteroalkyl or (C₃-C₈)cycloalkyl or (C₂-C₇)heterocycloalkyl; and
p is 0, 1, 2, or 3.

In other related embodiments, n is 1 of 2, and the R^{2b} at α-position to the nitrogen atom in the formula is hydrogen:

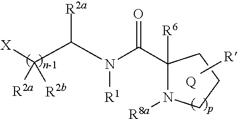

In related embodiments, X is (C₆)aryl, (C₄-C₅)heteroaryl, or(C₇-C₈)heteroaryl. In another related embodiments. X is substituted with

R⁵-A-R⁴

wherein
A is (C₆)aryl or (C₂-C₅)heteroaryl:
R⁴ is a single bond or (C₁-C₄)alkylene or (C₁-C₃)heteroalkylene; and
R⁵ is a group selected from:
   (C₂-C₂₀)alkyl, (C₂-C₂₀)alkylcycloalkyl, (C₂-C₂₀)alkylaryl,
   (C₁-C₁₉)heteroalkyl, and (C₁-C₁₉)heteroalkylcycloalkyl

In certain related embodiments,. R⁴ is a single bond; and A is (C₂-C₃)heteroaryl. In other related embodiments, A is selected from

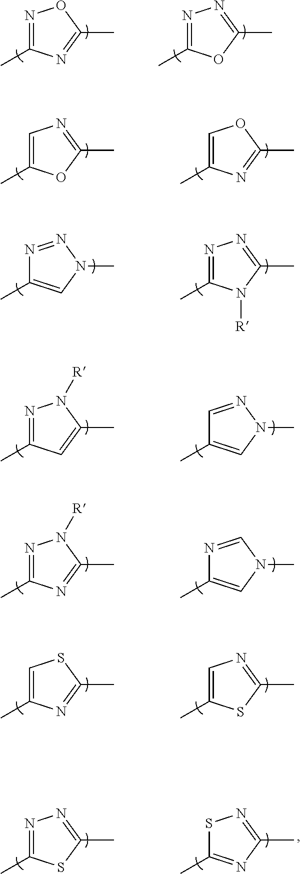

and all regioisomers thereof,
wherein
R′ is a group selected from the group of:
   (C₂-C₂₀)alkylcycloalkyl, (C₂-C₂₀)alkylaryl,
   (C₁-C₁₉)heteroalkyl, and (C₁-C₁₉)heteroalklylcycloalkyl.

In certain related embodiments. X is (C₆)aryl In other related embodiments, X is (C₄-C₅)heteroaryl. In still other related embodiments, X is (C₇-C₈)heteroaryl. In other embodiments, X is aryl. In still other embodiments, X is heteroaryl. In related embodiments. R⁶ is

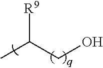

wherein
q is 0, 1, 2, 3, or 4;
R⁹ is hydrogen, halogen, hydroxy, amino, and (C₁-C₁₂)alkyl, (C₁-C₁₂)heteroalkyl, C₃-C₈)cycloalkyl or (C₂-C₇)heterocycloalkyl.

Another aspect of the invention herein provides a pharmaceutical composition comprising an amount of a compound of Formula I

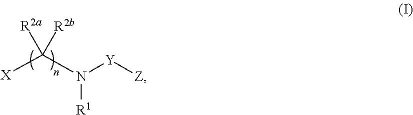

or a pharmaceutically acceptable salt, ester or pro-drug thereof, effective in the treatment or prevention of a disorder or condition selected from the group consisting of inflammation and immune-mediated disease, cancer, diabetes, inflammatory bowel disease, fibrosis, polycystic kidney disease, arteriosclerosis, pulmonary diseases, and viral infections or a related disorder or condition thereof in a mammal, including a human, and a pharmaceutically effective carrier, wherein
X is a (C₆-C₁₀)aryl or (C₂-C₉)heteroaryl group, wherein the (C₆-C₁₀)aryl or (C₂-C₉heteroaryl groups are optionally independently substituted by one or more groups selected from:
   (C₁-C₂₀)alkoxy, (C₁-C₂₀)alkoxy(C₁-C₂₀)alkyl,
   (C₁-C₂₀)alkyl, (C₁-C₂₀)alkyl(C₃-C₁₀)cycloalkyl, (C₁-C₂₀)alkyl(C₆-C₁₀)aryl, (C₁-C₂₀)alkyl(C₆-C₁₀)aryl(C₂-C₉)heteroaryl, (C₁-C₂₀)alkyl(C₂-C₉)heteroaryl, (C₁-C₂₀)alkyloxo(C₁-C₂₀)alkyl, (C₁-C₂₀)alkylthio(C₁-C₂₀)alkyl, (C₁-C₂₀)alkyloxo(C₆-C₁₀)aryl, (C₁-C₂₀)alkylthio(C₆-C₁₀)aryl, (C₁-C₂₀)alkyl(C₂-C₉)heteroaryl(C₁-C₂₀)alkyl, (C₁-C₂₀)alkylsulfonyl(C₁-C₂₀)alkyl,
   (C₃-C₁₀)cycloalkyl, (C₃-C₁₀)cycloalkyl(C₁-C₂₀)alkyl, (C₃-C₁₀)cycloalkyl(C₁-C₂₀)alkyl(C₂-C₉)heteroaryl, (C₃-C₁₀)cycloalkyl(C₂-C₉)heteroaryl,
   (C₆-C₁₀)aryl, (C₆-C₁₀)aryl(C₁-C₂₀)alkyl, (C₆-C₁₀)aryl(C₁-C₂₀)alkyl(C₂-C₉)heteroaryl, (C₆-C₁₀)aryl(C₃-C₁₀)cycloalkyl, (C₆-C₁₀)aryloxy(C₁-C₂₀)alkyl (C₆-C₁₀)aryl(C₆-C₁₀)aryl, (C₆-C₁₀)aryl(C₁-C₂₀)alkyl((C₆-C₁₀)aryl, (C₆-C₁₀)aryl(C₃-C₁₀)cycloalkyl(C₆-C₁₀)aryl, (C₆-C₁₀)aryl(C₁-C₂₀)alkyl(C₃-C₁₀)cycloalkyl,
   (C₂-C₉)heteroaryl, (C₂-C₉)heteroaryl(C₁-C-₂₀)alkyl, (C₂-C₉)heteroaryl(C₃-C₁₀)cycloalkyl, (C₂-C₉)heteroaryl(C₆-C₁₀)aryl, (C₂-C₉)heteroaryl(C₆-C₁₀)aryl(C₁-C₂₀)alkyl, (C₂-C₉)heteroaryl(C₁-C₂₀)alkyl(C₆-C₁₀)aryl, (C₂-C₉)heteroaryl(C₃-C₁₀)cycloalkyl(C₆-C₁₀)aryl, (C₂-C₉)heteroaryl(C₁-C₂₀)alkyl(C₃-C₁₀)cycloalkyl,
   hydroxyl,
   halogen,
   oxo(C₁-C₂₀)alkyl, and
   thio(C₁-C₂₀)alkyl;
   wherein the (C₁-C₂₀)alkyl, (C₃-C₁₀))cycloalkyl, (C₁-C₂₀)alkoxy, (C₆-C₁₀)aryl, (C₂-C₉)heteroaryl, oxo(C₁-C₂₀)alkyl, and thio(C₁-C₂₀)alkyl groups are optionally independently substituted by one or mote groups selected from: (C₁-C₂₀)alkyl, amino, hydroxyl, carbonyl, and halogen;
n is 0, 1, 2, or 3;
is carbonyl or —CH₂—;
Z is (C₁-C₂₀)alkyl, (C₃-C₁₀)cycloalkyl, or (C₂-C₉)heterocycloalkyl, wherein the (C₁-C₂₀)alkyl or (C₂-C₉)heterocycloalkyl groups ate optionally independently substituted by one or more groups selected from: amino, hydroxyl, carbonyl, —N[(C₁-C₂₀alkyl]₂, —NH[(C₁-C₂₀)alkyl], —N[(C₃-C₁₀)cycloalkyl]₂, —NH[(C₃-C₁₀)cycloalkyl], —N[(C₃-C₁₀cycloalkyl(C₁-C₂₀)alkyl]₂, —NH[(C₃-C₁₀)cycloalkyl(C₁-C₂₀)alkyl], and halogen;
R¹ is hydrogen, (C₁-C₁₂)alkyl, (C₃-C₁₀)cycloalkyl, or (C₂-C₉)heterocycloalkyl; and
R^{2a} R^{2b}
   are each independently hydrogen, halogen, hydroxyl, (C₁-C₁₂)alkyl, C₃-C₁₀)cycloalkyl, or (C₂-C₉)heterocycloalkyl wherein each R^{2a} and R^{2b} is not halogen or hydroxyl at the position α to the nitrogen atom in Formula I, and wherein optionally R^{2a} and R^{2b} may together form a 3- to 7-membered ring, and
      wherein optionally R¹ and one of R^{2a} and R^{2b} may together form a 3- to 7-membered ring.

In some related embodiments, the disorder or condition is an immune-mediated disease. In other related embodiments, the disorder or condition is cancer. In still other related embodiments, the disorder or condition is a type of diabetes. In still other related embodiments, the disorder or condition is a viral infection. In another related embodiment, the mammal is a human.

Another aspect of the invention herein provides a method of treating or preventing a disorder or condition in a mammal, including a human, including administering to a subject in need thereof an therapeutically effective amount of a pharmaceutical composition comprising a compound of Formula I

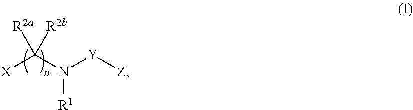

or a pharmaceutically acceptable salt, ester or pro-drug thereof,
wherein the disorder or condition is selected from the group consisting of inflammation and immune-mediated disease, cancer, diabetes, inflammatory bowel disease, fibrosis_{:} polycystic kidney disease, arteriosclerosis, pulmonary diseases, and viral infections or a related disorder or condition thereof, wherein:
X is a (C₆-C₁₀)aryl or (C₂-C₉)heteroaryl group, wherein the (C₆-C₁₀)aryl or (C₂-C₉)heteroaryl groups are optionally independently substituted by one or more groups selected from:
   (C₁-C₂₀)alkoxy, (C₁-C₂₀)alkoxy(C₁-C₂₀)alkyl,
   (C₁-C₂₀)alkyl, (C₁-C₂₀)alkyl(C₃-C₁₀)cycloalkyl, (C₁-C₂₀)alkyl(C₆-C₁₀)aryl, (C₁-C₂₀)alkyl(C₆-C₁₀)aryl(C₂-C₉)heteroaryl, (C₁_C₂₀)alkyl(C₂-C₉)heteroaryl, (C₁-C₂₀)alkyloxo(C₁-C₂₀)alkyl, (C₁-C₂₀)alkylthio(C₁-C₂₉)alkyl, (C₁-C₂₀)alkyloxo(C₆-C₁₀)aryl, (C₁-C₂₀)alkylthio(C₆-C₁₀)aryl, (C₁-C₂₀)alkyl(C₂-C₉)heteroaryl(C₁-C₂₀)alkyl, (C₁-C₂₀)alkylsulfonyl(C₁-C₂₀)alkyl,
   (C₃-C₁₀)cycloalkyl(C₃-C₂₀)cycloalkyl(C₁-C₂₀)alkyl, (C₃-C₁₀)cycloalkyl(C₁-C₂₀)alkyl(C₂-C₉)heteroaryl, (C₃-C₁₀)cycloalkyl(C₂-C₉)heteroaryl,
   (C₆-C₁₀)aryl, (C₆-C₁₀)aryl(C₁-C₂₀)alkyl, (C₆-C₁₀)aryl(C₁-C₂₀)alkyl(C₂-C₉)heteroaryl, (C₆-C₁₀)aryl(C₃-C₁₀)cycloalkyl, (C₆-C₁₀)aryloxy(C₁-C₂₀)alkyl (C₆-C₁₀)aryl(C₆-C₁₀)aryl, (C₆-C₁₀)aryl(C₁-C₂₀)alkyl(C₆-C₁₀)aryl, (C₆-C₁₀)aryl(C₃-C₁₀)cycloalkyl(C₆-C₁₀)aryl, (C₆-C₁₀)aryl(C₁-C₂₀)alkyl(C₃-C₁₀)cycloalkyl,
   (C₂-C₉)heteroaryl, (C₂-C₉)heteroaryl(C₁-C-₂₀)alkyl, (C₂-C₉)heteroaryl(C₃-C₁₀)cycloalkyl, (C₂-C₉)heteroaryl(C₆-C₁₀)aryl, (C₂-C₉)heteroaryl(C₆-C₁₀)aryl(C₁-C₂₀)alkyl, (C₂-C₉)heteroaryl(C₁-C₂₀)alkyl(C₆-C₁₀)aryl, (C₂-C₉)heteroaryl(C₃-C₁₀)cycloalkyl(C₆-C₁₀)aryl, (C₂-C₉)heteroaryl(C₁-C₂₀)alkyl(C₃-C₁₀)cycloalkyl,
   hydroxyl,
   halogen,
   oxo(C₁-C₂₀)alkyl, and
   thio(C₁-C₂₀)alkyl;
   wherein the (C₁_C₂₀)alkyl, (C₃-C₁₀)cycloalkyl, (C₁-C₂₀)alkoxy, (C₆-C₁₀)aryl, (C₂-C₉)heteroaryl, oxo(C₁-C₂₀)alkyl, and thio(C₁-C₂₀)alkyl groups are optionally independently substituted by one or mote groups selected from: (C₁-C₂₀)alkyl, amino, hydroxyl, carbonyl, and halogen;
   n is 0, 1, 2, or ‘3;
   Y is carbonyl or —CH₂—;
   Z is (C₁-C₂₀)alkyl, (C₃-C₁₀)cycloalkyl, or (C₂-C₉)heterocycloalkyl, wherein the (C₁-C₂₀)alkyl or (C₂-C₉)heterocycloalkyl groups ate optionally ⁻independently substituted by one or more groups selected from: amino, hydroxyl, carbonyl, —N[(C₁-C₂₀)alkyl]₂, —NH[(C₁-C₂₀)alkyl], —N[(C₃-C₁₀)cycloalkyl]₂, —NH[(C₃_C₁₀)cycloalkyl], —N[(C₃-C₁₀)cycloalkyl(C₁-C₂₀)alkyl]₂, —NH[(C₃-C₁₀)cycloalkyl(C₁-C₂₀)alkyl], and halogen;
R¹ is hydrogen, (C₁-C₁₂)alkyl, (C₃-C₁₀)cycloalkyl, or (C₂-C₉)heterocycloalkyl; and
R^{2a} R^{2b}
   are each independently hydrogen, halogen, hydroxyl, (C₁-C₁₂)alkyl, (C₃-C₁₀)cycloalkyl, or (C₂-C₉)heterocycloalkyl wherein each R^{2a} and R^{2b} is not halogen or hydroxyl at the position α to the nitrogen atom in Formula I, and wherein optionally R^{2a} and R^{2b} may together form a 3- to 7-membered ring, and
      wherein optionally R¹ and one of R^{2a} and R^{2b} may together form a 3- to 7-membered ring.

In related embodiments, the disorder or condition is an immune-mediated disease. In other related embodiments, the disorder or condition is cancer. In still other related embodiments, the disorder or condition is a type of diabetes. In still other related embodiments, the disorder or condition is a viral infection, e.g., HIV or HCV viral infections. In another related embodiment, the mammal is a human.

Another aspect of the invention herein provides a method for treating a disorder or condition mediated by sphingosine kinase-1, the method including administering to a subject in need thereof a therapeutically effective amount of a pharmaceutical composition comprising a compound of Formula I

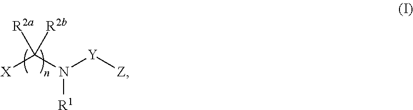

or a pharmaceutically acceptable salt, ester or pro-drug thereof,
wherein
X is a (C₆-C₁₀))aryl or (C₂-C₉)heteroaryl group, wherein the (C₆-C₁₀)aryl or (C₂-C₉)heteroaryl groups are optionally independently substituted by one or more groups selected from:
   (C₁-C₂₀)alkoxy, (C₁-C₂₀)alkoxy(C₁-C₂₀)alkyl,
   (C₁-C₂₀)alkyl, (C₁-C₂₀)alkyl(C₃-C₁₀)cycloalkyl, (C₁-C₂₀)alkyl(C₆-C₁₀)aryl, (C₁-C₂₀)alkyl(C₆-C₁₀)aryl(C₂-C₉)heteroaryl, (C₁-C₂₀)alkyl(C₂-C₉)heteroaryl, (C₁-C₂₀)alkyloxo(C₁-C₂₀)alkyl, (C₁-C₂₀)alkylthio(C₁-C₂₀)alkyl, (C₁-C₂₀)alkyloxo(C₆-C₁₀)aryl, (C₁-C₂₀)alkylthio(C₆-C₁₀)aryl, (C₁-C₂₀)alkyl(C₂-C₉)heteroaryl(C₁-C₂₀)alkyl, (C₁-C₂₀)alkylsulfonyl(C₁-C₂₀)alkyl,
   (C₃-C₁₀)cycloalkyl, (C₃-C₁₀)cycloalkyl(C₁-C₂₀)alkyl, (C₃-C₁₀)cycloalkyl(C₁-C₂₀)alkyl(C₂-C₉)heteroaryl, (C₃-C₁₀)cycloalkyl(C₂-C₉)heteroaryl,
   (C₆-C₁₀)aryl, (C₆-C₁₀)aryl(C₁-C₂₀)alkyl, (C₆-C₁₀)aryl(C₁-C₂₀)alkyl(C₂-C₉)heteroaryl, (C₆-C₁₀)aryl(C₃-C₁₀)cycloalkyl, C₆-C₁₀)aryloxy(C₁-C₂₀)alkyl (C₆-C₁₀)aryl(C₆-C₁₀)aryl, (C₆-C₁₀)aryl(C₁-C₂₀)alkyl(C₆-C₁₀)aryl(C₃-C₁₀)cycloalkyl(C₆-C₁₀)aryl, (C₆-C₁₀)aryl(C₁-C₂₀)alkyl(C₃-C₁₀)cycloalkyl,
   (C₂-C₉)heteroaryl, (C₂-C₉)heteroaryl(C₁-C₂₀)alkyl, (C₂-C₉)heteroaryl(C₃-C₁₀)cycloalkyl, (C₂-C₉)heteroaryl(C₆-C₁₀)aryl, (C₂-C₉)heteroaryl(C₁-C₁₀)aryl(C₁-C₂₀)alkyl, (C₂-C₉)heteroaryl(C₁-C₂₀)alkyl(C₆-C₁₀)aryl, (C₂-C₉)heteroaryl(C₃-C₁₀)cycloalkyl(C₆-C₁₀)aryl, (C₂-C₉)heteroaryl(C₁-C₂₀)alkyl(C₃-C₁₀)cycloalkyl,
   hydroxyl,
   halogen,
   oxo(C₁-C₂₀)alkyl, and
   thio(C₁-C₂₀)alkyl;
   wherein the (C₁-C₂₀)alkyl, (C₃-C₁₀)cycloalkyl, (C₁-C₂₀)alkoxy, (C₆-C₁₀)aryl, (C₂-C₉)heteroaryl, oxo(C₁-C₂₀)alkyl, and thio(C₁-C₂₀)alkyl groups are optionally independently substituted by one or more groups selected from: (C₁-C₂₀)alkyl, amino, hydroxyl, carbonyl, and halogen;
   is 0, 1, 2, or 3;
   Y is carbonyl or —CH₂—;
   Z is (C₁-C₂₀)alkyl, (C₃-C₁₀)cycloalkyl, or (C₂-C₉)heterocycloalkyl wherein the (C₁-C₂₀)alkyl or (C₂-C₉)heterocycloalkyl groups are optionally independently substituted by one or more groups selected from: amino, hydroxyl, carbonyl, —N[(C₁-C₂₀)alkyl]₂, —NH[(C₁-C₂₀)alkyl], —N[(C₃-C₁₀)cycloalkyl]₂, —NH[(C₃-C₁₀)cycloalkyl], —N[(C₃-C₁₀)cycloalkyl(C₁-C₂₀)alkyl]₂, —NH[(C₃-₁₀)cycloalkyl(C₁-C₂₀)alkyl], and halogen;
R¹ is hydrogen, (C₁-C₁₂)alkyl, (C₃-C₁₀)cycloalkyl, or (C₂-C₉)heterocycloalkyl; and
R^{2a}, R^{2b}
   are each independently hydrogen, halogen, hydroxyl, (C₁-C₁₂)alkyl, (C₃-C₁₀)cycloalkyl, or (C₂-C₉)heterocycloalkyl wherein each R^{2a} and R^{2b} is not halogen or hydroxyl at the position α to the nitrogen atom in Formula I, and wherein optionally R^{2a} and R^{2b} may together form a 3- to 7-membered ring, and
wherein optionally R¹ and one of R^{2a} and R^{2b} may together form a 3- to 7-membered ring,

### Definitions

Definitions of specific functional groups and chemical terms are described in more detail below. General principles of organic chemistry, as well as specific functional moieties and reactivity, are described in “Organic Chemistry”. Thomas Sorrell, University Science Books, Sausalito: 1999.

Certain compounds of the present invention may exist in particular geometric or stereoisomeric forms. The present invention contemplates all such compounds, including cis- and trans-isomers, R- and S-enantiomers, diastereomers, (D)-isomers, (L)-isomers, the racemic mixtures thereof, and other mixtures thereof, as falling within the scope of the invention. Additional asymmetric carbon atoms may be present in a substituent such as an alkyl group. All such isomers, as well as mixtures thereof are intended to be included in this invention,

Isomeric mixtures containing any of a variety of isomer ratios may be utilized in accordance with the present invention. For example, where only two isomers are combined, mixtures containing 50:50, 60:40, 70:30, 80:20, 90:10, 95:5, 96:4, 97:3, 98:2, 99:1; or 100:0 isomer ratios are contemplated by the present invention. Those of ordinary skill in the art will readily appreciate that analogous ratios are contemplated for more complex isomer mixtures.

If for instance, a particular enantiomer of a compound of the present invention is desired, it may be prepared by asymmetric synthesis, or by derivation with a chiral auxiliary, where the resulting diastereomeric mixture is separated and the auxiliary' group cleaved to provide the pure desired enantiomers. Alternatively, where the molecule contains a basic functional group, such as amino, or an acidic functional group, such as carboxyl, diastereomeric salts are formed with an appropriate optically-active acid or base, followed by resolution of the diastereomers thus formed by fractional crystallization or chromatographic methods well known in the art, and subsequent recovery of the pure enantiomers.

Given the benefit of this disclosure, one of ordinary skill in the art will appreciate that synthetic methods, as described herein, may utilize a variety of protecting groups. By the term “protecting group”, as used herein, it is meant that a particular functional moiety, e.g., O, S, or N, is temporarily blocked so that a reaction can be carried out selectively at another reactive site in a multifunctional compound. In preferred embodiments, a protecting, group reacts selectively in good yield to give a protected substrate that is stable to the projected reactions; the protecting group should be selectively removable in good yield by preferably readily available, non-toxic reagents that do not attack the other functional groups; the protecting group forms an easily separable derivative (more preferably without the generation of new stereogenic centers); and the protecting group has a minimum of additional functionality to avoid further sites of reaction. Oxygen, sulfur, nitrogen, and carbon protecting groups may be utilized. Examples of a variety of protecting groups can be found in Protective Groups in Organic Synthesis, Third Ed, Greene, T. W. and Wuts, P. G., Eds., John Wiley & Sons, New York: 1999.

It will be appreciated that the compounds, as described herein, may be substituted with any number of substituents or functional moieties.

The term “sphingosine kinase-1” or “SK1” refers to an enzyme that catalyzes the transformation of sphingosine to sphingosine-1-phosphate (S1P), i.e., phosphorylates sphingosine into SIP. Properties and activities of SK1, e.g., protein sequence of SK1, structural properties of SK1, biochemical properties of SK1, and regulation of SK1, are described, in Taha et al, (2006, journal of Biochemistry and. Molecular Biology, 39(2):113-131).

Certain compounds of the invention are potent inhibitors of SK1 activity. SK1 activity refers to the production, release, expression, function, action, interaction or regulation of SK1, including, e.g., temporal, site or distribution aspects. The activity of SK1 includes modifications, e.g., covalent or non-covalent modifications of SK1 polypeptide, covalent or non-covalent modifications that SK1 induces in other substances, changes in the distribution of SK1 polypeptide, and changes SK1 induces in the distribution of other substances.

Any aspect of SK1 activity can be evaluated. Methods and techniques known to those skilled, in the art can be found in various references, e.g., Ausubel et al., ed., Current Protocols in Mol. Biology, New York: John Wiley & Sons, 1990; Sambrook et al., Mol. Cloning, Cold Spring Harbor Laboratory Press, New York, N.Y. (1989). Examples of SK1 activity that can be evaluated include binding activity of SK1 polypeptide to a binding molecule; the effect of SK1 polypeptide on the posttranslational modification or stability of a target gene; the level of SK1 protein:, the level of SK1 mRNA; or the level of SK1 modification, e.g., phosphorylation, acetylation, methylation, carboxylation or glycosylation. By binding molecule is meant any’ molecule to which SK1 can bind, e.g., at nucleic acid, e.g., a DNA regulatory region, a protein, a metabolite, a peptide mimetic, a non-peptide mimetic, an antibody, or any other type of ligand. Binding can be shown, e.g., by electrophoretic mobility shift analysis (EMSA), by the yeast or mammalian two-hybrid or three-hybrid assays, by competition with dimethylspingosine photoaffinity label or biotin-SK1 binding. Transactivation of a target gene by SK1 can be determined, in a transient transfection assay in which the promoter of the target gene is linked to a reporter gene, e.g., β-galactosidase or luciferase, and co-transfected with a SK1 expression vector. Levels of SK1 protein, mRNA or modification, can, e.g., be measured in a sample, e.g., a tissue sample, e.g., endothelial cells in blood vessels, T and B lymphocytes from blood or lymph organs, heart, muscle or bone joints, in certain embodiments, the evaluations are done in vitro; in other embodiments the evaluations are done in vivo.

As used herein, the term “pharmaceutically acceptable salt” refers to either a pharmaceutically acceptable acid addition salt or a pharmaceutically acceptable base addition salt of as currently disclosed compound that may be administered without any resultant substantial undesirable biological effect(s) or any resultant deleterious interaction(s) with any other component of a pharmaceutical composition in which it may be contained.

As used herein, the term “pharmaceutically acceptable ester,” refers to esters that hydrolyze in vivo and include those that break down readily in the human body to leave the parent compound or a salt thereof. Suitable ester groups include, for example, those derived from pharmaceutically acceptable aliphatic carboxylic acids, particularly alkanoic, alkenoic, cycloalkanoic and alkanedioic acids, in which each alkyl or alkenyl moiety advantageously has not more than 6 carbon atoms. Examples of particular esters include formates, acetates, propionates, butyrates, acrylates and ethylsuccinates.

As used herein, the term “prodrug” refers to a pharmacological derivative of a parent drug molecule that requires biotransformation, either spontaneous or enzymatic, within the organism to release the active drug. For example, prodrugs are variations or derivatives of the compounds of Formula I that have groups cleavable under certain metabolic conditions, which when cleaved, become the compounds of Formula I. Such prodrugs then are pharmaceutically active in vivo, when they undergo solvolysis under physiological conditions or undergo enzymatic degradation. Prodrug compounds herein may be called single, double, triple, etc., depending on the number of biotransformation steps required to release the active drug within the organism, and the number of functionalities present in a precursor-type form.

Prodrug forms often offer advantages of solubility, tissue compatibility, or delayed release in the mammalian organism (See, Bundgard, Design of Prodrugs, pp. 7-9,21-24, Elsevier, Amsterdam 1985 and Silverman, The Organic Chemistry of Drug Design and Drug Action, pp. 352-401, Academic Press, San Diego, Calif, 1992). Prodrugs commonly known in the art include well-known acid derivatives, such as, for example, esters prepared by reaction of the parent acids with a suitable alcohol, amides prepared by reaction of the parent acid compound with an amine, basic groups reacted to form an acylated base derivative, etc. Of course, other prodrug derivatives may be combined with other features disclosed herein to enhance bioavailability. As such, those of skill in the art will appreciate that certain of the presently disclosed compounds having free amino, arnido, hydroxy or carboxylic groups can be converted into prodrugs. Prodrugs include compounds having an amino acid residue, or a polypeptide chain of two or more (e.g., two, three or four) amino acid residues which are covalently joined through peptide bonds to free amino, hydroxy or carboxylic acid groups of the presently disclosed compounds. The amino acid residues include the 20 naturally occurring amino acids commonly designated by three letter symbols and also include 4-hydroxyproline, hydroxylysine, demosine, isodemosine, 3-methylhistidine, norvalin, beta-alanine, gamma-aminobutyric acid, citrulline homocysteine, homoserine, ornithine and methionine sulfone. Prodrugs also include compounds having, a carbonate, carbamate, amide or alkyl ester moiety covalently bonded to any of the above substiruents disclosed herein.

As used herein, (Cₓ-C_{y}) refers in general to groups that have from x to y (inclusive) carbon atoms. Therefore, for example, C₁-C₆ refers to groups that have 1, 2, 3, 4, 5, or 6 carbon atoms, which encompass C₁-C₂, C₁-C₃, C₁-C₄, C₁-C₅, C₂-C₃, C₂-C₄, C₂-C₅, C₂-C₆, and all like combinations. (C₁-C₂₀) and the likes similarly encompass the various combinations between 1 and 20 (inclusive) carbon atoms, such as (C₁-C₆), (C₁-C₁₂) and (C₃-C₁₂).

As used herein, the term “(Cₓ-C_{y})alkyl” refers to a saturated linear or branched free radical consisting essentially of x to y carbon atoms, wherein x is an integer from 1 to about 10 and y is an integer from about 2 to about 20. Exemplary (CₓC_{y})alkyl groups include “(C₁-C₂₀)alkyl,” which refers to a saturated linear or branched free radical consisting essentially of 1 to 20 carbon atoms and a corresponding number of hydrogen atoms. Exemplary (C₁-C₂₀alkyl groups include methyl, ethyl, n-propyl, isopropyl, n-butyl, isobutyl, dodecanyl, etc. Of course, other C₁-C₂₀)alkyl groups will be readily apparent to those of skill in the art given the benefit of the present disclosure.

As used herein, the term “(Cₓ-C_{y})cycloalkyl” refers to a nonaromatic saturated free radical forming at least one ring consisting essentially of x to y carbon atoms, wherein x is an integer from 1 to about 10 and y is an integer from about 2 to about 20. As such, (Cₓ-C_{y})cycloalkyl groups may be monocyclic or multicyclic. Individual rings of such multicyclic cyclo alkyl groups can have different connectivities, e.g., fused, bridged, Spiro, etc. in addition to covalent bond substitution. Exemplary (Cₓ-C_{y})cycloalkyl groups include “(C₃-C₁₀)cycloalkyl,” which refers to a nonaromatic saturated free radical forming at least one ring consisting essentially of 3 to 10 carbon atoms and a corresponding number of hydrogen atoms. As such, (C₃-C₁₀)cycloalkyl groups can be monocyclic or multicyclic. Individual rings of such multicyclic cycloalkyl groups can have different connectivities, e.g., fused, bridged, spino, etc. in addition to covalent bond substitution. Exemplary (C₃-C₁₀)cycloalkyl groups include cyclopropyl, cyclobutyl, cyclopentyl, cyclohexyl, norbornyl, bicyclo[3.2.1]octanyl, octahydro-pentalenyl, spiro[4.5]decanyl, cyclopropyl substituted with cyclobutyl, cyclobutyl substituted with cyclopentyl, cyclohexyl substituted with cyclopropyl, etc. Of course, other (C₃-C₁₀)cycloalkyl groups will be readily apparent to those of skill in the art given the benefit of the present disclosure.

As used herein, the term “(Cₓ-C_{y})heterocycloalkyl” refers to a nonaromatic free radical having x+1 to y+1 atoms (i.e., ring atoms) that form at least one ring, wherein x to of the ring atoms are carbon atoms and the remaining ring atom(s) (i.e., hetero ring atom(s)) is selected from the group consisting of nitrogen, sulfur, and oxygen, and wherein x is an integer from 2 to about 5 and y is an integer from about 3 to about 12. For example, “(C₂-C₉)heterocycloalkyl” refers to a nonaromatic free radical having 3 to 10 atoms (i.e., ring atoms) that form at least one ring, wherein 2 to 9 of the ring atoms are carbon and the remaining ring atom(s) (i.e., hetero ring atom(s)) is selected from the group consisting of nitrogen, sulfur, and oxygen. As such, (C₂-C₉)heterocycloalkyl groups can be monocyclic or multicyclic. Individual rings of such multicyclic heterocycloalkyl groups can have different connectivities, e.g., fused, bridged, spiro, etc. in addition to covalent bond substitution. Exemplary (C₂-C₉)heterocycloalkyl groups include pyrrolidinyl, tetrahydrofuranyl, dihydrofuranyl, tetrahydropyranyl, pyranyl, thiopyranyl, aziridinyl, azetidinyl, oxiranyl, methylenedioxyl, chromenyl, barbituryl, isoxazolidinyl, 1,3-oxazolidin-3-yl, isothiazolidinyl, 1,3-thiazolidin-3-yl, 1,2-pyrazolidin-2-yl, 1,3-pyrazolidin-1-yl, piperidinyl, thiomorpholinyl, 1,2-tetrahydrothiazin-2-yl, 1,3-tetrahydrothiazin-3-yl, tetrahydrothiadiazinyl, morpholinyl, 1,2tetrahydrodiazin-2-yl, 1,3-tetrahydrodiazin-1-yl, tetrahydroazepinyl, piperazinyl, piperizin-2onyl, piperizin-3-onyl, chromanyl, 2-pyrrolinyl, 3-pyrrolinyl, imidazolidinyl, 2-imidazolidinyl, 1,4-dioxanyl, 8-azabicyclo[3.2.1]octanyl, 3-azabicyclo13.2.1]octanyl, 3.8diazabicyclo[3.2.1]octanyl, 2,5-diazabicyclo[2.2.1]heptanyl, 2,5-diazabicyclo[2.2.2]octanyl, octahydro-2H-pyrido[1,2-a]pyrazinyl, 3-azabicyclo[4.1.0]heptanyl, 3-azabicyclo[3.1.0]hexanyl 2-azaspiro[4.4]nonanyl, 7-oxo-1-aza-spiro[4.4]nonanyl, 7-azabicyclo[2,2.2]heptanyl, octahydro-1H-indolyl, etc.

In general, the (C₂-C₉)heterocycloalkyl group typically is attached to the main structure via a carbon atom or a nitrogen atom. In any event, the (C₂-C₉)heterocycloalkyl group is attached to the main structure via a ring atom. Of course, other (C₂-C₉)heterocycloalkyl groups will be readily apparent to those of skill in the art given the benefit of the present disclosure.

As used herein, the term “(Cₓ-C_{y})aryl” refers to an aromatic group consisting essentially of x to y carbon atoms in the aromatic ring(s), wherein x is an integer from about 6 to about 10 and y is an integer from about 10 to about 14. For example, “(C₆-C₁₀)aryl” refers to an aromatic group consisting essentially of 6 to 10 ring carbon atoms, e.g., phenyl and naphthyl.

As used herein, the term “(Cₓ-C_{y})heteroaryl” refers to an aromatic free radical having x+1 to y+1 atoms ring atoms) that form at least one ring, wherein x to y of the ring atoms are carbon atoms and the remaining ring atom(s) (i.e., hetero ring atom(s)) is selected from the group consisting; of nitrogen, sulfur, and oxygen, and wherein x is an integer from about 6 to about 10 and y is an integer from about 10 to about 20. For example, “(C₂-C₉)heteroaryl” refers to an aromatic free radical having 5 to 10 atoms (i.e., ring atoms) that thrill at least one ring, wherein 2 to 9 of the ring atoms are carbon and the remaining ring atom(s) (i.e., hetero ring atom(s)) is selected from the group consisting of nitrogen, sulfur, and oxygen. As such, (C₂-C₉)heteroaryl groups can be monocyclic or multicyclic. Individual rings of such multicyclic heteroaryl groups can have different connectivities, e.g., fused, etc. in addition to covalent bond substitution. Exemplary (C₂-C₉)heteroaryl groups include furyl, thienyl, thiazolyl, pyrazolyl, isothiazolyl, oxazolyl, isoxazolyl, pyrrolyl, triazolyl, tetrazolyl, imidazolyl, 1,3,5-oxadiazolyl, 1,2,4-oxadiazolyl, 1,2,3-oxadiazolyl, 1,3,5-thiadiazolyl, 1,2,3-thiadiazolyl, 1,2,4-thiadiazolyl, pyridyl, pyrimidyl, pyrazinyl, pyridazinyl, 1,2,4-triazinyl, 1,2,3-triazinyl, 1,3,5-triazinyl, pyrazolo[3,4-b]pyridinyl, cinnolinyl, pteridinyl, purinyl, benzo[b]thiophenyl, 5,6,7,8tetrahydro-quinolin-3-yl, benzoxazolyl, benzothiazolyl, benzisothiazolyl, benzisoxazolyl, benzimidazolyl, thianaphthenyl, isothianaphthenyl, benzofuranyl, isobenzofuranyl, isoindolyl, indolyl, indolizinyl, indazolyl, isoquinolyl, quinolyl, phthalazinyl, quinoxalinyl, quinazolinyl and benzoxazinyl, etc.

In general, the (C₂-C₉)heteroaryl group typically is attached to the main structure via a carbon atom, however, those of skill in the art will realize when certain other atoms, e.g., hetero ring atoms, can be attached to the main structure. In any event, the (C₂-C₉)heteroaryl group is attached to the main structure via a ring atom. Of course, other (C₂-C₉)heteroaryl groups will be readily apparent to those of skill in the art given the benefit of the present disclosure.

A used herein, the term, “(Cₓ-C_{y})alkoxy refers to a straight or branched chain alkyl group consisting essentially of form x to y carbon atoms that is attached to the main structure via an oxygen atom, wherein x is an integer from 1 to about 10 and y is an integer from about 2 to about 20. For example, “(C₁-C₂₀)alkoxy” refers to a straight or branched chain alkyl group having 1-20 carbon atoms that is attached to the main structure via an oxygen atom, thus having the general formula alkyl-O—, such as, for example, methoxy, ethoxy, propoxy, isopropoxy, n-butoxy, see-butoxy, tert-butoxy, pentoxy, 2-pentyl, isopentoxy, neopentoxy, hexoxy, 2-hexoxy, 3-hexoxy, and 3-methylpentoxy.

As used herein, various groups and moieties can be referred to collectively by combining, the corresponding names of the groups or moieties to create a chain of groups or moieties, and the chain is attached to the main molecular structure by the last group or moiety in the chain (reading left to right). Thus, for example, when group A is substituted bygroup B, which in turn is substituted by group C, the collective moiety may be referred to as ABC (or ABC-), which collectively attaches “ABC-” to the rest of the main molecular structure via an attachment pointon group C. Each of the groups may be further specified by adding a prefix such as (Cₓ-C_{y}), for example, (C₁-C₂₀)alkyl(C₆-C₁₀)aryl refers to a (C₁-C₂₀)alkyl bonding to an (C₆-C₁₀)aryl group with the collective moiety attaching to the rest of the molecule via an attachment point of the aryl group. Additional examples are provided below as further illustrations. Those of skill given the benefit of the present disclosure will appreciate how

As used herein, the term “thioalkyl” refers to a sulfur atom substituted by an alkyl group, wherein alkyl is defined as above. An exemplary structure is:

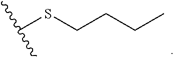

As used herein, the term “halo” refers to fluorine, chlorine, bromine, or iodine.

As used herein, the term “amino” refers to a free radical having a nitrogen atom (i) covalently bonded to two hydrogen atoms, or alternatively (ii) covalently bonded to one hydrogen atom and one carbon radical. As such, the term amino generally refers to primary and secondary amines. In embodiments where the free radical is covalently bonded to a carbon atom, the term “amino” also includes tertiary amines. Those of skill in the art given the benefit of the present disclosure will readily be able to identify when the term “amino” is interchangeably used to refer to primary, secondary, and tertiary amines. The term “animal,” as used herein, refers to humans as well as non-human animals, including, for example, mammals, birds, reptiles, amphibians, and fish. Preferably, the non-human animal is a mammal (e.g., a rodent, a mouse, a rat, a rabbit, a monkey, a dog, a cat, a primate, or a pig). A non-human animal may be a transgenic animal.

In general, the “effective amount” of an active agent refers to an amount sufficient to elicit the desired biological response. As will be appreciated by those of ordinary skill in this art, the effective amount of a compound of the invention may vary depending on such factors as the desired biological endpoint, the pharmacokinetics of the compound, the disease being treated, the mode of administration, and the patient.

### BRIEF DESCRIPTION OF THE DRAWINGS

FIG. 1 is a schematic illustrative representation showing the sphingolipid pathway. FIG. 1 panel A shows the pathway of sphingolipid metabolism with the sphingosine kinase 1 (SK1) enzyme shown in an inner circle, and the reaction it catalyzes in an outer circle. FIG. 1 panel B shows an enhanced view of the SK1 reaction, three lipids SK1 regulates, and biological effects of each lipid in cells.

### DETAILED DESCRIPTION

The invention provides novel inhibitors of sphingosine kinase 1 (SK1) that have useful properties, such as pharmaceutical properties. Certain inventive compounds disclosed herein are useful for treating disorders or conditions mediated by SK1, such as, inflammation and immune-mediated diseases, cancer, diabetes, inflammatory bowel disease (IBD), fibrosis, polycystic kidney disease (CPKD), arteriosclerosis, pulmonary diseases, and viral infections such as HIV and heptatitis C infections.

Research in the past years has revealed significant details in the biology of sphingolipids, which are a group of molecules including various lipid metabolites containing sphingosine moiety such as ceramide, sphingosine, sphigosine-1-phosphate, as well as a large collection of glycosphingolipids and phosphosphingolipids. It has been recognized that many of those sphingolipids play important rolls in the cellular responses, far beyond the confines of membranes (Hannun et al. (2001) Biochemistry 40:4893-4903). Increasing evidences indicate that the balance between cellular levels of sphingolipids is important to regulate cell functioning, as reported that ceramide and sphingosine induce apoptosis or growth arrest, and sphingosine 1 phosphate (S1P) mediates proliferation and angiogenesis (Maceyka et al, (2002) Biochim et Biophy. Acta, 1585:193-201: and Ogretmen et al. (2004) Nature Review 4:604-616),

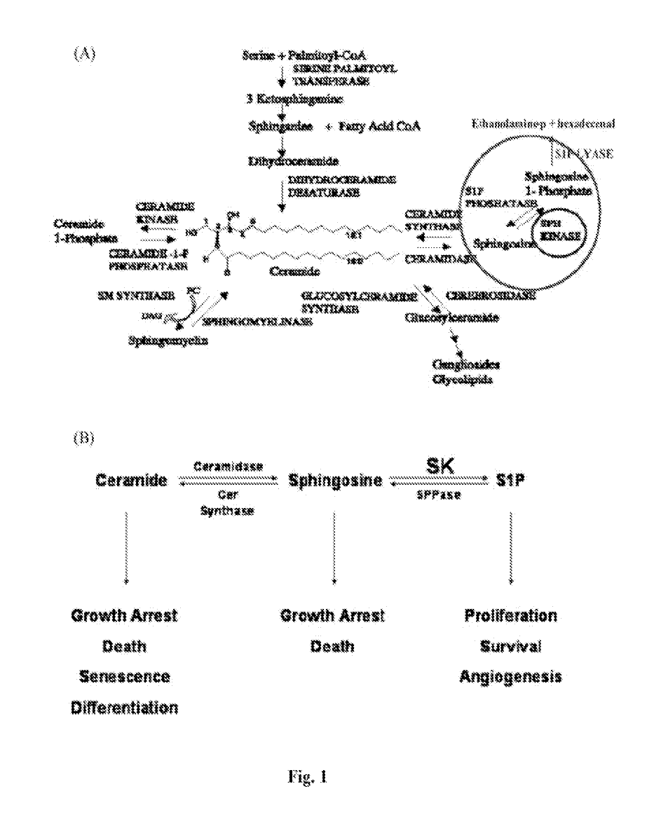

SK1 is an important enzyme in the sphingolipid metabolic pathway as it is a component of a checkpoint that regulates relative levels of S1P, sphingosine, and ceramide. FIG, 1 panel A schematically illustrates the pathway of sphingolipid metabolism with the SK1 enzyme shown in an inner circle, and the reaction it catalyzes in an outer circle. FIG. 1 panel B schematically illustrates an enhanced view of the SK1 reaction, showing that SK1 phosphorylates sphingosine to S1P (De Jonghe et al., (1999) Bioorg. Med. Chem. Lett, 9:3175-3180).

So far, two mammalian sphingosine kinases (SK1 and SK2) have been found, sequenced and characterized. Both SK1 and SK2 are capable of phosphorylating sphingosine to produce S1P (Kohama et al, (1998) J. Biol, Chem., 273: 23722-23728: and Melendez et al. (2000) Gene, 251:19-26). S1P can act as intracellular messenger and extracellular ligands for specific receptors, known as G-protein coupled receptors: S1P₁, S1P₂, S1P₃, S1P₄ and S1P₅ (Rosen et al, (2005) Nat. Rev., Immunol., 5:560). Engagement of S1P binding to its receptors are believed to have wide ranged cell functions, including proliferation and differentiation, chemotaxis and vascularization/angiogenesis (Spiegel et al. (2002) J. Biol, Chem., 277;25851-25854: Liu (2001) Am. J. Respir. Cell Mol. Biol., 24; and Liu et al. (2000) J. Clin. Invest. 106:951-961).

S1P is a bioactive sphingolipid found in high concentrations in human serum (Taha et al., 2006). S1P levels in the cell are controlled by the balance of activity between synthesizing enzymes (sphingosine kinases) and degradative enzymes (sphingosine phosphate phosphatases and sphingosine phosphate lyase). S1P exerts several effects on cells including proliferation, survival, regulation of cell motility, cytoskeletal reorganization, and yeast heat stress response. A response associated with function of S1P as an intracellular effector pertains to ability of S1P to regulate calcium homeostasis, as well as cell growth, proliferation, and inhibition of apoptosis. An additional effect that may be linked to an intracellular action of S1P is pro-survival and pro-growth.

Further, studies have indicated that S1P and its receptors are important for lymphocyte egress from the lymph organs (Jolly et al. (2002) Mol. Immunol. 38:1239-1245; and Mori et al. (2007) Int. Immunol. 19:745-753), and that blocking the engagement of S1P binding to its receptors leads to sequestration of lymphocytes in secondary lymphatic tissues, thus prevents their access to inflammatory lesions and graft sites (Brinkmann et al. (2001) Transplantation 72:764-7691. Furthermore, it has been reported that SK plays a roll in neutrophil activation including chemotaxis (Ibrahim et al. (2004) J. Biol. Chem 279:44802-44811), and activation of mast cells leads to activation of SK1, which could play important part in inducing, proinflammatory actions (Prieschl et al. (1999) J. Exp. Med. 190:1-8).

Without being limited by any theory or mechanism of action, it has been suggested that the balance of the levels between ceramide/sphingosine and S1P provides a rheostat mechanism that decides whether a cell is sent into the death pathway or is protected from apoptosis, and cancer cells take advantage of this rheostat by promoting conditions that favor the production of S1P (Sabbadini (2006) Brit. J. Cancer, 95:1131-1135). An important enzyme regulating this rheostat is SK. It has been reported that cancer cells over expressing S1P enhance metastasis (Takuwa (2002) Biochim Biophys Acta 1582:112-120; and Visentin et al. (2006) Cancer Cell 9:225-238), and tumors xonografted with S1P over expressing cancer cells into nude mice can produce resistance to cytotoxic chemotherapeutics (Pchejetski et al. (2005) Cancer Res 65:11667-11675).

SK1 and S1P are involved in several pathological states, such as inflammation and immune-mediated diseases, cancer, diabetes, inflammatory bowel disease (IBD), fibrosis, polycystic kidney disease (CPKD), arteriosclerosis, pulmonary diseases, and viral infections such as HIV and heptatitis C infections (Kim et. al. (2005) Bioorg, Med. Chem. 13:3475; Taha et al., 2006; Pettus et al. (2003) FASEB J. 17:141; Lee et al. (2004) J. Trauma., 57:955; Baumruker et al. (2005) Immunol. Lett. 96:175; and Pettus et al. (2005) Mol. Pharmacol., 68:330).

Exemplary inflammation and/or immune diseases include: sarcoldosis; fibroid lung; idiopathic interstitial pneumonia; obstructive airways disease, including conditions such as asthma, intrinsic asthma, extrinsic asthma, dust asthma, particularly chronic or inveterate asthma (for example late asthma and airway hyperreponsiveness); bronchitis, including bronchial asthma and infantile asthma; allergic rheumatoid arthritis; systemic lupus erythematosus; nephrotic syndrome lupus; Hashimoto's thyroiditis; multiple sclerosis; myasthenia gravis; type I diabetes mellitus and complications associated therewith; type II adult onset diabetes mellitus; uveitis; nephrotic syndrome; steroid dependent and steroid-resistant nephrosis; palmoplantar pustulosis; allergic encephalomyelitis; glomerulonephritis; psoriasis; psoriatic arthritis; atopic eczema (atopic dermatitis); contact dermatitis and further eczematous dermatitises; seborrheic dermatitis; lichen planus; pemphigus; bullous pemphigoid; epidermolysis bullosa; urticaria, angioedemas; vasculitides; erythemas; cutaneous eosinophilias; acne; alopecia areata; eosinophilic fasciitis; atherosclerosis; conjunctivitis; keratoconjunctivitis; keratitis; vernal conjunctivitis; uveitis associated with Behcet's disease; herpetic keratitis; conical cornea; dystorphia epithelialis corneae; keratoleukoma; ocular pemphigus; Mooren's ulcer; scleritis; Graves' ophthalmopathy; severe intraocular inflammation; inflammation of mucosa or blood vessels such as leukotriene B4-mediated diseases; gastric ulcers; vascular damage caused by ischemic diseases and thrombosis; ischemic bowel disease; inflammatory bowel disease (e.g. Crohn's disease and ulcerative colitis); necrofizing enterocolitis; renal diseases including interstitial nephritis, Goodpasture's syndrome, hemolytic uremic syndrome, and diabetic nephropathy; nervous diseases selected from multiple myositis, Guillain-Barre syndrome, Meniere's disease and radiculopathy; collagen disease including scleroderma, Wegener's granuloma and Sjogren' syndrome; chronic autoimmune liver diseases including autoimmune hepatitis, primary biliary cirrhosis and sclerosing cholangitis), partial liver resection, acute liver necrosis (e.g. necrosis caused by toxins, viral hepatitis, shock or anoxia), B-virus hepatitis, non-A/non-B hepatitis, and cirrhosis; fulminant hepatitis; pustular psoriasis; Behcet's disease; active chronic hepatitis; Evans syndrome; pollinosis; idiopathic hypoparathyroidism; Addison disease; autoimmune atrophic gastritis; lupoid hepatitis; tubulointerstitial nephritis; membranous nephritis; amyotrophic lateral sclerosis or rheumatic fever.

The role of SK1 and S1P in inflammatory and immune processes can be divided into effects of each on epithelial cells, hematopoeitic cells, and endothelial cells. In cells of the immune system, SK1 activation has been shown to occur following crosslinking of immunoglobulin surface receptors, a process important for downstream events in those cells (Taha et al., 2006). In epithelial cells, SK1 activation occurs in response to certain pro-inflammatory mediators, such as TNFα, IL-1a, and LPS, and SK1 mediates the activation of several proteins known to be important in inflammation, such as cyclooxygenase-2 and monocyte chemoattractant protein-1 (MCP-1) (Taha et al., 2006).

A specific role for the SK1/S1P pathway has emerged in regulating induction of cyclooxygenase 2 (Cox2) and the production of the inflammatory mediator PGE₂ in response to pro-inflammatory cytokines, for example TNFα, and IL-1 (Taha et al., 2006; Pettus et al., (2003) FASEB J., 17:1411-1421; and Baumrucker et al. (2004) Immunology Letters, 96:175-185). The formation of PGE₂ involves activation of phospholipase A2 which releases free arachidonate, followed by the action of Cox2. The SK1/S1P pathway selectively mediates the induction of Cox2.

Cell proliferation, differentiation, motility, and survival have been attributed to regulatory actions of S1P (Kee et al., (2005) Clinical and Experimental Pharmacology and Physiology, 32:153-161). While SK1 promotes inflammation via mediating the effects of pro-inflammatory mediators in epithelial and immune cells, the responses that S1P exerts on endothelial cells points towards a protective function. S1P increases the resistance of endothelial cells and enhances barrier integrity (Taha et al., 2006). S1P also reverses the thrombin mediated vascular dysfunction, and inhibits VEGF enhanced vascular permeability. Additionally, S1P induces cyclooxygenase activation and PGE₂ production in lung epithelial cells. It also mobilizes mast cell and monocyte responses acutely and can induce ensinophil chemotaxis. Therefore, the stimulatory effect of SK1 and/or S1P on monocytes, neutrophils, mast cells, and epithelial cells suggest a role for S1P in acute inflammation.

SK1 and S1P are also implicated in immune-modulation, response of a subject's immune system to an infection, e.g., a viral infection. Exemplary viral infections include human immunodeficiency virus (HIV), hepatitis C (HCV), lymphocytic choriomeningitis, meningitis, infections resulting from herpesviruses, infections resulting from influenza viruses, or infections resulting from encephalitis viruses.

S1P has been shown to be an immunosuppressant (anti-inflammatory agent) via its action on lymphocytes (Taha et al., 2006, and Kaneider et al., (2004) The FASEB Journal, 18:1309-1311). S1P is an important component for egress of lymphocytes from lymphoid organs to peripheral inflammatory sites, and exposure of lymphocytes to S1P can result in aberrant internalization of the S1P1 receptor and loss of the “egress” signal. Internalization of S1P1 has also been described in mast cells after SK1 overexpression, which then prevents degranulation, further indicating that acute stimulation of S1P receptors is pro-inflammatory whereas prolonged stimulation may be an anti-inflammatory signal.

SK1 and S1P are also implicated in cancer (Taha et al., 2006). SK mediates the growth response of several pro-growth agonists. SK1 overexpression in itself can enhance growth of cells even without extracellular stimulation. The enzyme has also been proposed as an oncogene activated by Ras. Targeting of SK1 to the plasma membrane, a common mechanism of activation by growth agonists, enhances foci formation and growth in soft agar. Expression levels of SK1 have been found to be higher in tumor tissue than in normal tissue, and S1P has been detected in ascites fluid of ovarian cancer patients.

Furthermore, inhibition of SK1 is anti-proliferative and pro-apoptotic to several tumor cell lines. Increased activity of SK1 and S1P and reduced levels of sphingosine and ceramide have been correlated with the resistance of tumor cells to death-inducing signals such as ceramide and FasL. Moreover, inhibition of SK1 activity enhances the sensitivity of cancer cells to chemotherapy. SK1 and S1P also mediate Cox-2 induction, which has been implicated in colon and breast cancers. SK1 message and protein levels are increased in human colon cancer tissues compared to normal colon tissue levels. SK1 induction has been correlated with Cox2 over expression in these tissues, and in tissue culture studies it has been shown that SK1 is important for basal and cytokine-induced Cox2.

SK1 and S1P also play a role in angiogenesis, as these mediators have been shown to be pro-angiogenic factors (Taha et al., 2006). S1P produces several effects on endothelial cells, which support its role as an angiogenic molecule. These effects include endothelial cell survival, chemotaxis, barrier enhancement, blood vessel stabilization via interactions with mural cells, angiogenesis, and vasculogenesis (Taha et al., 2006). Furthermore, S1P also causes tube narration in Matrigel by human umbilical vein endothelial cells (HUVECs) and in vivo Matrigel assays. Recent work has also shown that SK1 can be exported from cells to make S1P extracellularly, which can then promote vascular angiogenesis and maturation. The role of S1P on blood vessel formation has also been extended to implicate the lipid in vasculogenesis. It has been shown that S1P promotes de novo blood vessel formation in an allantois explant model more potently than VEGF and very comparable to serum (Taha et al., 2006).

Another pathological effect in which SK1 and S1P have been implicated is diabetes. The hyperproliferative role of SK1, and S1P has been proposed to contribute to the early stages of diabetic nephropathy in which streptozotocin (STZ)-induced diabetes enhances neutral ceramidase and SK1 activities to result in increased mesangial proliferation, an important event in the pathogenesis of the disease (Taha et al., 2006).

SK1 activation is also implicated in the pathogenesis of atherosclerosis (Taha et al., 2006). The involvement arises from studies showing that S1P is a component of HDL and LDL as well as effects of SK1 induction and S1P production on the expression of adherence molecules of endothelial cells, and the enhanced proliferation of smooth muscle cells, coupled to the growing role of SK1 and S1P in immune cell chemotaxis, Oxidized LDL is a major risk factor for atherosclerosis, and it can sequentially induce sphingomyelinase, ceramidase and SK1 in smooth muscle cells, resulting in S1P production and enhanced mitogenesis of these cells. Basic fibroblast growth factor (bFGF) also induces hyperproliferation in VSMCs via SK1 activation. In endothelial cells, TNFα induced ERK and NF-κB activities as well as Eselectin and VCAM expression are dependent on SK1 activation, and HDL inhibits these effects by interrupting SK1 activation by TNFα, supporting an anti-atherogenic role for HDL via inhibition of intracellular SK1 activation and S1P production by pro-inflammatory cytokines.

SK1 and S1P are also implicated in chronic obstructive pulmonary disease and asthma (Pfaff et al., (2005) Respiratory Research, 6:4862). In peripheral airways, acetylcholine induces contraction via activation of muscarinic M2-and M3-receptor subtypes (M2R and M3R). Cholinergic hypersensitivity is associated with chronic obstructive pulmonary disease and asthma. A pathway that has been shown to be activated via MR and to increase [Ca2+], includes the activation of SK1 and generation of SIP. It has been shown that the SK1/S1P signaling pathway contributes to cholinergic constriction of murine peripheral airways (Pfaff et al., (2005) Respiratory Research, 6:48-62).

Because SK1 and S1P have been associated with the above conditions, administering compounds of the invention that are inhibitors of SK1 brings a benefit in preventing, ameliorating, arresting development of or, in some cases, even eliminating, these disorders or conditions.

The invention herein provides novel inhibitors of SK1. In accordance with one aspect, the invention herein provides compounds of Formula I:

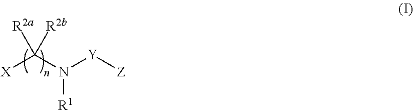

or a pharmaceutically acceptable salt, ester or prodrug thereof, wherein
X is a (C₆-C₁₀)aryl or (C₂-C₉)heteroaryl group, wherein the (C₆-C₁₀)aryl or (C₂-C₉)heteroaryl groups are optionally independently substituted by one or more groups selected from:
   (C₁-C₂₀)alkoxy, (C₁-C₂₀)alkoxy(C₁-C₂₀)alkyl,
   (C₁-C₂₀)alkyl, (C₁-C₂₀)alkyl(C₃-C₁₀)cycloalkyl, (C₁-C₂₀)alkyl(C₆-C₁₀)aryl, (C₁-C₂₀)alkyl(C₆-C₁₀)aryl(C₂-C₉)heteroaryl, (C₁-C₂₀)alkyl(C₂-C₉)heteroaryl, (C₁-C₂₀)alkyloxo(C₁-C₂₀)alkyl, (C₁-C₂₀)alkylthio(C₁-C₂₀)alkyl, (C₁-C₂₀)alkyloxo(C₆-C₁₀)aryl, (C₁-C₂₀)alkylthio(C₆-C₁₀)aryl, (C₁-C₂₀)alkyl(C₂-C₉)heteroaryl(C₁-C₂₀)alkyl, (C₁-C₂₀)alkylsulfonyl(C₁-C₂₀)alkyl,
   (C₃-C₁₀)cycloalkyl, (C₃-C₁₀)cycloalkyl(C₁-C₂₀)alkyl, (C₃-C₁₀)cycloalkyl(C₁-C₂₀)alkyl(C₂-C₉)heteroaryl, (C₃-C₁₀)cycloalkyl(C₂-C₉)heteroaryl,
   (C₆-C₁₀)aryl, (C₆-C₁₀)aryl(C₁-C₂₀)alkyl, (C₆-C₁₀)aryl(C₁-C₂₀)alkyl(C₂-C₉)heteroaryl, (C₆-C₁₀)aryl(C₃-C₁₀)cycloalkyl, (C₆-C₁₀)aryloxy(C₁-C₂₀)alkyl (C₆-C₁₀)aryl(C₆-C₁₀)aryl, (C₆-C₁₀)aryl(C₁-C₂₀)alkyl(C₆-C₁₀)aryl, (C₆-C₁₀)aryl(C₃-C₁₀)cycloalkyl(C₆-C₁₀)aryl, (C₆-C₁₀)aryl(C₁-C₂₀)alkyl(C₃-C₁₀)cycloalkyl,
   (C₂-C₉)heteroaryl, (C₂-C₉)heteroaryl(C₁-C₂₀)alkyl, (C₂-C₉)heteroaryl(C₃-C₁₀)cycloalkyl, (C₂-C₉)heteroaryl(C₆-C₁₀)aryl, (C₂-C₉)heteroaryl(C₆-C₁₀)aryl(C₁-C₂₀)alkyl, (C₂-C₉)heteroaryl(C₁-C₂₀)alkyl(C₆-C₁₀)aryl, (C₂-C₉)heteroaryl(C₃-C₁₀)cycloalkyl(C₆-C₁₀)aryl, (C₂-C₉)heteroaryl(C₁-C₂₀)alkyl(C₃-C₁₀)cycloalkyl,
   hydroxyl,
   halogen,
   oxo(C₁-C₂₀)alkyl, and
   thio(C₁-C₂₀)alkyl;
   wherein the (C₁-C₂₀)alkyl, (C₃-C₁₀)cycloalkyl, (C₁-C₂₀)alkoxy, (C₆-C₁₀)aryl, (C₂-C₉)heteroaryl, oxo(C₁-C₂₀)alkyl, and thio(C₁-C₂₀)alkyl groups are optionally independently substituted by one or more groups selected from: (C₁-C₂₀)alkyl, amino, hydroxyl, carbonyl, and halogen;
n is 0, 1, 2, or 3;
Y is carbonyl or —CH₂—;
Z is (C₁-C₂₀)alkyl, (C₃-C₁₀)cycloalkyl, or (C₂-C₉)heterocycloalkyl, wherein the (C₁-C₂₀)alkyl or (C₂-C₉)heterocycloalkyl groups are optionally independently substituted by one or more groups selected from: amino, hydroxyl, carbonyl, —N[(C₁-C₂₀)alkyl]₂, —NH[(C₁-C₂₀)alkyl], —N[(C₃-C₁₀)cycloalkyl]₂, —NH[(C₃-C₁₀)cycloalkyl], —N[(₃-C₁₀)cycloalkyl(C₁-C₂₀)alkyl]₂, —NH[(C₃-C₁₀)cycloalkyl(C₁-C₂₀)alkyl], and halogen;
R¹ is hydrogen, (C₁-C₁₂)alkyl, (C₃-C₁₀)cycloalkyl, or (C₂-C₉)heterocycloalkyl; and
R^{2a}, R^{2b}
   are each independently hydrogen, halogen, hydroxyl, (C₁-C₁₂)alkyl, (C₃-C₁₀)cycloalkyl, or (C₂-C₉)heterocycloalkyl wherein each R^{2a} and R^{2b} is not halogen or hydroxyl at the position α to the nitrogen atom in Formula I, and wherein optionally R^{2a} and R^{2b} may together form a 3- to 7-membered ring, and
wherein optionally R¹ and one of R^{2a} and R^{2b} may together form a 3- to 7-membered ring.

In some embodiments, one of R^{2a} and R^{2b} may together with atoms attached thereto form a 3- to 7-membered ring with X.

In some embodiments of the compounds of Formula I, Y is carbonyl. In other related embodiments of the compounds of Formula I, n is 0, 1, or 2. In yet other related embodiments of the compounds of Formula I, R¹ is hydrogen, (C₁-C₆)alkyl or (C₃-C₆)cycloalkyl. In still other related embodiments of the compounds of Formula I, R¹ is hydrogen or (C₁-C₃)alkyl. In certain related embodiments of the compounds of Formula I, R¹ is hydrogen. In other related embodiments of the compounds of Formula I, X is (C₆)aryl, (C₄-C₅)heteroaryl, or (C₇-C₈)heteroaryl. In other related embodiments of the compounds of Formula I, X is (C₆)aryl and n is 0 or 1. In still other related embodiments of the compounds of Formula I, X is (C₄-C₅)heteroaryl and n is 0 or 1. In still other related embodiments of the compounds of Formula I, X is (C₇-C₈)heteroaryl and n is 0 or 1.

In certain embodiments of the compounds of Formula I, X is:

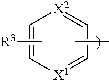

wherein:
X¹, X²
   are each independently N or —CH— wherein at least one of X¹ and X² is nitrogen;
R³ is one or more groups selected from:
   (C₁-C₂₀)alkoxy, (C₂-C₉)alkoxy, (C₃-C₁₈)alkoxy, (C₆-C₁₀)alkoxy, or (C₁₀-C₂₀)alkoxy), (C₁-C₂₀)alkoxyalkyl,
   (C₁-C₂₀)alkyl, (e.g., (C₂-C₁₉)alkyl, (C₃-C₁₈)alkyl, (C₆-C₁₀)alkyl, or (C₁₀-C₂₀)alkyl), (C₁-C₂₀)alkylcycloalkyl, (C₁-C₂₀)alkylaryl, (C₁-C₂₀)alkylarylheteroaryl, (C₁-C₂₀)alkylheteroaryl, (C₁-C₂₀)alkyloxoalkyl, (C₁-C₂₀)alkylthioalkyl, (C₁-C₂₀)alkyloxoaryl, (C₁-C₂₀)alkylthioaryl, (C₁-C₂₀)alkylheteroarylalkyl,
   (C₃-C₁₀)cycloalkyl, (e.g., (C₄-C₉)cycloalkyl, (C₅-C₈)cycloalkyl, or (C₄-C₆)cycloalkyl), (C₃-C₁₀)cycloalkylalkyl, (C₃-C₁₀)cycloalkylalkylheteroaryl, (C₃-C₁₀)cycloalkylheteroaryl,
   (C₆-C₁₀)aryl, (e.g. (C₆-C₉)aryl or (C₇-C₁₀)aryl), (C₆-C₁₀)arylalkyl, (C₆-C₁₀)arylcycloalkyl, (C₆-C₁₀)arylaryl, (C₆-C₁₀)arylalkylaryl, (C₆-C₁₀)arylcycloalkylaryl, (C₆-C₁₀)arylalkylcycloalkyl,
   (C₂-C₉)heteroaryl, (e.g., (C₂-C₈)heteroaryl, (C₃-C₉)heteroaryl, or (C₄-C₇)heteroaryl), (C₂-C₉)heteroarylalkyl, (C₂-C₀)heteroarylcycloalkyl, (C₂-C₉)heteroarylaryl, (C₂-C₉)heteroarylarylalkyl, (C₂-C₉)heteroarylalkylaryl, (C₂-C₉)heteroarylcycloalkylaryl, (C₂-C₉)heteroarylalkylcycloalkyl,
   hydroxyl,
   halogen,
   oxoalkyl, and
   thioalkyl;
   wherein the alkyl, cycloalkyl, alkoxy, aryl, heteroaryl, oxoalkyl, and thioalkyl groups are independently optionally substituted by one or more groups selected from: amino, hydroxyl, carbonyl, and halogen groups;

In certain other embodiments of the compounds of Formula I, X is:

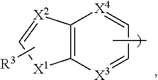

wherein:
X¹ is N or O;
X², X³, X⁴
   are each independently N or —CH— with the proviso that one of X², X³, and X⁴ is nitrogen;
R³ is one or more groups selected from:
   (C₁-C₂₀)alkoxy, (e.g., (C₂-C₁₉)alkoxy, (C₃-C₁₈)alkoxy, (C₆-C₁₀)alkoxy, or (C₁₀-C₂₀)alkoxy), (C₁-C₂₀)alkoxyalkyl,
   (C₂-C₂₀)alkyl, (e.g., (C₂-C₁₉)alkyl, (C₃-C₁₈)alkyl, (C₆-C₁₀)alkyl, or (C₁₉-C₂₀)alkyl), (C₁-C₂₀)alkylcycloalkyl, (C₁-C₂₀)alkylaryl, (C₁-C₂₀)alkylarylheteroaryl, (C₁-C₂₀)alkylheteroaryl, (C₁-C₂₀)alkyloxoalkyl, (C₁-C₂₀)alkylthioalkyl, (C₁-C₂₀)alkyloxoaryl, (C₁-C₂₀)alkylthioaryl, (C₁-C₂₀)alkylheteroarylalkyl,
   (C₃-C₁₀)cycloalkyl, (e.g., (C₄-C₉)cycloalkyl, (C₅-C₈)cycloalkyl, or (C₄-C₆)cycloalkyl), (C₃-C₁₀)cycloalkylalkyl, (C₃-C₁₀)cycloalkylalkylheteroaryl, (C₃-C₁₀)cycloalkylheteroaryl,
   (C₆-C₁₀)aryl, (e.g., (C₆-C₉)aryl or a (C₇-C₁₀)aryl), (C₆-C₁₀)arylalkyl, (C₆-C₁₀)arylcycloalkyl, (C₆-C₁₀)arylaryl, (C₆-C₁₀)arylalkylaryl, (C₆-C₁₀)arylcycloalkylaryl, (C₆-C₁₀)arylalkylcycloalkyl,
   (C₂-C₉)heteroaryl, (e.g., (C₂-C₈)heteroaryl, (C₃-C₉)heteroaryl, or (C₄-C₇)heteroaryl), (C₂-C₉)heteroarylalkyl, (C₂-C₉)heteroarylcycloalkyl, (C₂-C₉)heteroarylaryl, (C₂-C₉)heteroarylarylalkyl, (C₂-C₉)heteroarylalkylaryl, (C₂-C₉)heteroarylcycloalkylaryl, (C₂-C₉)heteroarylalkylcycloalkyl,
   hydroxyl,
   halogen,
   oxoalkyl, and
   thioalkyl;
   wherein the alkyl, cycloalkyl, alkoxy, aryl, heteroaryl, oxoalkyl, and thioalkyl groups are independently optionally substituted by one or more groups selected from: amino, hydroxyl, carbonyl, and halogen groups;

In related embodiments of the compounds of Formula I, X is substituted with a halogen, for example, F or Cl. In other related embodiments, X is an aryl or heteroaryl. In other related embodiments of the compounds of Formula I, X is substituted with

R⁵-A-R⁴,

wherein
A is (C₆)aryl or (C₂-C₅)heteroaryl, (e.g., (C₃-C₄)heteroaryl, or (C₃-C₅)heteroaryl);
R⁴ is a single bond or (C₁-C₄)alkylene, (C₂-C₄)alkylene or (C₃-C₄)alkylene), or (C₁-C₃)heteroalkylene; and
R⁵ is a group selected from:
   (C₂-C₂₀)alkyl, (e.g., (C₂-C₁₉)alkyl, (C₃-C₁₈)alkyl, (C₆-C₁₀(alkyl, or (C₁₀-C₂₀)alkyl), (C₂-C₂₀)alkylcycloalkyl, (C₂-C₂₀)alkylaryl, (C₁-C₁₉)heteroalkyl, (e.g., (C₂-C₁₈)heteroalkyl, (C₃-C₁₅)heteroalkyl, (C₈-C₁₀)heteroalkyl, or (C₉-C₁₉)heteroalkyl), and (C₁-C₁₉)heteroalkylcycloalkyl.

In related embodiments, R⁴ is a single bond. In other related embodiments, R⁵ is (C₂-C₁₂)alkyl, (C₂-C₁₀)alkyl, (C₃-C₈)alkyl, (C₆-C₁₀)alkyl, or (C₁₀-C₁₂)alkyl), or (C₁-C₁₁)heteroalkyl., e.g., (C₂-C₁₀)heteroalkyl, (C₃-C₈)heteroalkyl, (C₈-C₁₁)heteroalkyl, or (C₉-C₁₀)heteroalkyl). In other related embodiments, A is a (C₂-C₃)heteroaryl. In still other related embodiments, A is selected from:

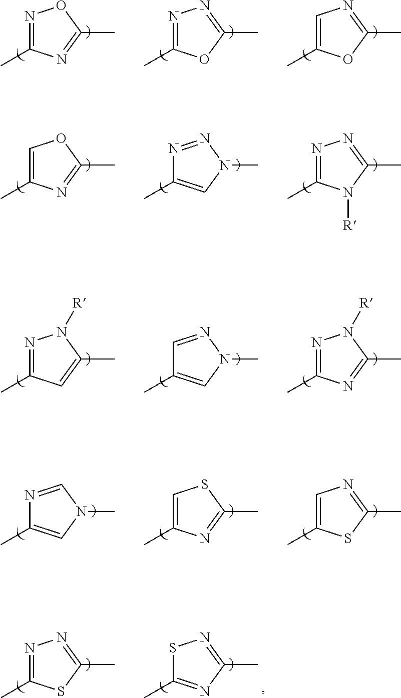

and all regioisomers thereof,
wherein
R′ is a group selected from the group of: (C₂-C₂₀)alkyl, (e.g., (C₂-C₁₉)alkyl, (C₃-C₁₈)alkyl, (C₆-C₁₀)alkyl, or (C₁₀-C₂₀)alkyl), (C₂-C₂₀)alkylcycloalkyl, (C₂-C₂₀)alkylaryl, and (C₁-C₁₉)heteroalkyl, (e.g., (C₂-C₁₈)heteroalkyl, (C₃-C₁₅)heteroalkyl, (C₈-C₁₀) heteroalkyl, or (C₉-C₁₉)heteroalkyl), and (C₁-C₁₉)heteroalkylcycloalkyl.

In certain embodiments of the compounds of Formula I, Z is (C₁-C₁₂)alkyl, (e.g., (C₂-C₁₀)alkyl, (C₃-C₈)alkyl, or (C₄-C₆)alkyl), optionally substituted by one or more groups selected from: amino, hydroxyl, carbonyl, and halogen. In other embodiments of the compounds of Formula I, Z is (C₂-C₉)heterocycloalkyl, e.g, (C₃-C₈)heterocycloalkyl, (C₄-C₆)heterocycloalkyl, or (C₅-C₉)heterocycloalkyl, optionally substituted by one or more groups selected from: amino, hydroxyl, carbonyl, and halogen. In other embodiments of the compounds of Formula I, Z is:

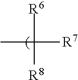

wherein,
R⁶, R⁷
   are each independently a hydrogen, halogen, hydroxyl,
   (C₁-C₁₀)alkyl, (e.g., (C₂-C₁₀)alkyl, (C₃-C₈)alkyl, or (C₄-C₆)alkyl), (C₁-C₁₀)heteroalkyl, or
   (C₃-C₈)cycloalkyl, (e.g., (C₄-C₆)cycloalkyl, (C₃-C₇)cycloalkyl, or (C₅-C₈)cycloalkyl) or
   (C₂-C₇)heterocycloalkyl, (e.g., (C₃-C₆)heterocycloalkyl, (C₄-C₆)heterocycloalkyl, or (C₅-C₇)heterocycloalkyl);
R⁸ is —B—(R^{8a})ₘ, wherein
   B is N, O, or S;
   m is I when B is O or S, or 2 when B is N;
   each R^{8a} is independently a hydrogen, (C₁-C₁₀)alkyl, e.g., (C₂-C₁₀)alkyl, (C₃-C₈)alkyl, or
   (C₄-C₆)alkyl); (C₁-C₁₀)heteroalkyl, or
   (C₃-C₈)cycloalkyl, e.g., (C₄-C₆)cycloalkyl, (C₃-C₇)cycloalkyl, or (C₅-C₈)cycloalkyl) or
   (C₂-C₇)heterocycloalkyl, (e,g, (C₃-C₆)heterocycloalkyl, (C₄-C₆)heterocycloalkyl, or (C₅-C₇)heterocycloalkyl);
   wherein, when B is N, a R^{8a} may together form a ring with the other R^{8a}, R⁶, or R⁷; and
   when B is O or S, R^{8a} may together form a ring with R⁶ or R⁷.

In related embodiments, B is N. In other related embodiments, R^{8a} together with R⁷ form a ring Q,

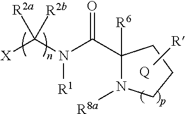

wherein
R′ is absent, one or more groups selected from amino, halogen, hydroxyl, carbonyl, and (C₁-C₁₀)alkyl, (e.g., (C₂-C₁₀)alkyl, (C₃-C₈)alkyl, or (C₄-C₆)alkyl); (C₁-C₁₀)heteroalkyl, or (C₃-C₈)cycloalkyl, (e.g., (C₄-C₆)cycloalkyl, (C₃-C₇)cycloalkyl, or (C₅-C₈)cycloalkyl) or (C₂-C₇)heterocycloalkyl, (e.g., (C₃-C₆)heterocycloalkyl, (C₄-C₆)heterocycloalkyl, or (C₅-C₇)heterocycloalkyl); and
is 0, 1, 2, or 3.

In other related embodiments, n is 1 or 2, and the R^{2b} at α-position to the nitrogen atom in the formula is hydrogen:

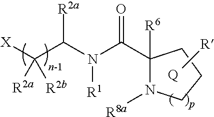

In related embodiments, X is (C₆)aryl, (C₄-C₅)heteroaryl, or (C₇-C₈)heteroaryl. In another related embodiments, X is substituted with

R⁵-A-R⁴—),

wherein
A is (C₆)aryl or (C₂-C₅)heteroaryl;
R¹ is a single bond or (C₁-C₄)alkylene or (C₁-C₃)heteroalkylene; and
R⁵ is a group selected from:
   (C₂-C₂₀)alkyl, (e.g., (C₂-C₁₉)alkyl, (C₃-C₁₈)alkyl, (C₆-C₁₀)alkyl, or (C₁₀-C₂₀)alkyl), (C₂-C₂₀)alkylcycloalkyl, (C₂-C₂₀)alkylaryl,
   (C₁-C₁₉)heteroalkyl, (e.g., (C₂-C₁₀)heteroalkyl, (C₃-C₈)heteroalkyl, (C₈-C₁₁)heteroalkyyl, or (C₉-C₁₀)heteroalkyl), and (C₁-C₁₉)heteroalkylcycloalkyl.

In certain related embodiments, R⁴ is a single bond; and A is (C₂-C₃₎heteroaryl. In other related embodiments, A is selected from:

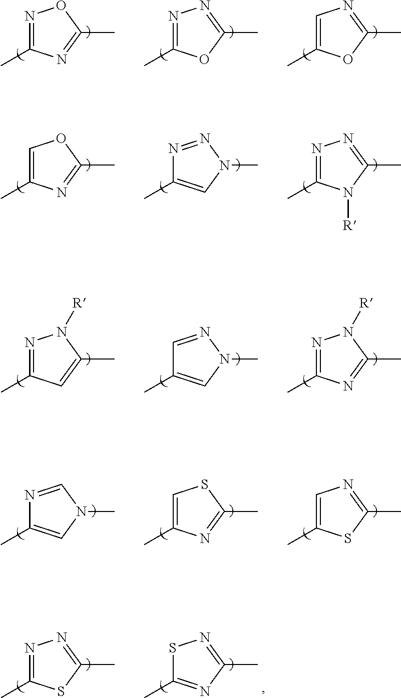

an all regioisomers thereof,
wherein
R′ is a group selected from the group of
   (C₂-C₂₀)alkyl, (e.g., (C₂-C₁₉)alkyl, (C₃-C₁₈)alkyl, (C₆-C₁₀)alkyl, or (C₁₀-C₂₀)alkyl), (C₂-C₂₀)alkylcycloalkyl, (C₂-C₂₀)alkylaryl,
   (C₁-C₁₉)heteroalkyl, (e.g., (C₂-C₁₀)heteroalky (C₃-C₈)heteroalkyl, (C₈-C₁₁)heteroalkyl,
   or (C₉-C₁₀)heteroalkyl), and (C₁-C₁₉)heteroalkylcycloalkyl.

In certain related embodiments, X is (C₆)aryl. In other related embodiments, X is (C₄-C₅)heteroaryl. In still other related embodiments, X is (C₇-C₈)heteroaryl. In related embodiments, R⁶ is

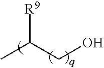

wherein
q is 0, 1, 2, 3, or 4;
R⁹ is hydrogen, halogen, hydroxy, amino, and (C₁-C₁₂)alkyl, (C₂-C₁₀)alkyl, (C₃-C₆)alkyl, or (C₄-C₆)alkyl), heteroalkyl, (C₃-C₈)cycloalkyl, (e.g., (C₄-C₆)cycloalkyl, (C₁-C₇)cycloalkyl, or (C₅-C₈)cycloalkyl) or (C₂-C₇)heterocycloalkyl, (e.g., (C₃-C₈)heterocycloalkyl, (C₄-C₆)heterocycloalkyl, or (C₅-C₇)heterocycloalkyl).

Another aspect of the invention herein provides a pharmaceutical composition comprising an amount of a compound of Formula I

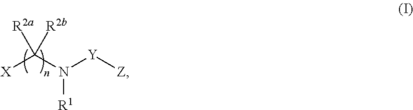

or a pharmaceutically acceptable salt, ester or pro-drug thereof, effective in the treatment or prevention of a disorder or condition selected from the group consisting of inflammation and immune-mediated disease, cancer, diabetes, inflammatory bowel disease, fibrosis, polycystic kidney disease, arteriosclerosis, pulmonary diseases, and viral infections or a related disorder or condition thereof in a mammal, including a human, and a pharmaceutically effective carrier, wherein
X is a (C₆-C₁₀)aryl or (C₂-C₉)heteroaryl group, wherein the (C₆-C₁₀)aryl or (C₂-C₉)heteroaryl groups are optionally independently substituted by one or more groups selected from
   (C₁-C₂₀)alkoxy, (C₁-C₂₀)alkoxy(C₁-C₂₀)alkyl,
   (C₁-C₂₀)alkyl, (C₁-C₂₀)alkyl(C₃-C₁₀)cycloalkyl, (C₁-C₂₀)alkyl(C₆-C₁₀)aryl, (C₁-C₂₀)alkyl(C₆-C₁₀)aryl(C₂-C₉)heteroaryl, (C₁-C₂₀)alkyl(C₂-C₉)heteroaryl, (C₁-C₂₀)alkyloxo(C₁-C₂₀)alkyl, (C₁-C₂₀)alkylthio(C₁-C₂₀)alkyl, (C₁-C₂₀)alkyloxo(C₆-C₁₀)aryl, (C₁-C₂₀)alkythio(C₆-C₁₀)aryl, (C₁-C₂₀)alkyl(C₂-C₉)heteroaryl(C₁-C₂₀)alkyl, (C₁-C₂₀)alkylsulfonyl(C₁-C₂₀)alkyl,
   (C₃-C₁₀)cycloalkyl, (C₃-C₁₀)cycloalkyl(C₁-C₂₀)alkyl, (C₃-C₁₀)cycloalkyl(C₁-C₂₀)alkyl(C₂-C₉)heteroaryl, (C₃-C₁₀)cycloalkyl(C₂-C₉)heteroaryl,
   (C₆-C₁₀)aryl, (C₆-C₁₀)aryl(C₁-C₂₀)alkyl, (C₆-C₁₀)aryl(C₁-C₂₀)alkyl(C₂-C₉)heteroaryl, (C₆-C₁₀)aryl(C₃-C₁₀)cycloalkyl, (C₆-C₁₀)aryloxy(C₁-₂₀)alkyl (C₆-C₁₀)aryl(C₆-C₂₀)aryl, (C₆-C₁₀)aryl(C₁-C₂₀)alkyl(C₆-C₁₀)aryl, (C₆-C₁₀)aryl(C₃-C₁₀)cycloalkyl(C₆-C₁₀)aryl, (C₆-C₁₀)aryl(C₁-C₂₀)alkyl(C₃-C₁₀)cycloalkyl,
   (C₂-C₉)heteroaryl, (C₆-C₉)heteroaryl(C₁-C₂₀)alkyl, (C₂-C₉)heteroaryl(C₃-C₁₀)cycloalkyl, (C₂-C₉)heteroaryl(C₂-C₁₀)aryl(C₂-C₉)heteroaryl(C₆-C₁₀)aryl(C₁-C₂₀)alkyl, (C₂-C₉)heteroaryl(C₁-C₂₀)alkyl(C₆-C₁₀)aryl, (C₂-C₉)heteroaryl(C₃-C₁₀)cycloalkyl(C₆-C₁₀)aryl, (C₂-C₉)heteroaryl(C₁-C₂₀)alkyl(C₃-C₁₀)cycloalkyl,
   hydroxyl,
   halogen,
   oxo(C₁-C₂₀)alkyl, and
   thio(C₁-C₂₀)alkyl;
   wherein the (C₁-C₂₀)alkyl, (C₃-C₁₀)cycloalkyl, (C₁-C₂₀)alkoxy, (C₆-C₁₀)aryl, (C₂-C₉)heteroaryl, oxo(C₁-C₂₀)alkyl, and thio(C₁-C₂₀)alkyl groups are optionally independently substituted by one or more groups selected front (C₁-C₂₀)alkyl, amino, hydroxyl, carbonyl, and halogen;
n is 0, 1, 2, or 3;
Y is carbonyl or —CH₂—;
Z is (C₁-C₂₀)alkyl, (C₃-C₁₀)cycloalkyl, or (C₂-C₉)heterocycloalkyl, wherein the (C₁-C₂₀)alkyl or (C₂-C₉)heterocycloalkyl groups are optionally independently substituted by one or more groups selected from: amino, hydroxyl, carbonyl, —N[(C₁-C₂₀)alkyl]₂, —NH[(C₁-C₂₀)alkyl], —N[(C₂-C₁₀)cycloalkyl]₂, —NH[(C₃-C₁₀)cycloalkyl], —N[(C₃-C₁₀)cycloalkyl(C₄-C₂₀)alkyl]₂, —NH[(C₃-C₁₀)cycloalkyl(C₁-C₂₀)alkyl], and halogen;
R¹ is hydrogen, (C₁-C₁₂)alkyl, (C₃-C₁₀)cycloalkyl, or (C₂-C₉)heterocycloalkyl; and
R^{2a}, R^{2b}
   are each independently hydrogen, halogen, hydroxyl, (C₁-C₁₂)alkyl, (C₃-C₁₀)cycloalkyl, or (C₂-C₉)heterocycloalkyl wherein each R^{2a} and R^{2b} is not halogen or hydroxyl at the position α to the nitrogen atom in Formula I, and wherein optionally R^{2a} and R^{2b} may together form a 3- to 7-membered ring, and
wherein optionally R¹ and one of R^{2a} and R^{2b} may together form a 3- to 7-membered ring.

In some related embodiments, the disorder or condition is an immune-mediated disease. In other related embodiments, the disorder or condition is cancer, In still other related embodiments, the disorder or condition is a type of diabetes. In still other related embodiments, the disorder or condition is a viral infection. In another related embodiment, the mammal is a human.

Another aspect of the invention herein provides a method of treating or preventing a disorder or condition in a mammal, including a human, including administering to a subject in need thereof an therapeutically effective amount of a pharmaceutical composition comprising, a compound of Formula I

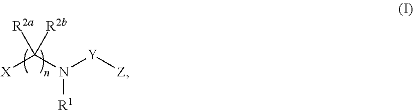

or a pharmaceutically acceptable salt, ester or pro-drug thereof,
wherein the disorder or condition is selected, from the group consisting of inflammation and immune-mediated disease, cancer, diabetes, inflammatory bowel disease, fibrosis, polycystic kidney disease, arteriosclerosis, pulmonary diseases, and viral infections or a related disorder or condition thereof, wherein:
X is a C₆-C₁₀)aryl or (C₂-C₉)heteroaryl group, wherein the (C₆-C₁₀)aryl or (C₂-C₉)heteroaryl groups are optionally independently substituted by one or more groups selected from:
   (C₁-C₂₀)alkoxy, (C₁-C₂₀)alkoxy(C₁-C₂₀)alkyl,
   (C₁-C₂₀)alkyl, (C₁-C₂₀)alkyl(C₃-C₁₀)cycloalkyl, (C₁-C₂₀)alkyl(C₆-C₁₀)aryl, (C₁-C₂₀)alkyl(C₆-C₁₀)aryl(C₂-C₉)heteroaryl, (C₁-C₂₀)alkyl(C₂-C₉)heteroaryl, (C₁-C₂₀)alkyloxo(C₁-C₂₀)alkyl, (C₁-C₂₀)alkythio(C₁-C₂₀)alkyl, (C₁-C₂₀)alkyloxo(C₆-C₁₀)aryl, (C₁-C₂₀)alkylthio(C₆-C₁₀)aryl, (C₁-C₂₀)alkyl(C₂-C₉)heteroaryl(C₁-C₂₀)alkyl, (C₁-C₂₀)alkylsulfonyl(C₁-C₂₀)alkyl,
   (C₃-C₁₀)cycloalkyl, (C₃-C₁₀)cycloalkyl(C₁-C₂₀)alkyl, (C₃-C₁₀)cycloalkyl(C₁-C₂₀)alkyl(C₂-C₉)heteroaryl, (C₃-C₁₀)cycloalkyl(C₂-C₉)heteroaryl,
   (C₆-C₁₀)aryl, (C₆-C₁₀)aryl(C₁-C₂₀)alkyl, (C₆-C₁₀)aryl(C₁-C₂₀)alkyl(C₂-C₉)heteroaryl, (C₆-C₁₀)aryl(C₃-C₁₀)cycloalkyl, (C₆-C₁₀)aryloxy(C₁-C₂₀)alkyl (C₆-C₁₀)aryl(C₆-C₁₀)aryl, (C₆-C₁₀)aryl(C₁-C₂₀)alkyl(C₆-C₁₀)aryl, (C₆-C₁₀)aryl(C₃-C₁₀)cycloalkyl(C₆-C₁₀)aryl, (C₆-C₁₀)aryl(C₁-C₂₀)alkyl(C₃-C₁₀)cycloalkyl,
   (C₂-C₉)heteroaryl, (C₂-C₉)heteroaryl(C₁-C₂₀)alkyl, (C₂-C₉)heteroaryl(C₃-C₁₀)cycloalkyl, (C₂-C₉)heteroaryl(C₆-C₁₀)aryl, (C₂-C₉)heteroaryl(C₆-C₁₀)aryl(C₁-C₂₀)alkyl, (C₂-C₉)heteroaryl(C₁-C₂₀)alkyl(C₆-C₁₀)aryl, (C₂-C₉)heteroaryl(C₃-C₁₀)cycloalkyl(C₆-C₁₀)aryl, (C₂-C₉)heteroaryl(C₁-C₂₀)alkyl(C₃-C₁₀)cycloalkyl,
   hydroxyl,
   halogen,
   oxo(C₁-C₂₀)alkyl, and
   thio(C₁-C₂₀)alkyl;
   wherein the (C₁-C₂₀)alkyl, (C₃-C₁₀)cycloalkyl, (C₁-C₂₀)alkoxy, (C₆-C₁₀)aryl, (C₂-C₉)heteroaryl, oxo(C₁-C₂₀)alkyl, and thio(C₁-C₂₀)alkyl groups are optionally independently substituted by one or more groups selected from (C₁-C₂₀)alkyl, amino, hydroxyl, carbonyl, and halogen;
n is 0, 1, 2, or 3;
Y is carbonyl or —CH₂—;
Z is (C₁-C₂₀)alkyl, (C₃-C₁₀)cycloalkyl, or (C₂-C₉)heterocycloalkyl, wherein the (C₁-C₂₀)alkyl or (C₂-C₉)heterocycloalkyl groups are optionally independently substituted by one or more groups selected from: amino, hydroxyl, carbonyl, —N[(C₁-C₂₀)alkyl]₂, —NH[(C₁-C₂₀)alkyl], —N[(C₃-C₁₀)cycloalkyl]₂, —N[(C₃-C₁₀)cycloalkyl], —N[(C₃-C₁₀)cycloalkyl(C₁-C₂₀)alkyl]₂, —NH[(C₃-C₁₀)cycloalkyl(C₁-C₂₀)alkyl], and halogen;
R¹ is hydrogen, (C₁-C₁₂)alkyl, (C₃-C₁₀)cycloalkyl, or (C₂-C₉)heterocycloalkyl; and
R^{2a}, R^{2b}
are each independently hydrogen, halogen, hydroxyl, (C₁-C₁₂)alkyl, (C₃-C₁₀)cycloalkyl, or (C₂-C₉)heterocycloalkyl wherein each R^{2a} and R^{2b} is not halogen or hydroxyl at the position α to the nitrogen atom in Formula I, and wherein optionally R^{2a} and R^{2b} may together form a 3- to 7-membered ring, and
wherein optionally R¹ and one of R^{2a} and R^{2b} may together term as 3- to 7-membered ring.

In related embodiments, the disorder or condition is an immune-mediated disease. In other related embodiments, the disorder or condition is cancer. In still other related embodiments, the disorder or condition is a type of diabetes. In still other related embodiments, the disorder or condition is a viral infection. In another related embodiment, the mammal is a human.

Another aspect of the invention herein provides a method for treating a disorder or condition mediated by sphingosine kinase-1, the method including administering to a subject in need thereof a therapeutically effective amount of a pharmaceutical composition comprising, a compound of Formula I

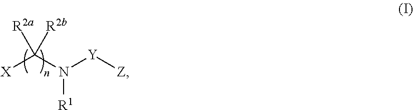

or a pharmaceutically acceptable salt, ester or pro-drug thereof,
wherein
X is a (C₆-C₁₀)aryl or (C₂-C₉)heteroaryl group, wherein the (C₆-C₁₀)aryl or (C₂-C₉)heteroaryl groups are optionally independently substituted by one or more groups selected from
   (C₁-C₂₀)alkoxy, (C₁-C₂₀)alkoxy(C₁-C₂₀)alkyl,
   (C₁-C₂₀)alkyl, (C₃-C₂₀)alkyl(C₃-C₁₀)cycloalkyl, (C₁-C₂₀)alkyl(C₆-C₁₀)aryl, (C₁-C₂₀)alkyl(C₆-C₁₀)aryl(C₂-C₉)heteroaryl, (C₁-C₂₀)alkyl(C₂-C₉)heteroaryl, (C₁-C₂₀)alkyloxo(C₁-C₂₀)alkyl (C₁-C₂₀)alkylthio(C₁-C₂₀)alkyl, (C₁-C₂₀)alkyloxo(C₆-C₁₀)aryl, (C₁-C₂₀)alkylthio(C₆-C₁₀)aryl, (C₁-C₂₀)alkyl(C₂-C₉)heteroaryl(C₁-C₂₀)alkyl, (C₁-C₂₀)alkylsulfonyl(C₁-C₂₀)alkyl,
   (C₃-C₁₀)cycloalkyl, (C₃-C₁₀)cycloalkyl(C₁-C₂₀)alkyl, (C₃-C₁₀)cycloalkyl(C₁-C₂₀)alkyl(C₂-C₉)heteroaryl, (C₃C₁₀)cycloalkyl(C₂-C₉)heteroaryl,
   (C₆-C₁₀)aryl, (C₆-C₁₀)aryl(C₁-C₂₀)alkyl, C₆-C₁₀)aryl(C₁-C₂₀) alkyl(C₂-C₉)heteroaryl, (C₆-C₁₀)aryl(C₃-C₁₀)cycloalkyl, (C₆-C₁₀)aryloxy(C₁-C₂₀)alkyl (C₆-C₁₀)aryl(C₆-C₁₀)aryl, (C₆-C₁₀)aryl(C₁-C₂₀)alkyl(C₆-C₁₀)aryl, (C₆-C₁₀)aryl(C₃-C₁₀)cycloalkyl(C₆-C₁₀)aryl, (C₆-C₁₀)aryl(C₁-C₂₀)alkyl(C₃-C₁₀)cycloalkyl,
   (C₂-C₉)heteroaryl, (C₂-C₉)heteroaryl(C₁-C₂₀)alkyl (C₂-C₉)heteroaryl(C₁-C₁₀)cycloalkyl, (C₂-C₉)heteroaryl(C₆-C₁₀)aryl, (C₂-C₉)heteroaryl(C₆-C₁₀)aryl(C₁-C₂₀)alkyl, (C₂-C₉)heteroaryl(C₁-C₂₀)alkyl(C₆-C₁₀)aryl, (C₂-C₉)heteroaryl(C₃-C₁₀)cycloalkyl(C₆-C₁₀)aryl, (C₂-C₉)heteroaryl(C₁-C₂₀)alkyl(C₃-C₁₀)cycloalkyl,
   hydroxyl,
   halogen,
   oxo(C₁-C₂₀)alkyl, and
   thio(C₁-C₂₀)alkyl;
   wherein the (C₁-C₂₀)alkyl, (C₃-C₁₀)cycloalkyl, (C₁-C₂₀)alkoxy, (C₆-C₁₀)aryl, (C₂-C₉)heteroaryl, oxo(C₁-C₂₀)alkyl, and thio(C₁-C₂₀)alkyl groups are optionally independently substituted by one or more groups selected from: (C₁-C₂₀)alkyl, amino, hydroxyl, carbonyl, and halogen;
n is 0, 1, 2, or 3;
Y is carbonyl or
Z is (C₁-C₂₀)alkyl, (C₃-C₁₀)cycloalkyl, or (C₂-C₉)heterocyeloalkyl, wherein the (C₁-C₂₀)alkyl or (C₂-C₉)heterocycloalkyl groups are optionally independently substituted by one or more groups selected from: amino, hydroxyl, carbonyl, —N[(C₁-C₂₀)alkyl]₂, —NH[(C₁-C₂₀)alkyl], —N[(C₃-C₁₀)cycloalkyl]₂—NH[(C₃-C₁₀)cycloalkyl], —N[(C₃-C₁₀)cycloalkyl(C₁-C₂₀)alkyl]₂, —NH(C₃-C₁₀)cycloalkyl(C₁-C₂₀)alkyl], and halogen;
R¹ is hydrogen, (C₁-C₁₂)alkyl, (C₃-C₁₀)cycloalkyl, or (C₂-C₉)heterocycloalkyl; and
R^{2a}, R^{2b}
   are each independently hydrogen, halogen, hydroxyl, (C₁-C₁₂)alkyl, (C₃-C₁₀)cycloalkyl, or (C₂-C₉)heterocycloalkyl wherein each R^{2a} and R^{2b} is not halogen or hydroxyl at the position α to the nitrogen atom in Formula I, and wherein optionally R^{2a} and R^{2b} may together form a 3- to 7-membered ring, and
      wherein optionally R¹ and one of R^{2a} and R^{2b} may together form a 3- to 7-membered ring.

Another aspect of the invention herein provides a compound selected from:

(2S,3S)-3-Hydroxy-N-(4-octylphenyl)pyrrolidine-2-carboxamide
(2S,3R)-3-Hydroxy-N-(4-octylphenyl)pyrrolidine-2-carboxamide
(R)-2-Amino-3-hydroxy-N-(4-octylphenyl)propanamide
(S)-2Amino-3-hydroxy-N-(4-octylphenyl)propanamide
(±)-erythro-DL-2-Amino-3-hydroxy-N-(4-octylphenyl)pentanamide
(2S,3R)-2-Amino-N-(4-decylphenyl)-3-hydroxybutanamide
(2S,3R)-2-Amino-3-hydroxy-N-(4-undecylphenyl)butanamide
(2S,3R)-2-Amino-N-(4-dodecylphenyl)-3-hydroxybutanamide
(2R,3S)-2-((4-Octylphenylamino)methyl)pyrrolidin-3-ol
(R)-2-Amino-3-(4-octylphenylamino)propan-1-ol
(2S,3R)-2-Amino-3-hydroxy-N-(3-octylphenyl)butanamide
(2S,3R)-2-Amino-N-(4-(heptyloxy)phenyl)-3-hydroxybutanamide
(+/−)-2-Amino-3-hydroxy-N-(4-nonylphenyl)propanamide
(2S,3R)-2-Amino-3-hydroxy-N-(4-nonlphenyl)butanamide
(2S,3R)-2-Amino-N-(4-(hexyloxymethyl)phenyl)-3-hydroxybutanamide
(2S,3R)-2-Amino-3-hydroxy-N-(4-(7-methyloctyl)phenyl)butanamide
(2S,3R)-2-Amino-N-(4-(7-(4-fluorophenoxy)heptyl)phenyl)-3-hydroxybutanamide
(2S,3R)-2-Amino-3-hydroxy-N-(4-(octyloxy)phenyl)butanamide
(2S,3S)-N-4-(Hexyloxymethyl)phenyl)-3-hydroxypyrrolidine-2-carboxamide
(2S,3R,4S)-3,4-Dihydroxy-N-(4-octylphenyl)pyrrolidine-2-carboxamide
(2S,3S)-3-Hydroxy-N-4-(octloxymethyl)phenyl)pyrrolidine-2-carboxamide
(2S,3R)-2-Amino-N-(4-(5-(4-fluorophenoxy)pentyl)phenyl-3-hydroxybutanamide
(2S,3S)-N-(4-(3-(4-Fluorophenoxy)propyl)phenyl)-3-hydroxypyrrolidine-2-carboxamide
(2S,3S)-N-(4-(5-(4-Fluorophenoxy)pentyl)phenyl)-3-hydroxypyrrolidine-2-carboxamide
(2S,3S)-N-(4-(7-(4-Fluorophenoxy)heptyl)phenyl)-3-hydroxypyrrolidine-2-carboxamide
(2S,3S)-3-Hydroxy-N-(6-octylpyridin-3-yl)pyrrolidine-2-carboxamide
(2S,3S)-3-Hydroxy-N-((S)-1-(4-octylphenyl)ethyl)pyrrolidine-2-carboxamide
(2S,3S)-N-(4-(5-(4-Fluorophenoxy)pentyl)benzyl)-3-hydroxypyrrolidine-2-carboxamide
(2S,3R)-2-Amino-3-hydroxy-N-(4-octylphenyl)butanamide
(2R,3S)-2-Amino-3-hydroxy-N-(4-octylphenyl)butanamide
(2R,3R)-2-Amino-3-hydroxy-N-(4-octylphenyl)butanamide
(2S,3S)-2-Amino-3-hydroxy-N-(4-octylphenyl)butanamide
(2S,3R)-2-amino-3-hydroxy-N-(4-(6-oxodecyl)phenyl)butanamide
(2S,3R)-2-amino-3-hydroxy-N-(4-(4-oxodecyl)phenyl)butanamide
(2S,3R)-2-amino-3-hydroxy-N-(4-(4-hydroxydecyl)phenyl)butanamide
(2S,3S)-3-hydroxy-N-(4-octylbenzyl)pyrrolidine-2-carboxamide
(2S,3S)-N-(4-heptylbenzyl)-3-hyroxypyrrolidine-2-carboxamide
(2S,3S)-3-hydroxy-N-(4-(6-oxodecyl)phenyl)pyrrolidine-2-carboxamide
(2S,3S)-N-(4-(hexylthiomethyl)phenyl)-3-hydroxypyrrolidine-2-carboxamide
(2S,3S)-N-(4-(2-(hexylthio)ethyl)phenyl)-3-hydroxypyrrolidine-2-carboxamide
(2S,3S)-N-(4-(4-(hexylthio)butyl)phenyl)-3-hydroxypyrrolidine-2-carboxamide
(2S,3S)-3-hydroxy-N-(4-(4-hydroxyundecyl)phenyl)pyrrolidine-2-carboxamide
(2S,3S)-N-(4-(heptylthiomethyl)benzyl)-3-hydroxypyrrolidine-2-carboxamide
(2S,3S)-3-hydroxy-N-(4-octylphenethyl)pyrrolidine-2-carboxamide
cis-N-(4-decylphenyl)-3-hydroxypiperidine-2-carboxamide
(2S,3S)-N-(4-(hexylsulfonylmethyl)phenyl)-3-hydroxypyrrolidine-2-carboxamide
(2S,3S)-2-(dipropylamino)-3-hydroxy-N-(4-octylphenyl)butanamide
(2S,3S)-2-(cyclohexylmethylamino)-3-hydroxy-N-(4-octylphenyl)butanamide
(2S,3S)-2-(dibenzylamino)-3-hydroxy-N-(4-octylphenyl)butanamide
(2S,3S)-N-(4-(3-cyclohexylpropyl)phenyl)-3-hydroxypyrrolidine-2-carboxamide
(S)-3-Hydroxy-2-(methylamino)-N-(4-octylphenyl)propanamide
(S)-2-Amino-6-hydroxy-N-(4-octylphenyl)hexanamide
(±)-2-Amino-4-hydroxy-N-(4-octylphenyl)butanamide
(S)-2-Amino-4-hydroxy-N-(4-octylphenyl)butanamide
(R)-2-Amino-4-hydroxy-N-(4-octylphenyl)butanamide
(±)-3-Amino-4-(4-octylphenylamino)butan-1-ol
(S)-2-Amino-4-hydroxy-N-(4-octylbenzyl)butanamide
(2S,3S)-3-hydroxy-N-(2-hydroxy-4-octylphenyl)pyrrolidine-2-
carboxamide hydrochloride
(2S,3R)-2-Amino-3-hydroxy-N-(4′-octylbiphenyl-4-yl)butanamide
(2R,3R)-3-Amino-4-(4′-octylbiphenyl-4-ylamino)butan-2-ol
(2S,3S)-N-((R)-5-(5-Heptyl-1,2,4-oxadiazol-3-yl)-2,3-dihydro-1H-inden-1-yl)-3-
hydroxypyrrolidine-2-carboxamide
(2S,3S)-N-((S)-5-(5-heptyl-1,2,4-oxadiazol-3-yl)-2,3-dihydro-1H-inden-1-yl)-3-
hydroxypyrrolidine-2-carboxamide
(2S,3S)-N-(4-(5-Cyclohexyl-1-methyl-1H-1,2,4-triazol-3-yl)benzyl)-3-
hydroxypyrrolidine-2-carboxamide
(2S,3S)-N-(4-(4-Heptyl-1H-1,2,3-triazol-1-yl)benzyl)-3-hydroxypyrrolidine-2-
carboxamide
(2S,3S)-3-Hydroxy-N-(4-(4-phenethyl-1H-1,2,3-triazol-1-yl)benzyl)pyrrolidine-2-
carboxamide
(2S,3S)-3-Hydroxy-N-((S)-1-(4-(4-phenethyl-1H-1,2,3-triazol-1-
yl)phenyl)propyl)pyrrolidine-2-carboxamide
(2S,3S)-N-(4-(4-(2-Cyclohexylethyl)-1H-1,2,3-triazol-1-yl)benzyl)-3-hydroxypyrrolidine-
2-carboxamide
(2S,3S)-N-(4-(4-(4-Flourophenethyl)-1H-1,2,3-triazol-1-yl)benzyl)-3-hydroxypyrrolidine-
2-carboxamide
(2S,3S)-3-Hydroxy-N-(4-(4-phenethyl-1H-pyrazol-1-yl)benzyl)pyrrolidine-2-
carboxamide
(2S,3S)-N-((S)-1-(4-(5-Heptyl-1,2,4-oxadiazol-3-yl)phenyl)-2-methylpropyl)-3-
hydroxypyrrolidine-2-carboxamide
(2S,3S)-N-(4-(5-Heptyl-1-methyl-1H-1,2,4-triazol-3-yl)benzyl)-3-hydroxypyrrolidine-2-
carboxamide
(2S,3S)-N-((S)-1-(4-(5-Heptyl-1,2,4-oxadiazol-3-yl)phenyl)ethyl)-3-hydroxypyrrolidine-
2-carboxamide
(2S,3S)-N-((R)-1-(4-(5-Heptyl-1,2,4-oxadiazol-3-yl)phenyl)ethyl)-3-hydroxypyrrolidine-
2-carboxamide
(2S,3S)-N-(4-(5-Heptyl-1,3,4-oxadiazol-2-yl)benzyl)-3-hydroxypyrrolidine-2-
carboxamide
(2S,3S)-N-((S)-1-(4-(5-Heptyl-1,2,4-oxadiazol-3-yl)phenyl)propyl)-3-
hydroxypyrrolidine-2-carboxamide
(2S,3S)-N-(4-(3-Heptyl-1,2,4-oxadiazol-5-yl)benzyl)-3-hydroxypyrrolidine-2-
carboxamide
(2S,3S)-N-((3-(4-Heptylphenyl)-1,2,4-oxadiazol-5-yl)methyl)-3-hydroxypyrrolidine-2-
carboxamide
(2S,3S)-3-Hydroxy-N-(4-(5-(4-isobutylphenyl)-1,2,4-oxadiazol-3-yl)benzyl)pyrrolidine-
2-carboxamide
(2S,3S)-N-(4-(5-Heptyl-1,2,4-oxadiazol-3-yl)benzyl)-3-hydroxypyrrolidine-2-
carboxamide
(2S,3S)-3-Hydroxy-N-(4-(5-octyl-1,2,4-oxadiazol-3-yl)benzyl)pyrrolidine-2-carboxamide
(2S,3S)-3-Hydroxy-N-(4-(5-(4-propylphenyl)-1,2,4-oxadiazol-3-yl)benzyl)pyrrolidine-2-
carboxamide
(2S,3S)-N-(4-(5-(4-Butylphenyl)-1,2,4-oxadiazol-3-yl)benzyl)hydroxypyrrolidine-2-
carboxamide
(S)-2-Amino-N-(4-(5-heptyl-1,2,4-oxadiazol-3-yl)benzyl)-4-hydroxybutanamide
(2S,3S)-3-Hydroxy-N-(4-5-pentyl-1,2,4-oxadiazol-3-yl)benzyl)pyrrolidine-2-
carboxamide
(2S,3S)-N-(4-(5-Hexyl-1,2,4-oxadiazol-3-yl)benzyl)-3-hydroxypyrrolidine-2-
carboxamide
(2S,3S)-N-(4-(5-Cyclohexyl-1,2,4-oxadiazol-3-yl)benzyl)-3-hydroxypyrrolidine-2-
carboxamide
(2S,3S)-3-Hydroxy-N-(4-(5-(1-methylcyclohexyl)-1,2,4-oxadiazol-3-
yl)benzyl)pyrrolidine-2-carboxamide
(2S,3S)-N-(4-(5-Cyclopentyl-1,2,4-oxadiazol-3-yl)benzyl)-3-hydroxypyrrolidine-2-
carboxamide
(2S,3S)-N-(4-(5-Cycloheptyl-1,2,4-oxadiazol-3-yl)benzyl)-3-hydroxypyrrolidine-2-
carboxamide
(2S,3S)-N-(4-(5-(2-Cyclopentylethyl)-1,2,4-oxadiazol-3-yl)benzyl)-3-hydroxypyrrolidine-
2-carboxamide
(2S,3S)-N-(4-(5-(2-Cyclopropylethyl)-1,2,4-oxadiazol-3-yl)benzyl)-3-
hydroxypyrrolidine-2-carboxamide
(2S,3S)-N-(4-(5-(4,4-Difluorocyclohexyl)-1,2,4-oxadiazol-3-yl)benzyl)-3
hydroxypyrrolidine-2-carboxamide
(2S,3S)-N-(4-(5-(2-Cyclohexylethyl)-1,2,4-oxadiazol-3-yl)benzyl)-3-hydroxypyrrolidine-
2-carboxamide
(2S,3S)-3-Hydroxy-N-(4-(5-phenethyl-1,2,4-oxadiazol-3-yl)benzyl)pyrrolidine-2-
carboxamide
(2S,3S)-N-((S)-1-(4-(5-(2-Cyclohexylethyl)-1,2,4-oxadiazol-3-yl)phenyl)propyl)-3-
hydroxypyrrolidine-2-carboxamide
(2S,3S)-N-((S)-1-(4-(5-(2-Cyclohexylethyl)-1,2,4-oxadiazol-3-yl)phenyl)ethyl)-3-
hydroxypyrrolidine-2-carboxamide hemi-tartrate salt
(2S,3S)-N-((S)-1-(4-(5-(2-Cyclopentylethyl)-1,2,4-oxadiazol-3-yl)phenyl)ethyl)-3-
hydroxypyrrolidine-2-carboxamide hemi-tartrate salt
(2S,3S)-N-((S)-1-(4-(5-(2-Cyclopentylethyl)-1,2,4-oxadiazol-3-yl)phenyl)propyl)-3-
hydroxypyrrolidine-2-carboxamide hemi-tartrate salt
(2S,3S)-N-((S)-1-(4-(5-(2-Cyclohexyl-1,2,4-oxadiazol-3-yl)phenyl)propyl)-3-
hydroxypyrrolidine-2-carboxamide hemi-tartrate salt
(2S,3S)-N-((S)-1-(4-(5-(2-Cyclobutylethyl)-1,2,4-oxadiazol-3-yl)phenyl)ethyl)-3-
hydroxypyrrolidine-2-carboxamide hemi-tartrate salt
(2S,3R)-2-amino-N-(4-(5-heptylpyridin-2-yl)phenyl)-3-hydroxybutanamide
(2S,4S)-N-(4-(3-heptyl-1,2,4-oxadiazol-5-yl)benzyl)-4-hydroxypiperidine-2-carboxamide
(2S,3S)-3-hydroxy-N-(4-(2-octylthiazol-4-yl)benzyl)pyrrolidine-2-carboxamide
(2S,3S)-N-(4-(5-heptylpyridin-2-yl)phenyl)-3-hydroxypyrrolidine-2-carboxamide
(2S,3S)-N-(4-(5-heptyl-1H-pyrazol-3-yl)benzyl)-3-hydroxypyrrolidine-2-carboxamide
(2S,3S)-3-hydroxy-N-(4-(5-octylthiazol-2-yl)benzyl)pyrrolidine-2-carboxamide
(2S,3S)-3-Hydroxy-N-((2-octylbenzofuran-5-yl)methyl)pyrrolidine-2-carboxamide
(2S,3S)-3-Hydroxy-N-((2-octyl-1H-indol-5-yl)methyl)pyrrolidine-2-carboxamide
(2S,3S)-3-Hydroxy-N-((1-methyl-2-octyl-1H-indol-5-yl)methyl)pyrrolidine-2-
carboxamide
(2S,3S)-N-((2-(3-(4-Fluorophenoxy)propyl)benzofuran-5-yl)methyl)-3-
hydroxypyrrolidine-2-carboxamide
(2S,3S)-N-((2-(2-(4-Fluorophenoxy)ethyl)benzofuran-5-yl)methyl)-3-hydroxypyrrolidine-
2-carboxamide
(2S,3S)-3-Hydroxy-N-((2-octylbenzofuran-6-yl)methyl)pyrrolidine-2-carboxamide
(2S,3S)-N-((2-Heptylbenzofuran-5-yl)methyl)-3-hydroxypyrrolidine-2-carboxamide
(2S,3S)-N-((2-Hexylbenzofuran-5-yl)methyl)-3-hydroxypyrrolidine-2-carboxamide
(2S,3S)-N-((2-cyclohexylbenzofuran-5-yl)methyl)-3-hydroxypyrrolidine-2-carboxamide
(2S,3S)-N-((2-(Cyclohexylmethyl)benzofuran-5-yl)methyl)-3-hydroxypyrrolidine-2-
carboxamide
(2S,3S)-N-((2-(2-Cyclohexylethyl)benzofuran-5-yl)methyl)-3-hydroxypyrrolidine-2-
carboxamide
(2S,3S)-N-((7-Fluoro-2-octylbenzofuran-5-yl)methyl)-3-hydroxypyrrolidine-2-
carboxamide
(2S,3S)-N-((6-Fluoro-2-octylbenzofuran-5-yl)methyl)-3-hydroxypyrrolidine-2-
carboxamide
(2S,3S)-N-((2-(Cyclopentylmethyl)-7-fluorobenzofuran-5-yl)methyl)-3-
hydroxypyrrolidine-2-carboxamide
(2S,3S)-N-((4-Fluoro-2-octylbenzofuran-5-yl)methyl)-3-hydroxypyrrolidine-2-
carboxamide
(2S,3S)-N-((2-(Cyclohexyl-7-fluorobenzofuran-5-yl)methyl)-3-hydroxypyrrolidine-2-
carboxamide
(2R,4R)-N-(4-decylphenyl)-4-hydroxypiperidine-2-carboxamide
(2S,4S)-N-(4-decylphenyl)-4-hydroxypiperidine-2-carboxamide

or a pharmaceutically acceptable salt, ester or prodrug thereof.

Pharmaceutically acceptable salts, esters, prodrugs, tautomers, hydrates and solvates of the compounds presently disclosed are also within the scope of the present disclosure.

Presently disclosed compounds that are basic in nature are generally capable of forming a wide variety of different salts with various inorganic and/or organic acids. Although such salts are generally pharmaceutically acceptable for administration to animals and humans, it may be desirable in practice to initially isolate a compound from the reaction mixture as a pharmaceutically unacceptable salt and then simply convert the latter back to the free base compound by treatment with an alkaline reagent, and subsequently convert the free base to a pharmaceutically acceptable acid addition salt. The acid addition salts of the base compounds can be readily prepared using conventional techniques, e.g., by treating the base compound with a substantially equivalent amount of the chosen mineral or organic acid in an aqueous solvent medium or in a suitable organic solvent such as, for example, methanol or ethanol. Upon careful evaporation of the solvent, the desired solid salt is obtained.

Acids which can be used to prepare the pharmaceutically acceptable acid-addition salts of the base compounds are those which can form non-toxic acid-addition salts, i.e., salts containing pharmacologically acceptable anions, such as chloride, bromide, iodide, nitrate, sulfate or bisulfate, phosphate or acid phosphate, acetate, lactate, citrate or acid citrate, tartrate or bitartrate, succinate, maleate, fumarate, gluconate, saccharate, benzoate, methanesulfonate and pamoate [i.e., 1,1′-methylene-bis-(2-hydroxy-3-naphthoate)] salts,

Presently disclosed compounds that are acidic in nature, e.g., contain a COOH or tetrazole moiety., are generally capable of forming a wide variety of different salts with various inorganic and/or organic bases. Although such salts are generally pharmaceutically acceptable for administration to animals and humans, it may be desirable in practice to initially isolate a compound from the reaction mixture as a pharmaceutically unacceptable salt and than simply convert the latter hack to the free acid compound by treatment with an acidic reagent, and subsequently convert the free acid to a pharmaceutically acceptable base addition salt. These base addition salts can be readily prepared using conventional techniques, e.g., by treating the corresponding acidic compounds with an aqueous solution containing the desired pharmacologically acceptable cations, and then evaporating the resulting solution to dryness, preferably under reduced pressure. Alternatively, they also can be prepared by mixing lower alkanolic solutions of the acidic compounds and the desired alkali metal alkoxide together, and then evaporating the resulting solution to dryness in the same manner as before. In either case, stoichiometric quantities of reagents are preferably employed in order to ensure completeness of reaction and maximum product yields of the desired solid salt.

Bases which can he used to prepare the pharmaceutically acceptable base-addition salts of the base compounds are those which can form non-toxic base-addition salts, i.e., salts containing pharmacologically acceptable cations, such as, alkali metal cations (e.g., potassium and sodium), alkaline earth metal cations (e.g., calcium and magnesium), ammonium or other water-soluble amine addition salts such as N-methylglucamine-(meglumine), lower alkanolammonium and other such bases of organic amines.

Isotopically-labeled compounds are also within the scope of the present disclosure. As used herein, an “isotopically-labeled compound” refers to a presently disclosed compound including pharmaceutical salts and prodrugs thereof, each as described herein, in which one or more atoms are replaced by an atom having an atomic mass or mass number different from the atomic mass or mass number usually find in nature. Examples of isotopes that can be incorporated into compounds presently disclosed include isotopes of hydrogen, carbon, nitrogen, oxygen, phosphorous, fluorine and chlorine, such as ²H, ³H, ¹³C, ¹⁴C, ¹⁵N, ¹⁸O, ¹⁷O, ³¹P, ³²P, ³⁵S, ¹⁸F, and ³⁶Cl, respectively,

By isotopically-labeling the presently disclosed compounds, the compounds may be useful in drug and/or substrate tissue distribution assays. Tritiated (³H) and carbon-14 (¹⁴C) labeled compounds are particularly preferred for their ease of preparation and detectability. Further, substitution with heavier isotopes such as deuterium (²H) can afford certain therapeutic advantages resulting from greater metabolic stability, for example increased in vivo half-life or reduced dosage requirements and, hence, may be preferred in some circumstances. Isotopically labeled compounds presently disclosed, including pharmaceutical salts, esters, and prodrugs thereof, can be prepared by any means known in the art.

Further, substitution of normally abundant hydrogen (¹H) with heavier isotopes such as deuterium can afibrd certain therapeutic advantages, e.g., resulting from improved absorption, distribution, metabolism and/or excretion (ADME) properties, creating drugs with improved efficacy, safety, and/or tolerability. Benefits may also be obtained from replacement of normally abundant ¹²C with ¹³C. See, WO 20071005641, WO 2007/005644, WO 2007/016361, and WO 2007/016431.

Stereoisomers (e.g., cis and trans isomers) and all optical isomers of a presently disclosed compound (e.g., R and S enantiomers), as well as racemic, diastereomeric and other mixtures of such isomers are within the scope of the present disclosure.

The compounds, salts, esters, prodrugs, hydrates, and solvates presently disclosed can exist in several tautomeric forms, including the enol and imine form, and the keto and enamine form and geometric isomers and mixtures thereof. Tautomers exist as mixtures of a tautomeric set in solution. In solid form, usually one tautomer predominates. Even though one tautomer may be described, all tautomers are within the scope of the present disclosure.

Atropisomers are also within the scope of the present disclosure. Atropisomers refer to compounds that can be separated into rotationally restricted isomers.

The present disclosure also provides pharmaceutical compositions comprising at least one presently disclosed compound and at least one pharmaceutically acceptable carrier. The pharmaceutically acceptable carrier can be any such carrier known in the art including those described in, for example, Remington's Pharmaceutical Sciences, Mack Publishing Co., A. R. Crennaro edit. 1985). Pharmaceutical compositions of the compounds presently disclosed may be prepared by methods known in the art including, for example, mixing at least one presently disclosed compound with a pharmaceutically acceptable carrier.

Presently disclosed pharmaceutical compositions can be used in an animal or human. Thus, a presently disclosed compound can be formulated as a pharmaceutical composition for oral, buccal, parenteral (e,g., intravenous, intramuscular or subcutaneous), topical, rectal or intranasal administration or in a form suitable for administration by inhalation or insufflation,

The compounds presently disclosed may also be Ibrinulated Ism sustained delivery according, to methods well known to those of ordinary skill in the art. Examples of such formulations can be found. in U.S. Pat. Nos. 3,119,742; 3,492397; 3,538,214; 4,060,598; and 4,173,626.

For oral administration, the pharmaceutical composition may take the form of, for example, a tablet or capsule prepared by conventional methods with a pharmaceutically acceptable excipient(s) such as a binding agent (e.g., pregelatinized maize starch, polyvinylpyrrolidone or hydroxylpropyl methylcellulose) filler (e.g., lactose, microcrystalline cellulose or calcium phosphate); lubricant (e,g., magnesium stearate, talc or silica); disintegrant (e.g., potato starch or sodium starch glycolate); and/or wetting agent (e.g., sodium lauryl sulphate). The tablets may be coated by methods well known in the art. Liquid preparations for oral administration may take the form of a, for example, solution, syrup or suspension, or they may be presented as a dry product for constitution with water or other suitable vehicle before use. Such liquid preparations may be prepared by conventional methods with a pharmaceutically acceptable additive(s) such as a suspending agent (e.g., sorbitol syrup, methyl cellulose or hydrogenated edible fats); emulsifying agent (e.g., lecithin or acacia); non-aqueous vehicle e.g., almond oil, oily esters or ethyl alcohol); and/or preservative (e.g., methyl or propyl p-hydroxybenzoates or sorbic acid).

For buccal administration, the composition may take the form of tablets or lozenges formulated in a conventional manner.

Presently disclosed compounds may be formulated for parenteral administration by injection, including using conventional catheterization techniques or infusion. Formulations fur injection may be presented in unit dosage form, e.g., in ampules or in multi-dose containers, with an added preservative. The compositions may take such forms as suspensions, solutions or emulsions in oily or aqueous vehicles, and may contain a formulating agent such as a suspending, stabilizing and/or dispersing agent recognized by those of skill in the art. Alternatively, the active ingredient may be in powder form for reconstitution with a suitable vehicle, e.g., sterile pyrogen-free water, before use.

For topical administration, a presently disclosed compound may be formulated as an ointment or cream.

Presently disclosed compounds may also be formulated in rectal compositions such as suppositories or retention enemas, e.g., containing conventional suppository bases such as cocoa butter or other glycerides.

For intranasal administration or administration by inhalation, presently disclosed compounds may be conveniently delivered in the form of a solution or suspension from a pump spray container that is squeezed or pumped by the patient or as an aerosol spray presentation from a pressurized container or a nebulizer, with the use of a suitable propellant, e.g., dichlorodifluoromethane, trichlorofluoromethane, dichlorotetrafluoroethane, carbon dioxide or other suitable gas. In the case of a pressurized aerosol, the dosage unit may be determined by providing a valve to deliver a metered amount. The pressurized container or nebulizer may contain a solution or suspension of the presently disclosed compound. Capsules and cartridges (made, for example, from gelatin) for use in an inhaler or insufflator may be formulated containing a powder mix of a presently disclosed compound and a suitable powder base such as lactose or starch.

A proposed dose of a presently disclosed compound for oral, parenteral or buccal administration to the average adult human for the treatment or prevention of an SK-related disease state is about 0.1 mg to about 2000 mg. In certain embodiments, the proposed dose is from about 0.1 mg to about 200 mg of the active ingredient per unit dose irrespective of the amount of the proposed dose, administration of the compound can occur, for example, 1 to 4 times per day.

Aerosol formulations for the treatment or prevention of the conditions referred to above in the average adult human are preferably arranged so that each metered dose or “puff” of aerosol contains about 20 μg to about 10,000 μg, preferably, about 20 μg to about 1000 μg of a presently disclosed compound. The overall daily dose with an aerosol will be within the range from about 100 μg to about 100 mg. In certain embodiments, the overall daily dose with an aerosol generally will be within the range from about 100 μg to about 10 mg. Administration may be several times daily, for example 2, 3, 4 or 8 times, giving for example, 1, 2 or 3 doses each time,

Aerosol combination formulations for the treatment or prevention of the conditions referred to above in the average adult human are preferably arranged so that each metered dose or “puff” of aerosol contains from about 0.01 mg to about 1000 mg of a combination comprising a presently disclosed compound. In certain embodiments, each metered dose or “puff” of aerosol contains about 0.01 mg to about 100 mg of a combination comprising a presently disclosed compound. In certain embodiments, each metered dose or “puff” of aerosol contains about 1 mg to about 10 mg of a combination comprising a presently disclosed compound. Administration may be several times daily, for example 2, 3, 4 or 8 times, giving for example, 2 or 3 doses each time.

Pharmaceutical compositions and methods of treatment or prevention comprising administering of at least one presently disclosed compound are also within the scope of the present disclosure.

### Incorporation by Reference

References to other documents, such as patents, patent applications, journals, books, etc., have been made throughout this disclosure. All such documents are hereby incorporated herein by reference in their entirety for all purposes.

### Equivalents

It is to be understood that the foregoing description is exemplary and explanatory in nature, and is intended to illustrate the presently disclosed general inventive concept and its preferred embodiments. Through routine experimentation, those of skill in the art given the benefit of the present disclosure may recognize apparent modifications and variations without departing from the spirit and scope of the present disclosure. Thus, the present disclosure is not limited by the above description, but rather by the following claims and their equivalents.

### EXAMPLES

Although specific embodiments of the present disclosure will now be described with reference to the preparations and schemes, it should be understood that such embodiments are by way of example only and merely illustrative of but a small number of the man possible specific embodiments that can represent applications of the principles of the present disclosure. Various changes and modifications will be obvious to those of skill in the art given the benefit of the present disclosure and are deemed to be within the spirit and scope of the present disclosure asd further defined in the appended claims.

Unless defined otherwise, technical and scientific terms used herein have the same meaning as commonly understood by one having ordinary skill in the art to which this disclosure belongs. Although other compounds or methods can be used in practice or testing, certain preferred methods are now described in the context of the following preparations and schemes.

¹H spectra were generated at 400 MHz (Varian Unity 400 instrument). Chemical shifts are given in parts per million relative to a tetramethylsilane internal standard. HPLC analyses Were performed using an Ace C8 column (4.6×50 min; particle size, 3 μM) and a graduating, two-phase eluant (A/B, 100%/0% to 10%/90% to 100%/0%; A=90% 25 mM ammonium acetate, 10% acewnitrile B=10% ammonium acetate/90% acetontrile). UPLC analyses were performed using a Waters BEH C18 column (2.1×50 mm; particle size, 1.8 μM) and a graduating, two-phase eluant (A/B, 95%/5% to 5%/95%; A=0.1% formic acid in water; B=0.1% formic acid in acetonitrile).

### Preparation A

### Preparation of (2S,3S)-3-Hydroxy-N-(4-octylphenyl)pyrrolidine-2-carboxamide

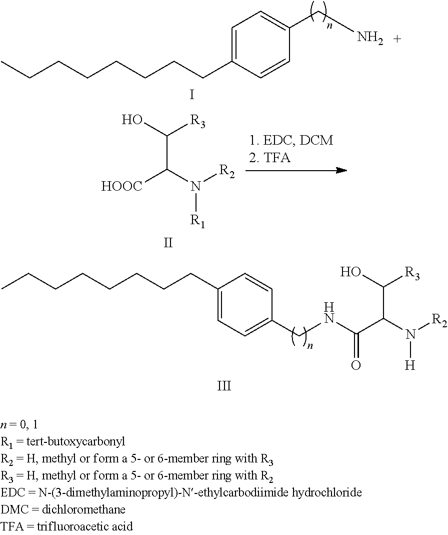

To a stirred solution of 4-octylaniline (0.480 g. 2.34 mmol) or other appropriate amines (I) and N-tert-butoxycarbonyl)-trans-3-hydroxy-L -proline (0.594 g, 2.57 mmol) or other amino cids (II) in methylene chloride (12 mL) or other suitable solvents such as dichloroethane or THF was added N-(3-dimethylaminopropyl)-N′-ethylcarbodiimide hydrochloride (0.493 g, 2.57 mmol). in some reactions, equal mole amount of DMAP (4-N′N′-dimethylpyridine) or/and HOBT (1-hydroxybenzotrizole) were added too. After overnight stirring, the mixture was diluted with water and extracted with methylene chloride. The combined extracts were dried (sodium saltine) and concentrated to yield crude amide product as an amber gum (1.01 g). This material was taken up in methylene chloride (8 mL) or another suitable solvent such as ethyl acetate or chloroform, stirred and treated trifluoroacetic acid (4 mL) or another suitable acid, such as aqueous hydrochloride. After 2 hours the reaction was concentrated. The residue was taken up in aqueous sodium bicarbonate solution and extracted repeatedly with methylene chloride, or another suitable solvent, such as ethyl acetate. The combined extracts were dried (sodium sulfate) and concentrated to afford a pale amber gum. This material was purified by flash chromatography over silica (19:1 methylene chloride/2 N methanolic ammonia) to afford 0.625 g (84%) of (2S,3S )-3-Hydroxy-N-(4-octylphenyl)pyrrolidine-2-carboxamide or other appropriate amide products (III).

The reaction was accomplished when, in addition of EDCI, an equal molar of DMAP (4-N,N′-dimethylaminopyridine) and HOBT (1-hydroxybenzotrizole) were added to the reaction solution.

### Example 1

### (2S,3S)-3-Hydroxy-N-(4-octylphenyl)pyrolidine-2-carboxamide

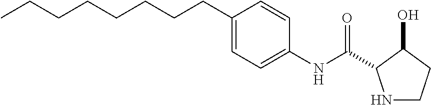

The compound of Example 1 was prepared using 4-octylaniline (0.480 g, 2.34 mmol) and N-(tert-butoxycarbonyl)-trans-3-hydroxy-L-proline (0.594 g, 2.57 mmol) and following the procedure described in Preparation A: ¹H NMR (DMSO-d₆) δ 9.85 (br s, 1H), 7.51 (d, J=7.8 Hz, 2H), 7.07 (d, J=7.8 Hz, 2H), 5.08-4.97 (m, 1H), 4.31-4.20 (m, 1H), 3.47 (br s, 1H), 3.20 (br s, 1H), 3.10-2.89 (m, 2H), 2.60-2.41 (m, 2H), 1.70-1.56 (m, 2H), 1.56-1.42 (m, 2H), 1.33-1.12 (m, 10H), 0.89-0.76 (m, 3H) ppm. MS (ESI) m/z 319 (M+H)⁺.

### Example 2

### (2S,3R)-3-Hydroxy-N-(4-octylphenyl)pyrrolidine-2-carboxamide

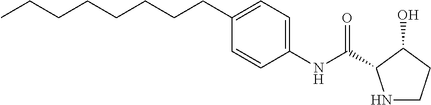

Utilizing a procedure similar to that described in Preparation A, the compound of Example 2 was prepared from 4-octylaniline and N-(tert-butoxycarbonyl)-cis-3-hydroxy-L-proline. Product was afforded as a white solid: ¹H NMR (CDCl₃) δ 9.68 (br s, 1H), 7.49 (d, J=8.5 Hz, 2H), 7.13 (d, J=8.5 Hz, 2H), 4.76-4.70 (m, 1H), 3.14 (d, J=5.9 Hz, 1H), 3.19-3.07 (m, 2H), 2.56 (t, J=7.6 Hz, 2H), 2.01-1.86 (m, 2H), 1.63-1.52 (m, 2H), 1.36-1.19 (m, 10H), 0.88 (t, J=6.9, 3H) ppm, MS (ESI) m/z 319 (M+H)⁺.

### Example 3

### (R)-2-Amino-3-hydroxy-N-(4-octylphenyl)propanamide

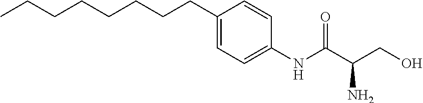

Utilizing a procedure similar to that described in Preparation A, the compound of Example 3 was prepared from 4-octylaniline and N-(tert-butoxycarbonyl)-D-serine. Product was afforded as a white solid: ¹H NMR (DMSO-d₆) δ 9.77 (br s, 1H), 7.51 (d, J=7.1 Hz, 2H), 7.07 (d, J=7.1 Hz, 2H), 4.79 (br s, 1H), 3.65-3.42 (m, 2H), 3.41-3.27 (m, 1H), 2.60-2.39 (m, 2H), 1.58-1.42 (m, 2H), 1.34-1.11 (m, 10H), 0.90-0.75 (m, 3H) ppm. MS (ESI) m/z 293 (M+H)⁺.

### Example 4

### (S)-2-Amino-3-hydroxy-N-(4-octylphenyl)propanamide

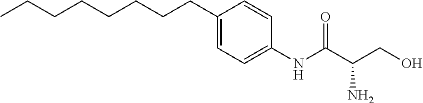

Utilizing a procedure similar to that described in Preparation A, the compound of Example 4 was prepared from 4-octylaniline and N-(tert-butoxycathonyl)-L-serine. Product was afforded as an off-while solid: ¹H NMR (DMSO-d₆) δ 9.79 (br s, 1H), 7.65-7.38 (m, 2H), 7.21-6.94 (m, 2H), 4.83 (br s, 1H), 3.66-3.42 (m, 2H), 3.42-3.22 (m, 1H), 2.60-2.40 (m, 2H), 1.94 (br s, 2H), 1.64-1.42 (m, 2H), 1.40-1.02 (m, 10H), 0.95-0.70 (m, 3H) ppm. MS (ESI) m/z 293 (M+H)⁺.

### Example 5

### (±)-erythro-DL-2-Amino-3-hydroxy-N-(4-octylphenyl)pentanamide

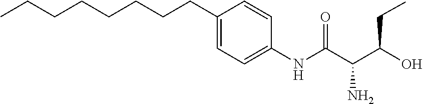

Utilizing a procedure similar to that described in Preparation A, the compound of Example 5 was prepared from 4-octylandine and N-(tert-butoxycarbonyl)-3-hydroxy-DL-norvaline. Product was afforded as a white solid: ¹H NMR (CDCl₃) δ 9.56 (br s, 1H), 7.48 (d, J=8.3 Hz, 2H), 7.13 (d, J=8.3 Hz, 2H), 4.24-4.18 (m, 1H), 3.38 (d, J=2.5 Hz, 1H), 2.56 (t, J=7.5 Hz, 2H), 1.64-1.46 (m, 4H), 1.36-1.19 (m, 10H), 1.01 (t, J=7.4 Hz, 3H), 0.88 (t, J=7.0 Hz, 3H) ppm. MS (ESI) m/z 321 (M+H)⁺.

### Example 6

### (2S,3R)-2-Amino-N-(4-decylphenyl)-3-hydroxybutanamide

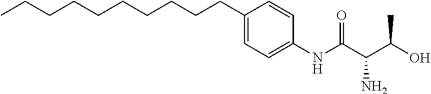

Utilizing a procedure similar to that described in Preparation A, the compound of Example 6 was prepared from 4-decylaniline and N-(tert-butoxycarbonyl)-L-threonine. Product was afforded as a white solid: ¹H NMR (DMSO-d₆) δ 9.79 (br s, 1H), 7.51 (d, J=8.4 Hz, 2H), 7.08 (d, J=8.4 Hz, 2H), 4.67 (d, J=4.1 Hz, 1H), 3.95-3.85 (m, 1H), 3.06 (d, J=4.4 Hz, 1), 2.52-2.44 (m, 2H), 2.01 (br s, 2H), 1.56-1.45 (m, 2H), 1.30-1.15 (m, 14H), 1.08 (d, J=6.4 Hz, 3H), 0.83 (t, 7.1 Hz, 3H) ppm. MS (ESI) m/z 335 (M+H)⁺.

### Example 7

### (2S,3R)-2-Amino-3-hydroxy-N-(4-undecylphenyl)butanamide

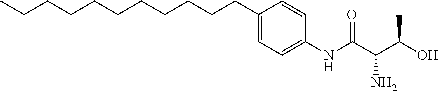

Utilizing a procedure similar to that described in Preparation A, the compound of Example 7 was prepared from 4-undecylamiline and N-(tert-botoxycarbonyl)-L-threonine. Product was afforded as a white solid: ¹H NMR (CDCl₃) δ 9.49 (br s, 1H), 7.48 (d, J=8.3 Hz, 2H), 7.13 (d, J=8.43 Hz, 2H), 4.51-4.43 (m, 1H), 3.3.5 (d, J=2.8, 1H), 2.56 (t, J=7.8 Hz, 1H), 1.63-1.53 (m, 2H), 1.35-1.20 (m, 19H), 0.88 (t, J=7.0 Hz, 3H) ppm. MS (ESI) m/z 349 M+H)⁺.

### Example 8

### (2S,3R)-2-Amino-N-(4-dodecylphenyl)-3-hydroxybutanamide

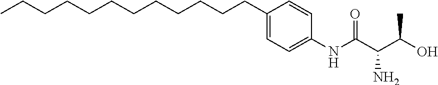

Utilizing a procedure similar to that described in Preparation A, the compound of Example 8 was prepared from 4-dodecylaniline and N-(tert-butoxycarbanyl)-L-threonine. Product was afforded as a white solid: ¹H NMR (DMSO-d₆) δ 9.78 (br s, 1H), 7.51 (d, J=8.3 Hz, 2H), 7.08 (d, J=8.3 Hz, 2H), 4.66 (d, J=4.1 Hz, 1H), 3.95-3.85 (m, 1H), 3.06 (d, J=4.3 Hz, 1H), 2.52-2.44 (m, 2H), 1.93 (br s, 2H), 1.56-1.45 (m, 2H), 1.30-1.15 (m, 18H), 1.08 (d, J=6.4 Hz, 3H), 0.83 (t, J=7.0 Hz, 3H) ppm. MS (ESI) m/z 363 (M+H)⁺.

### Preparation B

### Preparation of (2R,3S)-2-((4-Octylphenylamino)methyl)pyrolidin-3-ol

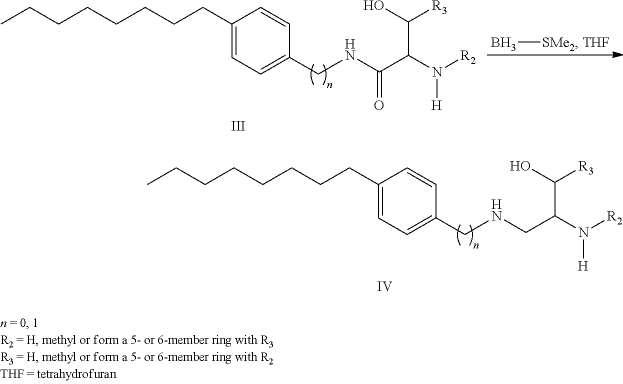

To a stirred solution of compound III, such as the compound of Example 1 (0.239 g, 0.751 mmol) in THE (10 mL) or another suitable solvent, such as dichloroethane, was added borane dimethyl sulfide complex (0.50 mL, 5.3 mmol) or another suitable reducing reagent, such as lithium aluminum hydride. The mixture was heated at reflux overnight and then concentrated. Methanol (10 mL) was slowly added to the residue and the resulting solution was heated at reflux for 30 minutes. The reaction was concentrated again, taken up in 6 N aqueous HCl and heated at reflux for 1 hour. After cooling to room temperature, the mixture was treated slowly with concentrated ammonium hydroxide (5 mL). The resulting off-white precipitate was filtered off, rinsed with water and vacuum oven-dried. This material was purified by flash chromatography over silica (9:1 methylene chloride/2 N methanolic ammonia) to afford 0.199 g (87%) of product (IV

### Example 9

### (2R,3S)-2-((4-Octylphenylamino)methyl)pyrrolidin-3-ol

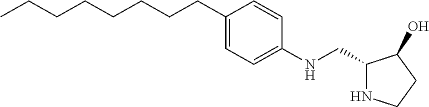

Utilizing the procedure described in Preparation B, the compound of Example 9 was prepared from Example 2 (0.239 g, 0.751 mmol) in THF (10 mL) and borane dimethyl sulfide complex (0.50 mL, 5.3 mmol) as an off-white ¹H NMR (CDCl₃) δ 6.99 (d, J=8.4 Hz, 2H), 6.59 (d, J=8.5 Hz, 2H), 4.18-4.13 (m, 1H), 3.26-3.20 (m, 1H), 3.20-3.11 (m, 2H), 3.03-2.94 (m, 2H), 2.48 (t, J=7.5 Hz, 2H), 2.13-2.02 (m, 1H), 1.81-1.71 (m, 1H), 1.60-1.49 (m, 2H), 1.36-1.19 (m, 10H), 0.87 (t, J=7.0 Hz, 3H) ppm. MS (ESI) m/z 305 (M+H)⁺.

### Example 10

### (R)-2-Amino-3-(4-octylphenylamino)propan-1-ol

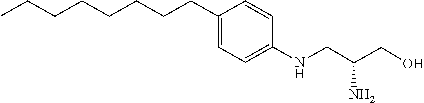

Utilizing a procedure similar to that described in Preparation B, the compound of Example 10 was prepared from (S)-2-Amino-3-hydroxy-N-(4-octylphenyl)propanamide of Example 4. Product was afforded as a white solid: ¹H NMR (CDCl₃) δ 6.99 (d, J=8.3 Hz, 2H), 6.58 (d, J=8.3 Hz, 2H), 3.69 (dd, J=10.7, 4.3 Hz, 1H), 3.53 (dd, J=10.7 5.8 Hz, 1H), 3.26-3.12 (m, 2H), 3.10-3.01 (m, 1H), 2.49 (t, J=7.6 Hz, 2H), 1.75 (br s, 2H), 1.60-1.48 (m, 2H), 1.37-1.17 (m, 10H), 0.87 (t, J=7.0 Hz, 3H) ppm. MS (ESI) m/z 279 (M+H)⁺.

### Example 11

### (2S,3R)-2-Amino-3-hydroxy-N-(3-octylphenyl)butanamide

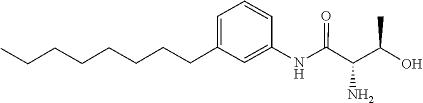

Utilizing a procedure similar to that described in Preparation A, the compound of Example 11 was prepared from 3-octylaniline and N-(tert-butoxycarbonyl)-L-threonine. The product was obtained a white solid: ¹H NMR (DMSO-d₆) δ 9.79 (br s, 1H), 7.60-7.32 (m, 2H), 7.16 (t, J=7.8 Hz, 1H), 6.84 (d, J=7.6 Hz, 1H), 4.66 (d, J=5.0 Hz, 1H), 3.97-3.87 (m, 1H), 3.07 (d, J=4.2 Hz, 1H), 2.50-2.45 (m, 2H), 1.94 (br s, 2H), 1.59-1.46 (m, 2H), 1.30-1.15 (m, 10H), 1.09 (d, 6.4 Hz, 3H), 0.83 (t, J=6.8 Hz, 3H) ppm. MS (ESI) m/z 307 (M+H)⁺.

### Example 12

### (2S,3R)-2-Amino-N-(4-(heptyloxy)phenyl)-3-hydroxybutanamide

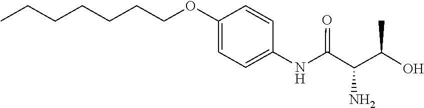

Utilizing a procedure similar to that described in Preparation A, the compound of Example 12 was prepared from 4-(heptyloxy)aniline and N-(tert-butoxycarbonyl)-L-threonine. The product was obtained as a white solid: ¹H NMR (CD₃Cl) δ 9.41 (br s 1H), 7.50-7.43 (m, 2H), 6.88-6.82 (m, 2H), 4.49-4.42 (m, 1H), 3.92 (t, J=6.6 Hz, 2H), 3.34 (d, J=2.9 Hz, 1H), 2.39 (br s, 2H), 1.80-1.71 (m, 3H), 1.48-1.22 (m, 1H), 0.89 (t, J=6.9 Hz, 3H) ppm. MS (ESI) m/z 309 (M+H)⁺.

### Example 13

### (±)-2-Amino-3-hydroxy-N-(4-nonylphenyl)propanamide

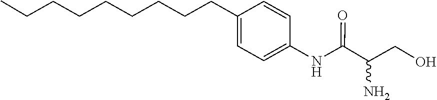

Utilizing a procedure similar to that described in Preparation A, the compound of Example 13 was prepared from 4-nonylaniline and N-(tert-butoxycarbonyl)-DL-serine. The product was obtained as a white solid: ¹H NMR (CD₃Cl) δ 9.43 (br s 1H), 7.47 (d, J=8.4 Hz, 2H), 7.14 (d, J=8.4 Hz, 2H), 4.00 (dd, J=4.9, 10.8 Hz, 1H), 3.79 (dd, J=5.6, 10.8 Hz, 1H), 3.56 (t, J=5.2 Hz, 1H), 2.75-2.42 (m, 3H), 1.72 (br s, 2H), 1.62-1.52 (m, 2H), 1.34-1.20 (m, 12H), 0.87 (t, J=6.8 Hz, 3H) ppm. MS (ESI) m/z 307 (M+H)⁺.

### Example 14

### (2S,3R)-2-Amino-3-hydroxy-N-(4-nonylphenyl)butanamide

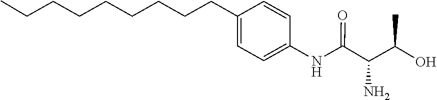

Utilizing a procedure similar to that described in Preparation A, the compound of Example 14 was prepared from 4-(heptyloxy)aniline and N-(tert-butoxycarbonyl)-L-threonine. The product was obtained as a white solid: ¹H NMR (CD₃Cl) δ 9.49 (br S, 1H), 7.48 (d, J=8.4 Hz, 2H), 7.13 (d, J=8.4 Hz, 2H), 4.47 (dd, J'2.8, 6.5 Hz, 1H), 3.35 (d, J=2.8 Hz, 1H), 2.66-2.42 (m, 2H,), 1.91-1.44 (m, 4H), 1.40-1.11 (m, 12H), 0.87 (t, J=6.8 Hz, 3H) ppm MS (ESI) m/z 321 (M+H)⁺.

### Example 15

### (2S,3R)-2-Amino-N-(4-(hexyloxymethyl)phenyl)-3-hydroxybutanamide

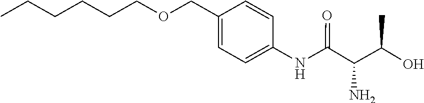

### Preparation of 4-(hexyloxymethyl)aniline

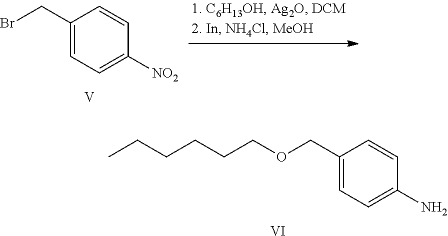

To a stirred solution of 4-nitrobenzyl bromide (V) (4.47 g, 20.69 mmol) and 1-hexanol (2.69 g, 26.29 mmol) in dichloromethane (40 mL) was added silver(I) oxide (5.28 g, 22.78 mmol). The reaction mixture was heated to reflux. After 18 hours, the reaction mixture was allowed to cool to room temperature and was filtered through Celite. The filtrate was concentrated to provide a colorless oil. Flash chromatography using an Isco Combiflash unit (1.20 g SiO₂ column, 5-10% ethyl acetate/hexanes) afforded 4.20 g, (86%) of 1-(hexyloxymethyl)-4-nitrobenzene as a colorless oil: ¹HNMR (CDCl₃) δ 8.20 (d, J=8.7 Hz, 2H), 7.50 (d, J=8.7 Hz, 2H), 4.59 (s, 2H), 3.51 (t, J=6.6 Hz, 2H), 1.64 (m, 2H), 1.35 (m, 2H), 1.35 (m, 6H), and 0.89 (t, J=6.9 Hz, 3H),

### Step 2) Preparation of 4-(hexyloxymethyl)aniline

To a stirred solution of 1-(hexyloxymethyl)-4-nitrobenzene (4.20 g, 17.70 mmol) in ethanol (100 mL) was added a saturated solution of ammonium chloride (30 mL) and indium metal (10.00 g, 87.09 mmol. The reaction mixture was heated to reflux. After 3 hours, the reaction mixture was allowed to cool to room temperature and stirring continued overnight. After 18 hours, the reaction mixture was diluted with water (600 mL), and the mixture filtered through Celite. The filtercake was washed with dichloromethane (300 mL). The phases of the filtrate were separated, and the aqueous phase made basic with 1N sodium hydroxide solution. This solution was extracted with dichloromethane. The combined organic phases were washed with brine, dried (magnesium sulfate), filtered, and concentrated to provide 2.68 g of a yellow oil. Flash chromatography using an Isco Combiflash unit (120 g SiO₂ column, 15-30% ethyl acetate/hexanes) afforded 1.18 g (32%) of 4-(hexyloxmethyl)aniline (VI) as a yellow oil: ¹H NMR (CDCl₃) δ 7.13 (d, J=8.3 Hz, 2H), 6.66 (d, J=8.3 Hz, 2H), 4.37 (s, 2H), 3.64 (br s, 2H), 341 (t, J=6.7 Hz, 2H), 1.58 (m, 2H), 1.46-1.17 (m, 6H), 0.88 (t, J=6.9 Hz, 3H) ppm.

Utilizing a procedure similar to that described in Preparation A, the compound of Example 15 was prepared from 4-(hexyloxymethyl)aniline and N-(tert-butoxycarbonyl)-L-threonine. The product was obtained as a white solid: ¹HNMR (CDCl₃) δ 9.59 (br s, 1H), 7.56 (d, J=8.4 Hz, 2H), 7.30 (d, J=8.4 Hz, 2H), 4.54-4.40 (m, 3H), 3.43 (t, J=6.7 Hz, 2H), 3.33 (d, J=2.8 Hz, 1H), 2.30-1.67 (br s, 2H), 1.65-1.53 (m, 2H), 1.41-1.19 (m, 10H), 0.88 (t, J=6.8 Hz, 3H) ppm. MS (ESI) m/z 309 (M+H)⁺,

### Example 16

### (2S,3R)-2-Amino-3-hydroxy-N-(4-(7-methyloctyl)-phenyl)butanamide

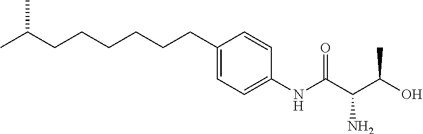

### Preparation of the intermediate 4-(7-methyloctyl)aniline

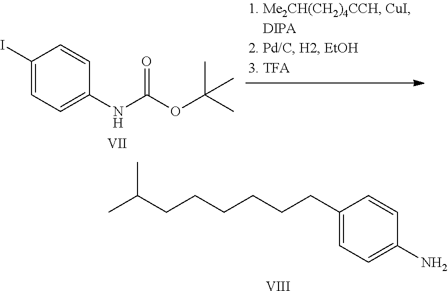

### Step 1) Preparation of tert-butyl 4-(7-methyloct-1-ynyl)phenylcarbamate

To a stirred solution of tert-butyl 4-iodophenylcarbamate (VII) (1.34 g, 4.20 mmol), 7-methyloct-1-yne (0.678 g, 5.46 mmol), and diisopropylamine (1.27 g, 12.60 mmol) in tetrahydrofuran (20 mL) was added copper(I) iodide (0.080 g, 0.420 mmol) and bis(triphenylphosphine)palladium(II) chloride (0.147 g, 0.210 mmol). The reaction mixture was allowed to stir at room temperature. After 3 hours, the reaction mixture was diluted with ethyl acetate and washed with 1N hydrochloric acid, 6N ammonium hydroxide, and brine. The organic phase was dried (magnesium sulfate), filtered, and concentrated to provide 2.02 g of a brown oil. Flash chromatography using an Isco Combiflash unit (90 g SiO₂ column, 5-10% ethyl acetate/hexanes) afforded 0.812 g (62%) of tert-butyl 4-(7-methyloct-1-ynyl)phenylcarbamate as a yellow oil: ¹H NMR (CDCl3) δ 7.34-7.27 (m, 4H), 6.45 (br s, 1H), 2.38 (t, j=7.1 Hz, 2H), 1.63-1.53 (m, 3H), 1.51 (s, 9H), 1.48-1.38 (m, 2H), 1.29-1.15 (m, 2H), 0.88 (d, J=6.6 Hz, 6H) ppm.

### Step 2) Preparation of tert-butyl 4-(7-methyloctyl)phenylcarbamate

To a stirred solution of tert-butyl 4-(7-methyloct-1-ynyl)phenylcarbamate (0.812 g, 2.57 mmol) in ethyl acetate (25 mL) was added 10% palladium on carbon (wet) (0.197 g). The reaction mixture was degassed under vacuum (ca. 30 mm Hg) and backfilled with nitrogen three times. After an additional evacuation, the atmosphere was replaced with hydrogen, and the reaction mixture allowed to stir at room temperature. After 18 hours, the remaining hydrogen was removed under vacuum and replaced with nitrogen. The reaction mixture was filtered through Celite, and the filtrate concentrated to provide 0.821 g (100%) of tert-butyl 4-(7-methyloctyl)phenylcarbamate as a yellow solid: ¹H NMR (CDCl3) δ 7.25 (d, J=8.4 Hz, 2H), 7.09 (d, J=8.4 Hz; 2H), 6.39 (br s, 1H,), 2.73-2.31 (m, 2H), 1.61-1.45 (m, 12H), 1.35-1.20 (m, 6H), 1.18-1.09 (m, 2H), 0.85 (d, J=6.6 Hz, 6H) ppm.

### Step 3) Preparation of 4-(7-methyloctyl)aniline (VIII)

To a stirred solution of tert-butyl 4-(7-methyloctyl)phenylcarbamate (0.821 g, 2.57 mmol) in dichloromethane (10 mL) was added trifluoroacetic acid (5 mL). The reaction mixture was allowed to stir at room temperature. After 1 hours, the reaction mixture was concentrated, and the residue dissolved in ethyl acetate. The solution was washed with 6N ammonium hydroxide and brine, dried (magnesium sulfate), filtered, and concentrated to provide 0.524 g (93%) of 4-(7-methyloctyl)aniline (VIII) as a brown oil: ¹H NMR (CDCl3) δ 6.97 (d, J=8.2 Hz, 2H), 6.62 (d, J=8.2 Hz, 2H), 3.53 (br s, 2H), 2.56-2.40 (m, 2H), 1.66-1.41 (m, 4H), 1.38-1.19 (m, 5H), 1.21-1.05 (m, 2H), 0.85 (d, J=6.6 Hz, 6H) ppm.

Utilizing a procedure similar to that described in Preparation A, the compound of Example 16 was prepared from 4-(7-methyloctyl)aniline and N-(tert-butoxycarbonyl)-L-threonine. The product was obtained as a white solid: ¹H NMR (CDCl3) δ 9.49 (br s, 1H), 7.48 (d, J=8.4 Hz, 2H), 7.13 (d, J=8.4 Hz, 2H), 4.47 (dd, J=2.9, 6.5 Hz, 1H), 3.35 (d, J=2.9 Hz, 1H), 2.65-2.47 (m, 2H), 1.87 (br s, 2H), 1.66-1.43 (m, 4H), 1.39-1.20 (m, 10H), 1.19-1.06 (m, 2H), 0.85 (d, J=6.6 Hz, 6H) ppm. MS (ESI) m/z 321 (M+H)⁺.

### Example 17

### (2S 3R)-2-Amino-N-(4-(7-(4-fluorophenoxy)heptyl)phenyl)-3-hydroxybutanamide

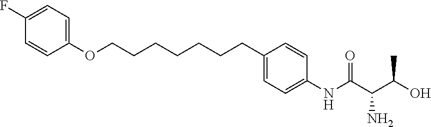

### Preparation of the intermediate 4-(7-(4-fluorophenoxy)heptyl)aniline

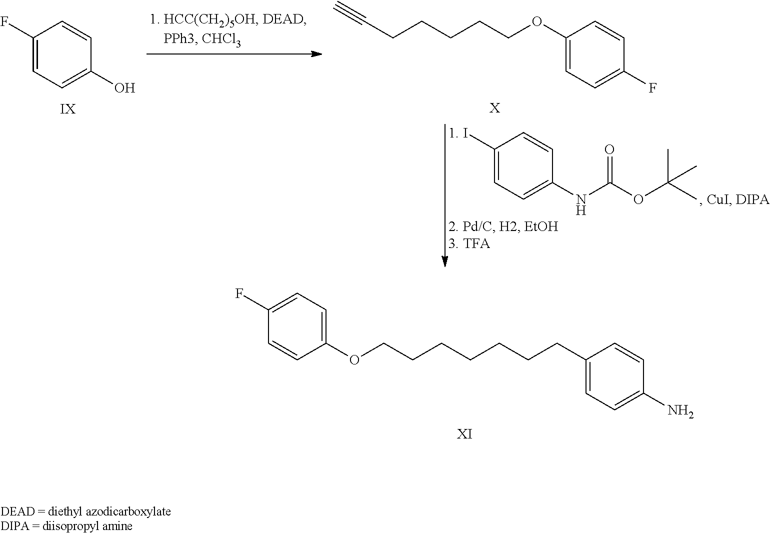

### Step 1) Preparation 1-fluoro-4-(hept-6-ynyloxy)benzene

To a stirred solution of 4-fluorophenol (IX) (3.79 g, 33.81 mmol), hept-6-yn-1-ol (4.24 g, 37.80 mmol), and triphenylphosphine (10.64 g. 40.57 mmol) in chloroform (100 mL, cooled to 0° C,) was added diethyl azodicarbox),late (7.65 g, 43.95 mmol) dropwise over 10 minutes. The resulting yellow solution was allowed to stir at 0° C. for 15 min. and then the reaction mixture allowed to warm to room temperature. After 17 hours, the reaction mixture was diluted with dichloromethane and washed with 1N hydrochloric acid, saturated sodium bicarbonate solution, and brine. The organic phase was dried (magnesium sulfate), filtered, and concentrated to provide a yellow semi-solid. The crude solid was triturated with hexanes to provide a yellow oil. Flash chromatography using an Isco Combiflash unit (120 g SiO₂ column, 1-10% ethyl acetatelhexanes) afforded 4.63 g (62%) of 1-fluoro-4-(hept-6-ynyloxy)benzene (X) as a yellow oil: ¹H NMR (CDCl3) δ 7.07-6.88 (m, 2H), 6.87-6.74 (m, 2H), 3.92 (t, J=6.4 Hz, 2H), 2.31-2.16 (m, 2H), 1.95 (t, J=2.6 Hz, 1H), 1.85-1.71 (m, 2H), 1.69-1.49 (m, 4H) ppm.

### Step 2) Preparation of tert-butyl 4-(7-(4-fluorophenoxy)hept-1-ynyl)phenylcarbanate

To a stirred solution of tert-butyl 4-iodophenylcarbamate (3.00 g, 9.40 mmol), 1-fluoro-4-(hept-6-ynyloxy)benzene (X) (2.13 g, 10.34 mmol), and diisopropylamine (2.85 g, 28.20 mmol) in tetrahydrofuran (50 mL) was added copper(I) iodide (0.179 g, 0.940 mmol) and bis(triphenylphosphine)palladium(II) chloride (0.330 g, 0.470 mmol). The reaction mixture was allowed to stir at room temperature. After 3 hours, the reaction mixture was diluted with ethyl acetate and washed with 1N hydrochloric acid 6N ammonium hydroxide, and brine. The organic phase was dried (magnesium sulfate), filtered, and concentrated to provide 5.25 g of an orange-brown solid. Flash chromatography using an Isco Combiflash unit (120 g SiO₂ column, 30-60% dichloromethane/hexanes) afforded 1.80 g (48%) of tert-butyl 4-(7-(4-fluorophenoxy)hept-1-ynyl)phenylcarbamate as an off-white solid: ¹H NMR (CDCl3) δ 7.34-7.2.7 (m, 4H), 7.00-6.92 (m, 2H), 6.86-6.79 (m, 2H), 6.46 (br s, 1H), 3.94 (t, J=6.4 Hz, 2H), 2.55-2.27 (m, 2H), 1.86-1.77 (m, 2H), 1.71-1.58 (m, 4H), 1.51 (s, 9H) ppm.

### Step 3) Preparation of tert-butyl 4-(7-(4-fluorophenoxy)heptyl)phenylcarbamate

To a stirred solution of tert-butyl 4-(7(4-fluorophenoxy)hept)1-ynyl)phenylcarbamate (1.80 g 4.53 mmol) in ethyl acetate (50 mL) was added 10% palladium on carbon (wet) (0.610 g). The reaction mixture was degassed under vacuum (ca. 30 mm Hg) and backfilled with nitrogen three times. After an additional evacuation, the atmosphere was replaced with hydrogen, and the reaction mixture allowed to stir at room temperature. After 18 hours, the remaining hydrogen was removed under vacuum and replaced with nitrogen. The reaction mixture was filtered through Celite, and the filtrate concentrated to provide 1.78 g (98%) of tert-butyl 4-(7-(4-fluorophenoxy)heptyl)phenylcarbarmate as an off-white solid: ¹H NMR (CDCl3) δ 7.26-7.22 (m, 2H), 7.12-7.04 (m, 2H), 7.00-6.91 (m, 2H), 6.85-6.77 (m, 2H), 6.40 (br s, 1H), 3.89 (t, J=6.5 Hz, 2H), 2.61-2.48 (m, 2H), 1.80-1.68 (m, 2H), 1.64-1.54 (m, 2H), 1.51 (s, 9H), 1.48-1.29 (m, 6H) ppm.

### Step 4) Preparation of 4-(7-(4-fluorophenoxy)heptyl)aniline

To a stirred solution of tert-butyl 4-(7-(4-fluorophenoxy)heptyl)phenylcarbamate (1.78 g, 4.43 mmol) in dichloromethane (20 mL) was added trifluoroacetic acid (10 mL). The reaction mixture was allowed to stir at room temperature. After 1 hour, the reaction mixture was concentrated, and the residue dissolved in ethyl acetate. The solution was washed with 6N ammonium hydroxide and brine, dried (magnesium sulfate), filtered, and concentrated to provide 1.34 g (100%) of 4-(7-(4-fluorophenoxy)heptyl)aniline (XI) as an orange solid ¹H NMR (CDCl3) δ 7.05-6.89 (m, 4H), 6.88-6.76 (m, 2H), 6.62 (d, J=8.3 Hz, 2H), 3.90 (t, J=6.5 Hz, 2H), 3.54 (br s, 2H), 2.58-2.43 (m, 2H), 1.84-1.68 (m, 2H), 1.66-1.51 (m, 2H), 1.51-1.28 (m, 6H) ppm.

Utilizing, a procedure similar to that described, in Preparation A, the compound of Example 17 was prepared from 4-(7-(4-fluorophenoxy)heptyl)aniline and N-(tert-butoxycarbonyl)-L-threonine. The product was obtained as a white solid: ¹H NMR (CDCl1) δ 9.50 (br s, 1H), 7.49 (d, J=8.4 Hz, 2H), 7.13 (d, J=8.4 Hz, 2H), 7.03-6.89 (m, 2H), 6.87-6.73 (m, 2H), 4.47 (m, 1H), 3.89 (t, J=6.5 Hz, 2H), 3.35 (d, 2.8 Hz, 1H), 2.67-2.49 (m, 2H), 2.28-1.81 (m, 3H), 1.80-1.67 (m, 2H), 1.66-1.53 (m, 2H), 1.51-1.30 (m, 6H), 1.26 (d, J=6.5 Hz, 3H) ppm. MS (ESI) m/z 403 (M+H)⁺.

### Example 18

### (2S,3R)-2-Amino-3-hydroxy-N-(4-(octyloxy)phenyl)-butanamide

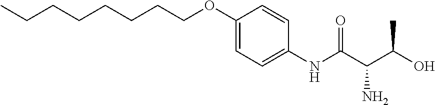

Utilizing a procedure similar to that described in Preparation A, the compound of Example 18 was prepared from 4-(octyloxy)aniline and N-(tert-butoxycarbonyl)-L-threonine. The product was obtained as a white solid: ¹H NMR (CDCl₃) δ 9.43 (br s, 1H), 7.47 (d, J=9.0 Hz, 2H), 685 (d, J=9.0 Hz, 2H), 4.46 (dd, J=2.8, 6.5 Hz, 1H), 3.92 (t, J=6.6 Hz, 2H), 3.34 (d, J=2.8 Hz, 1H), 2.30-1.82 (m, 2H), 1.81-1.65 (m, 2H), 1.53-1.37 (m, 2H), 1.37-1.15 (m, 12 H), 0.87 (m, 3H) ppm. MS (ESI) m/z 323 (M+H)⁺.

### Example 19

### (2S,3S)-N-(4-(Hexyloxymethyl)phenyl)-3-hydroxypyrrolidine-2-carboxamide

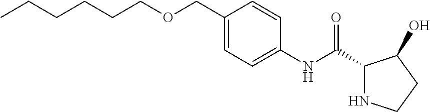

Utilizing a procedure similar to that described in Preparation A, the compound of Example 19 was prepared from 4-(hexyloxymethyl)aniline and N-(tert-butoxycarbonyl)-trans-3-hydroxy-L-proline. The product was obtained as a white solid: ¹H NMR (CDCl₃) δ 9.63 (br s, 1H), 7.56 (d, J=8.5 Hz, 2H), 7.30 (d, J=8.5 Hz, 2H), 4.72-4.59 (m, 1H), 4.46 (s, 2H), 3.74 (d, J=2.2 Hz, 1H), 3.43 (t, J=6.7 Hz, 2H), 3.37-3.28 (m, 1H), 3.13-3.04 (m, 1H), 2.40 (br s, 2H), 1.92-1.84 (m, 2H), 1.64-1.54 (m, 2H), 1.40-1.22 (m, 6H), 0.88 (t, J=6.9 Hz, 3H) ppm. MS (ESI) m/z 321 (M+H)⁺.

### Example 20

### (2S,3R,4S)-3,4-Dihydroxy-N-(4-octylphenyl)-pyrrolidine-2-carboxamide

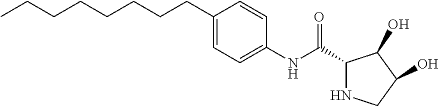

Utilizing a procedure similar to that described in Preparation A, the compound of Example 20 was prepared from 4-octylaniline and (2S,3R,4S)-1-(tert-butoxycarbonyl)-3,4-dihydroxypyrrolidine-2-carboxylic acid. The product was obtained as a white solid: ¹H NMR (CDCl₃) δ 9.47 (br s, 1H), 7.46 (d, J=8.4 Hz, 2H), 7.12 (d, J=8.4 Hz, 2H), 4.37-4.24 (m, 1H), 4.23-411 (m, 1H), 3.80 (d, J=6.3 Hz, 1H), 3.40 (br s, 2H), 3.20-3.08 (m, 1H), 3.05-2.93 (m, 1H), 1.66-1.48 (m, 2H), 1.38-1.12 (m, 9H), 0.87 (t, J=6.8 Hz, 3H) ppm. MS (ESI) m/z 335 (M+H)⁺.

### Example 21

### (2S,3S)-3-Hydroxy-N-(4-(octyloxymethyl)-phenyl)pyrrolidine-2-carboxamide

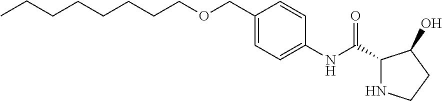

### Preparation of the intermediate 4-(octyloxymethyl)aniline

### Step 1) Preparation of 1-nitro-4-(octyloxymethyl)benzene

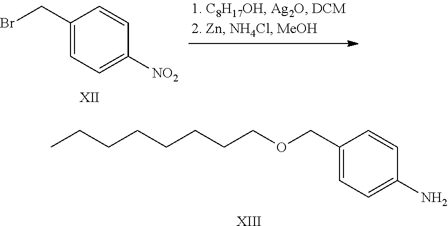

To a stirred solution of 4-nitrobenzyl bromide (XII) (4.38 g, 20.28 mmol) and 1-octanol (3.43 g, 26.37 mmol) in dichloromethane (40 mL) was added silver(II) oxide (5.17 g, 22.31 mmol). The reaction mixture was heated to reflux. After 18 hours, the reaction mixture was allowed to cool to room temperature and was filtered through Celite. The filtrate was concentrated to provide a yellow oil. Flash chromatography using an Isco Combiflash unit (120 g SiO₂ column, 5-10% ethyl acetate/hexanes) afforded 3.71 g (69%) of 1-nitro-4-(octyloxymethyl)benzene as a colorless oil: ¹H NMR (CDCl₃) δ 8.20 (d, J=8.8 Hz, 2H), 7.50 (d, J=8.8 Hz, 2H), 4.59 (s, 2H), 3.51 (t, J=6.6 Hz, 2H), 1.68-1.60 (m, 2H), 1.43-1.20 (m, 10H), 0.88 (t, J=6.9 Hz 3H) ppm.

### Step 2) Preparation of 4-(octyloxymethyl)aniline

To a stirred solution of 1-nitro-4-(octyloxymethyl)benzene (3.71 g, 13.98 mmol) and ammonium chloride (1.50 g. 27.96 mmol) in methanol (100 mL) was added zinc dust (6.63 g, 101.4 mmol). The reaction mixture was heated to reflux. After 1 hour, the reaction mixture was allowed to cool to room temperature and was filtered through Celite. The filtrate was concentrated to provide 5.89 g of a yellow semi-solid. Flash chromatography using an Isco Combiflash unit (120 g SiO₂ column, 10-20% ethyl acetate/hexanes) afforded 1.91 g (58%) of 4-(octyloxymethyl)aniline (XIII) as a yellow oil.

Utilizing a procedure similar to that described in Preparation A, the compound of Example 21 was prepared from 4-(octyloxymethyl)aniline and N-(tert-butoxycarbonyl)-trans-3-hydroxy-L-proline. The product was obtained as a white solid: ¹H NMR (CDCl₃) δ 9.66 (br s, 1H), 7.55 (d, J=8.5 Hz, 2H), 7.29 (d, J=8.5 Hz, 2H), 4.68-4.64 (m, 1H), 4.45 (s, 2H), 3.76 (d, J=2.1 Hz, 1H), 3.43 (t, J=6.7 Hz, 2H), 3.37-3.28 (m, 1H), 3.12-3.05 (m, 1H) 2.65 (br s, 2H), 1.91-1.83 (m, 2H), 1.66-1.53 (m, 2H), 1.41-1.18 (m, 10H), 0.87 (t, J=6.9 Hz, 3H) ppm. MS (ESI) m/z 349 (M+H)⁺.

### Example 22

### (2S,3R)-2-Amino-N-(4-(5-(4-fluorophenoxy)pentyl)-phenyl)-3-hydroxybutanamide

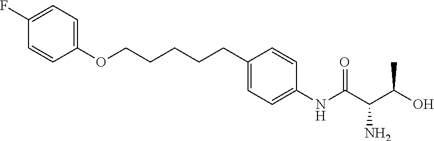

Utilizing procedures similar to that described in Example 5 the compound of Example 22 and the intermediate 4-(5-(4-fluorophenoxy)pentyl)aniline were prepared. The product was obtained as a white solid: ¹H NMR (CDCl₃) δ 9.51 (br s 1H), 7.49 (d, J=8.3 Hz, 2H), 7.14(d, J=8.3 Hz, 2H), 6.99-6.90 (m, 2H), 6.84-6.77 (m, 2H), 4.53-4.41 (m, 1H), 3.89 (t, J=6.5 Hz, 2H), 3.35 (d, J=2.8 Hz, 1H), 2.61 (t, J=7.6 Hz, 2H), 1.85-1.73 (m, 3H), 1.71-1.60 (m, 3H), 1.53-1.42 (m, 2H), 1.26 (d, J=6.5 HZ, 3H) ppm. MS (ESI) m/z 375 (M+H)⁺.

### Example 23

### (2S,3S)-N-(4-(3-(4-Fluorophenoxy)propyl)phenyl)-3-hydroxyprrolidine-2-carboxamide

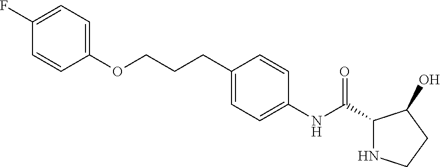

Utilizing procedures similar to that described in Example 15, the compound of Example 23 and the intermediate 4-(3-(4-fluorophenoxy)propyl)aniline were prepared. The product 23 was obtained as a white solid: ¹H NMR (CDCl₃) δ 9.63 (br s 1H), 7.49 (d, J=8.4 Hz, 2H), 7.15 (d, J=8.4 Hz, 2H), 6.95 (t, J=8.7 Hz, 2H), 6.87-6.75 (m, 2H), 4.69-4.63 (m, 1H), 3.89 (t, J=6.3 Hz, 2H), 3.77 (d, J=1.8 Hz, 1H), 3.37-3.28 (m, 1H), 3.13-3.03 (m, 1H), 3.02-2.72 (m, 4H), 2.11-1.99 (m, 2H), 1.93-1.79 (m, 2H) ppm, MS (ESI) m/z 359 (M+H)⁺.

### Example 24

### (2S,3S)-N-(4-(5-(4-Fluorophenoxy)pentyl)phenyl)-3-hydroxypyrrolidine-2-carboxamide

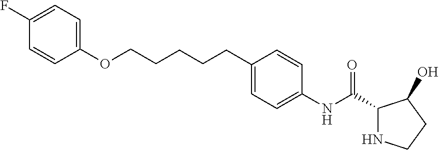

Utilizing a procedure similar to that described in Preparation A, the compound of Example 24 was prepared from 4-(5-(4-fluorophenoxy)pentyl)aniline and N-(tert-butoxycarbonyl)-trans-3-hydroxy-L-proline. The product was obtained as a white solid: ¹H NMR (CDCl₃) δ9.59 (br s, 1H), 7.48 (d, J=8.4 Hz, 2H), 7.13 (d, J=8.4 Hz, 2H), 6.95 (t, J=8.7 Hz, 2H), 6.85-6.76 (m, 2H), 4.69-4.63 (m, 1H), 3.89 (t, J=6.5 Hz, 2H), 3.75 (d, J=2.0 Hz, 1H), 3.38-126 (m, 1H), 3.13-3.04 (m, 1H), 2.70-2.50 (m, 4H), 1.92-1.83 (m, 2H), 1.82-1.73 (m, 2H), 1.71-1.60 (m, 2H), 1.53-1.41 (m, 2H) ppm. MS (ESI) m/z 387 (M+H)⁺.

### Example 25

### (2S,3S)-N-(4-(7-(4-Fluorophenoxy)heptyl)phenyl)3-hydroxypyrrolidine-2-carboxamide

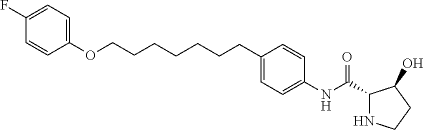

Utilizing a procedure similar to that described in Preparation A, the compound of Example 25 was prepared from 4-(7-(4-fluorophenoxy)heptyl)aniline and N-(tert-butoxycarbonyl)-trans-3-hydroxy-L-proline. The product was obtained as a white solid: ¹H NMR (CDCl₃) δ 9.59 (br s, 1H), 7.48 (d, J=8.4 Hz, 2H), 7.12 (d, J=8.4 Hz, 2H), 6.99-6.91 (m, 2H), 6.85-6.77 (m, 2H), 4.68-4.62 (m, 1H), 3.89 (t, J=6.5 Hz, 2H), 3.76 (d, J=2.1 Hz, 1H), 3.37-3.28 (m, 1H), 3.12-3.03 (m, 1H), 2.67-2.42 (m. 4H), 1.91-1.83 (m, 2H), 1.79-1.69 (m, 2H), 1.64-1.54 (m, 2H), 1.48-1.28 (m, 6H) ppm. MS (ESI) m/z 415 (M+H)⁺.

### Example 26

### (2S,3S)-3-Hydroxy-N-(6-octylpyridin-3-yl)pyrrolidine-2-carboxamide

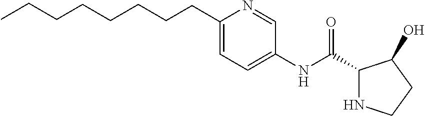

Utilizing a procedure similar to that described in Preparation A, the compound of Example 26 as prepared from 6-octylpyridin-3-amine and N-(tert-butoxycarbonyl)-trans-3-hydroxy-L-proline. The product was obtained as a white solid: ¹H NMR (CDCl₃) δ 9.73 (br s, 1H), 8.49 (d, J=2.5 Hz, 1H), 8.11 (dd, J=2.5, 8.4 Hz, 1H), 7.10 (d, J=8.4 Hz, 1H), 4.69-4.63 (m, 1H), 3.78 (d, J=1.9 Hz, 1H), 3.39-3.29 (m, 1H), 3.14-3.06 (m, 1H), 3.04-2.78 (m, 2H), 2.77-2.69 (m, 2H), 1.91-1.83 (m, 2H), 1.73-1.62 (m, 2H), 1.37-1.19 (m, 10H), 0.86 (t, J=6.9 Hz, 3H) ppm. MS (ESI) m/z 320 (M+H)⁺.

### Example 27

### (2S, 3S)-3-Hydroxy-N-((S)-1-(4-octylphenyl)ethyl)-pyrrolidine-2-carboxamide

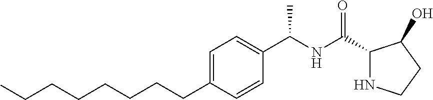

### Preparation of the Intermediate (S)-1-(4-octylphenyl)ethanamine

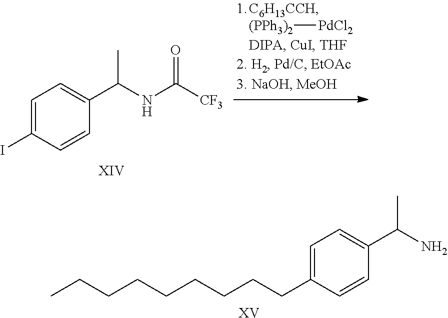

### Step 1) Preparation of (S)-2,2,2-trifluoro-N-(1-(4-oct-1-ynyl)phenyl)ethyl)ethanamide

To a stirred solution of (S)-2,2,2-trifluoro-N-(1-(4-iodophenyl)ethyl)ethanamide (XIV) (3.00 g, 8.74 mmol), 1-octyne (1.16 g, 10.49 mmol), and diisopropylamine (2.65 g, 26.23 mmol) in tetrahydrofuran (50 mL) was added copper(I) iodide (0.16 g, 0.874 mmol) and bis(triphenylphosphine)palladium(II) chloride (0.307 g, 0.437 mmol). The reaction mixture was allowed to stir at room temperature. After 65 hours, the reaction mixture was diluted with ethyl acetate and washed with 1N hydrochloric acid, 6N ammonium hydroxide, and brine. The organic phase was dried (magnesium sulfate), filtered, and concentrated to provide 3.41 g of a brown oil. Flash chromatography using an Isco Combiflash unit (120 g SiO₂ column, 15-30% ethyl acetate/hexanes) afforded 2.22 g (78%) of (S)-2,2,2-trifluoro-N-(1-(4-oct-1-ynyl)phenyl)ethyl)ethanamide as a white solid: ¹H NMR (CDCl₃) δ 7.39 (d, J=8.3 Hz, 2H), 7.23(d, J=8.3 Hz, 2H), 6.47-6.35 (m, 1H), 5.16-5.08 (m, 1H), 2.40(t, J=7.1 Hz, 2H), 1.64-1.54 (m, 5H), 1.49-1.38 (m, 2H), 1.37-1.26 (m, 4H), 0.90 (t, J=7.0 Hz, 3H) ppm.

### Step 2) Preparation of (S)-2,2,2-trifluoro-N-(1-(4-octylphenyl)ethyl)ethanamide

To a stirred solution of (S)-2,2,2-trifluoro-N-(1-(4-oct-1-ynyl)phenyl)ethyl)ethanamide (2.22 g, 6.82 mmol) in ethyl acetate (75 mL) was added 10% palladium on carbon (wet) (0.600 g). The reaction mixture was degassed under vacuum (ca. 30 mm Hg) and backfilled with nitrogen three times. After an additional evacuation, the atmosphere was replaced with hydrogen, and the reaction mixture allowed to stir at room temperature. After 18 hours, an additional portion of the catalyst (˜0.500 g) was added. After 21 hours, the remaining hydrogen was removed under vacuum and replaced with nitrogen. The reaction mixture was filtered through Celite, and the filtrate concentrated to provide 2.25 g (100%) of (S)-2,2,2-trifluoro-N-(1-(4-octylphenyl)ethyl)ethanamide as a white solid: ¹H NMR (CDCl₃) δ 7.25-7.16 (m, 4H), 6.54-6.36 (m, 1H), 5.17-5.07 (m, 1H), 2.63-2.56 (m, 2H), 1.64-1.54 (m, 5H), 1.36-1.21 (m, 10H), 0.88 (t, J=6.9 Hz, 3H) ppm.

### Step 3) Preparation of (S)-1-(4-octylphenyl)ethanamine (XV)

To a stirred solution of (S)-2,2,2-trifluoro-N-(1-(4-octylphenyl)ethyl)ethanamide (2.25 g, 6.83 mmol) in methanol (15 mL) was added 3N sodium hydroxide (23 mL). The reaction mixture was allowed to stir at room temperature. After 2 hours, the cloudy mixture was diluted with water and extracted three times with diethyl ether. The combined organic phases were dried (magnesium sulfate), filtered, and concentrated to provide 1.55 g of (S)-1-(4-octylphenyl)ethanamine (XV) as a yellow oil: ¹H NMR. (CDCl₃) δ 7.25 (d, J=8.1 Hz, 2H), 7.14 (d, J=8.1 Hz, 2H), 4.09 (q, J=6.6 Hz, 1H), 2.61-2.54 (m, 2H), 1.64-1.55 (m, 2H), 1.49 (br s, 2H), 1.40-1.20 (m, 13H), 0.88 (t, J=6.9 Hz, 3H) ppm.

Utilizing a procedure similar to that described in Preparation A, the compound of Example 27 was prepared from (S)-1-(4-octylphenyl)ethanamine and N-(tert-butoxycarbonyl)-trans-3-hydroxy-L-proline. The product was obtained as a white solid: ¹H NMR (CDCl₃) δ 7.87 (br d, J=8.7 Hz, 1H), 7.20 (d, J=8.1 Hz, 2H), 7.13 (d, J=8.1 Hz, 2H), 5.08-4.99 (m, 1H), 4.56-4.52 (m, 1H), 3.59 (d, J=2.3 Hz, 1H), 3.28-3.18 (m, 1H), 3.02-2.94 (m, 1H), 2.60-2.53 (m, 2H), 1.86-1.78 (m, 2H), 1.63-1.53 (m, 2H), 1.44 (d, J=6.9 Hz, 3H), 1.37-1.20 (m, 10H), 0.87 (t, J=6.9 Hz, 3H) ppm. MS (ESI) m/z 347 (M+H)⁺.

### Example 28

### (2S,3S)—N-(4-(5-(4-Fluorophenoxy)pentyl)benzyl)-3-hydroxypyrrolidine-2-carboxamide

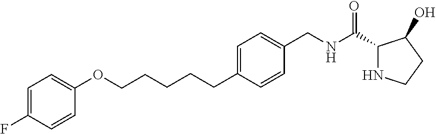

### Preparation of the Intermediate 4-(5-(4-fluorophenoxy)pentyl)phenyl)-methanamine

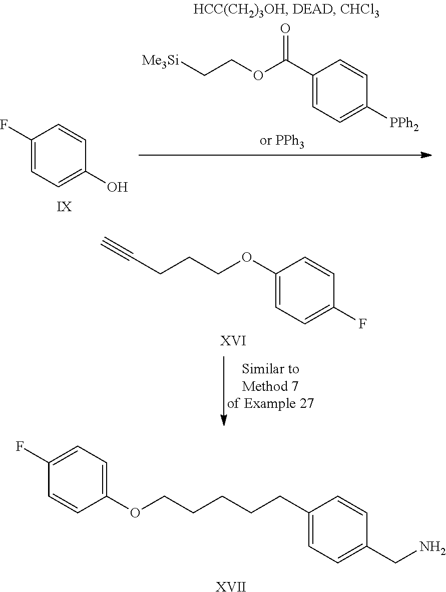

### Step 1) Preparation of 1-fluoro-4-(pent-4-ynyloxy)benzene (XVI)

To a stirred solution of 4-fluorophenol (4.80 g, 42.80 mmol), pent-4-yn-1-ol (IX) (100 g, 35.66 mmol), 2-(trimethylsilyl)ethyl 4-(diphenylphosphino)benzoate (0.5 M in tetrahydrofuran, 93 mL, 46.36 mmol) in tetrahydrofuran (25 mL) was added diisopropyl azodicarboxylate (9.37 g, 46.36 mmol) dropwise over ten minutes. The reaction mixture was allowed to stir at room temperature. After 2 hours, the reaction mixture was treated with 1.0 M tetrabutylammonium fluoride in tetrahydrofuran (100 mL, 100.0 mmol), and the reaction allowed to stir. After 1 hour, the reaction mixture was concentrated, and the residue dissolved in diethyl ether. The solution was washed with 1N sodium hydroxide, 1N hydrochloric acid, and brine. The organic phase was dried (magnesium sulfate), filtered, and concentrated to provide 17.25 of a yellow solid. Flash chromatography using an Isco Combiflash unit (330 g SiO₂ column, 1-5% ethyl acetate/hexanes) afforded 4.08 g (64%) of 1-fluoro-4-(pent-4-ynyloxy)benzene (XVI) as a colorless oil: ¹H NMR (CDCl₃) δ 7.00-6.93 (m, 2H), 6.87-6.80 (m, 2H), 4.03 (t, J=6.1 Hz, 2H), 2.40 (dt, J=2.6, 7.0 Hz, 2H), 2.04-1.93 (m, 3H) ppm.

### Step 2) Preparation of 2,2,2-trifluoro-N-(4-(5-(4-fluorophenoxy)pent-1-ynyl)benzyl)ethanamide

To a stirred solution of 2,2,2-trifluoro-N-(4-iodobenzyl)ethanamide (2.36 g, 7.16 mmol), 1-fluoro-4-(pent-4-ynyloxy)benzene (1.34 g, 7.52 mmol), and diisopropylamine (2.17 g, 21.48 mmol) in tetrahydrofuran (50 mL) was added copper(I) iodide (0.136 g, 0.716 mmol) and bis(triphenylphosphine)palladium(II) chloride (0.251 g, 0.358 mmol). The reaction mixture was allowed to stir at room temperature. After 1.5 hours, the reaction mixture was diluted with ethyl acetate and washed with 1N hydrochloric acid, 6N ammonium hydroxide, and brine. The organic phase was dried (magnesium sulfate), filtered, and concentrated to provide 4.69 g of an orange solid. Flash chromatography using an Isco Combiflash unit (330 g SiO₂ column, 10-30% ethyl acetate/hexanes) afforded 2.09 g (77%) of 2,2,2-trifluoro-N-(4-(5-(4-fluorophenoxy)pent-1-ynyl)benzyl)ethanamide as an off-white solid: ¹H NMR (CDCl₃) δ 7.38 (d, J=8.2 Hz, 2H), 7.20 (d, J=8.2 Hz, 2H), 7.01-6.93 (m, 2H), 6.89-6.81 (m, 2H), 6.52 (br s, 1H), 4.51 (d, J=5.9 Hz, 2H), 4.07 (t, J=6.1 Hz, 2H), 2.62 (t, J=7.0 Hz, 2H), 2.11-2.02 (m, 2H) ppm.

### Step 3a) Preparation of 2,2,2-trifluoro-N-(4-(5-(4-fluorophenoxy)pentyl)benzyl)ethanamide

To a stirred solution of 2,2,2-trifluoro-N-(4-(5-(4-fluorophenoxy)pent-1-ynyl)benzyl)ethanamide (2.09 g, 5.51 mmol) in ethyl acetate (100 mL) was added 10% palladium on carbon (wet) (1.20 g). The reaction mixture was degassed under vacuum (ca. 30 mm Hg) and backfilled with nitrogen three times. After an additional evacuation, the atmosphere was replaced with hydrogen, and the reaction mixture allowed to stir at room temperature. After 21 hours, the remaining hydrogen was removed under vacuum and replaced with nitrogen. The reaction mixture was filtered through Celite, and the filtrate concentrated to provide 1.78 g (84%) of 2,2,2-trifluoro-N-(4-(5-(4-fluorophenoxy)pentyl)benzyl)ethanamide as a white solid: ¹H NMR (CDCl₃) δ 7.23-7.17 (m, 4H), 6.99-6.92 (m, 2H), 6.85-6.78 (m, 2H), 6.49 (br s, 1H), 4.50 (d, J=5.7 Hz, 2H), 3.91 (t, J=6.5 Hz, 2H), 2.68-2.61 (m, 2H), 1.85-1.74 (m, 2H), 1.73-1.62 (m, 2H), 1.57-1.44 (m, 2H) ppm.

### Step 3b) Preparation of (4-(5-(4-fluorophenoxy)pentyl)phenyl)methanamine

To a stirred solution of 2,2,2-trifluoro-N-(4-(5-(4-fluorophenoxy)pentyl)benzyl)ethanamide (1.78 g, 4.64 mmol) in methanol (25 ml) was added 3N sodium hydroxide (23 mL). The reaction mixture was allowed to stir at room temperature. After 2 hours, the cloudy mixture was diluted with water and extracted three times with diethyl ether. The combined organic phases were dried (magnesium sulfate), filtered, and concentrated to provide 1.55 g of (4-(5-(4-fluorophenoxy)pentyl)phenyl)methanamine (XVII) as a yellow oil: ¹H NMR (CDCl₃) δ 7.29-7.20 (m, 2H), 7.18-7.11 (m, 2H), 7.00-6.91 (m, 2H), 6.85-6.77 (m, 2H), 3.90 (t, J=6.4 Hz, 2H), 3.84 (s, 2H), 2.63 (t, J=7.5 Hz, 2H), 1.84-1.74 (m, 2H), 1.73-1.62 (m, 2H), 1.55-1.37 (m, 4H) ppm.

Utilizing a procedure similar to that described in Preparation A, the compound of Example 28 was prepared from (4-(5-(4-fluorophenoxy)pentyl)phenyl)methanamine and N-(tert-butoxycarbonyl)-trans-3-hydroxy-L-proline. The product was obtained as a white solid: ¹H NMR (CDCl₃) δ 7.94-7.86 (m, 1H), 7.18-7.11 (m, 4H), 6.99-6.91 (m, 2H), 6.84-6.77 (m, 2H), 4.61-4.54 (m, 1H), 4.38 (d, J=6.0 Hz, 2H), 3.90 (t, J=6.5 Hz, 2H), 3.70-3.64 (m, 1H), 3.29-3.18 (m, 1H), 3.01-2.91 (m, 1H), 2.66-2.58 (m, 2H), 2.54-2.15 (m, 2H), 1.86-1.74 (m, 4H), 1.72-1.61 (m, 2H), 1.54-1.43 (m, 2H) ppm. MS (ESI) m/z 401 (M+H)⁺.

### Example 29

### (2S,3R)-2-Amino-3-hydroxy-N-(4-octylphenyl)butanamide

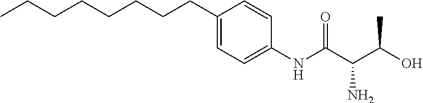

Utilizing a procedure similar to that described in Preparation A, the compound of Example 29 was prepared from 4-octylaniline and N-(tert-butoxycarbonyl)-L-threonine. The product was obtained as a white solid: ¹H NMR (DMSO-d₆) δ 9.84 (br s, 1H), 7.55-7.50 (m, 2H), 7.12-7.08 (m, 2H), 4.75 (m, 1H), 3.96-3.87 (m, 1H), 1.59-1.47 (m, 3H), 1.32-1.18 (m, 13H), 1.14-1.08 (m, 4H), 0.85 (t, J=6.8 Hz, 3H) ppm. MS (ESI) m/z 307 (M+H)⁺.

### Example 30

### (2R,3S)-2-Amino-3-hydroxy-N-(4-octylphenyl)butanamide

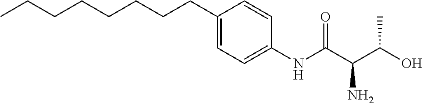

Utilizing a procedure similar to that described in Preparation A, the compound of Example 30 was prepared from 4-octylaniline and N-(tert-butoxycarbonyl)-D-threonine. The product was obtained as a white solid: ¹H NMR (DMSO-d₆) δ 9.87 (br s, 1H), 7.52 (d, J=8.4 Hz, 2H), 7.10 (d, J=8.4 Hz, 2H), 4.75-4.66 (m, 1H), 3.96-3.88 (m, 1H), 3.10 (d, J=4.1 Hz, 1H), 2.53-2.48 (m, 2H (obscured by residual DMSO)), 2.33 (br s, 2H), 1.58-1.47 (m, 2H), 1.31-1.19 (m, 10H), 1.10 (d, J=6.3 Hz, 3H), 0.85 (t, J=6.8 Hz, 3H) ppm. MS (ESI) m/z 307 (M+H)⁺.

### Example 31

### (2R,3R)-2-Amino-3-hydroxy-N-(4-octylphenyl)butanamide

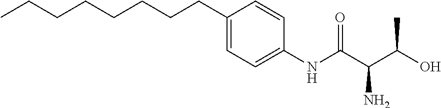

Utilizing a procedure similar to that described in Preparation A, the compound of Example 31 was prepared from 4-octylaniline and N-(tert-butoxycarbonyl)-D-allo-threonine. The product was obtained as a white solid: ¹H NMR (DMSO-d₆) δ 9.87 (br s, 1H), 7.52 (d, J=8.5 Hz, 2H), 7.09 (d, J=8.5 Hz, 2H), 4.78-4.72 (m, 1H), 3.88-3.79 (m, 1H), 3.28 (d, J=5.6 Hz, 1H), 2.53-2.48 (m, 2H (obscured by residual DMSO)), 2.09 (br s, 2H), 1.58-1.47 (m, 2H), 1.31-1.18 (m, 10H), 1.04 (d, J=6.3 Hz, 3H), 0.85 (t, J=6.9 Hz, 3H) ppm. MS (ESI) m/z 307 (M+H)⁺.

### Example 32

### (2S,3S)-2-Amino-3-hydroxy-N-(4-octylphenyl)butanamide

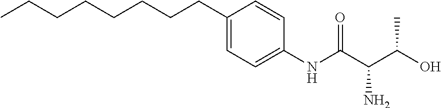

Utilizing a procedure similar to that described in Preparation A, the compound of Example 32 was prepared from 4-octylaniline and N-(tert-butoxycarbonyl)-D-allo-threonine. The product was obtained as a white solid: ¹H NMR (DMSO-d₆) δ 9.71 (br s, 1H), 7.52 (d, J=8.4 Hz, 2H), 7.09 (d, J=8.4 Hz, 2H), 4.78-4.71 (m, 1H), 3.88-3.79 (m, 1H), 3.28 (d, J=5.6 Hz, 1H), 2.53-2.48 (m, 2H (obscured by residual DMSO)), 2.02 (br s, 2H), 1.57-1.47 (m, 2H), 1.31-1.19 (m, 10H), 1.04 (d, J=6.3 Hz, 3H), 0.85 (t, J=6.9 Hz, 3H) ppm. MS (ESI) m/z 307 (M+H)⁺.

### Example 33

### (2S,3R)-2-amino-3-hydroxy-N-(4-(6-oxodecyl)phenyl)butanamide

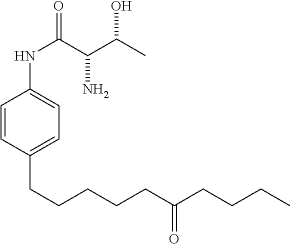

### Preparation of (2S,3R)-2-amino-3-hydroxy-N-(4-(6-oxodecyl)phenyl)butanamide

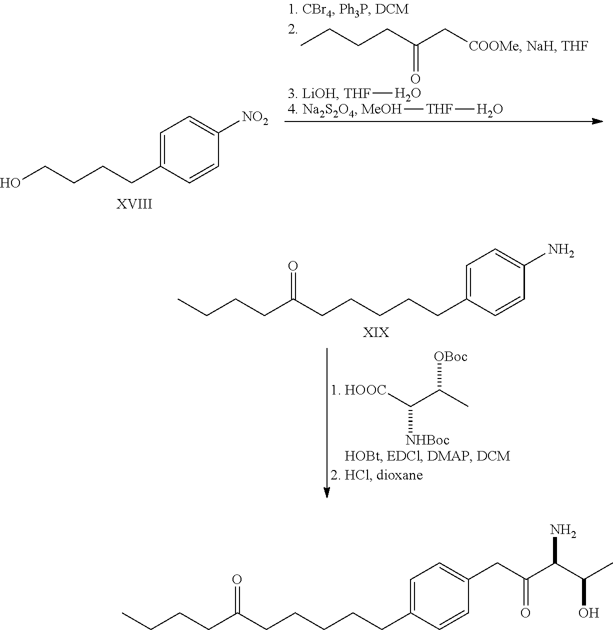

### Step 1) 1-(4-bromobutyl)-4-nitrobenzene

To a solution of 4-(4-nitrophenyl)butan-1-ol (XVIII) (5 g, 25.6 mmol) in CH₂Cl₂ was added PPh₃, followed by CBr₄ at room temperature. The mixture was stirred for 1 hour. To the mixture was added 40 g of silica gel, the solvent was evaporated, the residue was chromatographed by Combiflash using 0-20% EtOAc in hexanes to give 6.0 g (92%) of 1-(4-bromobutyl)-4-nitrobenzene as a colorless oil. ¹H NMR (CDCl₃): δ 8.15(d, J=8.7 Hz, 2H), 7.33(d, J=8.7 Hz, 2H), 3.43(t, J=8.7 Hz, 2H), 3.68 (t, J=6.3 Hz, 2H), 2.76 (t, J=7.4 Hz, 2H), 2.00-1.76(m, 4H), EIMS m/z 257 (M+), 259 (M+).

### Step 2) 10-(4-nitrophenyl)decan-5-one

To a suspension of NaH (60% oil suspention, 0.24 g, 6.0 mmol) in THF (10 mL) was added methyl 3-oxoheptanoate (0.95 g, 6.0 mmol) at 0° C. dropwise. The mixture was stirred for another 30 minutes. Then 1-(4-bromobutyl)-4-nitrobenzene was added. The mixture was stirred at 0° C. for 2 hours, then heated at 40° C. for 3 days. The mixture was diluted with 200 mL of EtOAc and washed with saturated NH₄Cl (100 mL), water, brine, dried over Na₂SO₄. After removal of solvent the residue was chromatographed with Combiflash (0-20% EtOAc in Hexanes) to give 1.2 g of desired pure ester, which was then dissolved in 5 mL of THF/5 mL of MeOH/10 mL of H₂O and added 0.24 g (6.0 mmol) of LiOH.H₂O. After stirring overnight, the starting material was completely gone, then 10 mL of 1M HCl was added, heated to refluxing. After 2 hours, all the acid was gone by TLC, the THF and MeOH was evaporated. The residue was diluted with 150 mL of EtOAc, washed with water, brine, dried over Na₂SO₄. After removal of solvent, a reddish liquid (1.0 g) was obtained. This crude product, 10-(4-nitrophenyl)decan-5-one, was directly used without further purification. ¹H NMR (CDCl₃): 8.10-8.05(m, 2H), 7.30-7.25(m, 2H), 2.70-2.62(m, 2H), 2.38-2.08(m, 4H), 1.62-1.42(m, 6H), 1.32-1.18(m, 4H), 0.89-0.80(m, 3H).

### Step 3) 10-(4-aminophenyl)decan-5-one (XIX)

To the above reddish oil, 10-(4-nitrophenyl)decan-5-one (1.0 g), was added 10 mL of THF, 10 mL of MeOH and 10 mL of H₂O, then Na₂S₂O₄ (1.98 g, 11.4 mmol). The mixture was stirred at 50° C. for 30 minutes until all the starting material was disappeared by TLC (20% EtOAc in Hexanes). The THE and MeOH was evaporated, the residue aqueous layer was extracted with EtOAc (100 mL), washed with water, brine, dried over Na₂SO₄. After removal of solvent the residue was chromatographed by CombiFlash with gradiant (XIX) of 0-40% EtOAc Hexanes to give yellowish oil, 10-(4-aminophenyl)decan-5-one (0.63 g, 54% for two steps). ¹H NMR(CDCl₃): 6.90-6.85(m, 2H), 6.58-6.53(m, 2H), 3.50-3.40(bs, 2H), 2.45-2.40(m, 2H), 2.35-2.25(m, 4H), 1.58-1.42(m, 6H), 1.30-1.18(m, 4H), 0.90-0.80(m, 3H).

### Step 4) tert-butyl(2S,3R)-3-hydroxy-1-oxo-1-(4-(6-oxodecyl)phenylamino)butan-2-ylcarbamate

To the above aniline (0.10 g, 0.4 mmol) in CH₂Cl₂ (2 mL) was added Boc protected L-threonine (0.11 g, 0.48 mmol), EDC (0.11 g, 0.60 mmol), DMAP (0.07 g, 0.60 mmol), HOBT (0.08 g, 0.60 mmol). The mixture was stirred at room temperature overnight. To the mixture was added 1 g of silica gel and the solvent was evaporated. The residue was loaded on CombiFlash to give 0.19 g of yellowish foam, tert-butyl(2S,3R)-3-hydroxy-1-oxo-1-(4-(6-oxodecyl)phenylamino)butan-2-ylcarbamate. ¹H NMR(CD₃OD): 7.52-7.46(m, 2H), 7.20-7.14(m, 2H), 4.25-4.10(m, 2H), 2.70-2.60(m, 2H), 2.53-2.42(m, 4H), 1.73-1.45(m, 15H), 1.40-1.25(m, 7H), 1.00-0.90(m, 3H).

The compound of Example 33 was made by adding to the above yellowish foam (0.19 g, 0.4 mmol) 1 mL of 4N HCl in 1,4-dioxane. The mixture was stirred at room temperature overnight. The solvent was evaporated and to the residue was added 2N ammonia in methanol. The solvent was evaporated and to the residue was added 1 g of silica gel, 2 mL of CH₂Cl₂. The solvent was evaporated again and the residue was chromatographed by CombiFlash on a 12 g column using 0-10% methanol in methylene chloride to give 0.09 g of desired product, (2S,3R)-2-amino-3-hydroxy-N-(4-(6-oxodecyl)phenyl)butanamide. ¹H NMR (CDCl₃): 9.40(bs, 1H), 7.42-7.38(m, 2H), 7.10-7.06(m, 2H), 4.42-4.35(m, 1H), 3.30-3.26(m, 1H), 2.52-2.43(m, 2H), 2.35-2.25(m, 4H), 1.60-1.40(m, 6H), 1.30-1.18(m, 7H), 0.90-0.80(m, 3H).

### Example 34

### (2S,3R)-2-amino-3 -hydroxy-N-(4-(4-oxodecyl)phenyl)butanamide

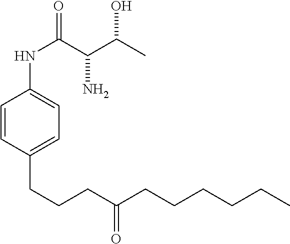

Utilizing a procedure similar to that described in Example 33, the compound of Example 34 was prepared from tert-butyl 4-(4-hydroxybutyl)phenylcarbamate and (2S,3R)-2-(tert-butoxycarbonylamino)-3-hydroxybutanoic acid (BOC protected L-threonine). Product was afforded as a pale yellow foam, ¹HNMR (CD₃OD): 7.55-7.50(m, 2H), 7.20-7.15(m, 2H), 4.10-4.02(m, 1H), 3.30-3.27(m, 1H), 2.61-2.57(m, 2H), 2.50-2.38(, 4H), 1.90-1.80(m, 2H), 1.40-1.20(m, 7H), 0.95-0.90(m, 3H).

### Example 35

### (2S,3R)-2-amino-3-hydroxy-N-(4-(4-hydroxydecyl)phenyl)butanamide

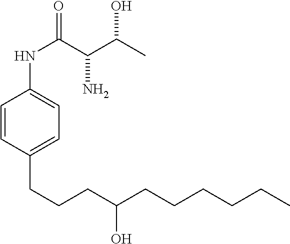

Utilizing a procedure similar to that described in Example 33, the compound of Example 35 was prepared from tert-butyl 4-(4-hydroxybutyl)phenylcarbamate and L-threonine. Product was afforded as colorless solid, ¹HNMR (CD₃OD): 7.50-7.45(m, 2H), 7.15-7.10(m, 2H), 4.05-3.99(m, 1H), 3.53-3.45(m, 1H), 2.60-2.50(m, 2H), 1.78-1.50(m, 2H), 1.42-1.19(m, 16H), 0.90-0.82(m, 3H).

### Example 36

### (2S,3S)-3-hydroxy-N-(4-octylbenzyl)pyrrolidine-2-carboxamide

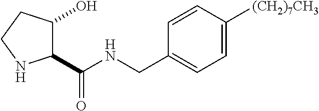

### Preparation of the Intermediate (4-octylphenyl)methanamine

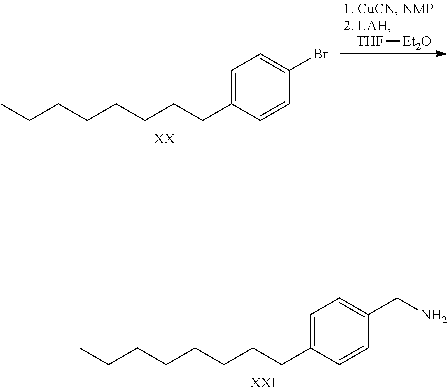

### Step 1) 4-octylbenzonitrile

To a solution of 1-bromo-4-ootylbenzene (XX) (2.69 g, 10.0 mmol) in NMP (20 mL) was added CuCN (1.34 g, 15 mmol) and the mixture was heated to reflux for 2 hours. The mixture was cooled to rt, diluted with 200 mL of EtOAc and hexanes (1:1). The resulting mixture was washed with water (three times), brine, dried over sodium sulfate. After removal of solvent the residue was chromatographed with CombiFlash (0-20% EtOAc in hexanes) to give 4-octylbenzonitrile as a colorless solid (2.1 g, 99%). ¹NNMR (CDCl₃): 7.60-7.55(m, 2H), 7.32-7.25(m, 2H), 2.72-2.65(m, 2H), 1.70-1.68(m, 2H), 1.40-1.20(m, 10H), 0.95-0.87(m, 3H).

### Step 2) (4-octylphenyl)methanamine (XXI)

To a suspension of LAH (1.06 g, 27.9 mmol) in THF:Et₂O (15 mL, 1:10) was added dropwise of 4-octylbenzonitrile (2.0 g, 9.3 mmol) in ether (10 mL) at room temperature. After the addition the mixture was stirred for another 30 min, then slowly quenched with 15% NaOH (50 mL). The mixture was extracted with EtOAc (30 mL×5). The combined organic layers were washed with water, brine, dried over Na₂SO₄. After removal of solvent the residue, (4-octylphenyl)methanamine (XXI) was directly used for next step without further purification. ¹HNMR (CDCl₃): 7.24-7.20(m, 2H), 7.18-7.14(m, 2H), 3.80(s, 2H), 2.62-2.58(m, 2H), 1.62-1.55(m, 2H), 1.40-1.20(m, 10H), 0.95-0.87(m, 3H).

Utilizing a procedure similar to that described in Preparation A, the compound of Example 36 was prepared from (4-octylphenyl)methanamine (0.131 g, 0.6 mmol) and (2S,3S)-1-(tert-butoxycarbonyl)-3-hydroxypyrrolidine-2-carboxylic acid (0.115 g, 0.5 mmol). Product was afforded as a colorless solid, ¹HNMR (CD₃OD): 7.20-7.12(m, 4H), 4.39-4.35(m, 3H), 3.56(s, 1H), 3.20-3.03(m, 2H), 2.62-2.57(m, 2H), 1.88-1.78(m, 2H), 1.63-1.58(m, 2H), 1.39-1.25(m, 10H), 0.95-0.90(m, 3H).

### Example 37

### (2S,3S)—N-(4-heptylbenzyl)-3-hydroxypyrrolidine-2-carboxamide

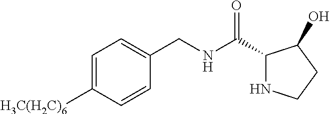

Utilizing a procedure similar to that described in Preparation A, the compound of Example 37 was prepared from (4-heptylphenyl)methanamine (0.122 g, 0.6 mmol) and (2S,3S)-1-(tert-butoxycarbonyl)-3-hydroxypyrrolidine-2-carboxylic acid (0.115 g, 0.5 mmol). Product was afforded as a colorless solid. ¹HNMR (CD₃OD): 7.25-7.15(m, 4H), 4.48(m, 1H), 4.40(s, 2H), 3.89(s, 1H), 3.38-3.30(m, 2H), 2.68-2.59(m, 2H), 2.00-1.93(m, 2H), 1.70-1.60(m, 2H), 1.40-1.28(m, 8H), 0.97-0.90(m, 3H).

### Example 38

### (2S,3S)-3-hydroxy-N-(4-(6-oxodecyl)phenyl)pyrrolidine-2-carboxamide

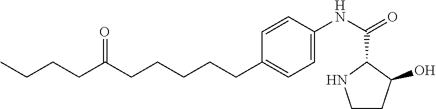

Utilizing a procedure similar to that described in Preparation A, the compound of Example 38 was prepared from 10-(4-aminophenyl)decan-5-one (0.060 g, 0.25 mmol) and (2S,3S)-1-(tert-butoxycarbonyl)-3-hydroxypyrrolidine-2-carboxylic acid (0.058 g, 0.25 mmol). Product was afforded as a colorless solid. ¹HNMR (CD₃OD): 7.52-7.48(m, 2H), 7.20-7.15(m, 2H), 4.50-4.45(m, 1H), 3.68-3.64(m, 1H), 3.23-3.15(m, 2H), 2.63-2.58(m, 2H), 2.52-2.45(m, 4H), 1.92-1.85(m, 2H), 1.70-1.50(m, 6H), 1.40-1.29(m, 4H), 0.99-0.91(m, 3H).

### Example 39

### (2S,3S)—N-(4-(hexylthiomethyl)phenyl)-3-hydroxypyrrolidine-2-carboxamide

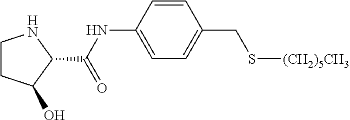

### Preparation of the Intermediate, 4-(hexylthiomethyl)aniline

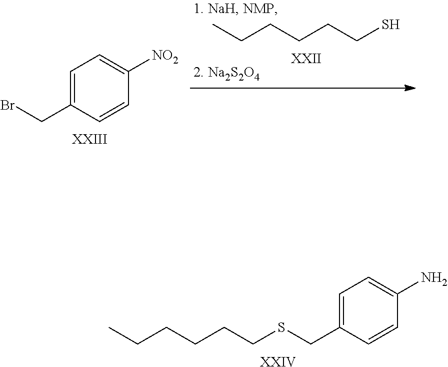

### Step 1) hexyl-(4-nitrobenzyl)-sulfane

To a solution of hexane-1-thiol (XXII) (6.0 mmol, 0.71 g) in 5 mL of NMP was added NaH (60%, 0.24 g, 6.0 mmol). After the evolution of H2 was stopped, 1-(bromomethyl)-4-nitrobenzene (XXIII) (1.08 g, 5.0 mmol) was added in one portion. The solution quickly turned to deep reddish, then yellowish. The mixture was stirred overnight, then diluted with 200 mL of 1:1 EtOAc:Hexanes, washed with water, brine, dried over Na₂SO₄. After removal of solvent the residue was chromatographed by CombiFlash using 0-20% EtOAc in hexanes to give hexyl-(4-nitrobenzyl)-sulfane (1.0 g, 80%) as pale yellow liquid. ¹HNMR (CDCl₃): 8.22-18(m, 2H), 7.52-7.48(m, 2H), 3.79 (s, 2H), 2.44-2.38(m, 2H), 1.60-1.50(m, 2H), 1.40-1.20(m, 6H), 0.90-0.84(m, 3H).

### Step 2) 4-(hexylthiomethyl)aniline (XXIV)

To a solution of hexyl-(4-nitrobenzyl)-sulfane (1.0 g, 4.0 mmol) THF (10 mL), water (10 mL), MeOH (3 mL) was added Na₂S₂O₄ (2.1 g, 12.0 mmol). The mixture was heated to 50° C. for 1 hour. THF and MeOH was evaporated and the aqueous layer was extracted with EtOAc (50 mL×4). The combined organic layers were washed with water, brine, and dried over Na₂SO₄. After removal of solvent the residue was chromatographed with 0-30% EtOAc in hexanes by Combiflash to give 0.31 g of 4-(hexylthiomethyl)aniline (XXIV) as colorless oil. ¹HNMR (CDCl₃): 7.15-7.08(m, 2H), 6.64-6.60(m, 2H), 3.70-3.55 (m, 4H), 2.42-2.35(m, 2H), 1.60-1.50(m, 2H), 1.40-1.20(m, 6H), 0.95-0.87(m, 3H).

Utilizing a procedure similar to that described, in Preparation A, the compound of Example 39 was prepared from 4-(hexylthiomethyl)aniline (0.060 g, 0.25 mmol) and (2S,3S)-1-(tert-butoxycarbonyl)-3-hydroxypyrrolidine-2-carboxylic acid (0.058 g, 0.25 mmol). Product was afforded as a colorless solid. ¹HNMR (CD₃OD): 7.60-7.55(m, 2H), 7.36-7.31(m, 2H), 4.50-4.47(m, 1H), 3.73-3.69(m, 3H), 3.28-3.17(m, 2H), 2.45-2.39(m, 2H), 1.98-1.85(m, 2H), 1.60-1.55(m, 2H), 1.40-1.24(m, 6H), 0.96-0.90(m, 3H).

### Example 40

### (2S,3S)—N-(4-(2-(hexylthio)ethyl)phenyl)-3-hydroxypyrrolidine-2-carboxamide

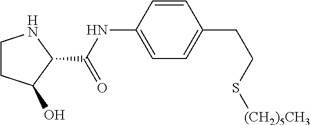

Utilizing a procedure similar to that described in Preparation A, the compound of Example 40 was prepared from 4-(2-(hexylthio)ethyl)aniline (0.062 g, 0.25 mmol) and (2S,3S)-1-(tert-butoxycarbonyl)-3-hydroxypyrrolidine-2-carboxylic acid (0.058 g, 0.25 mmol). The final product was afforded as a colorless solid. ¹HNMR (CD₃OD): 7.52-7.47(m, 2H), 7.22-7.17(m, 2H), 4.42-4.40(m, 1H), 3.63-3.60(m, 1H), 3.22-3.10(m, 2H), 2.90-2.80(m, 2H), 2.78-2.70(m, 2H), 2.56-2.47(m, 2H), 1.92-1.80(m, 2H), 1.60-1.55(m, 2H), 1.42-1.24(m, 6H), 0.96-0.90(m, 3H).

The intermediate, 4-(2-(hexylthio)ethyl)aniline, was prepared using similar method as 4-(hexylthiomethyl)aniline described in Example 39.

Hexyl(4-nitrophenethyl)sulfane, ¹HNMR (CDCl₃): 8.22-8.18(m, 2H), 7.40-7.38(m, 2H), 3.05-2.98 (m, 2H), 2.85-2.78(m, 2H), 2.45-2.38(m, 2H), 1.65-1.55(m, 2H), 1.40-1.20(m, 6H), 0.90-0.84(m, 3H).

4-(2-(hexylthio)ethyl)aniline. ¹HNMR (CDCl₃): 7.02-6.97(m, 2H), 6.65-6.61(m, 2H), 3.65-3.55 (b, 2H), 2.80-2.70(m, 4H), 2.55-2.45(m, 2H), 1.62-1.55(m, 2H), 1.40-1.20(m, 6H), 0.95-0.89(m, 3H).

### Example 41

### (2S,3S)—N-(4-(4-(hexylthio)butyl)phenyl)-3-hydroxypyrrolidine-2-carboxamide

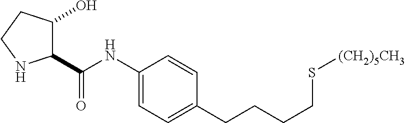

Utilizing, a procedure similar to that described in Preparation A, the compound of Example 41 was prepared from 4-(4-(hexylthio)butyl)aniline (0.27 g, 1.0 mmol) and (2S,3S)-1-(tert-butoxycarbonyl)-3-hydroxypyrrolidine-2-carboxylic acid (0.23 g, 1.0 mmol). Product was afforded as a colorless solid. ¹HNMR (CD₃OD): 7.52-7.47(m, 2H), 7.20-7.15(m, 2H) 4.45-4.42(m, 1H), 3.63-3.60(m, 1H), 3.22-3.10(m, 2H) 2.64-2.42(m, 6H), 1.92-1.80(m, 2H), 1.78-1.68(m, 2H), 1.63-1.50(m, 4H), 1.42-1.24(m, 6H), 0.96-0.90(m, 3H).

The intermediate, 4-(4-(hexylthio)butyl)aniline, was prepared using similar method as 4-(hexylthiomethyl)aniline described in Example 39.

hexyl(4-(4-nitrophenyl)butyl)sulfane. ¹HNMR (CDCl₃): 8.20-8.16(m, 2H), 7.38-7.34(m, 2H), 2.80-2.72 (m, 2H), 2.60-2.47(m, 2H), 1.82-1.75(m, 2H), 1.70-1.50(m, 4H), 1.42-1.22(m, 6H), 0.95-0.88(m, 3H).

4-(4-(hexylthio)butyl)aniline. ¹HNMR (CDCl₃): 7.00-7.95(m, 2H), 7.68-7.63(m, 2H), 3.70-3.30(b, 2H), 2.60-2.47 (m, 6H), 1.82-1.75(m, 2H), 1.72-1.55(m, 6H), 1.42-1.22(m, 6H), 0.95-0.88(m, 3H).

### Example 42

### (2S,3S)-3-hydroxy-N-(4-(4-hydroxyundecyl)phenyl)pyrrolidine-2-carboxamide

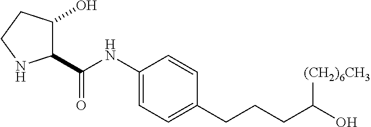

Utilizing a procedure similar to that described in Preparation A, the compound of Example 42 was prepared from 1-(4-aminophenyl)decan-4-ol (0.125 g, 0.5 mmol) and (2S,3S)-1-(tert-butoxycarbonyl)-3-hydroxypyrrolidine-2-carboxylic acid (0.115 g, 0.5 mmol). Product was afforded as a colorless solid. ¹HNMR (CD₃OD): 7.52-7.45 m, 2H), 7.20-7.13(m, 2H), 4.50-4.45(m, 1H), 3.78-3.72(m, 1H), 3.58-3.50 (m, 1H), 3.30-3.20(m, 2H), 2.70-2.55(m, 2H), 1.98-1.57(m, 4H), 1.50-1.25(m, 14H), 0.96-0.90(m, 3H).

### Example 43

### (2S,3S)—N-(4-(heptylthiomethyl)benzyl)-3-hydroxypyrrolidine-2-carboxamide

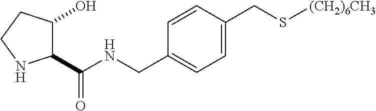

Utilizing a procedure similar to that described in Preparation A, the compound of Example 43 was prepared from (4-(heptylthiomethyl)phenyl)methanamine (0.10 g, 0.40 mmol) and (2S,3S)-1-(tert-butoxycarbonyl)-3-hydroxypyrrolidine-2-carboxylic acid (0.12 g, 0.5 mmol). Product was afforded as a colorless solid. ¹HNMR (CD₃OD): 7.32-7.20(m, 4H), 4.40-4.32(m, 3H), 3.72-3.68(m, 2H), 3.57-3.52(m, 1H), 3.20-3.05(m, 2H), 2.42-2.37(m, 2H), 1.90-1.78(m, 2H), 1.60-1.50(m, 2H), 1.40-1.22(m, 8H), 0.96-0.90(m, 3H).

### Example 44

### (2S,3S)-3-hydroxy-N-(4-octylphenethyl)pyrrolidine-2-carboxamide

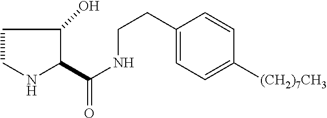

Utilizing a procedure similar to that described in Preparation A, the compound of Example 44 was prepared from 2-(4-octylphenyl)ethanamine (0.23 g, 1.00 mmol) and (2S,3S)-1-(tert-butoxycarbonyl)-3-hydroxypyrrolidine-2-carboxylic acid (0.23 g, 1.00 mmol). Product was afforded as a colorless solid. ¹HNMR (CD₃OD): 7.20-7.14(m, 4H), 4.40-438(m, 1H), 4.15-4.13(m, 1H), 3.70-3.40(m, 4H), 2.95-2.80(m, 2H), 2.65-2.60(m, 2H), 2.05-1.90(m, 2H), 1.68-1.59(m, 2H), 1.42-1.13(m, 10H), 0.95-0.90(m, 3H).

### Example 45

### cis-N-(4-decylphenyl)-3-hydroxypiperidine-2-carboxamide

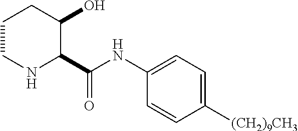

Utilizing a procedure similar to that described in Preparation A, the compound of Example 45 was prepared from 4-decylaniline (0.23 g, 1.0 mmol) and cis-1-(tert-butoxycarbonyl)-3-hydroxypiperidine-2-carboxylic acid (0.25 g, 1.0 mmol). Product was afforded as a colorless solid. ¹HNMR (CD₃OD): 7.52-7.47(m, 2H), 7.20-7.15(m, 2H), 4.48-4.42(m, 1H), 3.97-3.96(m, 1H) 3.40-3.31(m, 1H), 3.10-3.00(m, 1H), 2.60-2.55(m, 2H), 2.20-1.98(m, 2H), 1.90-1.58(m, 4H), 1.40-1.26(m, 14H), 0.96-0.90(m, 3H).

### Example 46

### (2S,3S)—N-(4-(hexylsulfonylmethyl)phenyl)-3-hydroxypyrrolidine-2-carboxamide

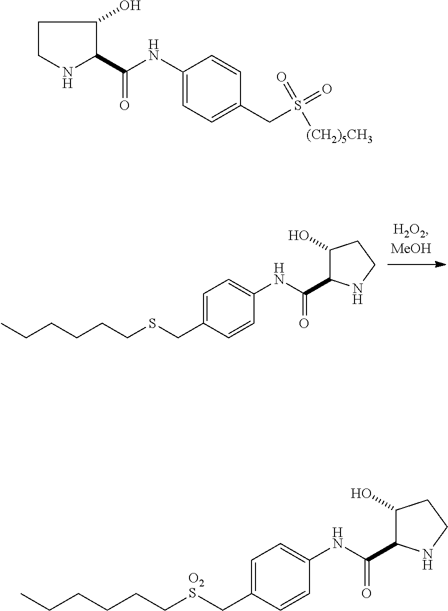

To a solution of (2S,3S)-tert-butyl 2-(4-(hexylthiomethyl)phenylcarbamoyl)-3-hydroxypyrrolidine-1-carboxylate (Example 39) (0.2 g, 0.46 mmol) in MeOH (2 mL) was added H₂O₂ (0.051 mL, 0.50 mmol) at room temperature. The mixture was stirred overnight. After removal of solvent, to the residue was added 2 mL of TFA+CH₂Cl₂ (1:1) at room temperature. The mixture was stirred for another 2 hours and the starting material was completely gone by TLC. The solvent was evaporated and the residue was chromatographed by HPLC to give a 0.08 g of colorless solid (Example 46) (48%). ¹HNMR (CO₃OD): 7.70-7.65(m, 2H), 7.45-7.40(m, 2H), 4.50-4.44(m, 1H), 4.42(s, 2H), 3.70-3.66(m, 1H), 3.25-3.18(m, 2H), 3.05-2.95(m, 2H), 1.97-1.72(m, 4H), 1.49-1.28(m, 6H), 0.96-0.90(m, 3H).

### Example 47

### (2S,3S)-2-(dipropylamino)-3-hydroxy-N-(4-octylphenyl)butanamide

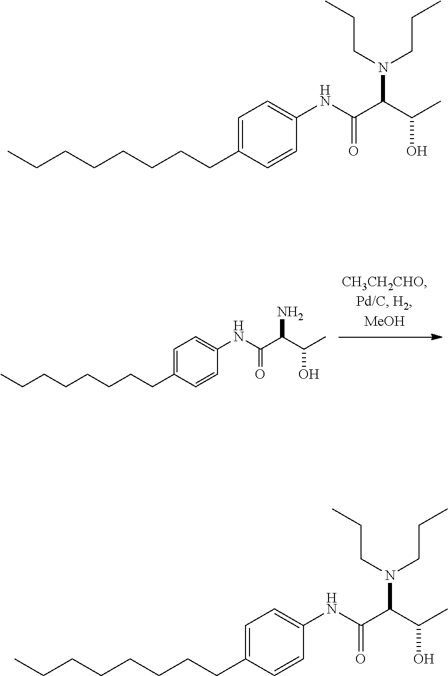

A solution of (2S,3S)-2-amino-3-hydroxy-N-(4-octylphenyl)butanamide (0.2 g, 0.65 mmol), 0.5 mL of propaldehyde (0.3 mL) in MeOH (2 mL) was degassed with N₂ for 10 minutes, Pd/C (10%, wet, 0.2 g) was added and H₂ balloon was equipped. The mixture was stirred at room temperature overnight. The solvent was then removed and the residue was chromatographed with 0-10% MeOH in CH₂Cl₂ (0.5% NH3OH) to give 0.15 g of desired product, as a colorless oil. ¹HNMR (CD₃OD): 7.43-7.39 (m, 2H), 7.18-7.14(m, 2H), 4.02-3.97(m, 1H), 3.10-3.07(m, 1H), 2.90-2.80(m, 2H), 2.60-2.50(m, 4H), 1.63-1.40(m, 6H), 1.38-1.22(m, 10H), 1.20-1.16(m, 3H), 0.98-0.87 (m, 9H).

### Example 48

### (2S,3S)-2-(cyclohexylmethylamino)-3-hydroxy-N-(4-octylphenyl)butanamide

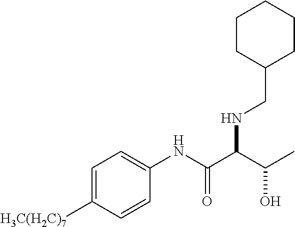

Utilizing a procedure similar to that described in Example 47, the compound of Example 48 was prepared from (2S,3S)-2-amino-3-hydroxy-N-(4-octylphenyl)butanamide (0.20 g, 0.65 mmol) and cyclohexyl aldehyde (0.4 mL). Product was afforded as a pale yellow foam.

¹HNMR (CD₃OD), 7.44-7.40 (m, 2H) 7.18-7.14(m, 2H), 3.92-3.86(m, 1H), 2.94-3.90 (m, 1H), 2.52-2.30 (m, 4H), 1.85-1.40(m, 8H), 1.30-1.12(m, 16H), 1.00-0.80(m, 5H).

### Example 49

### (2S,3S)-2-(dibenzylamino)-3-hydroxy-N-(4-octylphenyl)butanamide

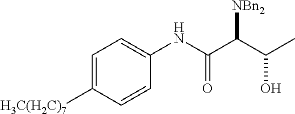

Utilizing a procedure similar to that described in Example 47, the compound of Example 49 was prepared from (2S,3S)-2-amino-3-hydroxy-N-(4-octylphenyl)butanamide (0.20 g, 0.65 mmol) and benzaldehyde (0.4 mL). Product was afforded as a pale yellow foam. ¹HNMR (CO₃OD): 7.60-7.57 (m, 2H), 7.42-7.05(m, 12H), 4.60(s, 2H), 4.44-4.60(m, 1H), 4.44-4.41.(m, 1H), 3.40-3.37(m, 1H), 2.55-2.49 (m, 1H), 1.63-1.57(m, 2H), 1.56-1.50(m, 3H), 1.40-1.20(m, 12H), 0.96-0.90(m, 3H).

### Example 50

### (2S,3S)—N-(4-(3-cyclohexylpropyl)phenyl)-3-hydroxypyrrolidine-2-carboxamide

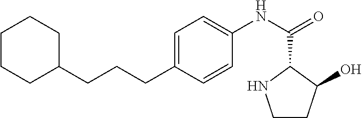

Utilizing a procedure similar to that described in Preparation A, the compound of Example 50 was prepared from 4-(3-cyclohexylpropyl)aniline (0.11 g, 0.5 mmol) and (2S,3S)-1-(tert-butoxycarbonyl)-3-hydroxypyrrolidine-2-carboxylic acid (0.13 g, 0.55 mmol). Product was afforded as a colorless solid. ¹HNMR (CD₃OD): 7.78-7.70 (d, 2H), 7.21-7.18 (d, 2H), 4.67(bs, 1H), 4.35 (s, 1H), 3.70-3.50 (m, 2H), 2.61-2.58(m, 2H), 2.21-2.04(m, 2H), 1.80-1.60(m, 7H), 1.34-1.15(m, 6H), 0.98-0.87 (m, 2H).

### Example 51

### (S)-3-Hydroxy-2-(methylamino)-N-(4-octylphenyl)propanamide

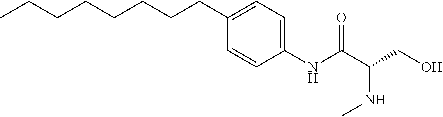

Utilizing a procedure similar to that described in Preparation A, the compound of Example 51 was prepared from 4-octylaniline (1.0 g, 4.87 mmol) and (S)-2-(tert-butoxycarbonyl(methyl)amino)-3-hydroxypropanoic acid (1.0 g, 4.56 mmol) to afford 104 mg (38%) of product as a white solid: ¹H NMR (DMSO-d₆) δ 9.70 (br s, 1H), 7.52 (d, J=8.4 Hz, 2H), 7.08 (d, J=8.4 Hz, 2H), 4.82 (s, 1H), 3.71-3.40 (m, 2H), 3.30 (br s, 1H), 3.17-2.98 (m, 1H), 2.54-2.44 (m, 2H), 2.28 (s, 3H), 1.64-1.38 (m, 2H), 1.34-1.06 (m, 10H), 0.83 (t, J=6.8 Hz, 3H) ppm. MS (ESI) m/z 307.20 (M+H)⁺.

### Example 52

### (S)-2-Amino-6-hydroxy-N-(4-octylphenyl)hexanamide

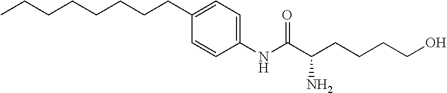

Utilizing a procedure similar to that described in Preparation A, the compound of Example 52 was prepared from 4-octylaniline and (S)-2-(tert-butoxycarbonylamino)-6-hydroxyhexanoic acid. Deprotection of the intermediate tert-butyl 6-hydroxy-1-(4-octylphenylamino)-1-oxohexan-2-ylcarbamate was carried out with 1:4 trifluoroacetic acid/methylene chloride instead of 4 M hydrogen chloride in dioxane. The product was obtained as a white solid: ¹H NMR (CD3OD) δ 7.46 (d, J=8.4 Hz, 2H), 7.12 (d, J=8.4 Hz, 2H), 3.56 (t, J=6.3 Hz, 2H), 3.43 (t, J=6.6 Hz, 1H), 2.57 (t, J=8.0 Hz, 2H), 1.85-1.71 (m, 1H), 1.70-1.37 (m, 7H), 1.36-1.19 (m, 10H), 0.89 (t, J=6.8 Hz, 3H) ppm. MS (ESI) m/z 335.10 (M+H)⁺.

### Preparation C

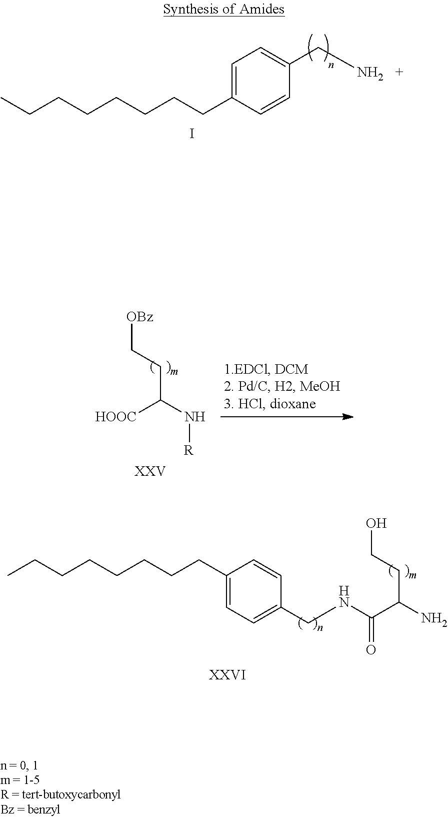

To a stirred mixture of 4-octylaniline (1.0 g, 4.87 mmol) and a racemic or chiral protected amino acid, such as 4-(benzyloxy)-2-(tert-butoxycarbonylamino)butanoic acid (1.0 g, 3.23 mmol) in methylene chloride or another suitable organic solvent (12 mL) was added N-(3-dimethylaminopropyl)-N′-ethylcarbodiimide hydrochloride (0.87 g, 4.56 mmol) in another suitable coupling reagent. After stirring overnight at room temperature the mixture was concentrated and the residue was purified by silica gel chromatography (19:1 to 4:1 methylene chloride/ethyl acetate) to afford 1.31 g (81%) of tert-butyl 4-(benzyloxy)-1-(4-octylphenylamino)-1-oxobutan-2-ylcarbamate.

A solution of the amide (513 mg, 1.0 mmol) in a I:I mixture of methanol and ethyl acetate (30 mL) was added to a suspension of 5% palladium on charcoal (500 mg) in methanol (5 mL) or another suitable solvent. The mixture was stirred in a hydrogen atmosphere for 16 hours at room temperature then filtered through Celite. The filtrate was concentrated and then purified h silica gel chromatography (1:1 hexane/ethyl acetate) to yield 389 mg (95%) tert-butyl 4-hydroxy-1-(4-octylphenylamino)-1-oxobutan-2-ylcarbamate of as a colorless gum. The gum was dissolved in methylene chloride (5 mL) and 4 M hydrogen chloride in dioxane (5 mL) then stirred at room temperature for 2 hours. The resulting suspension was diluted with ethyl acetate and slowly washed with saturated aqueous sodium bicarbonate. The aqueous layer was extracted with ethyl acetate and the organic layers were combined, washed twice with saturated aqueous sodium chloride, dried (magnesium sulfate) and concentrated. The residue was purified by silica gel chromatography (98:2 to 9:1 methylene chloride/methanol) to afford 67 mg (23%) of the desired product.

### Example 53

### (±)-(2-Amino-4-hydroxy-N-(4-octylphenyl)butanamide

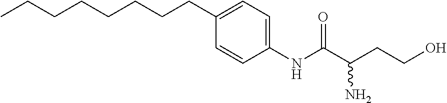

Utilizing a procedure similar to that described in Preparation C, the compound of Example 53 was prepared from 4-octylaniline (1.0 g, 4.87 mmol) and racemic 4-(benzyloxy)-2-(tert-butoxycarbonylamino)butanoic acid (1.0 g, 3.23 mmol)) to afford 67 mg (23%) as a white solid: ¹H NMR (CD₃OD) δ 7.45 (d, J=8.5 Hz, 2H), 7.12 (d, J=8.5 Hz, 2H), 3.80-3.63 (m, 2H), 3.56 (dd, J₁=7.9 Hz, J₂=5.4 Hz, 1H), 2.65-2.45 (m, 2H), 2.09-1.88 (m, 1H), 1.86-1.68 (m, 1H), 1.66-1.47 (m, 2H), 1.40-1.13 (m, 10H), 0.88 (t, J=6.9, 3H) ppm. MS (ESI) m/z 307.20 (M+H)⁺.

### Example 54

### (S)-2-Amino-4-hydroxy-N-(4-octylphenyl)butanamide

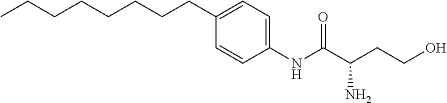

Utilizing a procedure similar to that described in Preparation C, the compound of Example 54 was prepared from 4-octylaniline and (S)-4-(benzyloxy)-2-(tert-butoxycarbonylamino)butanoic acid. Product was afforded as a white solid: ¹N NMR (CD₃OD) δ 7.46 (d, J=8.4 Hz, 2H), 7.13 (d, J=8.4 Hz, 2H), 3.78-3.64 (m, 2H), 3.56 (dd, J₁=7.9 Hz, J₂=5.4 Hz, 1H), 2.57 (t, J=7.6 Hz, 2H), 2.05-1.93 (m, 1H), 1.85-1.70 (m, 1H), 1.66-1.50 (m, 2H), 1.39-1.19 (m, 10H), 0.88 (t, J=6.8 Hz, 3H) ppm. MS (ESI) m/z 307.20 (M+H)⁺.

### Example 55

### (R)-2-Amino-4-hydroxy-N-(4-octylphenyl)butanamide

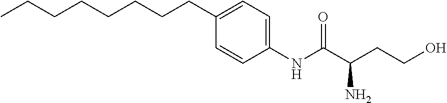

Utilizing a procedure similar to that described in Preparation C, the compound of Example 55 was prepared from 4-octylaniline and (R)-4-(benzyloxy)-2-(tert-butoxycarbonylamino)butanoic acid. Product was afforded as a white solid: ¹H NMR (CD₃OD) δ 7.46 (d, J=8.4 Hz, 2H), 7.13 (d, J=8.4 Hz, 2H), 3.78-3.65 (m, 2H), 3.57 (dd, 1H, J₁=7.9 Hz, J₂=5.4 Hz), 2.57 (t, J=8.0 Hz, 2H), 2.06-1.90 (m, 1H), 1.86-1.70 (m, 1H), 1.66-1.51 (m, 2H), 1.41-1.16 (m, 10H), 0.89 (t, J=6.8 Hz, 3H) ppm. MS (ESI) m/z 307.20 (M+H)⁺.

### Example 56

### (±)-3-Amino-4(4-octylphenylamino)butan-1-ol

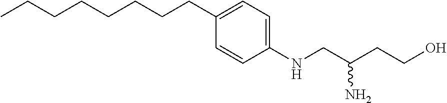

Utilizing a procedure similar to that described in Preparation B, the compound of Example 56 was prepared from 3-amino-4-(4-octylphenylamino)-4-oxobutanoic acid hydrochloride (190 mg, 0.53 mmol) with borane dimethyl sulfide complex (2 mL, 20 mmol) to afford 57 mg (38%) of product as a tan solid: ¹H NMR (CD₃OD) δ 6.92 (d, J=8.4 Hz, 2H), 6.59 (d, J=8.4 Hz, 2H), 3.71 (t, J=6.3 Hz, 2H), 3.20-3.04 (m, 2H), 3.02-2.88 (m, 1H), 2.45 (t, J=7.6, 2H), 1.84-1.68 (m, 1H), 1.66-1.45 (m, 3H), 1.39-1.18 (m, 10H), 0.89 (t, J=6.8, 3H) ppm. MS (ESI) m/z 293.20 (M+H)⁺.

### Example 57

### (S)-2-Amino-4-hydroxy-N-(4-octylbenzyl)butanamide

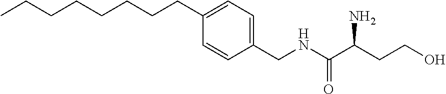

Utilizing, a procedure similar to that described in Preparation C, the compound of Example 57 was prepared from (4-octylphenyl)methanamine and (S)-4-(benzyloxy)-2-(tert-butoxycarbonylamino)butanoic acid as a white solid: ¹H NMR (CD₃OD) δ 7.20 (d, J=8.1 Hz, 2H), 7.13 (d, J=8.1 Hz, 2H), 4.42-4.27 (m, 2H), 3.73-3.58 (m, 2H), 3.46 (dd, J₁=7.8 Hz, J₂=5.5 Hz, 1H), 2.65-2.50 (m, 2H), 1.99-1.83 (m, 1H), 1.80-1.65 (m, 1H), 1.64-1.51 (m, 2H), 1.40-1.17 (m, 10H), 0.89 (t, J=6.9 Hz) ppm. MS (ESI) m/z 321.20 (M+H)⁺.

### Example 58

### (2S,3S)—N-(2-amino-5-octylphenyl)-3-hydroxypyrrolidine-2-carboxamide hydrochloride

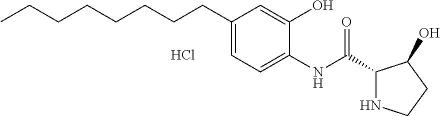

### Preparation of the Intermediate 2-amino-4-octylphenol

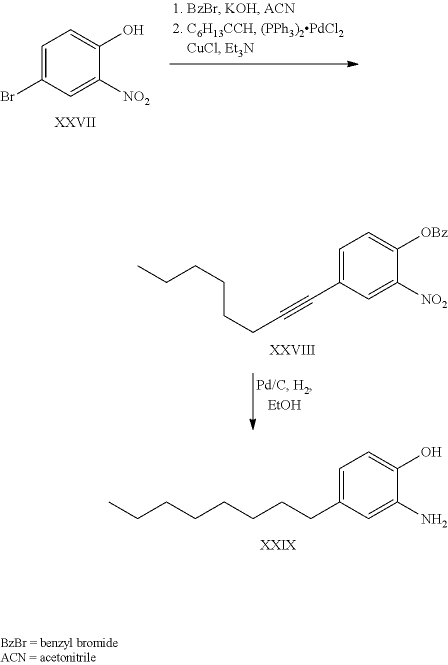

### Step 1) Preparation of 1-(benzyloxy)-4-bromo-nitrobenzene

A mixture of 4-bromo-2-nitrophenol (XXVII) (10.56 g, 46.5 mmol) benzyl bromide (8.2 mL, 69.0 mmol), potassium carbonate (9.7 g, 70.2 mmol), acetonitrile (250 mL) and acetone (135 mL) was heated at reflux for 19 hours. The mixture was filtered and the filtrate was concentrated to yield a moist solid which was purified by silica gel chromatography (17:3 to 3:7 hexane/methylene chloride) to afford 14.3 g (99%) of 1-(benzyloxy)-4-bromo-2-nitrobenzene. ¹H NMR (CDCl₃) δ 7.96 (d, J=2.5 Hz, 1H), 7.57 (dd, J₁=8.9 Hz, J₂=5 Hz, 1H) 7.48-7.28 (m, 5H), 7.00 (d, 1H, J=8.9 Hz, 5.21 (s, 2H) ppm.

### Step 2) Preparation 1-(benzyloxy)-2-nitro-4-(oct-1-ynyl)benzene (XXVIII)

A mixture of 1-(benzyloxy)-4-bromo-2-nitrobenzene (3.17 g, 10.3 mmol), 1-octyne (1.8 mL, 12.2 mmol), bis(triphenylphosphine)palladium(II) dichloride (145 mg, 0.21 mmol), triethylamine (10 mL) and copper (I) iodide (78 mg, 0.41 mmol) was heated at 60° C. for 16 hours. The reaction mixture was purified by silica gel chromatography (19:1 to 4:1 hexane/ethyl acetate) to afford 3.17 g (91%) of 1-(benzyloxy)-2-nitro-4-(oct-1-ynyl)benzene as a dark oil: ¹H NMR (CDCl₃) δ 7.87 (d, J=2.1 Hz, 1H), 7.52-7.29 (m, 6H), 7.02 (d, J=8.7 Hz, 1H), 5.23 (s, 2H), 2.38 (t, J=7.1, Hz, 2H), 1.69-1.51 (m, 3H), 1.49-1.20 (m, 6H), 0.90 (t, J=7.0 Hz, 3H) ppm.

A suspension of 1-(benzyloxy)-2-nitro-4-(oct-1-ynyl)benzene (3.17 g, 9.4 mmol) and 10% palladium on charcoal (475 mg) in ethanol (20 mL) was stirred in a hydrogen atmosphere at room temperature. After 24 hours the reaction was filtered through Celite filter aid. The filtrate was concentrated to give 2-amino-4-octylphenol (XXIX) as a brown solid: ¹H NMR (CDCl₃) δ 6.64 (d, J=7.9 Hz, 1H), 6.59 (d, J=1.9 Hz, 1H), 6.49 (dd, J₁=7.9 Hz, J₂=1.9 Hz, 1H), 2.87 (br s, 3H), 2.50-2.37 (m, 2H), 1.66-1.43 (m, 2H), 1.30-1.11 (m, 10H), 0.88 (t, J=6.9, 3H) ppm.

Utilizing a procedure similar to that described in Preparation A, the compound of Example 58 was prepared from 2-amino-4-octylphenol and (2S,3S)-1-(tert-butoxycarbonyl)-3-hydroxypyrrolidine-2-carboxylic acid (5.09 g, 22.0 mmol) in methylene chloride (30 mL) to afford 230 mg of (2S,3S)—N-(2-amino-5-octylphenyl)-3-hydroxypyrrolidine-2-carboxamide hydrochloride as a tan solid: ¹H NMR (DMSO-d₆ with D₂O) δ 7.64 (s, 1H), 6.87-6.79 (m, 2H), 4.53-4.43 (m, 1H), 4.38 (d, J=3.0 Hz, 1H), 3.50-3.29 (m, 2H), 2.45 (t, J=7.5 Hz, 2H), 2.10-1.87 (m, 2H), 1.58-1.39 (m, 2H), 1.33-1.13 (m, 10H), 0.85 (t, J=6.8 Hz, 3H) ppm. MS (ESI) m/z 335.20 (M+H)⁺.

### Example 59

### (2S,3R)-2-Amino-3-hydroxy-N-(4′-octylbiphenyl-4-yl)butanamide

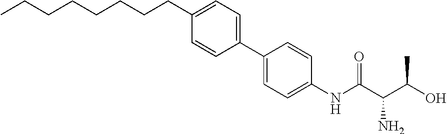

Utilizing a procedure similar to that described in Preparation A, the compound of Example 59 was prepared from 4-amino-4′-octylbiphenyl and N-(tert-butoxycarbonyl)-L-threonine. Product was afforded as a white solid: ¹H NMR (CDCl₃) δ 9.64 (br s, 1H), 7.65 (d, J=8.5 Hz, 2H), 7.55 (d, J=8.5 Hz, 2H), 7.49 (d, J=8.0 Hz, 2H), 7.23 (d, J=8.0 Hz, 2H), 4.56-4.47 (m, 1H), 3.37 (d, 2.7 Hz, 1H), 2.63 (t, J=7.7 Hz, 2H), 1.69-1.58 (m, 2H), 1.41-1.19 (m, 13H), 0.88 (t, J=7.0 Hz, 3H) ppm. MS (ESI) m/z 383 (M+H)⁺.

### Example 60

### (2R,3R)-3-Amino-4-(4′-octylbiphenyl-4-ylamino)butan-2-ol

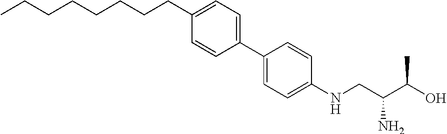

Utilizing a procedure similar to that described in Preparation B, the compound of Example 60 was prepared from the compound of Example 59. Product was afforded as a white solid: ¹H NMR (CDCl₃) δ 7.48-7.37 (m, 4H), 7.20 (d, J=8.2 Hz, 2H), 6.70 (d, J=8.5 Hz, 2H), 4.11 (br s, 1H), 3.75-3.66 (m, 1H), 3.32 (dd, J=12.5, 4.3 Hz, 1H), 3.03 (dd, J=12.5, 7.9 Hz, 1H), 2.92-2.85 (m, 1H), 2.631 (t, J=7.7 Hz, 2H), 1.78 (br s, 2H), 1.68-1.56 (m, 2H), 1.39-1.18 (m, 10H), 0.88 (t, J=7.0 Hz, 3H) ppm. MS (ESI) m/z 369 (M+H)⁺.

### Example 61

### (2S,3S)—N—((R)-5-(5-Heptyl-1,2,4-oxadiazol-3-yl)-2,3-dihydro-1H-inden-1-yl-3-hydroxypyrrolidine-2-carboxamide

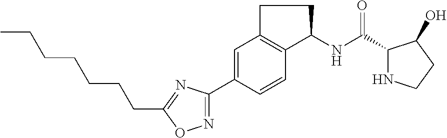

Utilizing a procedure similar to that described in Preparation A, the compound of Example 61 was prepared from ((±)-5-(5-heptyl-1,2,4-oxadiazol-3-yl)-2,3-dihydro-1H-inden-1-amine and N-(tert-butoxycarbonyl)-trans-3-hydroxy-L-proline. The crude mixture of diastereomers was separated using an Isco Combiflash unit (80 g SiO₂ column, 10-20% (1:1 methanol/acetonitrile)/dichloromethane). The desired product eluted first: ¹H NMR (CDCl₃) δ 7.96-7.84 (m, 3H), 7.23(d, J=7.8 Hz, 1H), 5.52-5.43 (m, 1H), 4.66 (t, J=5.2 Hz, 1H), 3.70 (d, J=2.2 Hz, 1H), 3.30-3.20 (m, 1H), 3.09-2.86 (m, 6H), 2.70-2.20 (m, 3H), 1.91-1.81 (m, 4H), 1.46-1.23 (m, 8H), 0.88 (t, J=6.9 Hz, 3H) ppm. MS (ESI) m/z 413 (M+H)⁺.

### Example 62

### (2S,3S)—N—((S)-5-(5-heptyl-1,2,3-oxadiazol-3-yl)-2,3-dihydro-1H-inden-1-yl)-3-hydroxypyrrolidine-2-carboxamide

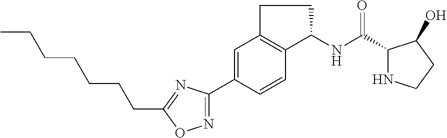

### Preparation of the intermediate ((±)-5-(5-heptyl-1,2,4-oxadiazol-3-yl)-2,3-dihydro-1H-inden-1-amine

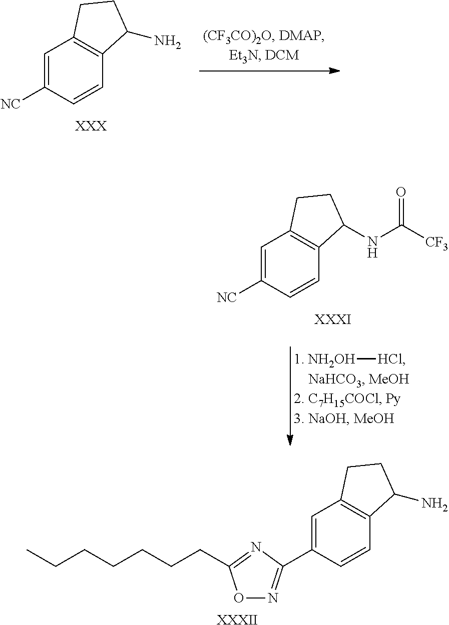

### Step 1) Preparation of (±)-N-(5-cyano-2,3-dihydro-1H-inden-1-yl)-2,2,2-trifluoroethanamide

To a stirred solution of (±)-1-amino-2,3-dihydro-1H-indene-5-carbonitrile (XXX) (1.03 g, 6.51 mmol) triethylamine (0.856 g, 8.46 mmol), and 4-(dimethylamino)pyridine (˜0.020 g) in dichloromethane (30 mL) was added trifluoroacetic anhydride (1.50 g, 7.16 mmol) dropwise over 5 minutes. The resulting solution was allowed to stir at room temperature. After 1.5 hours, the reaction mixture was treated with methanol (2 mL) and allowed to stir for 10 Minutes. The mixture was washed with 1N hydrochloric acid and brine, dried (magnesium sulfate), filtered, and concentrated to provide 1.52 g of a yellow solid. Flash chromatography using an Isco Combiflash unit (120 g SiO₂ column, 20-50% ethyl acetate/hexanes) afforded 1.34 g (81%) of (±)-N-(5-cyano-2,3-dihydro-1H-inden-1-yl)-2,2,2-triflooroethanamide (XXXI) as an off-white solid: ¹H NMR (CDCl₃) δ 7.57-7.52 (m, 2H), 7.39 (d, J=7.7 Hz, 1H), 6.46 (br s, 1H), 5.56 (q, J=8.0 Hz, 1H), 3.17-3.04 (m, 1H), 3.03-2.92 (m, 1H), 2.78-2.67 (m, 1H), 2.04-1.91 (m, 1H) ppm.

### Step 2) Preparation of (±)-2,2,2-trifluoro-N-(5-(N′-hydroxycarbamimidoyl)-2,3-dihydro-1H-inden-1-yl)ethanamide

To a stirred suspension of (±)-N-(5-cyano-2,3-dihydro-1H-inden-1-yl)-2,2,2-trifluoroethanamide (XXXI) (1.34 g, 5.27 mmol) and sodium bicarbonate (1.77 g, 21.08 mmol) in methanol (30 mL) was added hydroxylamine hydrochloride (0.733 g, 10.54 mmol). The reaction mixture was heated to reflux. After 17 hours, the reaction mixture was allowed to cool to room temperature and was concentrated. The residue was partitioned between ethyl acetate and water. The phases were separated, and the aqueous phase extracted with ethyl acetate. The combined organic phases were washed with brine, dried (magnesium sulfate), filtered, and concentrated to provide 1.05 g (70%) of (±)-2,2,2-trifluoro-N-(5-(N′-hydroxycarbamimidol)-2,3-dihydro-1H-inden-1-yl)ethanamide as a white solid: ¹H NMR (DMSO-d₆) δ 9.80 (s, 1H), 9.56 (s, 1H), 7.59-7.49 (m, 2H), 7.15 (d, J=7.9 Hz, 1H), 5.75 (s, 2H), 5.36 (q, J=7.9 Hz, 1H), 3.04-2.93 (m, 1H), 2.90-2.77 (m, 1H), 2.46-2.37 (m, 1H (obscured by residual DMSO), 2.04-1.91 (m, 1H) ppm.

### Step 3) Preparation of (±)-2,2,2-trifluoro-N-(5-(5-heptyl-1,2,4-oxadiazol-3-yl)-2,3-dihydro-1H-inden-1-yl)ethanamide

To a stirred solution of (±)-2,2,2-trifluoro-N-(5-(N′-hydroxycarbamimidoyl)-2,3-dihydro-1H-inden-1-yl)ethanamide (1.05 g, 3.66 mmol) in pyridine (20 mL) was added octanoyl chloride (0.654 g, 4.02 mmol). The reaction mixture was heated to reflux. After 2 hours, the reaction mixture was allowed to cool to room temperature and was diluted with water. The mixture was extracted with diethyl ether. The organic phase was washed with 1N hydrochloric acid, saturated sodium bicarbonate, and brine. The organic phase was dried (magnesium sulfate), filtered, and concentrated to provide 1.30 g of a tan solid. Flash chromatography using an Isco Combiflash unit (80 g SiO₂ column, 10-25% ethyl acetate/hexanes) afforded 0.969 g (67%) of (±)-2,2,2-trifluoro-N-(5-(5-heptyl-1,2,4-oxadiazol-3-yl-2,3-dihydro-1H-inden-1-yl)ethanamide as a white solid: ¹H NMR (CDCl₃) δ 8.01-7.94 (m, 2H), 7.39 (d, J=7.8 Hz, 1H), 6.45 (d, J=7.7 Hz, 1H), 5.56 (q, J=7.7 Hz, 1H), 3.17-3.06 (m, 1H), 3.04-2.90 (m, 3H), 2.78-2.67 (m, 1H), 2.03-1.81 (m, 3H), 1.47-1.23 (m, 8H), 0.89 (t, J=6.8 Hz, 3H) ppm.

### Step 4) Preparation of (±)-5-(5-heptyl-1,2,4-oxadiazol-3-yl)-2,3-dihydro-1H-inden-1-amine

To a stirred solution of (±)-2,2,2-trifluoro-N-(5-(5-heptyl-1,2,4-oxadiazol-3-yl)-2,3-dihydro-1H-inden-1-yl)ethanamide (0.969 g, 2.45 mmol) in methanol (30 mL) was added 3N sodium hydroxide (30 mL). The reaction mixture was allowed to stir at room temperature. After 2 hours, the reaction mixture was concentrated, and the residue diluted with water. The solution was extracted twice with diethyl ether. The combined organic phases were washed with brine, dried (magnesium sulfate), filtered, and concentrated to provide 0.696 g (95%) of (±)-5-(5-heptyl-1,2,4-oxadiazol-3-yl)-2,3-dihydro-1H-inden-1-amine as a colorless oil: ¹H NMR (CDCl₃) δ 7.97-7.90 (m, 2H), 7.42 (d, J=7.8 Hz, 1H), 4.40 (t, J=7.6 Hz, 1H), 3.05-2.96 (m, 1H), 2.96-2.90 (m, 2H), 2.90-2.80 (m, 1H), 2.60-2.50 (m, 1H), 1.91-1.82 (m, 2H), 1.79-1.68 (m, 1H), 1.55 (br s, 2H), 1.47-1.24(m, 8H), 0.88 (t, J=6.9 Hz, 3H) ppm.

Utilizing: a procedure similar to that described in Preparation A, the compound of Example 62 was prepared from ((±)-5(5-heptyl-1,2,4-oxadiazol-3-yl)-2,3-dihydro-1H-inden-1-amine and N-(tert-butoxycarbonyl)-trans-3-hydroxy-L-proline. The crude mixture of diastereomers was separated using an Isco Combiflash unit (80 g SiO₂ column, 10-20%. (1:1 methanol/acetonitrile)/dichloromethane). The desired product eluted second: ¹H NMR (CDCl₃) δ 7.96-7.89 (m, 2H), 7.85 (d, J=8.8 Hz, 1H), 7.35 (d, J=7.8 Hz, 1H), 5.51-5.41 (m, 1H), 4.62-4.56 (m, 1H), 3.71 (d, J=2.5 Hz, 1H), 3.28-3.20 (m, 1H), 3.08-2.87 (m, 5H), 2.67-2.53 (m, 1H), 2.50-2.08 (m, 2H), 1.91-175 (m, 5H), 1.47-1.23 (m, 8H), 0.88 (t, J=6.9 Hz, 3H) ppm. MS (ESI) m/z 413 (M+H)⁺.

### Example 63

### (2S,3S)—N-(4-(5-Cyclohexyl-1-methyl-1H-1,2,4-triazol-3-yl)benzyl)-3-hydroxypyrrolidine-2-carboxamide

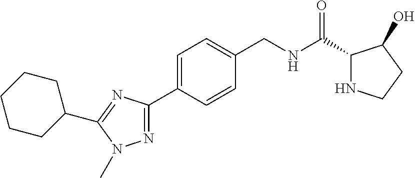

### Preparation of the Intermediate (4-(5-cyclohexyl-1-methyl-1H-1,2,4-triazol-3-yl)phenyl)methanamine

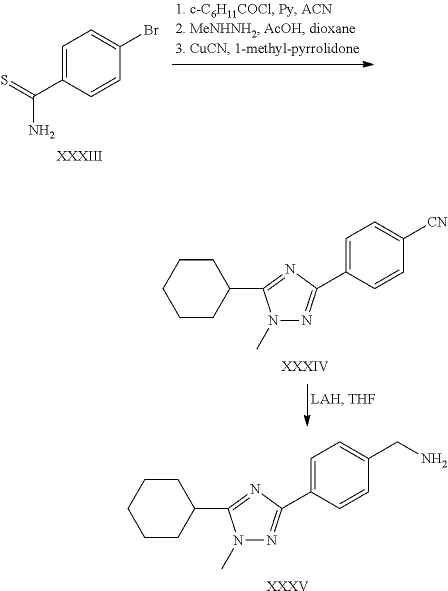

### Step 1) Preparation of N-(4-bromophenylcarbonothioyl)cyclohexanecarboxamide

To a stirred solution of 4-bromothiobenzamide (XXXIII) (5.00 g, 23.14 mmol) in acetone (100 mL) was added pyridine (2.01 g, 25.45 mmol) and cyclohexanecarbonyl chloride (3.73 g, 25.45 mmol). The resulting red solution was heated to reflux. After 1 hour, an additional portion of pyridine (0.366 g, 4.62 mmol) and cyclohexanecarbonyl chloride (0.689 g, 4.70 mmol) was added, and refluxing continued. After 2 hours, an additional portion of pyridine (0.734 g, 9.27 mmol) and cyclohexanecarbonyl chloride (1.18 g, 8.09 mmol) was added, and refluxing continued. After 3 hours, the reaction mixture was allowed to cool to room temperature and stir overnight. The mixture was concentrated and the residue dissolved in dichloromethane. The solution was washed with 1 N hydrochloric acid, saturated sodium bicarbonate solution, and brine. The organic phase was dried (magnesium sulfate), filtered, and concentrated to provide an orange solid. Flash chromatography using an Isco Combiflash unit (330 g SiO₂ column, 1:1 hexanes/dichloromethane to 100% dichloromethane to 100% methanol) afforded 5.58 g (74%) of N-(4-bromophenylcarbonothioyl)cyclohexanecarboxamide as an orange solid: ¹H NMR (DMSO-d₆) δ 12.18 (s, 1H), 7.60-7.53 (m, 2H), 7.48-7.41 (m, 2H), 2.62-2.52 (m, 1H), 1.90-1.81 (m, 2H), 1.76-1.68 (m, 2H), 1.36-1.10 (m, 6H) ppm.

### Step 2) Preparation of 3-(4-bromophenyl)-5-cyclohexyl-1-methyl-1H-1,2,4-triazole

To a stirred solution of N-(4-bromophenylcarbonothioyl)cyclohexanecarboxamide (3.00 g, 9.20 mmol) in a 1:1 mixture of 1,4-dioxane/glacial acetic acid (40 mL) was added methylhydrazine (0.445 g, 9.66 mmol). The reaction mixture was heated to reflux. After 3 hours, the reaction mixture was allowed to cool to room temperature and was diluted with water. The mixture was extracted with ethyl acetate, and the phases were separated. The organic phase was washed with 6N ammonium hydroxide and brine, dried (magnesium sulfate), filtered, and concentrated to provide 3.05 g (>100%) of 3-(4-bromophenyl)-5-cyclohexyl-1-methyl-1H-1,2,4-triazole a yellow solid. The crude mixture of regioisomers was used without purification in the next reaction: ¹H NMR (CDCl₃) δ 7.95-7.90 (m, 2H), 7.55-7.49 (m, 2H), 3.85 (s, 3H), 2.79-2.67 (m, 1H), 1.96-1.53 (m, 10H) ppm.

### Step 3) Preparation of 4-(5-cyclohexyl-1-methyl-1H-1,2,4-triazol-3-yl)benzonitrile

To a stirred solution of 3-(4-bromophenyl)-5-cyclohexyl-1-methyl-1H-1,2,4-triazole (2.94 g, 9.20 mmol, contaminated with 5-(4-bromophenyl)-3-cyclohexyl-1-methyl-1H-1,2,4-triazole) in 1-methyl-2-pyrrolidinone (30 mL) was added copper(I) cyanide (1.44 g, 16.09 mmol). The reaction mixture was heated to reflux. After 8 hours, the reaction mixture was allowed to cool to room temperature and stir continued. After 63 hours, the reaction mixture diluted with ethyl acetate and 6N ammonium hydroxide. The mixture was filtered through Celite, and the phases were separated. The aqueous phase was extracted with ethyl acetate. The combined organic phases were washed with brine, dried (magnesium sulfate), filtered, and concentrated to provide as brown oil. Flash chromatography using an Isco Combiflash unit (120 g SiO₂ column, 10-40% ethyl acetate/hexanes) afforded 0.976 g (40%) of 4-(5-cyclohexyl-1-methyl-1H-1,2,4-triazol-3-yl)benzonitrile as a white solid: ¹H NMR (CDCl₃) δ 8.17 (d, J=8.3 Hz, 2H), 7.68 (d, J=8.3 Hz, 2H), 3.88 (s, 3H), 2.79-2.70 (m, 1H), 1.96-1.87 (m, 4H), 1.81-1.65 (m, 3H), 1.47-1.31 (m, 3H) ppm.

### Step 4) Preparation of (4-(5-cyclohexyl-1-methyl-1H-1,2,4-triazol-3-yl)phenyl)methanamine

To a stirred suspension of lithium aluminum hydride (0.198 g, 5.21 mmol) in tetrahydrofuran (15 mL) was added 4-(5-cyclohexyl-1-methyl-1H-1,2,4-triazol-3-yl)benzonitrile (0.925 g, 3.47 mmol) in tetrahydrofuran (15 mL) over 10 minutes. The reaction mixture was allowed to stir at room temperature for 15 minutes, and then it was warmed to 45°C. After 1 hour, the reaction mixture treated with water (198 μL), 1N sodium hydroxide (198 μL), and water (594 μL). The resulting mixture was allowed to stir at room temperature for 0.5 hour, and then was filtered through Celite with the aid of ethyl acetate. The filtrate was washed with saturated sodium potassium tartrate solution and brine, dried. (magnesium sulfate), filtered, and concentrated to provide 0.674 g (99%) of (4-(5-cyclohexyl-1-methyl-1H-1,2,4-triazol-3-yl)phenyl)methanamine as a yellow oil: ¹H NMR (CDCl₃) δ 8.02 (d, J=8.2 Hz, 2H), 7.34 (d, J=8.2 Hz, 2H), 3.92-3.83 (m, 5H), 2.80-2.69 (m, 1H), 1.97-1.68 (m, 9H), 1.46-1.31 (m, 3H) ppm.

Utilizing: a procedure similar to that described in Preparation A, the compound of Example 63 was prepared from (4-(5-cyclohexyl-1-methyl-1H-1,2,4-triazol-3-yl)phenyl)methanamine and N-(tert-butoxycarbonyl)-trans-3-hydroxy-L-proline. The product was obtained as a white solid: ¹H NMR (CDCl₃) δ 8:03-7.93 (m, 3H), 7.29-7.24 (m, 2H (obscured by residual CHCl3)), 4.60-4.55 (m, 1H), 4.50-4.36 (m, 2H), 3.86 (s, 3H), 3.71 (d, J=2.3 Hz, 1H), 3.29-3.20 (m, 1H), 3.01-2.93 (m, 1H), 2.77-2.69 (m, 1H), 2.47 (br s, 2H), 1.96-1.67 (m, 9H), 1.46-1.30 (m, 3H) ppm. MS (ESI) m/z 384 (M+H)⁺. The purified product was isolated as the hydrochloride salt by treatment of an ethereal solution of the free base with anhydrous hydrogen chloride.

### Example 64

### (2S,3S)—N-(4-(4-Heptyl-1H-1,2,3-triazol-1-yl)benzyl)-3-hydroxypyrrolidine-2-carboxamide

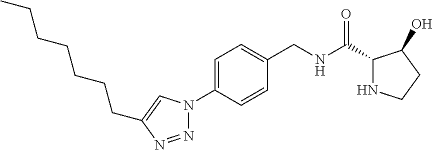

### Preparation of the Intermediate (4-(4-heptyl-1H-1,2,3-triazol-1-yl)phenyl)methanamine

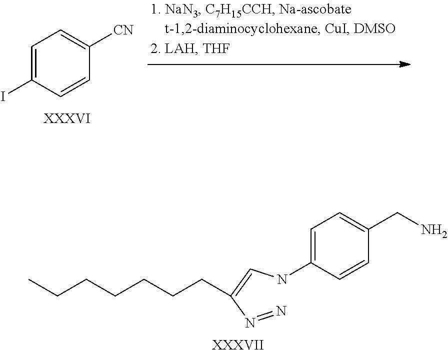

### Step 1) Preparation of 4-(4-heptyl-1H-1,2,3-triazol-1-yl)benzonitrile

To a stirred solution of 4-iodobenzonitrile (XXXVI) (2.29 g, 10.00 mmol), sodium azide (0.683 g, 10.50 mmol), 1-nonyne (1.24 g, 10.00 mmol), sodium ascorbate (0.198 g, 1.00 mmol), trans-1,2-diaminocyclohexane (0.171 g, 1.50 mmol) in dimethylsulfoxide (25 mL) and water (5 mL) was added copper(I) iodide (0.190 g, 1.00 mmol). A slight exothem was noted, and after 10 min, a precipitate had formed. After 1 hour, the reaction mixture was diluted with water and extracted with ethyl acetate. The organic phase was washed with 2N ammonium hydroxide, 1N hydrochloric acid, and brine. The organic phase was dried (magnesium sulfate), filtered, and concentrated to provide 2.66 g of a yellow solid. Flash chromatography using an Isco Combiflash unit (120 g SiO₂ column, 10-30% ethyl acetate/hexanes) afforded 1.69 g (63%) of 4-(4-heptyl-1H-1,2,3-triazol-1-yl)benzonitrile as an off-white solid: ¹H NMR (CDCl₃) δ 7.93-7.87 (m, 2H), 7.85-7.80 (m, 2H), 7.77 (s, 1H), 2.80 (t, J=7.7 Hz, 2H), 1.78-1.68 (m, 2H), 1,45-1.23 (m, 8H), 0.88 (t, J=6.8 Hz, 3H) ppm.

### Step 2) Preparation of (4-(4-heptyl-1H-1,2,3-triazol-1-yl)phenyl)methanamine (XXXVII)

To a stirred suspension of lithium aluminum hydride (0.358 g, 9.43 mmol) in tetrahydrofuran (15 mL) was added 4-(4-heptyl-1H-1,2,3-triazol-1-yl)benzonitrile (1.69 g, 6.30 mmol) in tetrahydrofuran (20 mL) over 10 minutes. The reaction mixture was allowed to stir at room. After 1 hour, the reaction mixture treated with water (358 μL), 1N sodium hydroxide (358 μL), and water (1.1 ml). The resulting, mixture was allowed to stir at room temperature for 0.5 hour, and then it was filtered through Celite with the aid of ethyl acetate. The filtrate was washed with saturated sodium potassium tartrate solution and brine, dried (magnesium sulfate), filtered, and concentrated to provide 1.70 g of a yellow oil. Flash chromatography using an Isco Combiflash unit (80 g SiO₂ column, 1-5% 2M ammonia in methanol/dichloromethane) afforded 0.827 g (48%) of (4-(4-heptyl-1H-1,2,3-triazol-1-yl)phenyl)methanamine as a yellow solid: ¹H NMR (CDCl₃) δ 7.71-7.65 (m, 3H), 7.48-7.43 (m, 2H), 3.94 (s, 2H), 2.82-2.73 (m, 2H), 1.78-1.67 (m, 2H), 1.48-1.22 (m, 10H), 0.8 (t, J=6.8 Hz, 3H) ppm.

Utilizing a procedure similar to that described in Preparation A, the compound of Example 64 was prepared from (4-(4-heptyl-1H-1,2,3-triazol-1-yl)phenyl)methanamine and N-(tert-butoxycarbonyl)-trans-3-hydroxy-L-proline. The product was obtained as a white solid: ¹H NMR (CDCl₃) δ 8.07-7.99 (m, 1H), 7.71-7.64 (m, 3H), 7.42-7.35 (m, 2H), 4.61-4.56 (m, 1H), 4.48 (d, J=6.2 Hz, 2H), 3.69 (d, J=2.3 Hz, 1H), 3.30-3.22 (m, 1H), 3.02-2.94 (m, 1H), 2.82-2.75 (m, 2H), 2.38 (br s, 2H), 1.8-1.80 (m, 2H), 1.77-1.68 (m, 2H), 1.44-1.23 (m, 8H), 0.88 (t, J=6.8 Hz, 3H) ppm. MS (ESI) m/z 386 (M+H)⁺.

### Example 65

### (2S,3S)-3-Hydroxy-N-(4-(4-phenethyl-1H-1,2,3-triazol-1-yl)benzyl)pyrrolidine-2-carboxamide

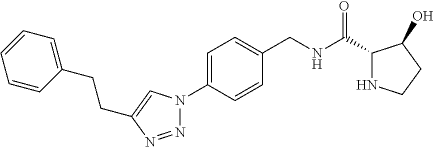

Utilizing a procedure similar to that described in Preparation A, the compound of Example 65 was prepared from (4-(4-phenethyl-1H-1,2,3-triazol-1-yl)phenyl)methanamine and N-(tert-butoxycarbonyl)-trans-3-hydroxy-L-proline. The product was obtained as a white solid: ¹H NMR (CDCl₃) δ 8.12-8.05 (m, 1H), 7.62 (d, J=8.5 Hz, 2H), 7.52 (s, 1H), 7.37 (d, J=8.5 Hz, 2H), 7.32-7.27 (m, 2H), 7.24-7.18 (m, 3H), 4.61-4.55 (m, 1H), 4.47 (d, J=6.2 Hz, 2H), 3.74 (d, J=2.2 Hz, 1H), 3.32-3.21 (m, 1H), 3.17-2.94 (m, 5H), 2.38 (br s, 2H), 1.89-1.80 (m, 2H) ppm. MS (ESI) m/z 392 (M+H)⁺.

The intermediate, (4-(4-phenethyl-1H-2,3-triazol-1-yl)phenyl)methanamine, was prepared using procedures similar to that described in Example 64 from 4-iodobenzonitrile (2.00 g, 8.73 mmol), sodium azide (0.596 g, 9.17 mmol), but-3-ynylbenzene (1.14 g, 8.73 mmol), and then reduction with Lilium aluminum hydride in THF to afforded 0.696 g (46%) of (4-(4-phenethyl-1H-1,2,3-triazol-1-yl)phenyl)methanamine as a yellow solid: ¹H NMR (CDCl₃) δ 7.64 (d, J=8.5 Hz, 2H), 7.54 (s, 1H), 7.45 (d, J=8.5 Hz, 2H), 7.33-7.27 (m, 2H), 7.24-7.18 (m, 3H), 3.95 (s, 2H), 3.17-3.03 (m, 4H), 1.47 (br s, 2H) ppm.

### Example 66

### (2S,3S)-3-Hydroxy-N—((S)-1-(4-(4-phenethyl-1H-1,2,3-triazol-1-yl)phenyl)propyl)pyrrolidine-2-carboxamide

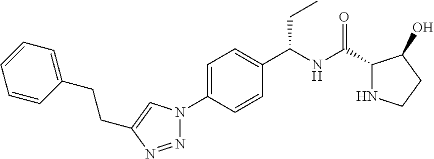

### Preparation of the Intermediate (S)-1-(4-(4-phenethyl-1H-1,2,3-triazol-1-yl)phenyl)propan-1-amine

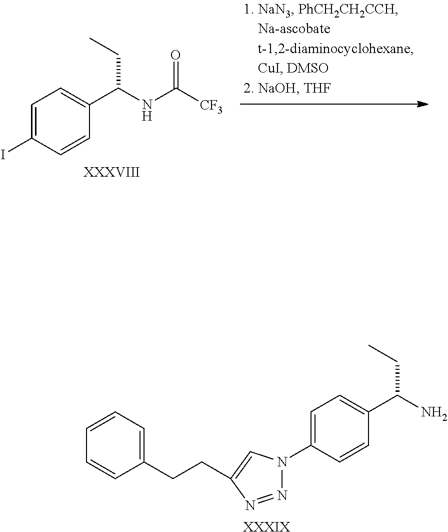

### Step 1) Preparation of (S)-2,2,2-trifluoro-N-(1-(4-(4-phenethyl-1H-1,2,3-triazol-1-yl)phenyl)propyl)ethanamide

To a stirred solution of (S)-2,2,2-trifluoro-N-(1-(4-iodophenyl)propyl)ethanamide (XXXVIII) (2.83 g, 8.65 mmol), sodium azide (0.591 g, 9.09 mmol), but-3-ynylbenzene (1.13 g, 8.65 mmol), sodium ascorbate (0.171 g, 0.865 mmol), trans-N,N′-dimethylcyclohexane-1,2-diamine (0.185 g, 1.30 mmol) in dimethylsulfoxide (25 mL) and water (5 mL) was added copper(I) iodide (0.165 g, 0.865 mmol). The reaction mixture was allowed to stir at room temperature. After 4 hours, the reaction mixture was diluted with water and extracted with ethyl acetate. The organic phase was washed with 2N ammonium hydroxide, 1N hydrochloric acid, and brine. The organic phase was dried (magnesium sulfate), filtered, and concentrated to provide as brown oil. Flash chromatography using an Isco Combiflash unit (120 g SiO₂ column, 1-5% methanol/dichloromethane) afforded 2.24 g (64%) of (S)-2,2,2-trifluoro-N-(1-(4-(4-phenethyl-1H-1,2,3-triazol-1-yl)phenyl)propyl)ethanamide as an off-white solid: ¹H NMR (DMSO-d₆) δ 9.86 (d, J=8.2 Hz, 1H), 8.51 (s, 1H), 7.82 (d, J=8.6 Hz, 2H), 7.52 (d, J=8.6 Hz, 2H), 7.30-7.21 (m, 4H.), 7.20-7.13 (m, 1H), 4.85-4.75 (m, 1H), 3.03-2.94 (m, 4H), 1.92-1.74 (m, 2H), 0.87 (t, J=7.3 Hz, 3H) ppm.

### Step 2) Preparation of (S)-1-(4(4-phenethyl-1H-1,2,3-triazol-1-yl)phenyl)propan-1-amine (XXXIX)

To a stirred solution of (S)-2,2,2-trifluoro-N-(1-(4-(4-phenethyl-1H-1,2,3-triazol-1-yl)phenyl)propyl)ethanamide (2.24 g, 5.57 mmol) in methanol (30 mL) and tetrahydrofuran (15 mL) was added 5N sodium hydroxide (30 mL). The reaction mixture was allowed to stir at room temperature. After 2 hours, the reaction mixture was concentrated, and the residue diluted with water. The solution was extracted twice with diethyl ether. The combined organic phases were washed with brine, dried (magnesium sulfate), filtered, and concentrated to provide 1.51 g (88%) of (S)-1-(4-(4-phenethyl-1H-1,2,3-triazol-1-yl)phenyl)propan-1-amine (XXXIX) as a yellow solid: ¹H NMR (CDCl₃) δ 7.62 (d, J=8.4 Hz, 2H), 7.53 (s, 1H), 7.45 (d, J=8.4 Hz, 2H), 7.33-7.26 (m, 2H), 7.24-7.17 (m, 3H), 3.93-3.84 (m, 1H), 3.17-3.01 (m, 4H), 1.78-1.62 (m, 2H), 1.48 (br s, 2H), 0.88 (t, J=7.4 Hz, 3H) ppm.

Utilizing a procedure similar to that described in Preparation A, the compound of Example 66 was prepared from (S)-1-(4-(4-phenethyl-1H-1,2,3-triazol-1-yl)phenyl)propan-1-amine and N-(tert-butoxycarbonyl)-trans-3-hydroxy-L-proline. The product was obtained as a white solid: ¹H NMR (CDCl₃) δ 7.96 (d, J=8.7 Hz, 1H), 7.62 (d, J=8.5 Hz, 2H), 7.50 (s, 1H), 7.39 (d, J=8.5 Hz, 2H), 7.32-7.26 (m, 2H), 7.24-7.18 (m, 3H), 4.90-4.81 (m, 1H), 4.60-4.54 (m, 1H), 3.60 (d, J=2.3 Hz, 1H), 3.33-3.23 (m, 1H), 3.16-2.98 (m, 5H), 2.67-2.00 (m, 2H), 1.93-1.75 (m, 4H), 0.90 (t, J=7.4 Hz, 3H) ppm. MS (ESI) m/z 420 (M+H)⁺.

### Example 67

### (2S,3S)-(4-(4-(2-Cyclohexylethyl)-1H-1,2,3-triazol-1-yl)benzyl)-3-hydroxypyrrolidine-2-carboxamide

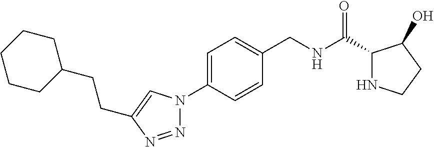

Utilizing a procedure similar to that described in Preparation A, the compound of Example 67 was prepared from (4-(4-(2-cyclohexylethyl)-1H-1,2,3-triazol-1-yl)phenyl)methanamine and N-(tert-butoxycarbonyl)-trans-3-hydroxy-L-proline. The product was obtained as an off-white solid: ¹H NMR (CDCl₃) δ 8.08-8.01 (m, 1H), 7.70-7.65 (m, 3H), 7.38 (d, J=8.5 Hz, 2H), 4.60-4.56 (m, 1H), 4.47 (d, J=6.2 Hz, 2H), 3.69 (d, J=2.3 Hz, 1H), 3.30-3.22 (m, 1H), 3.01-2.94 (m, 1H), 2.83-2.76 (m, 2H), 2.01 (br s, 2H), 1.87-1.58 (m, 9H), 1.38-1.10 (m, 4H), 1.01-0.89 (m, 2H) ppm. MS (ESI) m/z 398 (M+H)⁺.

The intermediate, (4-(4-(2-cyclohexylethyl)-1H-1,2,3-triazol-1-yl)phenyl)methanamine, was prepared using procedures similar to that described in Example 66 from 2,2,2-trifluoro-N-(4-iodobenzyl)ethanamide (2.42 g, 7.41 mmol) and but-3-ynylcyclohexane (1.01 g, 7.41 mmol) and afforded as a white solid: ¹H NMR (CDCl₃) δ 7.72-7.64 (m, 3H), 7.46 (d, J=8.3 Hz, 2H), 3.95 (s, 2H), 2.85-2.76 (m, 2H), 1.84-1.58 (m, 7H), 1.44 (br s, 2H), 1.39-1.08 (m, 4H), 1.02-0.89 (m, 2H) ppm.

### Example 68

### (2S,3S)—N-(4-(4-(4-Fluorophenethyl)-1H-1,2,3-triazol-1-yl)benzyl)-3-hydroxypyrrolidine-2-carboxamide

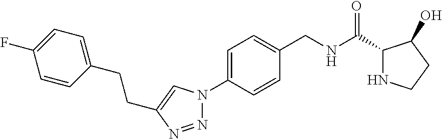

Utilizing a procedure similar to that described in Preparation A, the compound of Example 68 was prepared from (4-(4-(4-fluorophenethyl)-1H-1,2,3-triazol-1-yl)phenyl)methanamine and N-(tert-butoxycarbonyl)-trans-3-hydroxy-L-proline. The product was obtained as an off-white solid: ¹H NMR (CDCl₃) δ 8.07-8.01 (m, 1H), 7.63 (d, J=8.5 Hz, 2H), 7.53 (s, 1H), 7.38 (d, J=8.5 Hz, 2H), 7.19-7.13 (m, 2H), 7.00-6.94 (m, 2H), 4.61-4.55 (m, 1H), 4.47 (d, J=6.2 Hz, 2H), 3.69 (d, J=2.4 Hz, 1H), 3.31-3.21 (m, 1H), 3.13-2.94 (m, 6H), 2.50-1.60 (m, 3H) ppm. MS (ESI) m/z 410 (M+H)⁺.

The intermediate, (4-(4-(4-fluorophenethyl)-1H-1,2,3-triazol-1-yl)phenyl)methanamine, was prepared using procedures similar to that described in Example 66 from 2,2,2-trifluoro-N-(4-iodobenzyl)ethanamide (2.42 g, 7.41 mmol) and 1-(but-3-ynyl)-4-fluorobenzene (1.10 g, 7.41 mmol) and afforded as an off-white solid: ¹H NMR (CDCl₃) δ 7.64 (d, J=8.5 Hz, 2H), 7.54 (s, 1H), 7.46 (d, J=8.5 Hz, 2H), 7.19-7.12 (m, 2H), 7.00-6.93 (m, 2H), 3.94 (2H), 3.12-3.00 (m, 4H), 1.50 (br s, 2H) ppm.

### Example 69

### (2S,3S)-3-Hydroxy-N-(4-(4-phenethyl-1H-pyrazol-1-yl)benzyl)pyrrolidine-2-carboxamide

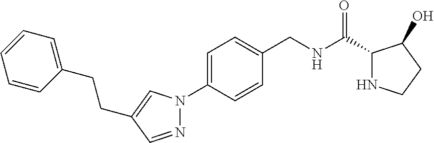

### Preparation of the Intermediate (4-(4-phenethyl-1H-pyrazol-1-yl)phenyl)-methanamine

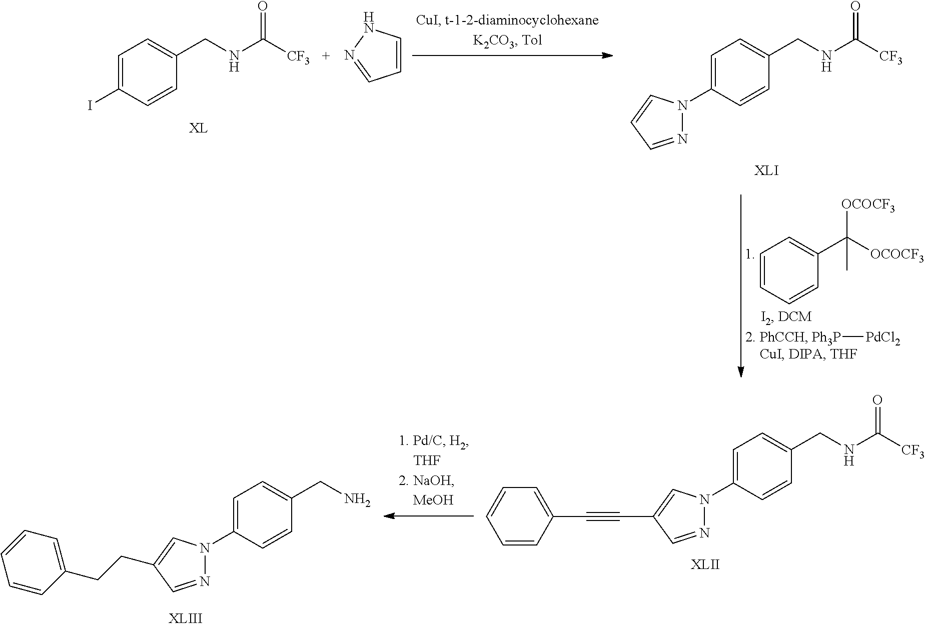

### Step 1) Preparation of N-(4-(1H-pyrazol-1-yl)benzyl)-2,2,2-trifluoroethanamide (XLI)

To a stirred solution of 2,2,2-trifluoro-N-(4-iodobenzyl)ethanamide (XL) (3.12 g, 9.54 mmol), pyrazole (0.649g, 9.54 mmol), trans-N,N′-dimethylcyclohexane-1,2-diamine (0.271 g, 1.91 mmol), and potassium carbonate (2.77 g, 20.03 mmol) in toluene (10 mL) was added copper(I) iodide (0.091 g, 0.477 mmol). The reaction mixture was heated to reflux. After 6 hours, additional portions of trans-N,N′-dimethylcyclohexane-1,2-diamine (0.271 g, 1.91 mmol) and copper(I) iodide (0.091 g, 0.477 mmol) were added, and reflux continued. After 23 hours, the reaction mixture was allowed to cool to room temperature and stir. After 43 hours, the reaction mixture was filtered through Celite with the aid of ethyl acetate. The filtrate was washed with 3N ammonium hydroxide, 1N hydrochloric acid, and brine. The organic phase was dried (magnesium sulfate), filtered, and concentrated to provide 2.27 g (88%) of N-(4-(1H-pyrazol-1-yl)benzyl)-2,2,2-trifluoroethanamide(XLI) as a light yellow solid: ¹H NMR (CDCl₃) δ 7.92 (s, 1H), 7.72 (s, 1H), 7.70-7.60 (m, 2H), 7.38-7.28 (m, 2H), 6.82 (br s, 1H), 6.42 (s, 1H), 4.59-4.50 (m, 2H) ppm.

### Step 2) Preparation of 2,2,2-trifluoro-N-(4-(4-iodo-1H-pyrazol-1-yl)benzyl)ethanamide

To a stirred solution of N-(4-(1H-pyrazol-1-yl)benzyl)-2,2,2-trifluoroethanamide (XLI) (2.27 g, 8.43 mmol) in dichloromethane (50 mL) was added iodine (2.25 g, 8.85 mmol) and [bis(trifluoroacetoxy)iodo]benzene (3.81 g, 8.85 mmol). The purple reaction mixture was allowed to stir at room temperature. After 1 hour, the reaction mixture was diluted with dichloromethane and washed with saturated sodium thiosulfate solution, saturated sodium bicarbonate solution, and brine. The organic phase was dried (magnesium sulfate), filtered, and concentrated to provide 4.66 g of a yellow solid. Trituration of the crude solid with hexanes provided 3.12 g (94%) of 2,2,2-trifluoro-N-(4-(4-iodo-1H-pyrazol-1-yl)benzyl)ethanamide as an off-white solid: ¹H NMR (CDCl₃) δ 7.96 (s, 1H), 7.72 (s, 1H), 7.68-7.64 (m, 2H), 7.41-7.37 (m, 2H), 6.60 (br s, 1H), 4.57 (d, J=6.0 Hz, 2H), 1.55 (br s, 2H) ppm.

### Step 3) Preparation of 2,2,2-trifluoro-N-(4-(4-(phenethynyl)-1H-pyrazol-1-yl)benzyl)ethanamide (XLII)

To a stirred solution of 2,2,2-trifluoro-N-(4-(4-iodo-1H-pyrazol-1-yl)benzyl)ethanamide (1.52 g, 3.85 mmol), ethynylbenzene (0.432 g, 4.23 mmol), and diisopropylamine (1.17 g, 11.54 mmol) in tetrahydrofuran (30 mL) was added copper(I) iodide (0.073 g, 0.385 mmol) and bis(triphenylphosphine)palladium(II) chloride (0.135 g, 0.192 mmol). The reaction mixture was heated to 50° C. After 17 hours, the reaction mixture was allowed to cool to room temperature and was diluted with ethyl acetate. The solution was washed with 1N hydrochloric acid, 3N ammonium hydroxide, and brine. The organic phase was dried (magnesium sulfate), filtered, and concentrated to provide 1.54 g of an orange solid. Trituration of the crude solid with hexanes provided 1.37 g (96%) of 2,2,2-trifluoro-N-(4-(4-(phenethynyl)-1H-pyrazol-1-yl)benzyl)ethanamide (XLII) as a yellow solid: ¹H NMR (CDCl₃) δ 8.10 (s, 1H), 7.86 (s, 1H), 7.70 (d, J=8.6 Hz, 2H), 7.54-7.47 (m, 2H), 7.43-7.30 (m, 5H), 6.64 (br s, 1H), 4.57 (d, 5.9 Hz, 2H) ppm.

### Step 4) Preparation of 2,2,2-trifluoro-N-(4-(4-(phenethyl)-1H-pyrazol-1-yl)benzyl)ethanamide

To a stirred solution of 2,2,2-trifluoro-N-(4-(4-(phenethynyl)-1H-pyrazol-1-yl)benzyl)ethanamide (1.37 g, 3.71 mmol) in methanol (75 mL) and tetrahydrofuran (25 mL) was added 10% palladium on carbon (wet) (1.00 g). The reaction mixture was degassed under vacuum (ca. 30 mm Hg) and backfilled with nitrogen three times. After an additional evacuation, the atmosphere was replaced with hydrogen, and the reaction mixture allowed to stir at room temperature. After 3 hours, the remaining hydrogen was removed under vacuum and replaced with nitrogen. The reaction mixture was filtered through Celite, and the filtrate concentrated to provide 1.27 g (92%) of 2,2,2-trifluoro-N-(4-(4-(phenethyl)-1H-pyrazol-1-yl)benzyl)ethanamide as a tan solid: ¹H NMR (CDCl₃) δ 7.65-7.57 (m, 3H), 7.51 (s, 1H), 7.36-7.27 (m, 41.1), 7.24-7.18 (m, 3H), 6.64 (br s, 1H), 4.54 (d, J=5.9 Hz, 2H), 2.96-2.82 (m, 4H) ppm.

### Step 5) Preparation of (4-(4-phenethyl-1H-pyrazol-1-yl)phenyl)methanamine (XLIII)

To a stirred solution of 2,2,2-trifluoro-N-(4-(4-(phenethyl)-1H-pyrazol-1-yl)benzyl)ethanamide (1.27 g, 3.40 mmol) in methanol (30 mL) was added 5N sodium hydroxide (20 mL). The reaction mixture was allowed to stir at room temperature. After 80 hours, the reaction mixture was concentrated, and the residue diluted with water. The solution was extracted three times with chloroform. The combined organic phases were washed with brine, dried (magnesium sulfate), filtered, and concentrated to provide 0.891 g (94%) of (4-(4-phenethyl-1H-pyrazol-1-yl)phenyl)methanamine (XLIII) as an orange-brown solid: ¹H NMR (CDCl₃) δ 7.61-7.57 (m, 3H), 7.51 (s, 1H), 7.40-7.35 (m, 2H), 7.33-7.27 (m, 2H), 7.24-7.18 (m, 3H), 3.90 (s, 2.96-2.83 (m, 4H), 1.51 (br s, 2H) ppm.

Utilizing a procedure similar to that described in Preparation A, the compound of Example 69 was prepared from (4-(4-phenethyl-1H-pyrazol-1-yl)phenyl)methanamine and N-(tert-butoxycarbonyl)-trans-3-hydroxy-L-proline. The product was obtained as a white solid: ¹H NMR (CDCl₃) δ 8.00-7.93 (m, 1H), 7.60-7.55 (m, 3H), 7.51 (s, 1H), 7.27 (m, 4H), 7.24-7.17 (m, 3H), 4.60-4.55 (m, 1H), 4.45-4.40 (d, J=6.1 Hz, 2H), 3.68 (d, J=2.3 Hz, 1H), 3.29-3.20 (m, 1H), 3.00-2.81 (m, 6H), 2.21-1.68 (m, 3H) ppm. MS (ESI) m/z 391 (M+H)⁺.

### Example 70

### (2S,3S)—N—((S)-1-(4-(5-Heptyl-1,2,4-oxadizol-3-yl)phenyl)-2-methylpropyl)-3-hydroxypyrrolidine-2-carboxamide

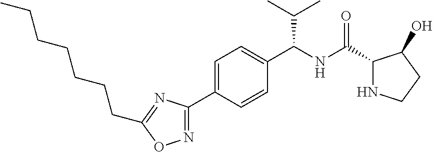

### Preparation of the intermediate ((S)-1-(4-(5-heptyl-1,2,4-oxadizol-3-yl)phenyl)-2-methylpropan-1-amine

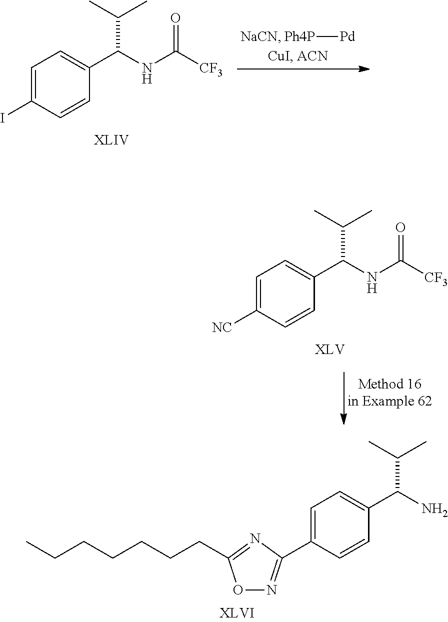

### Step 1) Preparation of (S)—N—(1-(4-cyanophenyl)-2-methylpropyl)-2,2,2-trifluoroethanamide

To a mixture of (S)-2,2,2-trifluoro-N-(1-(4-iodophenyl)-2-methylpropyl)ethanamide (XLIV) (2.00 g, 5.39 mmol), sodium cyanide (0.528 g, 10.78 mmol), copper(I) iodide (0.103 g, 0.539 mmol) and tetrakis(triphenylphosphine)palladium (0.311 g, 0.269 mmol) was added acetonitrile (15 mL, degassed by N₂ sparging for 0.5 hour prior to addition). The reaction mixture was heated to reflux and stirred. After 3 hours, the reaction mixture was allowed to cool to room temperature and was partitioned between ethyl acetate and concentrated ammonium hydroxide solution. The phases were separated, and the organic phase washed with brine, dried (magnesium sulfate), filtered, and concentrated to provide 1.79 g of an orange solid. Flash chromatography using an Isco Combiflash unit (40 g SiO₂ column, 10-20% ethyl acetate/hexanes) afforded 1.35 g (92%) of (S)—N-(1-(4-cyanophenyl)-2-methylpropyl)-2,2,2-trifluoroethanamide (XLV) as a light yellow solid: ¹H NMR (CDCl₃) δ 7.69-7.64 (m, 2H), 7.38-7.33 (m, 2H), 6.60-6.52 (m, 1H), 4.74 (t, J=8.3 Hz, 1H), 2.18-2.07 (m, 1H), 1.01 (d, J=6.7 Hz, 3H), 0.87 (d, J=6.7 Hz, 3H) ppm.

Preparation of the intermediate ((S)-1-(4-(5-heptyl-1,2,4-oxadizol-3-yl)phenyl)-2-methylpropan-1-amine (XLVI) as completed from (S)—N-(1-(4-cyanophenyl)-2-methylpropyl)-2,2,2-trifluoroethanamide (XLV) using the procedures similar to that described in Example 62 as a colorless oil: ¹H NMR (CDCl₃) δ 8.04-7.99 (m, 2H), 7.42-7.38 (m, 2H), 3.69 (d, J=7.0 Hz, 1H), 2.93 (t, J=7.6 Hz, 2H), 1.93-1.82 (m, 3H), 1.49 (br s, 2H), 1.46-1.25 (m, 8H), 0.98 (d, J=6.7 Hz, 3H), 0.88 (t, J=6.9 Hz, 3H), 0.80 (d, J=6.7 Hz, 3H) ppm.

Utilizing a procedure similar to that described in Preparation A, the compound of Example 70 was prepared from ((S)-1-(4-(5-heptyl-1,2,4-oxadizol-3-yl)phenyl)-2-methylpropan-1-amine and N-(tert-butoxycarbonyl)-trans-3-hydroxy-L-proline. The product was obtained as a white solid: ¹H NMR (CDCl₃) δ 8.17 (d, J=9.5 Hz, 1H), 8.02 (d, J=8.3 Hz, 2H), 7.33 (d, J=8.3 Hz, 2H), 4.77-4.71 (m, 1H), 4.61-4.56 (m, 1H), 3.61 (d, J=2.0 Hz, 1H), 3.34-3.26 (m, 1H), 3.09-3.02 (m, 1H) 2.93 (t, J=7.6 Hz, 2H), 2.56 (br s, 2H), 2.11-2.01 (m, 1H), 1.90-1.81 (m, 4H), 1.46-1.23 (m, 8H), 0.93-0.81 (m, 9H) ppm. MS (ESI) m/z 429 (M+H)⁺.

### Example 71

### (2S,3S)—N-(4-(5-Heptyl-1-methyl-1H-1,2,4-triazol-3-yl)benzyl)-3-hydroxypyrrolidine-2-carboxamide

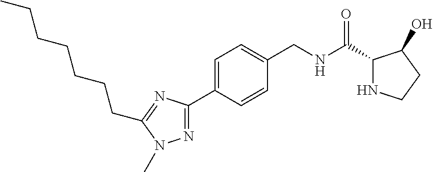

Utilizing a procedure similar to that described in Preparation A, the compound of Example 71 was prepared from (4-(5-heptyl-1-methyl-1H-1,2,4-triazol-3-yl)phenyl)methanamine and N-(tert-butoxycarbonyl)-trans-3-hydroxy-L-proline. The product was obtained as a white solid: ¹H NMR (CDCl₃) δ 8.00 (d, J=8.2 Hz, 2H), 7.97-7.90 (m, 1H), 7.28 (d, J=8.2 Hz, 2H), 4.60-4.56 (m, 1H), 4.50-4.38 (m, 2H), 3.85 (s, 3H), 3.68 (d, J=2.2 Hz, 1H), 3.27-3.19 (m, 1H), 2.99-2.92 (m, 1H), 2.79-2.73 (m, 2H), 2.71-2.25 (m, 2H), 1.85-1.73 (m, 4H), 1.45-1.23 (m, 8H), 0.88 (t, J=6.8 Hz, 3H) ppm. MS (ESI) m/z 400 (M+H)⁺.

The intermediate, (4-(5-heptyl-1-methyl-1H-1,2,4-triazol-3-yl)phenyl)methanamine, was prepared using procedures similar to that describe in Example 63 starting with 4-bromothiobenzamide and octanoyl chloride. (4-(5-heptyl-1-methyl-1H-1,2,4-triazol-3-yl)phenyl)methanamine was obtained as a yellow solid: ¹H NMR (CDCl₃) δ 8.01 (d, J=8.2 Hz, 2H), 7.35 (d, J=8.2 Hz, 2H), 3.89 (s, 2H), 3.84 (s, 3H), 2.79-2.72 (m, 2H), 1.86-1.72 (m, 4H), 1.46-1.23 (m, 8H), 0.87 (t, J=6.9 Hz, 3H) ppm.

### Example 72

### (2S,3S)—N-((S)-1-(4-(5-Heptyl-1,2,4-oxadizol-3-yl)phenyl)ethyl)-3-hydroxypyrrolidine-2-carboxamide

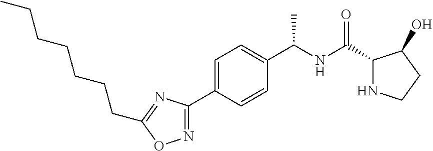

Utilizing a procedure similar to that described in Preparation A, the compound of Example 72 was prepared from ((S)-1-(4-(5-heptyl-1,2,4-oxadizol-3-yl)phenyl)ethanamine and N-(tert-butoxycarbonyl)-trans-3-hydroxy-L-proline. The product was obtained as a white solid: ¹H NMR (CDCl₃) δ 8.03 (d, J=8.3 Hz, 2H), 7.93 (d, J=7.6 Hz, 1H), 7.40 (d, J=8.3 Hz, 2H), 5.16-5.06 (m, 1H), 4.58-4.53 (m, 1H), 3.61 (d, J=2.3 Hz, 1H), 3.29-3.21 (m, 1H), 3.04-2.97 (m, 1H) 2.93 (t, J=7.6 Hz, 2H), 2.82-2.29 (m, 2H), 1.91-1.80 (m, 4H), 1.48 (d, J=7.0 Hz, 3H), 1.45-1.22 (m, 8H), 0.88 (t, J=6.9 Hz, 3H) ppm. MS (ESI) m/z 401 (M+H)⁺.

The intermediate, ((S)-1-(4-(5-heptyl-1,2,4-oxadizol-3-yl)phenyl)ethanamine, was prepared using procedures similar to that describe in Example 70 starting with (S)-2,2,2-trifluoro-N-(1-(4-iodophenyl)ethyl)ethanamide, (S)-1-(4-(5-heptyl-1,2,4-oxadizol-3-yl)phenyl)ethanamine was obtained as a white solid: ¹H NMR (CDCl₃) δ 8.03 (d, J=8.3 Hz, 2H), 7.46 (d, J=8.3 Hz, 2H), 4.18 (q, J=6.6 Hz, 1H), 2.93 (t, J=7.6 Hz, 2H), 1.91-1.81 (m, 2H), 1.51 (br s, 2H), 1.46-1.23 (m, 11H), 0.88 (t, J=6.9 Hz, 3H) ppm.

### Example 73

### (2S,3S)—N—((R)-1-(4-(5-Heptyl-1,2,4-oxadizol-3-yl)phenyl)ethyl)-3-hydroxypyrrolidine-2-carboxamide

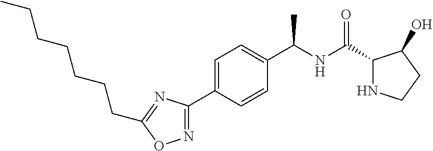

Utilizing a procedure similar to that described in Preparation A, the compound of Example 73 was prepared from ((R)-1-(4-(5-heptyl-1,2,4-oxadizol-3-yl)phenyl)ethanamine and N-(tert-butoxycarbonyl)-trans-3-hydroxy-L-proline. The product was obtained as a white solid: ¹H NMR (CDCl₃) δ 8.02 (d, J=8.3 Hz, 2H), 7.97 (d, J=8.5 Hz, 1H), 7.35 (d, J=8.3 Hz, 2H), 5.15-5.06 (m, 1H), 4.52-4.47 (m, 1H), 3.65 (d, J=1.9 Hz, 1H), 3.28-3.20 (m, 1H), 2.99-2.89 (m, 3H), 2.54 (br s, 2H), 1.91-1.66 (m, 4H), 1.50 (d, J=7.0 Hz, 3H), 1.45-1.22 (m, 8H), 0.88 (t, J=6.8 Hz, 3H) ppm. MS (ESI) m/z 401 (M+H)⁺.

The intermediate, ((R)-1-(4-(5-heptyl-1,2,4-oxadizol-3-yl)phenyl)ethanamine, was prepared using procedures similar to that describe in Example 70 starting with (R)-2,2,2-trifluoro-N-(1-(4-iodophenyl)ethyl)ethanamide. (S)-1-(4-(5-heptyl-1,2,4-oxadizol-3-yl)phenyl)ethanamine was obtained as a white solid: ¹H NMR (CDCl₃) δ 8.03 (d, J=8.3 Hz, 2H), 7.46 (d, 8.3 Hz, 2H), 4.18 (q, J=6.6 Hz, 1H), 2.93 (t, J=7.6 Hz, 2H), 1.91-1.81 (m, 2H) 1.51 (br s, 2H), 1.46-1.23 (m, 11H), 0.88 (t, J=6.9 Hz, 3H) ppm.

### Example 74

### (2S,3S)—N-(4-(5-Heptyl-1,3,4-oxadiazol-2-yl)benzyl)-3-hydroxypyrrolidine-2-carboxamide

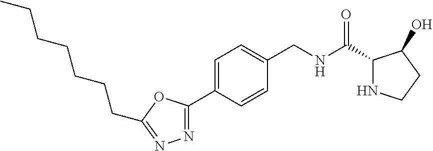

### Preparation of the intermediate (4-(5-heptyl-1,3,4-oxadiazol-2-yl)phenyl)methanamine (L)

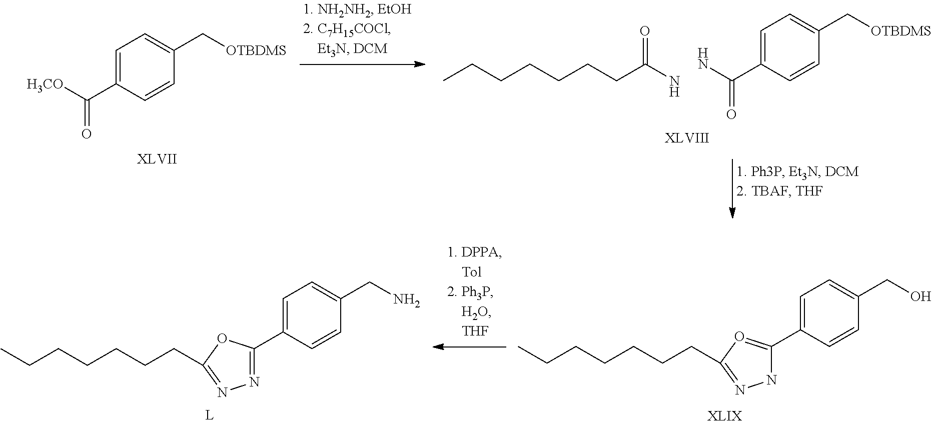

### Step 1) Preparation of 4-((tert-butyldimethylsilyloxy)methyl)benzohydrazide

To a stirred solution of methyl 4-((tert-butyldimethylsilyloxy)methyl)benzoate (XLVII) (16.88 g, 60.18 mmol) in ethanol (120 mL) was added hydrazine hydrate (18 mL). The reaction mixture was allowed to stir at room temperature. After 1.5 hours, an additional portion of hydrazine hydrate (18 mL) and sodium cyanide (0.2 g) were added, and the reaction mixture was heated to 70° C. After 18 hours, the reaction mixture was allowed to cool to room temperature aired Was diluted with water. The mixture was cooled to 10° C. and stirred. The resulting, precipitate was isolated by filtration, washed with water, and dried to provide 14.58 g (86%) of 4-((tert-butyldimethylsilyloxy)methyl)benzohydrazide as a white solid: ¹H NMR (CDCl₃) δ 7.71 (d, J=8.2 Hz, 2H), 7.43 (br s, 1H), 7.39 (d, J=8.1 Hz, 2H), 4.78 (s, 4.11 (s, 2H), 0.94 (s, 9H), 0.10 (s, 6H) ppm.

### Step 2) Preparation of 4-((tert-butyldimethylsilyloxy)methyl)-N′-octanoylbenzohydrazide (XLVIII)

To a stirred solution of 4-((tert-butyldimethylsilyloxy)methyl)benzohydrazide (2.87 g, 10.23 mmol) and triethylamine (1.24 g, 12.28 mmol) in dichloromethane (75 mL) was added octanoyl chloride (1.66 g, 10.23 mmol). A mild exotherm was noted upon addition. The reaction mixture was allowed to stir at room temperature. After 0.5 hour, the reaction mixture was diluted with dichloromethane and washed with 1N hydrochloric acid, saturated sodium bicarbonate solution, and brine. The organic phase was dried (magnesium sulfate), filtered, and concentrated to provide 4.02 g (97%) of 4-((tert-butyldimethylsilyloxy)methyl)-N′-octanoylbenzohydrazide (XLVIII) as a foamy white solid: ¹H NMR (CDCl₃) δ 9.71 (d, J−5.3 Hz, 1H), 9.53 (d, J=5.3 Hz, 1H), 7.80 (d, J=8.3 Hz, 2H), 7.34 (d, J=8.3 Hz, 2H), 4.75 (s, 2H), 2.33-2.27 (m, 2H), 1.68-1.60 (m, 2H), 1.33-1.21 (m, 8H), 0.93 (s, 9H), 0.86 (t, J=6.9 Hz, 3H), 0.09 (s, 6H) ppm.

### Step 3) Preparation of 2-(4-((tert-butyldimethylsilyloxy)methyl)phenyl)-5-heptyl-1,3,4-oxadiazole

To a stirred solution of 4-((tert-butyldimethylsilyloxy)methyl)-N′-octanoylbenzohydrazide (4.02 g, 9.89 mmol), triphenylphosphine (4.07 g, 15.52 mmol), and triethylamine (1.57 g, 15.52 mmol) in dichloromethane (100 mL) was added carbon tetrachloride (7.60 g, 49.43 mmol). The reaction mixture was heated to reflux. After 6 hours, the reaction mixture was allowed to cool to room temperature and stirring, continued. After 18 hours, the reaction mixture was washed with 1N hydrochloric acid, saturated sodium bicarbonate solution, and brine. The organic phase was dried (magnesium sulfate), filtered, and concentrated to provide a sticky white solid. Flash chromatography using an Isco Combiflash unit (120 g SiO₂ column, 10-25% ethyl acetate/hexanes) afforded 2.14 g (56%) of 2-(4-((tert-butyldimethylsilyloy)methyl)phenyl)-5-heptyl-1,3,4-oxadiazole as a white solid: ¹H NMR (CDCl₃) δ 7.99 (d, J=8.3 Hz, 2H), 7.45 (d, J=8.3 Hz, 2H), 4.80 (s, 2H), 2.91 (t, J=7.6 Hz, 2H), 1.89-1.79 (m, 2H), 1.47-1.24 (m, 8H), 0.95 (s, 9H), 0.88 (t, J=6.9 Hz, 3H), 0.11 (s, 6H) ppm.

### Step 4) Preparation of (4-(5-heptyl-1,3,4-oxadiazol-2-yl)phenyl)methanol (XLIX)

To a stirred solution of 2-(4-((tert-butyldimethylsilyloy)methyl)phenyl)-5-heptyl-1,3,4-oxadiazole (2.14 g, 5.50 mmol) in tetrahydrofuran (50 mL) was added 1.0 M tetrabutylammonium fluoride in tetrahydrofuran (8.2 mL, 8.20 mmol). The reaction mixture was allowed to stir at room temperature. After 1 hour, the reaction mixture was diluted with ethyl acetate and washed with 1N hydrochloric acid and brine. The organic phase was dried (magnesium sulfate), filtered, and concentrated to provide 1.88 g (>100%) of (4-(5-heptyl-1,3,4-oxadiazol-2-yl)phenyl)methanol as a crude white solid: ¹H NMR (CDCl₃) δ 8.02 (d, J=8.1 Hz, 2H), 7.50 (d, J=8.1 Hz, 2H), 4.78 (s, 2H), 2.91 (t, J=7.6 Hz, 2H), 1.98 (br s, 1H), 1.89-1.79 (m, 2H), 1.48-1.23 (m, J=0.92-0.84 (m, 3H) ppm.

### Step 5) Preparation of 2-(4-(azidomethyl)phenyl)-5-heptyl-1,3,4-oxadiazole

To a stirred solution of the crude (4-(5-heptyl-1,3,4-oxadiazol-2-yl)phenyl)methanol (1.51 g, 5.51 mmol) and diphenylphosphoryl azide (1.82 g, 6.61 mmol) in toluene (20 mL) was added 1,8-diazabicycloundec-7-ene (1.00 g, 6.61 mmol). The reaction mixture was allowed to stir at room temperature. After 18 hours, the reaction mixture was concentrated, and the residue dissolved in diethyl ether. The solution was washed with 1N hydrochloric acid, 1N sodium hydroxide, and brine. The organic phase was dried (magnesium sulfate), filtered, and concentrated to provide 1.95 g (>100%) of 5-(azidomethyl)-3-(heptylphenyl)-1,2,4-oxadiazole as a crude yellow oil: ¹H NMR (CDCl₃) δ 8.06 (d, J=8.3 Hz, 2H), 7.46 (d, J=8.3 Hz, 2H), 4.43 (s 2H), 2.92 (t, 7.6 Hz, 2H), 1.91-1.78 (m, 2H), 1.48-1.22 (m, 8H), 0.94-0.84 (m, 3H) ppm.

### Step 6) Preparation of (4-(5-heptyl-1,3,4-oxadiazol-2-yl)phenyl)methanamine (L)

To a stirred solution of the crude 5-(azidomethyl)-3-(heptylphenyl)-1,2,4-oxadiazole (1.65 g, 5.51 mmol) and water (1 mL) in tetrahydrofuran (20 mL) was added triphenylphosphine (1.81 g, 7.16 mmol). The reaction mixture was allowed to stir at room temperature. After 18 hours, the reaction mixture was diluted with ethyl acetate and washed with saturated sodium bicarbonate solution and brine. The organic phase was dried (magnesium sulfate), filtered, and concentrated to provide a yellow oil. Flash chromatography using an Isco Combiflash unit (80 g SiO₂ column, 1-5% 2M ammonia in methanol/dichloromethane) afforded 1.05 g (70% from step 3) of (4-(5-heptyl-1,3,4-oxadiazol-2-yl)phenyl)methanamine (L) as a white solid: ¹H NMR (CDCl₃) δ 8.00 (d, J=8.3 Hz, 2H), 7.45 (d, J=8.3 Hz, 2H), 3.95 (s, 2H), 2.91 (t, J=7.6 Hz, 2H), 1.89-1.78 (m, 2H), 1.54-1.23 (m, 10H), 0.88 (t, J=6.9 Hz, 3H) ppm.

Utilizing a procedure similar to that described in Preparation A, the compound of Example 74 was prepared from (4-(5-heptyl-1,3,4-oxadiazol-2-yl)phenyl)methanamine and N-(tert-butoxycarbonyl)-trans-3-hydroxy-L-proline. The product was obtained as a white solid: ¹H NMR (CDCl₃) δ 8.11-8.04 (m, 1H), 7.98 (d, J=8.3 Hz, 2H), 7.36 (d, J=8.3 Hz, 2H), 4.62-4.57 (m, 1H), 4.48 (d, J=6.2 Hz, 2H), 3.71 (d, 2.1 Hz, 1H), 3.30-3.22 (m, 1H, 3.02-2.94 (m, 1H), 2.91 (t, J=7.6 Hz, 2H), 2.80-2.24(m, 2H), 1.89-1.79 (m, 4H), 1.48-1.23 (m, 8H), 0.88 (t, J=6.8 Hz, 3H) ppm. MS (ESI) m/z 387 (M+H)⁺.

### Example 75

### (2S,3S)—N—((S)-1-(4-(5-Heptyl-1,2,4-oxadiazol-3-yl)phenyl)propyl)-3-hydroxypyrrolidine-2-carboxamide

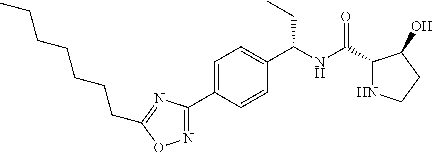

Utilizing a procedure similar to that described in Preparation A, the compound of Example 75 was prepared from (S)-1-(4-(5-heptyl-1,2,4-oxadizol-3-yl)phenyl)propan-1-amine and N-(tert-butoxycarbonyl)-trans-3-hydroxy-L-proline. The product was obtained as a white solid: ¹H NMR (CDCl₃) δ 8.03 (d, J=8.3 Hz, 2H), 7.98 (d, J=8.9 Hz, 1H), 7.37 (d, J=8.3 Hz, 2H), 4.93-4.83 (m, 1H), 4.60-4.54 (m, 1H), 3.61 (d, J=2.3 Hz, 1H), 3.32-3.23 (m, 1H), 3.07-2.99 (m, 1H), 2.93 (t, J=7.6 Hz, 2H), 2.47 (br s, 2H), 1.92-1.74 (m, 6H), 1.47-1.23 (m, 8H), 0.93-0.84 (m, 6H) ppm. MS (ESI) m/z 415 (M+H)⁺.

The intermediate, (S)-1-(4-(5-heptyl-1,2,4-oxadizol-3-yl)phenyl)propan-1-amine, was prepared using procedures similar to that described in Example 70 starting with (R)-2,2,2-trifluoro-N-(1-(4-iodophenyl)propylyl)ethanamide. (S)-1-(4-(5-heptyl-1,2,4-oxadizol-3-yl)phenyl)propan-1-amine was obtained as a colorless oil: ¹H NMR (CDCl₃) δ 8.03 (d, J=8.3 Hz, 2H), 7.42 (d, 8.3 Hz, 2H), 3.88 (t, J=6.8 Hz, 1H), 2.93 (t, J=7.6 Hz, 2H), 1.91-1.82 (m, 2H) 1.76-1.65 (m, 2H), 1.50 (br s, 2H), 1.46-1.24 (m, 8H), 0.91-0.83 (m, 6H) ppm.

### Example 76

### (2S,3S)—N-(4-(3-Heptyl-1,2,4-oxadiazol-5-yl)benzyl)-3-hydroxypyrrolidine-2-carboxamide

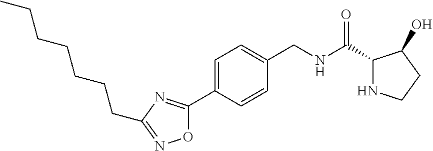

### Preparation of the intermediate (4-(3-heptyl-1,2,4-oxadiazol-5-yl)phenyl)methanamine

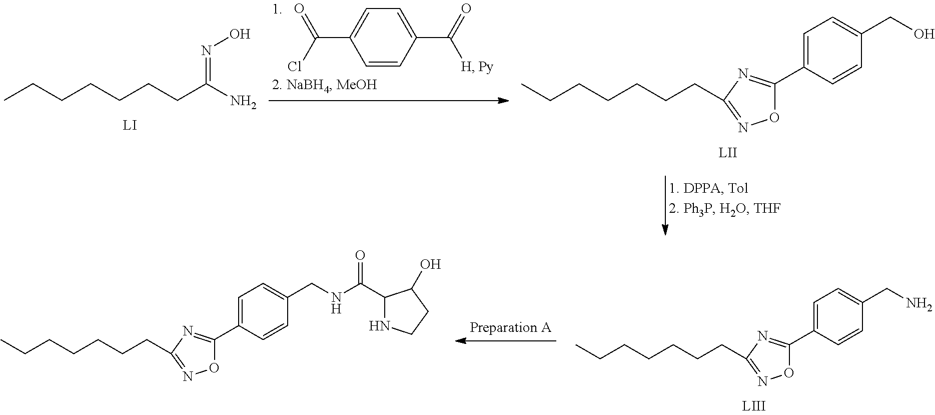

### Step 1) Preparation of 4-(3-heptyl-1,2,4-oxodiazol-5-yl)benzaldehyde

To a stirred solution of N′-hydroxyoctanimidamine (LI) (2.00 g, 12.64 mmol) in pyridine (20 mL) was added 4-formylbenzoyl chloride (2.34 g, 13.90 mmol). The reaction mixture was heated to reflux. After 3 hours, the reaction mixture was allowed to cool to room temperature and was diluted with water. The mixture was extracted with diethyl ether. The organic phase was washed with 1N hydrochloric acid, saturated sodium bicarbonate, and brine. The organic phase was dried (magnesium sulfate), filtered, and concentrated to provide 2.27 g of a brown solid. Flash chromatography using an Isco Combiflash unit (120 g SiO₂ column, 5-10% ethyl acetate/hexane) afforded 1.55 g (45%) of 4-(3-heptyl-1,2,4-oxadiazol-5-yl)benzaldehyde as a white solid: ¹H NMR (CDCl₃) δ 10.12 (s, 8.30 (d, J=8.3 Hz, 2H), 8.04 (d, J=8.3 Hz, 2H), 2.85-2.78 (m, 2H), 1.87-1.76 (m, 2H), 1.47-1.23 (m, 8H), 0.88 (t, J−6.9 Hz, 3H) ppm.

### Step 2) Preparation of 4-(3-heptyl-1,2,4-oxadiazol-5yl)phenyl)methanol (LII)

To a stirred solution of 4-(3-heptyl-1,2,4-oxadiazol-5-yl)benzaldehyde (1.55 g, 5.69 mmol) in methanol (20 mL) was added sodium borohydride (0.323 g, 8.54 mmol). Gas evolution was noted along With a mild exotherm. The reaction mixture was allowed to stir at room temperature. After 3 hours, the reaction mixture was quenched with 1N hydrochloric acid and concentrated. The residue was dissolved in diethyl ether and washed with 1N hydrochloric acid, 1N sodium hydroxide, and brine. The organic phase was dried (magnesium sulfate), filtered, and concentrated to provide 1.56 g (100%) of 4-(3-heptyl-1,2,4-oxadiazol-5-yl)phenyl)methanol (LII) as a white solid: ¹H NMR (CDCl₃) δ 8.10 (d, J=8.3 Hz, 2H), 7.51 (d, J=8.3 Hz, 2H), 4.79 (s. 2H), 2.82-2.75 (m, 2H), 1.99 (br s, 1H), 1.85-1.75 (m, 2H), 1.45-1.23 (m, 8H), 0.88 (t, J=6.9 Hz, 3H) ppm.

### Step 3) Preparation of (4-(3-heptyl-1,2,4-oxadizol-5-yl)phenyl)methanamine (LIII)

(4-(3-heptyl-1,2,4-oxadizol-5-yl)phenyl)methanamine (LIII) was prepared using procedures similar to that described in the steps of 5 and 6 in Example 74. (4-(3-heptyl-1,2,4-oxadizol-5-yl)phenyl)methanamine (LIII) was obtained as a white solid: ¹H NMR (CDCl₃) δ 8.08 (d, J=8.2 Hz, 2H), 7.48 (d, J=8.2 Hz, 2H), 3.97 (s, 2H), 2.82-2.74 (m, 2H), 1.85-1.75 (m, 2H), 1.51-1.23 (m, 10H), 0.88 (t, J=6.9 Hz, 3H) ppm.

Utilizing a procedure similar to that described, in Preparation A, the compound of Example 76 was prepared from (4-(3-heptyl-1,2,4-oxadizol-5-yl)phenyl)methanamine and N-(tert-butoxycarbonyl)-trans-3-hydroxy-L-proline. The product was obtained as a white solid: ¹H NMR (CDCl₃) δ 8.14-8.03 (m, 3H), 7.38 (d, J=8.3 Hz, 2H), 4.62-4.56 (m, 1H), 4.49 (d, J=6.2 Hz, 2H), 3.71 (d, J=2.1 Hz, 1H), 3.31-3.21 (m, 1H), 3.02-2.93 (m, 1H), 2.82-2.73 (m, 2H), 2.71-2.08 (m, 2H), 1.88-1.74 (m, 4H), 1.45-1.22 (m, 8H), 0.87 (t, J=6.9 Hz, 3H) ppm. MS (ESI) m/z 387 (M+H)⁺.

### Example 77

### (2S,3S)—N-((3-(4-Heptylphenyl)-1,2,4-oxadiazol-5-yl)methyl)-3-hydroxypyrrolidine-2-carboxamide

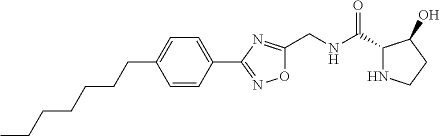

### Preparation of the intermediate (3-(4-heptylphenyl)-1,2,4-oxadiazol-5-yl)methanamine

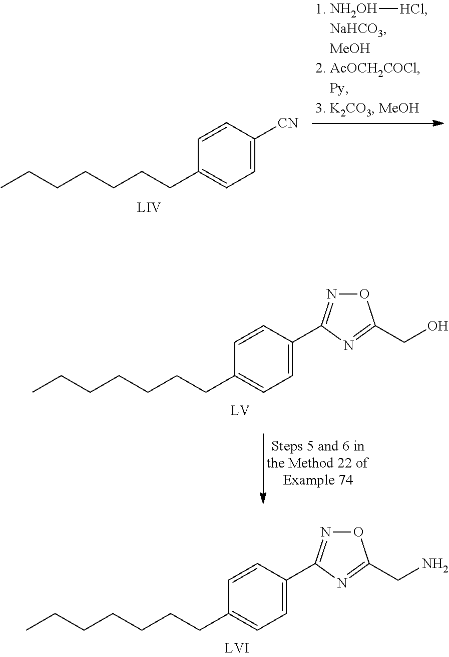

### Step 1) Preparation of 4-heptyl-N′-hydroxybenzimidamine

To as stirred suspension of 4-heptylbenzonitrile (LIV) (4.70 g, 23.35 mmol) and sodium bicarbonate (7.85 g, 93.39 mmol) in methanol (75 mL) was added hydroxylamine hydrochloride (3.24 g, 46.69 mmol). The reaction mixture was heated to reflux. After 17 hours, the reaction mixture was allowed to cool to room temperature and was filtered. The filtrate was concentrated, and the residue suspended in water. The suspension was filtered, the filter cake was washed with water, and the solid dried to provide 5.69 g of 4-heptyl-N′-hydroxybenzimidamine as a white solid: ¹H NMR (DMSO-d₆) δ 9.50 (s, 1H), 7.55 (d, J=7.8 Hz, 2H), 7.16 (d, J=7.8 Hz, 2H), 5.71 (s, 2H), 2.55 (t, J=7.5 Hz, 2H), 1.61-1.48 (m, 2H), 1.32-1.16 (m, 8H), 0.83 (t, J=6.5 Hz, 3H) ppm.

### Step 2) Preparation of (3-(4-heptylphenyl)-1,2,4-oxadiazol-5-yl)methyl ethanoate

To a stirred solution of 4-heptyl-N′-hydroxybenzimidamine (2.00 g, 8.53 mmol) in pyridine (20 mL) was added acetoxyacetyl chloride (1.40 g, 10.24 mmol). The reaction mixture was heated to reflux. After 17 hours, the reaction mixture was allowed to cool to room temperature and was diluted with water. The mixture was extracted with diethyl ether. The organic phase was washed with 1N hydrochloric acid, saturated sodium bicarbonate, and brine. The organic phase was dried (magnesium sulfate), filtered, and concentrated to provide a brown oil. Flash chromatography using an Isco Combiflash unit (120 g SiO₂ column, 10-20% ethyl acetate/hexanes) afforded 1.76 g (65%) of (3-(4-heptylphenyl)-1,2,4-oxadiazol-5-yl)methyl ethanoate as a colorless oil: ¹H NMR (CDCl₃) δ 7.98 (d, J=8.3 Hz, 2H), 7.29 (d, J=8.3 Hz, 2H), 5.35 (s, 2H), 2.69-2.62 (m, 2H), 2.22 (s, 3H), 1.69-1.58 (m, 2H), 1.37-1.21 (m, 8H), 0.88 (t, J=6.9 Hz, 3H) ppm.

### Step 3) Preparation of (3-(4-heptylphenyl)-1,2,4-oxadiazol-5-yl)methanol (LV)

To a stirred solution of of (3-(4-heptylphenyl)-1,2,4-oxadiazol-5-yl)methyl ethanoate (1.76 g, 5.56 mmol) in methanol (20 mL) was added potassium carbonate (0.20 g). The reaction mixture was allowed to stir at room temperature. After 0.5 hour, the reaction mixture was diluted with water and was extracted twice with diethyl ether. The combined organic phases were washed with brine, dried (magnesium sulfate), filtered, and concentrated to provide 1.50 g of (3-(4-heptylphenyl)-1,2,4-oxadiazol-5-yl)methanol (LV) as a white solid: ¹H NMR (CDCl₃) δ 7.98 (d, J=8.1 Hz, 2H), 7.29 (d, J=8.1 Hz, 2H), 4.95 (d, J=6.5 Hz, 2H), 2.79 (t, J=6.5 Hz, 1H), 2.70-2.62 (m, 2H), 1.69-1.58 (m, 2H), 1.38-1.20 (m, 8H), 0.88 (t, J=6.9 Hz, 3H) ppm.

### Step 4) Preparation of (3-(4-heptylphenyl)-1,2,4-oxadiazol-5-yl)methanamine (LVI)

(3-(4-heptylphenyl)-1,2,4-oxadiazol-5-yl)methanamine (LVI) was prepared using procedures similar to that described in the steps of 5 and 6 in the Example 74. (3-(4-heptylphenyl)-1,2,4-oxadiazol-5-yl)methanamine was obtained as a white solid (co-obtained with about 10% triphenylphosphine oxide): ¹H NMR (CDCl₃) δ 7.98 (d, J=8.2 Hz, 2H), 7.29 (d, J=8.2 Hz, 2H), 4.14 (s, 2H), 2.69-2.62 (m, 2H), 1.72-1.58 (m, 4H), 1.39-1.21 (m, 8H), 0.88 (t, J=6.9 Hz, 3H) ppm.

Utilizing a procedure similar to that described in Preparation A, the compound of Example 77 was prepared from (3-(4-heptylphenyl)-1,2,4-oxadiazol-5-yl)methanamine and N-(tert-butoxycarbonyl)-trans-3-hydroxy-L-proline. The product was obtained as a white solid: ¹H NMR (CDCl₃) δ 8.41-8.33 (m, 1H), 7.94 (d, J=8.2 Hz, 2H), 7.28 (d, J=8.2 Hz, 2H), 4.82-4.63 (m, 2H), 4.62-4.57 (m, 1H), 3.74 (d, J=1.7 Hz, 1H), 3.34-3.25 (m, 1H), 3.12-3.03 (m, 1H), 2.70-2.44 (m, 4H), 1.97-1.80 (m, 2H), 1.68-1.58 (m, 2H), 1.38-1.19 (m, 8H), 0.87 (t, J=6.9 Hz, 3H) ppm. MS (ESI) m/z 387 (M+H)⁺.

### Example 78

### (2S,3S)-3-Hydroxy-N-(4-(5-(4-isobutylphenyl)-1,2,4-oxadiazol-3-yl)benzyl)pyrrolidine-2-carboxamide

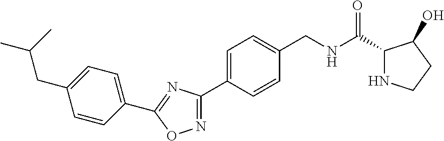

Utilizing a procedure similar to that described in Preparation A, the compound of Example 78 was prepared from (4-(5-(4-isobutylphenyl)-1,2,4-oxadiazol-3-yl)phenyl)methanamine and N-(tert-butoxycarbonyl)-trans-3-hydroxy-L-proline. The product was obtained as a white solid: ¹H NMR (DMSO-d₆) δ 8.55 (t, 6.3 Hz, 1H), 8.10 (d, J=8.3 Hz, 2H), 8.04 (d, J=8.3 Hz, 2H), 7.49-7.39 (m, 4H), 4.97 (d, J=3.9 Hz, 1H), 4.36 (d, J=6.2 Hz, 2H), 4.25-4.20 (m, 1H), 3.44-3.41 (m, 1H), 3.08-2.99 (m, 1H), 2.94-2.86 (m, 1H), 2.58 (d, J=7.2 Hz, 2H), 1.98-1.85 (m, 1H), 1.65-1.57 (m, 2H), 0.90 (d, J=6.6 Hz, 6H) ppm. MS (ESI) m/z 421 (M+H)⁺.

The intermediate, (4-(5-(4-isobutylphenyl)-1,2,4-oxadiazol-3-yl)phenyl)methanamine, was prepared using procedures similar to that describe in the Steps 5 and 6 in Example 74 starting with (4-(5-(4-isobutylphenyl)-1,2,4-oxadiazol-3-yl)phenyl)methanol. (4-(5-(4-isobutylphenyl)-1,2,4-oxadiazol-3-yl)phenyl)methanamine was obtained as a white solid: ¹H NMR (CDCl₃) δ 8.18-8.04 (m, 4H), 7.46 (d, J=8.1 Hz, 2H), 7.32 (d, J=8.1 Hz, 2H), 3.96 (br s, 2H), 2.57 (d, J=6.8 Hz, 2H), 1.99-1.87 (m, 1H), 1.48 (br s, 2H), 0.93 (d, J=6.6 Hz, 6H) ppm.

### Example 79

### (2S,3S)—N-(4-(5-Heptyl-1,2,4-oxadiazol-3-yl)benzyl)-3-hydroxypyrrolidine-2-carboxamide

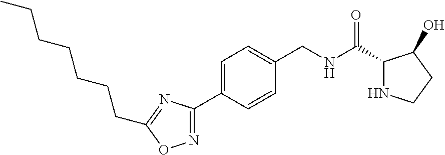

### Preparation of the intermediate (4-(5-heptyl-1,2,4-oxadiazol-3-yl)phenyl)methanamine (LX)

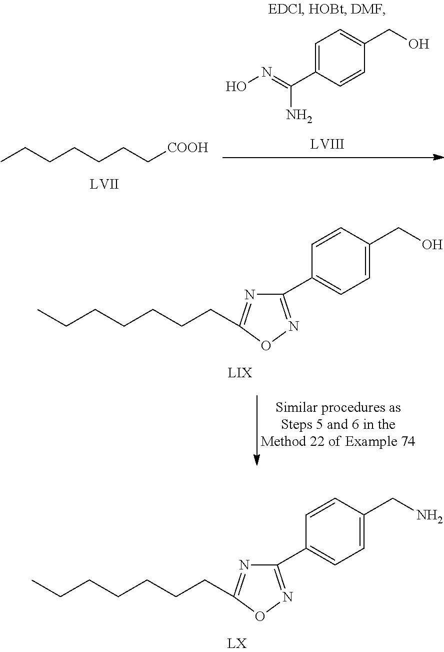

### Step 1) Preparation of (4-(5-heptyl-1,2,4-oxadiazol-3-yl)phenyl)methanol (LIX)

To a stirred solution of octanoic acid (LVII) (1.15 g, 8.00 mmol) and 1-hydroxybenztriazole hydrate (1.10 g, 8.40 mmol) in N,N-dimethylformamide (10 mL) was added N-(3-dimethylaminopropyl)-N′-ethylcarbodiimide hydrochloride (1.61 g, 8.40 mmol). The reaction mixture was allowed to stir at room temperature for 15 minutes, and then N′-hydroxy-4-(hydroxymethyl)benzimidamide (LVIII) (1.40 g, 8.40 mmol) was added. The reaction mixture was heated to 140° C. and stirred. After 2.5 hours, the reaction mixture was allowed to cool to room temperature and was diluted with water. The mixture was extracted three times with ethyl acetate. The combined organic phases were dried (sodium sulfate), filtered, and concentrated to provide a yellow solid. Flash chromatography using an Isco Combiflash unit (40 g SiO₂ column, 50% ethyl acetate/hexanes) afforded 1.34 g (61%) of (4-(5-heptyl-1,2,4-oxadiazol-3-yl)phenyl)methanol as a yellow solid: ¹H NMR (CDCl₃) δ 8.05 (d, J=8.2 Hz, 2H), 7.46 (d, J=8.2 Hz, 2H), 4.76 (s, 2H), 2.94 (t, J=7.7 Hz, 2H), 1.92-1.81 (m, 2H), 1.37-1.23 (m, 9H), 0.89 (t, J=6.9 Hz, 3H) ppm.

### Step 2) Preparation (4-(5-heptyl-1,2,4-oxadiazol-3-y)phenyl)methanamine (LX)

(4-(5-heptyl-1,2,4-oxadiazol-3-yl)phenyl)methanamine (LX) was prepared using procedures similar to that describe in the Steps 5 and 6 in Example 74, starting with (4-(5-heptyl-1,2,4-oxadiazol-3-yl)phenyl)methanol (LIX) obtained in the Step 1, as a white solid: ¹H NMR (CDCl₃) δ 8.07-8.02 (m, 2H), 7.46-7.40 (m, 2H), 3.94 (s, 2H), 2.94 (t, J=7.6 Hz, 2H), 1.93-1.82 (m, 2H), 1.50-1.24 (m, 10H), 0.89 (t, J=6.9 Hz, 3H) ppm.

Utilizing a procedure similar to that described in Preparation A, compound of Example 79 was prepared from (4-(5-heptyl-1,2,4-oxadiazol-3-yl)phenyl)methanamine and N-(tert-butoxycarbonyl)-trans-3-hydroxy-L-proline. The product was obtained as to white solid: ¹H NMR (CDCl₃) δ 8.06-8.01 (m, 3H), 7.37-7.32 (m, 2H), 4.62-4.58 (m, 4.48 (d, j=6.2 Hz, 2H), 3.72 (d, J=2.3 Hz, 1H), 3.30-3.22 (m, 1H), 3.02-2.90 (m, 3H), 2.44 (br s, 2H), 1.91-1.81 (m, 4H), 1.46-1.22 (m, 8H), 0.89 (t, J=6.9 Hz, 3H) ppm. MS (ESI) m/z 387 (M+H)⁺.

### Example 80

### (2S,3S)-3-Hydroxy-N-(4-(5-octyl-1,2,4-oxadiazol-3-yl)benzyl)pyrrolidine-2-carboxamide

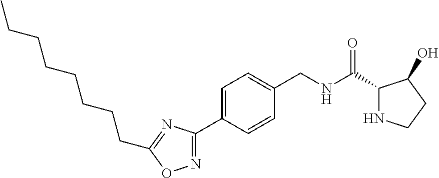

Utilizing a procedure similar to that described in Preparation A, the compound of Example 80 was prepared from (4-(5-octyl-1,2,4-oxadiazol-3-yl)phenyl)methanamine and N-(tert-butoxycarbonyl)-trans-3-hydroxy-L-proline. The product was obtained as a white solid: MS (ESI) m/z 401 (M+H)⁺.

The intermediate, (4-(5-octyl-1,2,4-oxadiazol-3-yl)phenyl)methanamine, was prepared using procedures similar to that describe in Example 79 starting with nonanoic acid and N′-hydroxy-4-(hydroxymethyl)benzimidamide (4-(5-octyl-1,2,4-oxadiazol-3-yl)phenyl)methanamine was obtained as a white solid: ¹H NMR (CDCl₃) δ 8.07-8.02 (m, 2H), 7.45-7.40 (m, 2H), 3.94 (s, 2H), 2.93 (t, J=7.6 Hz, 2H), 1.92-1.81 (m, 2H), 1.50-1.22 (m, 12H), 0.88 (t, J=6.9 Hz, 3H) ppm.

### Example 81

### (2S,3S)-3-Hydroxy-N-(4-(5-(4-propylphenyl)-1,2,4-oxadiazol-3-yl)benzyl)pyrrolidine-2-carboxamide

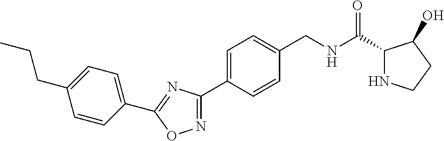

Utilizing a procedure similar to that described in Preparation A, the compound of Example 81 was prepared from (4-(5-(4-propylphenyl)-1,2,4-oxadiazol-3-yl)phenyl)methanamine and N-(tert-butoxycarbonyl)-trans-3-hydroxy-L-proline. The product was obtained as a white solid: ¹H NMR (CDCl₃) δ 8.28-8.23 (m, 1H), 8.14-8.07 (m, 4H), 7.39-7.32 (m, 4H), 5.71-5.48 (m, 2H), 4.61-4.55 (m, 1H), 4.48 (d, J=6.0 Hz, 2H), 3.87 (d, J=2.0 Hz, 1H), 3.35-3.25 (m, 1H), 3.10-3.02 (m, 1H), 2.71-2.64 (m, 2H), 1.91-1.82 (m, 2H), 1.74-1.63 (m, 2H), 0.97 (t, J=7.3 Hz, 3H) ppm. MS (ESI) m/z 407 (M+H)⁺.

The intermediate, (4-(5-(4-propylphenyl)-1,2,4-oxadiazol-3-yl)phenyl)methanamine, was prepared using procedures similar to that describe in Example 79 starting with 4-propylbenzoic acid and N′-hydroxy-4-(hydroxymethyl)benzimidamide. (4-(5-(4-propylphenyl)-1,2,4-oxadiazol-3-yl)phenyl)methanamine was obtained as a white solid: ¹H NMR (CDCl₃) δ 8.17-8.10 (m, 4H), 7.48-7.44 (m, 2H), 7.38-7.33 (m, 2H) 3.96 (s, 2H), 2.72-2.65 (m, 2H), 1.76-1.64 (m, 2H), 1.60 (br s, 2H), 0.97 (t, J=7.3 Hz, 3H) ppm.

### Example 82

### (2S,3S)—N-(4-(5-(4-Butylphenyl)-1,2,4-oxadiazol-3-yl)benzyl)-3-hydroxypyrrolidine-2-carboxamide

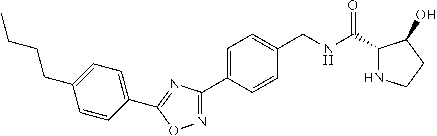

Utilizing a procedure similar to that described in Preparation A, the compound of Example 82 was prepared from (4-(5-(4-butylphenyl)-1,2,4-oxadiazol-3-yl)phenyl)methanamine and N-(tert-butoxycarbonyl)-trans-3-hydroxy-L-proline. The product was obtained as a white solid: ¹H NMR (CDCl₃) δ 8.28-8.23 (m, 1H), 8.15-8.09 (m, 4H), 8.07-8.01 (m, 1H), 7.41-7.33 (m, 4H), 4.61-4.58 (m, 1H), 4.55-4.44 (m, 2H), 3.71 (d, J=2.3 Hz, 1H), 3.31-3.22 (m, 1H), 3.03-2.95 (m, 1H), 2.74-2.67 (m, 2H), 2.44 (br s, 2H), 1.88-1.81 (m, 2H), 1.69-1.60 (m, 2H), 1.44-1.33 (m, 2H), 0.95 (t, J=7.3 Hz, 3H) ppm. MS (ESI) m/z 421 (M+H⁺.

The intermediate, (4-(5-(4-butylphenyl)-1,2,4-oxadiazol-3-yl)phenyl)methanamine, was prepared using procedures similar to that describe in Example 79 starting with 4-butyl]benzoic acid and N′-hydroxy-4-(hydroxymethyl)benzimidamide. (4-(5-(4-butylphenyl)-1,2,4-oxadiazol-3-yl)phenyl)methanamine was obtained as a yellow solid: ¹H NMR (CDCl₃) δ 8.17-8.10 (m, 4H), 7.49-7.44 (m, 2H), 7.38-7.33 (m, 2H) 3.96 (s, 2), 2.75-2.67 (m, 2H), 1.71-1.60 (m, 2H), 1.51 (br s, 2H), 1.43-1.33 (m, 2H), 0.95 (t, J=7.3 Hz, 3H) ppm.

### Example 83

### (S)-2-Amino-N-(4-(5-heptyl-1,2,4-oxadiazol-3-yl)benzyl)-4-hydroxybutanamide

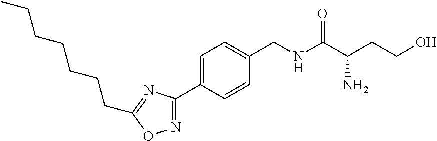

### Synthesis of (S)-2-Amino-N-(4-(5-heptyl-1,2,4-oxadiazol-3-yl)benzyl)-4-hydroxybutanamide

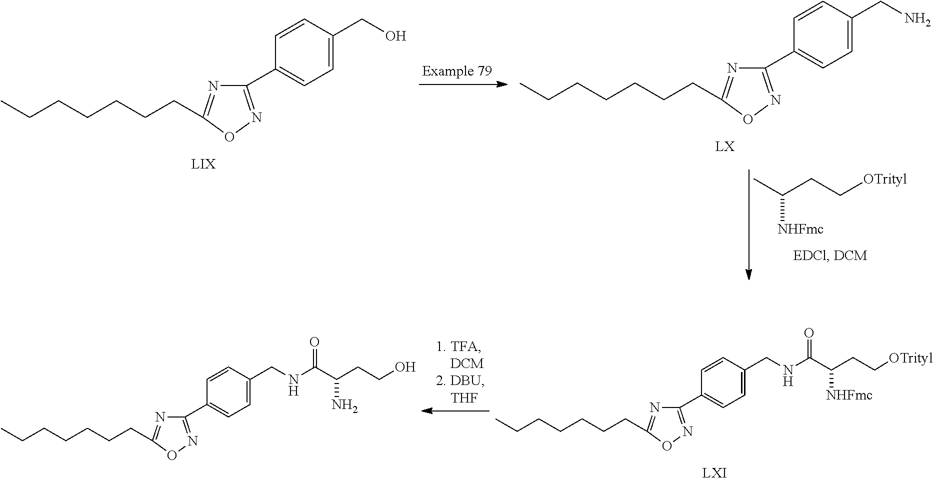

### Step 1) Preparation of (S)-(9H-fluoren-9-yl)methyl 1(4-(5-heptyl-1,2,4-oxadiazol-3-yl)benzylamino)-1-oxo-4-(trityloxy)butan-2-ylcarbamate (LXI)

To a stirred solution of (4-(5-heptyl-1,2,4-oxadiazol-3-yl)phenyl)methanamine (LX, prepared with procedures similar to that described in Example 79) (0.290 g, 1.20 mmol) and (S)-2-(((9H-fluoren-9-yl)methoxy)carbonylamino)-4-(trityloxy)butanoic acid (0.841 g, 1.44 mmol) in dichloromethane (6 mL) was added N-(3-dimethylaminopropyl)-N′-ethylcarbodiimide hydrochloride (0.282 g, 1.44 mmol). The reaction mixture was allowed to stir at room temperature. After 3 hours, the reaction mixture was diluted with dichloromethane and washed with 1N hydrochloric acid. The organic phase was dried (sodium sulfate), filtered, and concentrated to provide a yellow oil. Flash chromatography using an Isco Combiflash unit (40 g SiO₂ column, 30-50% ethyl acetate/hexanes) afforded 0.807 g (81%) of (S)-(9H-fluoren-9-yl)methyl 1-(4-(5-heptyl-1,2,4-oxadiazol-3-yl)benzylamino)-1-oxo-4-(trityloxy)butan-2-ylcarbamate (LXI) as a yellow solid: ¹H NMR (CDCl₃) δ 8.00-7.95 (m, 2H), 7.76-7.70 (m, 2H), 7.53-7.47 (m, 2H), 7.42-7.33 (m, 8H), 7.30-7.17 (m, 13H), 6.56 (br s, 1H), 6.04 (d, J=6 Hz, 1H), 4.45-4.28 (m, 5H), 4.19-4.12 (m, 1H), 3.40-3.24 (m, 2H), 2.93 (t, J=7.6 Hz, 2H), 2.19-2.01. (m, 2H), 1.92-1.81 (m, 2H), 1.48-1.23 (m, 0.89 (t, J=6.9 Hz, 3H) ppm.

### Step 2) Preparation of (S)-(9H-fluoren-9-yl)methyl 1-(4-(5-heptyl-1,2,4-oxadiazol-3-yl)benzylamino)-4-hydroxy-1-oxobutan-2-ylcarbamate

To a stirred solution of (S)-(9H-fluoren-9-yl)methyl 1-(4-(5-heptyl-1,2,4-oxadiazol-3-yl)benzylamino)-1-oxo-4-(trityloxy)butan-2-ylcarbamate (0.780 g, 0.93 mmol) in dichloromethane (2 mL) was added trifluoroacetic acid (0.8 mL). After 1 hour, the dark brown reaction mixture was concentrated, and the residue suspended in saturated sodium bicarbonate solution. The mixture was extracted three times with dichloromethane. The combined organic phases were dried (sodium sulfate), filtered, and concentrated to provide a yellow oil. Flash chromatography using an Isco Combiflash unit (40 g SiO₂ column, 1-10% 2M ammonia in methanol/dichloromethane) afforded 0.377 g (68%) of (S)-(9H-fluoren-9-yl)methyl 1-(4-(5-heptyl-1,2,4-oxadiazol-3-yl)benzylamino)-4-hydroxy-1-oxobutan-2-ylcarbamate as a white solid. ¹H NMR consistent with assigned structure.

### Step 3) Preparation of (S)-2-Amino-N-(4-(5-heptyl-1,2,4-oxadiazol-3-yl)benzyl)-4-hydroxybutanamide

To a stirred solution of (S)-(9H-fluoren-9-yl)methyl 1-(4-(5-heptyl-1,2,4-oxadiazol-3-yl)benzylamino)-4-hydroxy-1-oxobutan-2-ylcarbamate (0.377 g, 0.632 mmol) in tetrahydrofuran (2 mL) was added 1,8-diazabicycloundec-7-ene (0.115 g, 0.758 mmol). The reaction mixture was allowed to stir at room temperature. After 18 hours, the reaction mixture was diluted with dichloromethane and washed with brine. The organic phase was dried (sodium sulfate), filtered, and concentrated to provide a yellow oil. Flash chromatography using an Isco Combiflash unit (40 g SiO₂ column, 5-100% 2M ammonia in methanol/dichloromethane) afforded 0.229 g (97%) of (S)-2-amino-N-(4-(5-heptyl-1,2,4-oxadiazol-3-yl)benzyl)-4-hydroxybutanamide as a white solid: ¹H NMR (CDCl₃) δ 8.02 (d, J=8.3 Hz, 2H), 7.41 (d, J=8.3 Hz, 2H), 4.53-4.44 (m, 2H), 3.98-3.93 (m, 1H), 3.80-3.75 (m, 2H), 3.29-3.16 (m, 6H), 2.97-2.91 (m, 2H), 1.96-1.82 (m, 2H), 1.48-1.24 (m, 8H), 0.89 (t, J=6.9 Hz, 3H) ppm. MS (ESI) m/z 375 (M+H)⁺.

### Example 84

### (2S,3S)-3-Hydroxy-N-(4-(5-pentyl-1,2,4-oxadiazol-3-yl)benzyl)pyrrolidine-2-carboxamide

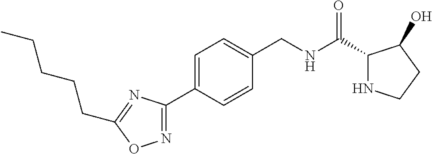

Utilizing a procedure similar to that described in Preparation A, the compound of Example 84 was prepared from partially purified (4-(5-pentyl-1,2,4-oxadiazol-3-yl)phenyl)methanamine and N-(tert-butoxycarbonyl)-trans-3-hydroxy-L-proline. The product was obtained as a white solid: MS (ESI) m/z 359 (M+H)⁺.

The intermediate, (4-(5-pentyl-1,2,4-oxadiazol-3-yl)phenyl)methanamine, was prepared using procedures similar to that described in Example 79 starting with hexanoyl chloride and N′-hydroxy-4-(hydroxymethyl)benzimidamide in pyridine (12 mL).

### Example 85

### (2S,3S)—N-(4-(5-Hexyl-1,2,4-oxadiazol-3-yl)benzyl)-3-hydroxypyrrolidine-2-carboxamide

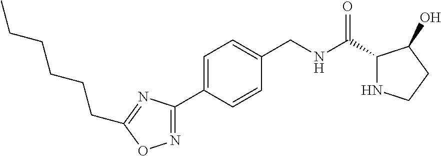

Utilizing a procedure similar to that described in Preparation A, the compound of Example 85 was prepared from (4-(5-hexyl-1,2,4-oxadiazol-3-yl)phenyl)methanamine and N-(tert-butoxycarbonyl)-trans-3-hydroxy-L-proline. The product was obtained as a white solid; ¹H NMR (CDCl₃) δ 8.14-8.08 (m, 1H), 8.03 (d, J=8.3 Hz, 2H), 7.35(d, J=8.3 Hz, 2H), 4.62-4.56 (m, 1H), 4.47 (d, J=6.1 Hz, 2H), 3.78 (d, J=2.0 Hz, 1H), 3.33-3.22 (m, 1H), 3.06-2.97 (m, 1H), 2.94 (t, J=7.6 Hz, 2H), 2.71 (br s, 2H), 1.91-1.80 (m, 4H), 1.47-1.28 (m, 6H), 0.90 (t, J=7.1 Hz, 3H) ppm. MS (ESI) m/z 373 (M+H)⁺.

The intermediate, (4-(5-hexyl-1,2,4-oxadiazol-3-yl)phenyl)methanamine, was prepared using procedures similar to that described in Example 79 starting with heptanoyl chloride and N′-hydroxy-4-(hydroxymethyl)benzimidamide in pyridine. (4-(5-hexyl-1,2,4-oxadiazol-3-yl)phenyl)methanamine was obtained as a white solid: ¹H NMR (CDCl₃) δ 8.07-8.02 (m, 2H), 7.45-7.41 (m, 2H), 3.94 (s, 2H), 2.97-2.90 (m, 2H), 1.92-1.82 (m, 2H), 1.60 (br s, 2H), 1.48-1.28 (m, 6H), 0.93-0.86 (m, 3H) ppm.

### Example 86

### (2S,3S)—N-(4-(5-Cyclohexyl-1,2,4-oxadiazol-3-yl)benzyl)-3-hydroxypyrrolidine-2-carboxamide

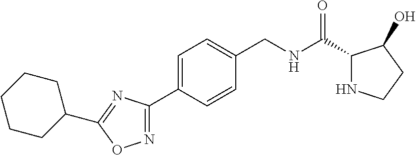

Utilizing a procedure similar to that described in Preparation A, the compound of Example 86 was prepared from (4-(5-cyclohexyl-1,2,4-oxadiazol-3-yl)phenyl)methanamine and N-(tert-butoxycarbonyl)-trans-3-hydroxy-L-proline. In this case, the carbamate protection was accomplished using anhydrous hydrogen chloride in 1,4-dioxane. The product was obtained as a white solid: ¹H NMR (CDCl₃) δ 8.10-8.00 (m, 3H), 7.37-7.31 (m, 2H), 4.61-4.55 (m, 1H), 4.47(m 2H), 3.72 (d, J=2.2 Hz, 1H), 3.31-3.21 (m, 1H), 3.05-2.94 (m, 2H), 2.34 (br s, 2H), 2.17-2.09 (m, 2H), 1.92-1.79 (m, 4H), 1.77-1.64 (m, 3H), 1.49-1.30 (m, 3H) ppm. MS (ESI) m/z 371 (M+H)⁺.

The intermediate, (4-(5-cyclohexyl-1,2,4-oxadiazol-3-yl)phenyl)methanamine, was prepared using procedures similar to that described in Example 79 starting with cyclohexanecarbonyl chloride and N′-hydroxy-4-(hydroxymethyl)benzimidamide in pyridine, (4-(5-cyclohexyl-1,2,4-oxadiazol-3-yl)phenyl)methanamine was obtained as a yellow solid: ¹H NMR (CDCl₃) δ 8.07-8.01 (m, 2H), 7.44-7.39 (m, 2H), 3.93 (s, 2H), 3.05-2.95 (m, 1H), 2.18-2.09 (m, 2H), 2.02 (br s, 2H), 1.90-1.82 (m, 2H), 1.77-1.64 (m, 3H), 1.49-1.26 (m, 3H) ppm.

### Example 87

### (2S,3S)-3-Hydroxy-N-(4-(5-(1-methylcyclohexyl)-1,2,4-oxadiazol-3-yl)benzyl)pyrrolidine-2-carboxamide

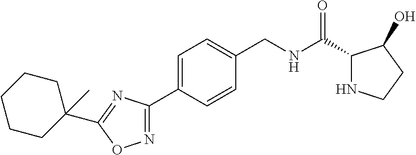

Utilizing a procedure similar to that described in Preparation A, the compound of Example 87 was prepared from (4-(5-(1-methylcyclohexyl)-1,2,4-oxadiazol-3-yl)phenyl)methanamine and N-(tert-butoxycarbonyl)-trans-3-hydroxy-L-proline. The product was obtained as a white solid: MS (ESI) m/z 385 (M+H)⁺.

The intermediate, (4-(5-(1-methylcyclohexyl)-1,2,4-oxadiazol-3-yl)phenyl)methanamine, was prepared using procedures similar to that described in Example 79 starting with 1-methylcyclohexanecarboxylic acid, N-(3-dimethylaminopropyl)-N′-ethylcarbodiimide hydrochloride and N′-hydroxy-4-(hydroxymethyl)benzimidamide in dichloromethane. (4-(5-(1-methylcyclohexyl)-1,2,4-oxadiazol-3-yl)phenyl)methanamine was obtained as a yellow oil: ¹H NMR (CDCl₃) δ 8.09-8.04 (m, 2H), 7.44-7.40 (m, 2H), 3.94 (s, 2H), 2.33-2.26 (m, 2H), 1.69-1.36 (m, 13H) ppm.

### Example 88

### (2S,3S)—N-(4-(5-Cyclopentyl-1,2,4-oxadiazol-3-yl)benzyl)-3-yl)benzyl)-3-hydroxypyrrolidine-2-carboxamide

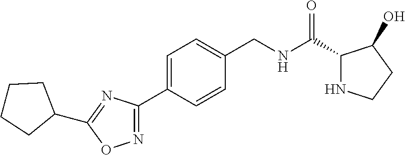

Utilizing a procedure similar to that described in Preparation A, the compound of Example 88 was prepared from (4-(5-cyclopentyl-1,2,4-oxadiazol-3-yl)phenyl)methanamine and N-(tert-butoxycarbonyl)-trans-3-hydroxy-L-proline. The product was obtained as a white solid: MS (ESI) m/z 357 (M+H)⁺.

The intermediate, from (4-(5-cyclopentyl-1,2,4-oxadiazol-3-yl)phenyl)methanamine, was prepared using procedures similar to that described in Example 79 starting with N′-hydroxy-4-(hydroxymethyl)benzimidamide and cyclopentanecarbonyl chloride in pyridine. (4-(5-cyclopentyl-1,2,4-oxadiazol-3-yl)phenyl)methanamine as a yellow oil, ¹H NMR consistent with assigned structure.

### Example 89

### (2S,3S)—N-(4-(5-Cycloheptyl-1,2,4-oxadiazol-3-yl)benzyl)-3-hydroxypyrrolidine-2-carboxamide

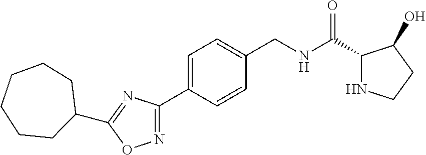

Utilizing a procedure similar to that described in Preparation A, the compound of Example 89 was prepared from (4-(5-cycloheptyl-1,2,4-oxadiazol-3-yl)phenyl)methanamine and N-(tert-butoxycarbonyl)-trans-3-hydroxy-L-proline. The product was obtained as a white solid: MS (ESI) m/z 385 (M+H)⁺.

The intermediate, from (4-(5-cycloheptyl-1,2,4-oxadiazol-3-yl)phenyl)methanamine, was prepared using procedures similar to that described in Example 79 starting with N′-hydroxy-4-(hydroxymethyl)benzimidamide and 3-cyclopentylpropanoyl chloride in pyridine. (4-(5-cycloheptyl-1,2,4-oxadiazol-3-yl)phenyl)methanamine was obtained, as a yellow oil: ¹H NMR (CDCl₃) δ 8.06-8.02 (m, 2H), 7.44-7.40 (m, 2H), 3.93 (s, 2H), 3.24-3.15 (m, 1H), 2.21-2.11 (m, 2H), 1.98-1.77 (m, 6H), 1.72-1.54 (m, 6H) ppm.

### Example 90

### (2S,3S)—N-(4-(5-(2-Cyclopentylethyl)-1,2,4-oxadiazol-3-yl)benzyl)-3-hydroxypyrrolidine-2-carboxamide

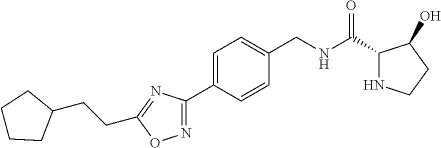

Utilizing a procedure similar to that described in Preparation A, the compound of Example 90 was prepared from (4-(5-(2-cyclopentylethyl)-1,2,4-oxadiazol-3-yl)phenyl)methanamine and N-(tert-butoxycarbonyl)-trans-3-hydroxy-L-proline. The product was obtained as a white solid: ¹H NMR (CDCl₃) δ 8.06-7.99(m, 3H), 7.37-7.33 (m, 2H), 4.61-4.56 (m, 1H), 4.48 (d, J=6.2 Hz, 2H), 3.71 (d, J=2.3 Hz, 1H), 3.30-3.21 (m, 1H), 3.02-2.92 (m, 3H), 2.15 (br s, 2H), 1.93-1.71 (m, 7H) 1.68-1.49 (m, 4H), 1.21-1.10 (m, 2H) ppm. MS (ESI) m/z 385 (M+H)⁺.

The intermediate, from (4-(5-cycloheptyl-1,2,4-oxadiazol-3-yl)phenyl)methanamine, was prepared using procedures similar to that described in Example 79 starting with N′-hydroxy-4-(hydroxymethyl)benzimidamide and added 3-cyclopentylpropanoyl chloride in Pyridine, (4-(5-(2-cyclopentylethyl)-1,2,4-oxadiazol-3-yl)phenyl)methanamine was obtained after flash chromatography using an Isco Combiflash unit (120 g SiO₂ column, 10% methanol/dichloromethane): ¹H NMR (CDCl₃) δ 8.06-8.01 (m, 2H), 7.45-7.40 (m, 2H), 3.94 (s, 2H), 2.98-2.92 (m, 2H), 1.93-1.71 (m, 5H), 1.78-1.71 (br s, 2H), 1.67-1.49 (m, 4H), 1.22-1.10 (m, 2H) ppm.

### Example 91

### (2S,3S)—N-(4-(5-(2-Cyclopropylethyl)-1,2,4-oxadiazol-3-yl)benzyl)-3-hydroxypyrrolidine-2-carboxamide

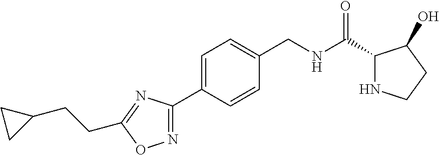

Utilizing a procedure similar to that described in Preparation A, the compound of Example 91 was prepared from (4-(5-(2-cyclopropylethyl)-1,2,4-oxadiazol-3-yl)phenyl)methanamine and N-(tert-butoxycarbonyl)-trans-3-hydroxy-L-proline. In this case, the carbamate deprotection was accomplished using anhydrous hydrogen chloride in 1,4-dioxane. The product was obtained as a white solid ¹H NMR (CDCl₃) δ 8.10-8.01 (m, 3H), 7.37-7.32 (m, 2H), 4.61-4.55 (m, 1H), 4.52-4.40 (m, 2H), 3.72 (d, J=2.1 Hz, 1H), 1.31-1.20 (m, 1H), 3.08-2.93 (m, 3H), 2.68 (br s, 2H), 1.87-1.72 (m, 4H), 0.86-0.75 (m, 1H), 0.54-0.40 (m, 2H), 0.16-0.04 (m, 2H) ppm. MS (ESI) m/z 357 (M+H)⁺.

The intermediate, (4-(5-(2-cyclopropylethyl)-1,2,4-oxadiazol-3-yl)phenyl)methanamine, was prepared using procedures similar to that described in Example 79 starting with N′-hydroxy-4-(hydroxymethyl)benzimidamide and 3-cyclopropylpropanoic acid in Pyridine. (4-(5-(2-cyclopropylethyl)-1,2,4-oxadiazol-3-yl)phenyl)methanamine was obtained as a yellow oil: ¹H NMR (CDCl₃) δ 8.07-8.02 (m, 2H), 7.46-7.40 (m, 2H), 3.94 (s, 2H), 3.10-3.00 (m, 2H), 1.83-1.72 (m, 2H), 1.50 (br s, 2H) 0.86-0.76 (m, 1H), 0.52-0.44 (m, 2H), 0.13-0.07 (m, 2H) ppm.

### Example 92

### (2S,3S)—N-(4-(5-(4,4-Difluorocyclohexyl)-1,2,4-oxadiazol-3-yl)benzyl)-3-hydroxypyrrolidine-2-carboxamide

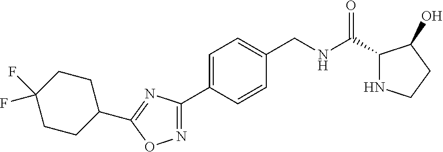

Utilizing a procedure similar to that described in Preparation A, the compound of Example 92 was prepared from (4-(5-(4,4-difluorocyclohexyl)-1,2,4-oxadiazol-3-yl)phenyl)methanamine and N-(tert-butoxycarbonyl)-trans-3-hydroxy-L-proline. In this case, the carbamate deprotection was accomplished using anhydrous hydrogen chloride in 1,4-dioxane. The product was obtained as a white solid: ¹H NMR (CDCl₃) δ 8.10-8.00 (m, 3H), 7.37-7.32 (m, 2H), 4.62-4.57 (m, 1H), 4.54-4.42 (m, 2H), 3.72 (d, J=2.1 Hz, 1H), 3.31-3.21 (m, 1H), 3.17-3.08 (m, 1H), 3.02-2.94 (m, 1H), 2.62 (br s, 2H), 2.30-2.04 (m, 6H), 2.00-1.77 (m, 4H) ppm. MS (ESI) m/z 407 (M+H)⁺.

The intermediate, (4-(5-(4,4-difluorocyclohexyl)-1,2,4-oxadiazol-3-yl)phenyl)methanamine, was prepared using procedures similar to that described in Example 79 starting with 4,4-difluorocyclohexanecarboxylic acid, N-(3-dimethylaminopropyl)-N′-ethylcarbodiimide hydrochloride and N′-hydroxy-4-(hydroxymethyl)benzimidamide in dichloromethane. (4-(5-(4,4-difluorocyclohexyl)-1,2,4-oxadiazol-3-yl)phenyl)methanamine was obtained as a white solid: ¹H NMR (CDCl₃) δ 8.07-8.01 (m, 2H), 7.46-7.41 (m, 2H), 3.95 (s, 2H), 3.18-3.08 (m, 1H), 2.30-2.06 (m, 5H), 2.01-1.84 (m, 2H), 1.53 (br s, 2H) ppm.

### Example 93

### (2S,3S)—N-(4-(5-(2-Cyclohexylethyl)-1,2,4-oxadiazol-3-yl)benzyl)-3-hydroxypyrrolidine-2-carboxamide

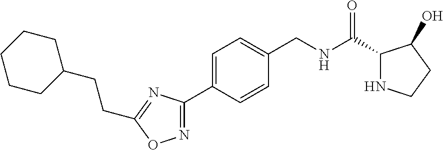

Utilizing a procedure similar to that described in Preparation A, the compound of Example 93 was prepared from (4-(5-(2-cyclohexylethyl)-1,2,4-oxadiazol-3-yl)phenyl)methanamine and N-(tert-butoxycarbonyl)-trans-3-hydroxy-L-proline. In this case, the carbamate deprotection was accomplished using anhydrous hydrogen chloride in methanol. The product was obtained as a white solid: ¹H NMR (CDCl₃) δ 8.08-8.00 (m, 3H), 7.37-7.32 (m, 2H), 4.62-4.57 (m, 1H), 4.53-4.41 (m, 2H), 3.73 (d, J=2.2 Hz, 1H), 3.31-3.22 (m, 1H), 3.02-2.91 (m, 3H), 2.58 (br s, 2.11), 1.89-1.62 (m, 9H), 1.40-1.12 (m, 4H), 1.03-0.89 (m, 2H) ppm. MS (ESI) m/z 399 (M+H)⁺.

The intermediate, (4-(5-(2-cyclohexylethyl)-1,2,4-oxadiazol-3-yl)phenyl)methanamine, was prepared using procedures similar to that described in Example 79 starting with 3-cyclohexylpropanoic acid, N-(3-dimethylaminopropyl)-N′-ethylcarbodiimide hydrochloride and N′-hydroxy-4-(hydroxymethyl)benzimidamide in dichloromethane. (4-(5-(2-cyclohexylethyl)-1,2,4-oxadiazol-3-yl)phenyl)methanamine was obtained as a yellow ¹H NMR (CDCl₃) δ 8.06-8.01 (m, 2H), 7.45-7.40 (m, 2H), 3.94 (s, 2H), 2.98-2.91 (m, 2H), 1.83-1.51 (m, 9H), 1.41-1.13 (m, 4H), 1.02-0.90 (m, 2H) ppm.

### Example 94

### (2S,3S)-1-Hydroxy-N-(4-(5-phenethyl-1,2,4-oxadiazol-3-yl)benzyl)pyrrolidine-2-carboxamide

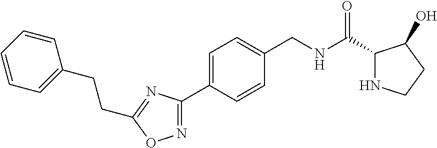

Utilizing a procedure similar to that described in Preparation A, the compound of Example 94 was prepared from (4-(5-phenethyl-1,2,4-oxadiazol-3-yl)phenyl)methanamine and N-(tert-butoxycarbonyl)-trans-3-hydroxy-L-proline. In this case, the carbamate deprotection was accomplished using anhydrous hydrogen chloride in methanol. The product was obtained as a white solid: ¹H NMR (CDCl₃) δ 8.08-8.00 (m, 3H), 7.38-7.28 (m, 4H), 7.27-7.20 (m, 3H), 4.63-4.57 (m, 1H), 4.53-4.43 (m, 2H), 3.73 (d, J=2.2 Hz, 1H), 3.30-3.16 (m, 5H), 3.02-2.93 (m, 1H), 2.54 (br s, 2H), 1.88-1.80 (m, 2H) ppm. MS (ESI) m/z 393 (M+H)⁺.

The intermediate, (4-(5-(2-phenylethyl)-1,2,4-oxadiazol-3-yl)phenyl)methanamine, was prepared using procedures similar to that describe in Example 79 starting with 3-phenylpropanoic acid, N-(3-dimethylaminopropyl)-N′-ethylcarbodiimide hydrochloride and N′-hydroxy-4-(hydroxymethyl)benzimidamide in dichloromethane. (4-(5-phenethyl-1,2,4-oxadiazol-3-yl)phenyl)methanamine was obtained as a yellow solid: ¹H NMR (CDCl₃) δ 8.07-8.01 (m, 2H), 7.46-7.41 (m, 2H), 7.35-7.28 (m, 2H), 7.27-7.20 (m, 3H) 3.94 (s, 2H), 3.28-3.19 (m, 4H), 1.63 (br s, 2H) ppm.

### Example 95

### (2S,3S)—N—((S-1-(4-(5-(2-Cyclohexylethyl-1,2,4-oxadiazol-3-yl)phenyl)propyl)-3-hydroxypyrrolidine-2-carboxamide

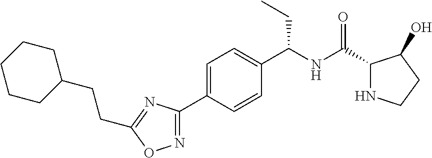

### Preparation of the intermediate (S)-1-(4-(5-(2-cyclohexylethyl)-1,2,4-oxadiazol-3-yl)phenyl)propan-1-amine

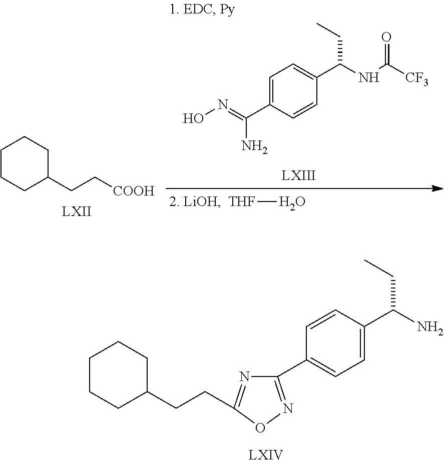

To a stirred solution of 3-cyclohexylpropanoic acid (LXII) (0.323 g, 2.07 mmol) in dichloromethane (4 mL) was added N-(3-dimethylaminopropyl)-N′-ethylcarbodiimide hydrochloride (0.400 g, 2.09 mmol). The reaction mixture was allowed to stir at room temperature for 1.5 hours and was then concentrated. The residue was treated with (S)-2,2,2-trifluoro-N-(1-(4-(N′-hydroxycarbamimidoyl)phenyl)propyl)ethanamide (LXIII) (0.502 g, 1.74 mmol) in pyridine (4 mL). The reaction mixture was heated to reflux and stirred. After 18 hours, the reaction mixture was allowed to cool to room temperature and was diluted with 1N hydrochloric acid. The mixture was extracted three times with ethyl acetate. The combined organic phases were dried (sodium sulfate), filtered, and concentrated. The residue was dissolved in tetrahydrofuran (4 mL) and water (4 mL) and was treated with lithium hydroxide monohydrate (0.388 g, 9.25 mmol). The reaction mixture was allowed to stir at room temperature. After 65 hours, the reaction mixture was diluted with brine, and extracted three times with ethyl acetate. The combined organic phases were dried (sodium sulfate), filtered, and concentrated. Flash chromatography using an Isco Combiflash unit (40 g SiO₂ column, 10-20% methanol/dichloromethane) afforded 0.399 g (74%) of (S)-1-(4-(5-(2-cyclohexylethyl)-1,2,4-oxadiazol-3-yl)phenyl)propan-1-amine (LXIV) as a yellow solid. This material was used ‘as is’ in the next reaction.

Utilizing a procedure similar to that described in Preparation A, the compound of Example 95 was prepared from (S)-1-(4-(5-(2-cyclohexylethyl)-1,2,4-oxadiazol-3-yl)phenyl)propan-1-amine and N′-(tert-butoxycarbonyl)-trans-1-hydroxy-L-proline. In this case, the carbamate deprotection was accomplished using anhydrous hydrogen chloride in methanol. The product was obtained as a white solid: ¹H NMR (CDCl₃) δ 8.03 (d, J=8.3 Hz, 2H), 7.97 (d, J=8.9 Hz, 2H), 7.37 (d, J=8.1 Hz, 2H), 4.93-4.83 (m, 1H), 4.61-4.54 (m, 1H), 3.61 (d, J=2.3 Hz, 1H), 3.33-3.23 (m, 1H), 3.07-2.99 (m, 2H), 2.38 (br s, 2H), 1.91-1.61 (m, 11H), 1.39-1.11 (m, 4H), 1.01-0.86 (m, 5H) ppm. MS (ESI) m/z 427 (M+H)⁺.

### Example 96

### (2S,3S)—N—((S)-1-(4-(5-(2-Cyclohexylethyl)-1,2,4-oxadiazol-3-yl)phenyl)ethyl)-3-hydroxypyrrolidine-2-carboxamide hemi-tartrate salt

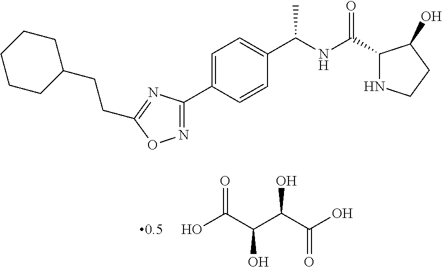

Utilizing a procedure similar to that described in Preparation A, the compound of Example 96 was prepared from (S)-1-(4-(5-(2-cyclohexylethyl)-1,2,4-oxadiazol-3-yl)phenyl)ethanamine and N-(tert-butoxycarbonyl)-trans-1-hydroxy-L-proline. In this case, the carbamate deprotection was accomplished using anhydrous hydrogen chloride in methanol. The product, a white solid, was isolated as the hemi-tatrate salt from aqueous ethanol by lyophilization): MS (ESI) m/z 413 (M+H)⁺.

The intermediate, (S)-1-(4-(5-(2-cyclohexylethyl)-1,2,4-oxadiazol-3-yl)phenyl)ethanamine, was prepared using procedures similar to that described in Example 95 starting with 3-cyclohexylpropanoic acid, N-(3-dimethylaminopropyl)-N′-ethylcarbodiimide hydrochloride and (S)-2,2,2-trifluoro-N-(1-(4-(N′-hydroxycarbamimidoyl)phenyl)ethyl)ethanamide, (S)-1-(4-(5-(2-cyclohexylethyl)-1,2,4-oxadiazol-3-yl)phenyl)ethanamine as a yellow solid. This material was used ‘as is’ in the next reaction.

### Example 97

### (2S,3S)—N—((S)-1-(4-(5-(2Cyclopentylethyl)-1,2,4-oxadiazol-3-yl)phenyl)ethyl)-3-hydroxypyrrolidine-2-carboxamide hemi-tartrate salt

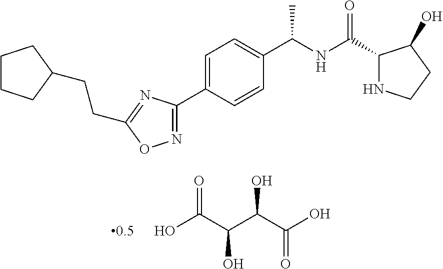

### Preparation of the intermediate (S)-1-(4-(5-(2-cyclopentylethyl)-1,2,4-oxadiazol-3-yl)phenyl)ethanamine

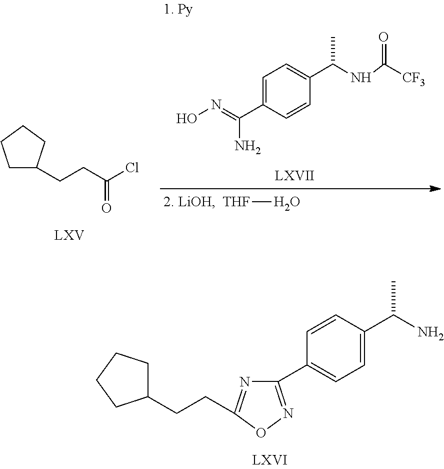

To a stirred solution of (S)-2,2,2-trifluoro-N-(1-(4-(N′-hydroxycarbamimidoyl)phenyl)ethyl)ethanamide (LXVII) (1.33 g, 4.83 mmol) in pyridine (10 mL) was added 3-cyclopentylpropanoyl chloride (LXV) (0.932 g, 5.80 mmol). The reaction mixture was heated to reflux and stirred. After 18 hours, the reaction mixture was allowed to cool to room temperature and diluted with 1N hydrochloric acid. The mixture was extracted three times with ethyl acetate. The combined organic phases were dried (sodium sulfate), filtered, and concentrated. The residue was dissolved in tetrahydrofuran (5 mL) and water (5 mL) and was treated with lithium hydroxide monohydrate (2.45 g, 58.39 mmol). The reaction mixture was allowed to stir at room temperature. After 18 hours, the reaction mixture was diluted with brine and extracted three times with ethyl acetate. The combined organic phases were dried (sodium sulfate), filtered, and concentrated. Flash chromatography using an Isco Combiflash unit (40 g SiO₂ column, 10-20% methanol/dichloromethane) afforded 0.498 g (36%) of (S)-1-(4-(5-(2-cyclopentylethyl)-1,2,4-oxadiazol-3-yl)phenyl)ethanamine (LXVI) as an orange semi-solid. This material was used ‘as is’ in the next reaction.

Utilizing a procedure similar to that described in Preparation A, the compound of Example 97 was prepared from (S)-1-(4-(5-(2-cyclopentylethyl)-1,2,4-oxadiazol-3-yl)phenyl)ethanamine and N-(tert-butoxycarbonyl)-trans-3-hydroxy-L-proline. In this case, the carbamate deprotection was accomplished using anhydrous hydrogen chloride in methanol. The product, a white solid, was isolated as the hemi-tatrate salt (from aqueous ethanol by lyophilization): ¹H NMR (free base, CDCl₃) δ 8.03 (d, J=8.4 Hz, 2H), 7.96 (d, J=8.4 Hz, 2H), 7.40 (d, J=8.3 Hz, 2H), 5.15-5.06 (m, 1H), 4.58-4.53 (m, 1H), 3.63 (d, J=2.3 Hz, 1H), 3.31-3.21 (m, 1H), 3.06-2.91 (m, 3H), 2.71 (br s, 2H), 1.92-1.76 (m, 6H), 1.70-1.45 (m, 7H), 1.21-1.09 (m, 2H) ppm. MS (ESI) m/z 399.

### Example 98

### (2S,3S)—N—((S)-1-(4-(5-(2-Cyclopentylethyl)-1,2,4-oxadiazol-3-yl)phenyl)propyl)-3-hydroxypyrrolidine-2-carboxamide hemi-tartrate salt

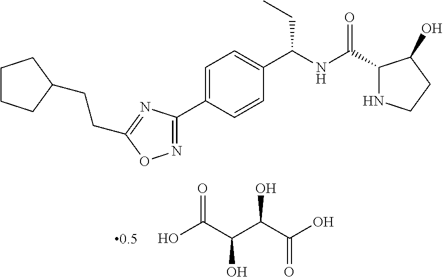

Utilizing a procedure similar to that described in Preparation A, the compound of Example 98 was prepared from (S)-1-(4-(5-(2-cyclopentylethyl)-1,2,4-oxadiazol-3-yl)phenyl)propan-1-amine and N-(tert-butoxycarbonyl)-trans-3-hydroxy-L-proline. In this case, the carbamate deprotection was accomplished using anhydrous hydrogen chloride in methanol. The product, a white solid, was isolated as the hemi-tatrate salt (from aqueous ethanol by lyophilization): ¹H NMR (free base, CDCl₃) δ 8.05-7.98 (m, 3H), 7.39-7.34 (m, 2H), 4.92-4.82 (m, 1H), 4.61-4.54 (m, 1H), 3.62 (d, J=2.2 Hz, 1H), 3.33-3.23 (m, 1H), 3.08-2.99 (m, 1H), 2.98-291 (m, 2H), 2.67 (br s, 2H), 1.93-1.75 (m, 9H), 1.69-1.47 (m, 4H), 1.21-1.08 (m, 2H), 0.89 (t, 7.4 Hz, 3H) ppm. MS (ESI) m/z 413,

The intermediate, (S)-1-(4-(5-(2-cyclopentylethyl)-1,2,4-oxadiazol-3-yl)phenyl)propan-1-amine, was prepared using, procedures similar to that described in Example 97 starting with (S)-2,2,2-trifluoro-N-(1-(4-(N′-hydroxycarbamimidoyl)phenyl)propyl)ethanamide and 3-cyclopentylpropanoyl chloride in Pyridine. (S)-1-(4-(5-(2-cyclopentylethyl)-1,2,4-oxadiazol-3-yl)phenyl)propan-1-amine was obtained as a yellow solid: ¹H NMR (CDCl₃) δ 8.03 (d, J=8.3 Hz, 2H), 7.43 (d, J=8.3 Hz, 2H), 3.89 (t, J=6.9 Hz, 1H), 2.99-2.92 (m, 2H), 2.76 (br s, 2H), 1.94-1.48 (m, 11H), 1.21-1.10 (m, 2H), 0.87 (t, J=7.4 Hz, 3H) ppm.

### Example 99

### (2S,3S)—N—((S)-1-(4-(5-Cyclohexyl-1,2,4-oxadiazol-3-yl)phenyl)propyl)-3-hydroxypyrrolidine-2-carboxamide hemi-tartrate salt

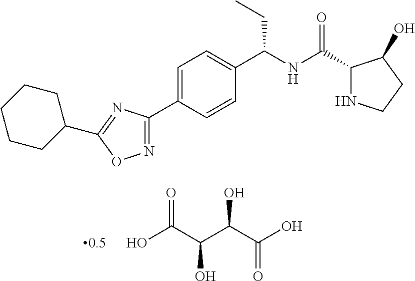

Utilizing a procedure similar to that described in Preparation A, the compound of Example 99 was prepared from (S)-1-(4-(5-cyclohexyl-1,2,4-oxadiazol-3-yl)phenyl)propan-1-amine and N-(tert-butoxycarbonyl)-trans-3-hydroxy-L-proline. The carbamate deprotection was accomplished using anhydrous hydrogen chloride in methanol. The product, a white solid, was isolated as the hemi-tatrate salt (from aqueous ethanol by lyophilization,): MS (ESI) m/z 399.

The intermediate, (S)-1-(4-(5-cyclohexyl-1,2,4-oxadiazol-3-yl)phenyl)propan-1-amine, was prepared using procedures similar to that describe in Example 95 starting with 3-cyclohexylpropanoic acid, N-(3-dimethylaminopropyl)-N′-ethylcarbodiimide hydrochloride and (S)-2,2,2-trifluoro-N-(1-(4-(N′-hydroxycarbamimidoyl)phenyl)propyl)ethanamide.

### Example 100

### (2S,3S)—N—((S)-1-(4-(5-(2-Cyclobutylethyl)-1,2,4-oxadiazol-3-yl)phenyl)ethyl)-3-hydroxypyrrolidine-2-carboxamide hemi tartrate salt

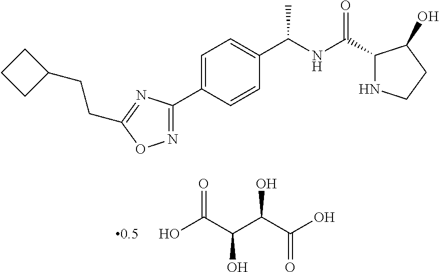

Utilizing a procedure similar to that described in Preparation A, the compound of Example 100 was prepared from (S)-1-(4-(5-(2-cyclobutylethyl)-1,2,4-oxadiazol-3-yl)phenyl)ethanamine and N-(tert-butoxycarbonyl)-trans-3-hydroxy-L-proline. In this case, the carbamate deprotection was accomplished using anhydrous hydrogen chloride in methanol. The product, a white solid, was isolated as the hemi-tatrate salt (from aqueous ethanol by lyophilization); MS (ESI) m/z 385.

### Example 101

### (2S,3R)-2-amino-N-(4-(5-heptylpyridin-2-yl)phenyl)-3-hydroxybutanamide

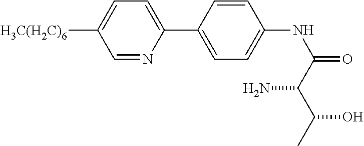

### Preparation of the intermediate 4-(5-heptylpyridin-2-yl)aniline

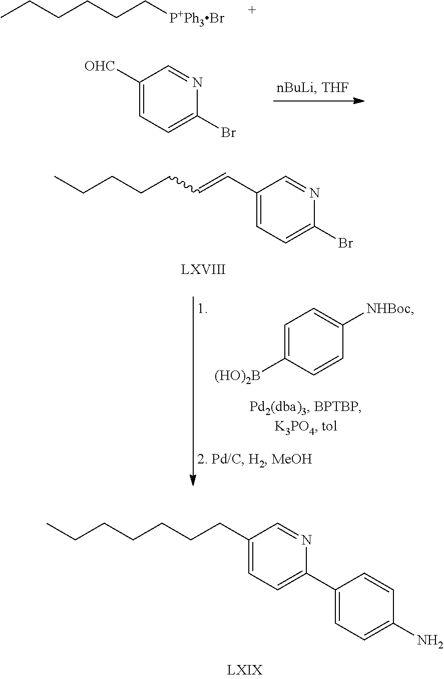

### Step 1) 2-bromo-5-(oct-1-enyl)pyridine (LXVIII)

To a solution of n-Hexyl triphenylphosphonium bromide (10.34 g, 24.2 mmol) in THF (100 mL) was added n-butyllithium (2.5M in hexanes, 9.7 mL, 24.2 mmol) at −78 ° C. The mixture was stirred at this temperature for 1 hour, then 6-bromonicotinaldehyde (3.0 g, 16.13 mmol) was added dropwise. The mixture was slowly warmed to room temperature. After 4 hours, 3 g of NH₄Cl along with 1 mL of H₂O was added and stirred for 10 minutes. The solvent was evaporated, the residue was suspended in CH₂Cl₂, followed by 10 g of silica gel. The solvent was evaporated and the residue was loaded on CombiFlash and washed with 0-20% EtOAc in hexanes to give a colorless liquid, 2-bromo-5-(oct-1-enyl)pyridine (LXVIII) as a mixture of cis and trans isomers (3.3 g, 81%). ¹H NMR(CDCl₃): 8.20(s, 1H), 7.45-7.30(m, 2H), 6.23-6.18(m, 1H), 5.80-5.70(m, 1H), 2.22-2.10(m, 2H), 1.42-1.30(m, 2H), 1.28-1.18(m, 4H), 0.88-0.78(m, 3H).

### Step 2) tert-butyl 4-(5-(hept-1-enyl)pyridin-2yl)phenylcarbamate

A suspension of 4-(tert-butoxycarbonylamino)phenylboronic acid (1.19 g, 5.0 mmol), 2-bromo-5-(oct-1-enyl)pyridine (LXVIII) (1.40 g, 5.5 mmol), K₃PO₄ (2.12 g, 10.0 mmol), Pd₂(dba)₃ (0.069 g, 0.075 mmol), biphenyl-2-yldi-tert-butylphosphine (0.12 g, 0.375 mmol) in 5 mL of toluene was sealed in a microwave reaction vial (20 mL) and the mixture was heated at 80° C. overnight. The mixture was then diluted with EtOAc (100 mL) and washed with water, brine, dried over sodium sulfate. After removal of solvent the residue was chromatographed to give 1.52 g of yellowish oil, tert-butyl 4-(5-(hept-1-enyl)pyridin-2-yl)phenylcarbamate. ¹H NMR(CDCl₃): 8.60(s, 1H), 8.00-7.90(m, 2H), 7.70-7.58(m, 2H), 7.65-7.58(m, 2H), 6.60(s, 1H), 6.40-6.28(m, 1H), 5.80-5.70(m, 1H), 2.40-2.12(m, 1.60-1.45(m, 11H), 1.40-1.28(m, 4H), 0.90-0.80(m, 3H).

### Step 3) tert-butyl 4-(5-heptylpyridin-2-yl)phenylcarbamate

To a solution of tert-butyl 4-(5-(hept-1-enyl)pyridin-2-yl)phenylcarbamate (1.50g, 4.1 mmol) was bubbled N₂ for 10 mM, then added 10% Pd/C (wet, 1.0 g). H₂ balloon was equipped and the mixture was stirred at room temperature overnight. The mixture was filtered over celite and the filter cake was washed with EtOAc. The solvent was removed to give a colorless oil, 1.1 g (73%). The crude product, tert-butyl 4-(5-heptylpyridin-2-yl)phenylcarbamate was used for next step without further purification.

### Step 4) 4-(5-heptylpyridin-2-yl)aniline (LXIX)

To a solution of tert-butyl 4-(5-heptylpyridin-2-yl)phenylcarbamate in CH₂Cl₂ (10 mL) was added 5 mL of CF₃COOH. The mixture was stirred at room temperature for 2 hours. The solvent was then removed and the residue was dissolved in 20 mL of 2N NH₃ in MeOH. The solvent was removed again and the residue was dissolved in 20 mL of CH₂Cl₂ and 5 g of silica gel was added. The solvent was removed, the residue was loaded on CombiFlash and washed with 0-50% of EtOAc in hexanes to give 4-(5-heptylpyridin-2-yl)aniline (LXIX) as a colorless oil, 0.6 g obtained (75%). 8.41(s, 1H), 7.85-7.78(m, 2H), 7.60-7.50(m, 2H), 6.80-6.74(m, 2H), 4.00-3.70(bs, 2H), 2.65-2.58(m, 2H), 1.72-1.60m, 2H), 1.40-1.22(m, 8H), 0.92-0.85(m, 3H).

Utilizing a procedure similar to that described in the steps 4 and 5 in Example 33, the compound of Example 101 was prepared from 4-(5-heptylpyridin-2-yl)aniline and (L)-threonine. Product was afforded as colorless solid. ¹HNMR (CD₃OD): 8.73(s, 1H), 8.04-7.99(m, 2H), 7.90-7.80(m, 4H), 4.23-4.17(m, 1H), 2.83-2.77(m, 2H), 1.82-1.75(m, 2H), 1.55-1.38(m, 12H), 1.05-0.99(m, 3H).

### Example 102

### (2S,4S)—N-(4-(3-heptyl-1,2,4-oxadiazol-5-yl)benzyl)-4-hydroxypiperidine-2-carboxamide

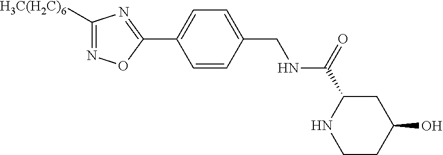

Utilizing a procedure similar to that described in Preparation A, the compound of Example 102 was prepared from (4-(3-heptyl-1,2,4-oxadiazol-5-yl)phenyl)methanamine (LIII) and phenylmethanamine (2S,4S)-1-(tert-butoxycarbonyl)-4-hydroxypiperidine-2-carboxylate (0.35 g, 1.0 mmol). Product was afforded as a pale yellow foam.

¹HNMR (CD₃OD): 8.10-8.04(m, 2H), 7.52-7.46(m, 2H), 4.54(s, 2H), 4.20(s, 1H), 4.12-4.05(m, 1H), 3.35-3.15(m, 2H), 3.05-2.98(m, 2H), 2.20-2.10(m, 1H), 2.00-1.80(m, 5H), 1.52-1.35(m, 8H), 1.00-0.93(m, 3H).

The intermediate (4-(3-heptyl-1,2,4-oxadiazol-5-yl)phenyl)methanamine (LIII) was prepared in Example 76.

### Example 103

### (2S,3S)-3-hydroxy-N-(4-(2-octylthiazol-4-yl)benzyl)pyrrolidine-2-carboxamide

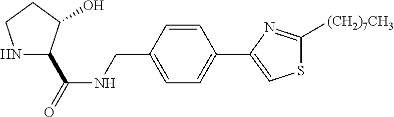

### Preparation of the intermediate (4-(2-octylthiazol-4-yl)phenyl)methanamine (LXXII)

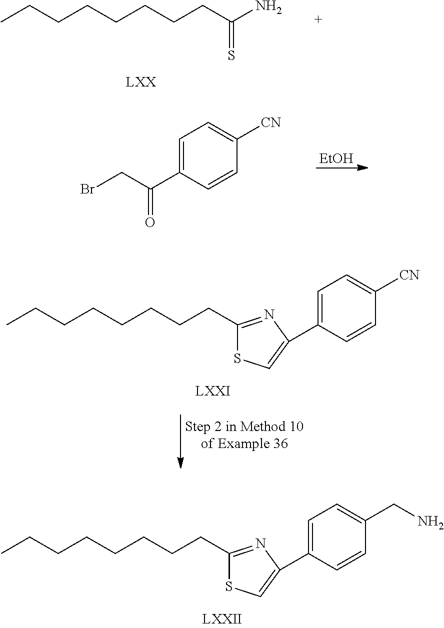

### Step 1) 4-(2-octylthiazol-4-yl)benzonitrile (1.12 g, 5.0 mmol) in ethanol

To a solution of 4-(2-bromoacetyl)benzonitrile (1.12 g, 5.0 mmol) in ethanol (15 mL) was added nonanethioamide (0.866 g, 5.0 mmol). The mixture was refluxed for 6 hours. The solvent was removed and the residue was chromatographed by combiFlash using 0-50% EtOAc in hexanes to give 0.45 g (30%) of 4-(2-octylthiazol-4-yl)benzonitrile (LXXI). ¹HNMR (CDCl₃): 8.02-7.98(m, 2H), 7.75-7.70(m, 2H), 7.50(s, 1H), 3.12-3.03 (m, 2H), 1.90-1.82(m, 2H), 1.52-1.22(m, 10H), 0.95-0.88(m, 3H).

### Step 2) (4-(2-octylthiazol-4-yl)phenyl)methanamine (LXXII)

Utilizing a procedure similar to that described in the Step 2 in Example 46 of Example 36, (4-(2-octylthiazol-4-yl)phenyl)methanamine (0.36 g, 79%) was prepared from 4-(2-octylthiazol-4-yl)benzonitrile (0.45 g, 1.51 mmol) and LAH (0.17 g, 4.53 mmol). ¹HNMR (CDCl₃): 7.80-7.75(m, 2H), 7.32-7.28(m, 2H), 7.22(s, 1H), 3.82(s, 2H), 3.00-2.95 (m, 2H), 1.82-1.75(m, 2H), 1.40-1.18(m, 10H), 0.90-0.80(m, 3H).

Utilizing a procedure similar to that described in Preparation A, the compound of Example 103 was prepared from (4-(2-octylthiazol-4-yl)phenyl)methanamine (0.10 g, 0.33 mmol) and (2S,3S)-1-(tert-butoxycarbonyl)-3-hydroxypyrrolidine-2-carboxylic acid (0.084 g, 0.33 mmol). Product was afforded as a colorless solid. ¹HNMR (CD₃OD): 7.90-7.85(m, 2H), 7.63(s, 1H), 7.40-7.35(m, 2H), 4.45-4.38(m, 3H), 3.59-3.55(m, 1H), 3.20-3.05(m, 4H), 1.95-1.78(m, 4H), 1.54-1.30(m, 10H), 0.96-0.90(m, 3H).

### Example 104

### (2S,3S)—N-(4-(5-heptylpyridin-2-yl)phenyl)-3-hydroxypyrrolidine-2-carboxamide

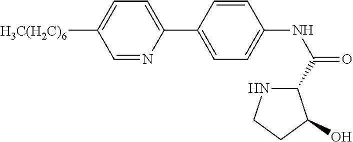

Utilizing a procedure similar to that described in Preparation A, the compound of Example 104 was prepared from 4-(5-heptylpyridin-2-yl)aniline (0.133 g, 0.5 mmol) and (2S,3S)-1-(tert-butoxycarbonyl)-3-hydroxypyrrolidine-2-carboxylic acid (0.115 g, 0.5 mmol). Product was afforded as a colorless solid, ¹HNMR (CD₃OD): 8.63(s, 1H), 8.35-8.30(m, 1H), 8.20-8.16(m, 1H), 8.02-7.91(m, 4H), 4.78-4.70(m, 1H), 4.46-4.41(m, 2H), 2.92-2.88(m, 1H), 2.23-2.15(m, 2H), 1.85-1.78(m, 2H), 1.58-1.32(m, 10H), 0.99-0.93(m, 3H).

The intermediate from 4-(5-heptylpyridin-2-yl)aniline was prepared in accordance with Example 101.

### Example 105

### (2S,3S)—N-(4-(5-heptyl-1H-pyrazol-3-yl)benzyl)-3-hydroxypyrrolidine-2-carboxamide

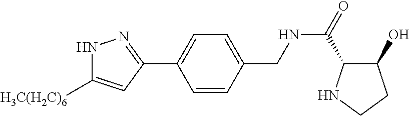

### Preparation of the intermediate (4-(5-heptyl-1H-pyrazol-3-yl)phenyl)methanamine

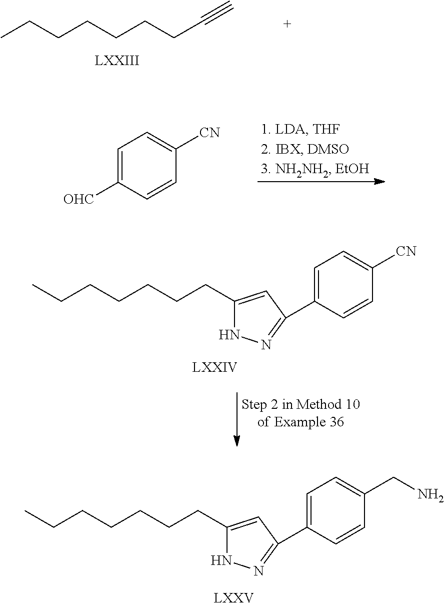

### Step 1) 4-(1-hydroxydee-2-ynyl)benzonitrile

To a solution of nonyne (LXIII) 3.94 mL, 24.0 mmol) in THF (100 mL) was added LDA (2.0M in THF, 12 mL, 24.0 mmol) dropwise at −78° C. After the addition the mixture was warmed to 0° C. for 10 minutes. The mixture was recooled to −78° C. and 4-formylbenzonitrile (2.62 g, 20.0 mmol) was added. The mixture was then slowed warm to room temperature and stirred overnight, quenched with saturated NH₄Cl. The mixture was then extracted with ethyl acetate. The combined organic extracts were washed with 1N HCl, water, brine, dried over Na₂SO₄. After removal of solvent the residue was chromatographed using combiflashed with 0-50% ethyl acetate in hexanes to give 3.5 g (69%) of 4-(1-hydroxydee-2-ynyl)benzonitrile as a colorless: oil. ¹HNMR (CDCl₃): 7.70-7.65(m, 4H), 5.52(s, 1H), 2.32-2.28(m, 2H), 1.60-1.50(m, 2H), 1.42-1.23(m, 8H), 0.92-0.85(m, 3H).

### Step 2) 4-dec-2-ynoylbenzonitrile

To a solution of o-iodoxybenzoic acid (IBX) (8.17 g, 27.6 mmol) in DMSO (30 mL) was added 4-(1-hydroxydee-2-ynyl)benzonitrile (3.5 g, 13.8 mmol) at room temperature. The mixture was stirred for 3 hours, then diluted with 200 mL of ethyl acetate/hexanes (2:1). The mixture was washed with water, brine, dried over sodium sulfate. After removal of solvent the residue, 4-dec-2-ynoylbenzonitrile, was directly used in the next step without further purification.

### Step 3) 4-(3-heptyl-1H-pyrazol-5-yl)benzonitrile (LXXIV)

To a solution of 4-dec-2-ynoylbenzonitrile (0.83 g, 3.28 mmol) in EtOH (10 mL) was added NH₂NH₂ 2HCl (0.41 g, 3.93 mmol). The mixture was refluxed for 2 hours until all the starting material was gone. The solvent was removed and the residue was chromatographed by combiflash with 0-50% EtOAc in hexanes to give 0.32 g (37%) of 4-(3-heptyl-1H-pyrazol-5-yl)benzonitrile. ¹HNMR (CDCl₃): 10.4-10.3(bs, 1H), 8.00-7.90(m, 2H), 7.80-7.70(m, 2H), 6.58(s, 1H), 2.82-2.75(m, 2H), 1.80-1.70(m, 2H), 1.42-1.23(m, 8H), 0.92-0.85(m 3H).

### Step 4) (4-(2-octylthiazol-4-yl)phenyl)methanamine (LXXV)

Utilizing a procedure similar to that described in Step 2 in Example 36, (4-(2-octylthiazol-4-yl)phenyl)methanamine (LXXV) was prepared from 4-(3-heptyl-1H-pyrazol-5-yl)benzonitrile (0.30 g, 92%). ¹HNMR (CDCl₃): 7.72-7.65(m, 2H), 7.40-7.33(m, 2H), 6.40(s, 1H), 3.92(s, 2H), 2.73-2.65(m, 2H), 1.80-1.70(m, 2H), 1.45-1.25(m, 8H), 0.92-0.85(m, 3H).

Utilizing a procedure similar to that described in Preparation A, the compound of Example 105 was prepared from (4-(5-heptyl-1H-pyrazol-3-yl)phenyl)methanamine (0.10 g, 0.37 mmol) and (2S,3S)-1-(tert-butoxycarbonyl)-3-hydroxypyrrolidine-2-carboxylic acid (0.10 g, 0.44 mmol). Product was afforded as a colorless solid. ¹HNMR (CD₃OD): 8.15-8.10(m, 1H), 7.76-7.70(m, 2H), 7.40-7.35(m, 2H), 7.01-6.95(m, 1H), 6.42-6.39(m, 1H), 4.58-4.55(m, 1H), 4.46-4.41(m, 2H), 4.21-4.19(m, 1H), 2.72-2.64(m, 2H), 2.13-2.03(m, 2H), 1.78-1.62(m, 2H), 1.42-1.28(m, 8H), 0.99-0.93(m, 3H).

### Example 106

### (2S,3S)-3-hydroxy-N-(4-(5-octylthiazol-2-yl)benzyl)pyrrolidine-2-carboxamide

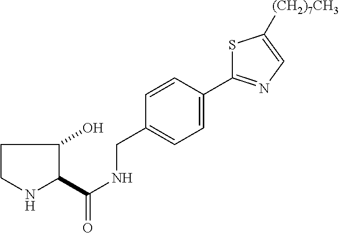

### Preparation of the intermediate (4-(5-octylthiazol-2-yl)phenyl)methanamine

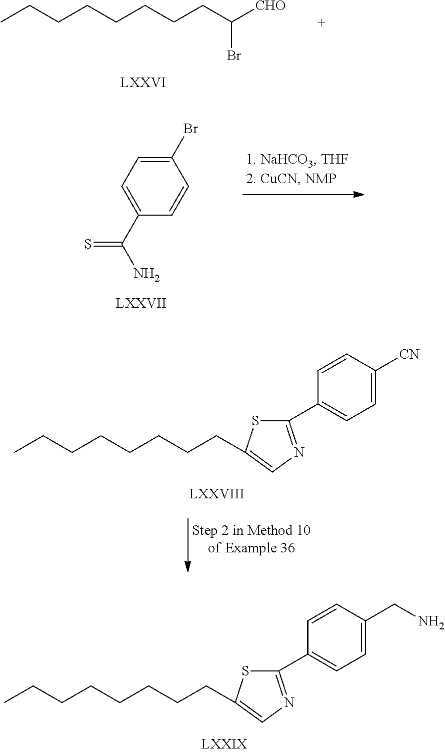

### Step 1) 2-(4-Bromophenyl)-5-octyl-4,5-dihydrothiazol-4-ol

To a boiling solution of 4-bromobenzothioamide (LXXVII)(1.02 g, 5.0 mmol), NaHCO₃ (1.68 g, 20.0 mmol) in THF (8.0 mL) and water (2.0 mL) was added 2-bromodecanal (LXXVI)(1.1 g, 5.0 mmol, prepared based on Organic Precess Research & Development, 3(6), 480-484, 1999) dropwise over 30 min. After the addition the mixture was refluxed for 3 hours, another portion (0.22 g, 1.0 mmol) of 2-bromodecanal was added and the mixture was refluxed overnight. The mixture was diluted, with 150 mL of EtOAc, washed with water, brine, dried over Na₂SO₄. After removal of solvent the residue was chromatographed by CombiFlash using 0-50% of ethyl acetate in hexanes to give 1.1 g (62%) of 2-(4-bromophenyl)-5-octyl-4,5-dihydrothiazol-4-ol as colorless solid. ¹HNMR (CDCl₃):7.79-7.73 (m, 2H), 7.62-7.56(m, 2H), 5.86-5.82(m, 1H), 3.90-3.82(m, 1H), 1.92-1.82(m, 2H), 1.74-1.62(m, 2H), 1.55-1.25(m, 8H), 0.95-0.88(m, 3H).

### Step 2) 4-(5-Octylthiazol-2-yl)benzonitrile (LXXVIII)

A mixture of 2-(4-bromophenyl)-5-octyl-4,5-dihydrothiazol-4-ol (1.0 g, 3.07 mmol), CuCN (0.41 g, 4.60 mmol) and NMP (6 mL) was heated to reflux for 1 hour. The mixture was then cooled to room temperature and diluted with 200 mL of ethyl acetatehexanes (2:1), washed with water, brine, dried over Na₂SO₄. After removal of solvent the residue was chromatographed with CombiFlash (0-20% EtOAc in Hexanes) to give 0.8 g of 4-(5-octylthiazol-2-yl)benzonitrile as colorless solid (0.8 g, 96%). ¹HNMR (CDCl₃): 8.02-7.98(m, 2H), 7.74-7.70(m, 2H), 7.59(s, 1H), 2.90-2.82(m, 2H), 1.77-1.70(m, 2H), 1.42-1.20(m, 8H), 0.92-0.84(m, 2H).

### Step 3) (4-(5-Octylthiazol-2-yl)phenyl)methanamine (LXXIX)

Utilizing a procedure similar to that described in Step 2 in Example 36, (4-(5-octylthiazol-2-yl)phenyl)methanamine was obtained from 4-(5-Octylthiazol-2-yl)benzonitrile as a pale yellow solid (0.54 g, 60%). ¹HNMR, ¹HNMR (CDCl₃): 7.90-7.80 (m, 2H), 7.50 (s, 1H), 7.40-7.30 (m, 2H), 3.92(s, 2H), 2.86-2.80(m, 2H), 1.76-1.62(m, 2H), 1.42-1.20(m, 8H), 0.92-0.85(m, 3H).

Utilizing a procedure similar to that described in Preparation A, the compound of Example 106 was prepared from (4-(5-octylthiazol-2-yl)phenyl)methanamine (0.14 g, 0.5 mmol) and cis-1-(tert-butoxycarbonyl)-3-hydroxypiperidine-2-carboxylic acid (0.14 g, 0.6 mmol). Product was afforded as a pale yellow foam. ¹HNMR (CD₃OD): 7.76-7.70(m, 2H), 7.52(s, 1H), 7.40-7.35(m, 2H), 4.40(s, 2H), 4.38-4.34(m, 1H), 3.61-3.58(m, 1H), 3.20-3.05(m, 2H), 2.95-2.90(m, 2H), 1.93-1.78(m, 4H), 1.50-1.30(m, 10H), 0.99-0.93(m, 3H).

### Example 107

### (2S,3S)-3-Hydroxy-N-((2-octylbenzofuran-5-yl)methyl)pyrrolidine-2-carboxamide

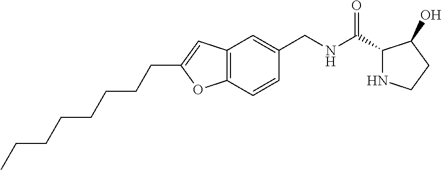

### Preparation of the intermediate (2-octylbenzonfuran-5-yl)-methanamine

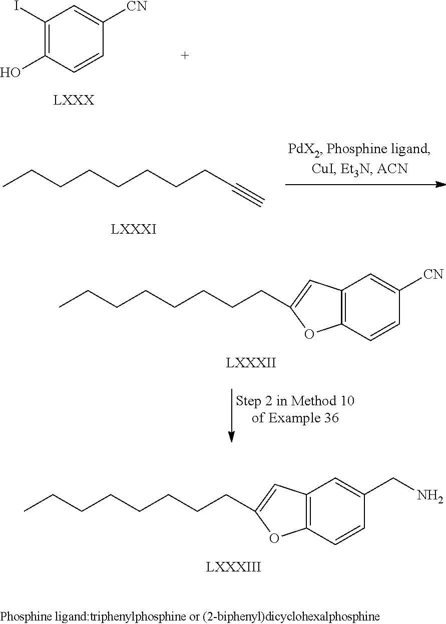

### Step 1) Preparation of 2-octylbenzofuran-5-carbonitrile (LXXXII)

To a stirred solution of 4-hydroxy-3-iodobenzonitrile (LXXX) (4.55 g, 18.57 mmol), 1-decyne (LXXXI) (2.57 g, 18.57 mmol), (2-biphenyl)dicyclohexylphosphine (0.325 g, 0.928 mmol), (or using same moles of triphenylphosphine) and triethylamine (5.64 g, 55.71 mmol) in acetonitrile (100 mL) was added palladium(II) acetate (0.208 g, 0.928 mmol) and copper(I) iodide (0.354 g, 1.86 mmol). The reaction mixture was heated to 60° C. After 18 hours, the reaction mixture was allowed to cool to room temperature and was concentrated. The residue was dissolved in ethyl acetate and washed with 1N hydrochloric acid, 6N ammonium hydroxide, and brine. The organic phase was dried (magnesium sulfate), filtered, and concentrated to provide 5.29 g of a brown oil. Flash chromatography using an Isco Combiflash unit (90 g SiO₂ column, 10-20% ethyl acetate/hexanes) afforded 2.62 g (55% yield) of 2-octylbenzofaran-5-carbonitrile (LXXXII) as a yellow solid: ¹H NMR (CDCl₃) δ 7.80 (s, 1H), 7.52-7.43 (m, 2H), 6.43 (s, 1H), 2.78 (t, J=7.6 Hz, 2H), 1.80-1.69 (m, 2H), 1.44-1.20 (m, 10H), 0.88 (t, J=6.9 Hz, 3H) ppm.

### Step 2) Preparation of (2-octylbenzonfuran-5-yl)methanamine (LXXXIII)

Utilizing a procedure similar to that described in Step 2 of Example 36, (2-octylbenzonfuran-5-yl)methanamine (LXXXIII) was prepared from 2-octylbenzofuran-5-carbonitrile, and obtained as a yellow semi-solid: ¹H NMR (CDCl₃) δ 7.41 (s, 1H), 7.35 (d, J=8.4 Hz, 1H), 7.16-7.12 (m, 1H), 6.34 (s, 1H), 3.92 (s, 2H), 2.75 (t, J=7.5 Hz, 2H), 1.78-1.68 (m, 2H), 1.46-1.21 (m, 12H), 0.88 (t, J=6.9 Hz, 3H) ppm.

Utilizing a procedure similar to that described in Preparation A, the compound of Example 107 was prepared from 2-octylbenzonfuran-5-yl)methanamine and N-(tert-butoxycarbonyl)-trans-3-hydroxy-L-proline. The product was obtained as a white ¹H NMR (CDCl₃) δ 7.95-7.87 (m, 1H), 7.38-7.32 (m, 2H), 7.10-7.05 (m, 1H), 6.33 (s, 1H), 4.62-4.55 (m, 1H), 4.51-4.40 (m, 2H), 3.68 (d, J=2.2 Hz, 1H), 3.27-3.17 (m, 1H), 2.99-2.90 (m, 1H), 2.79-2.40 (m, 4H), 1.85-1.78 (m, 2H), 1.77-1.67 (m, 2H), 1.42-1.20 (m, 10H), 0.88 (t, J=6.8 Hz, 3H) ppm. MS (ESI) m/z 373 (M+H)⁺.

### Example 108

### (2S,3S)-3-Hydroxy-N-((2-octyl-1H-indol-5-yl)methyl)pyrrolidine-2-carboxamide

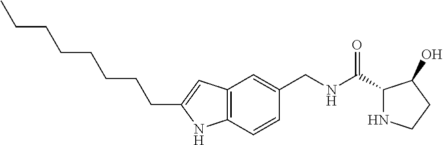

### Preparation of the intermediate (2-octyl-1H-indol-5-yl)methanamine

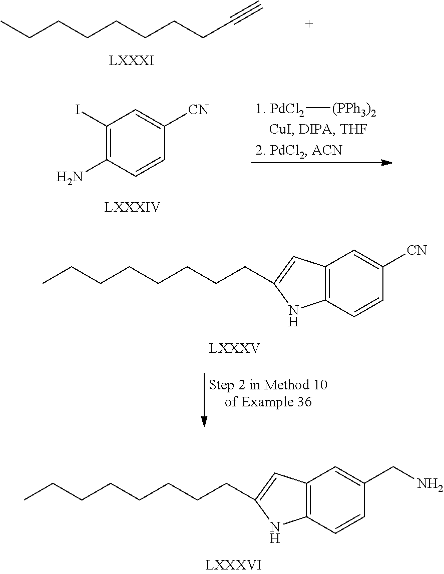

### Step 1) Preparation of 4-amino-3-(dec-1-ynyl)benzonitrile

To a stirred solution of 4-amino-3-iodobenzonitrile (LXXXIV) (7.20 g, 29.50 mmol), 1-decyne (LXXXI) (5.30 g, 38.36 mmol), anti diisopropylamine (8.96 g, 88.51 mmol) in tetrahydrofuran (100 mL) was added copper(I) iodide (0.302 g, 1.59 mmol) and bis(triphenylphosphine)palladium(II) chloride (0.557 g, 0.790 mmol). The reaction mixture was allowed to stir at room temperature. After 18 hours, the reaction mixture was diluted with ethyl acetate and washed with 1N hydrochloric acid, 6N ammonium hydroxide, and brine. The organic phase was dried (magnesium sulfate), filtered, and concentrated to provide 9.62 g of a brown oil. Flash chromatography using an Isco Combiflash unit (330 g SiO₂ column, 15-30% ethyl acetate/hexanes) afforded 6.51 g (87%) of 4-amino-3-(dec-1-ynyl)benzonitrile as a light brown solid: ¹H NMR (CDCl₃) δ 7.50 (d, J=1.9 Hz, 1H), 7.31 (dd, J=1.9, 8.5 Hz, 1H), 6.65 (d, J=8.5 Hz, 1H), 4.65 (s, 2H), 2.46 (t, J=7.1 Hz, 2H), 1.67-1.57 (m, 2H), 1.50-1.39 (m, 2H), 1.37-1.22 (m, 8H), 0.89 (t, J=5.9 Hz, 3H) ppm.

### Step 2) Preparation of 2-octyl-1H-indole-5-carbonitrile (LXXXV)

To a stirred solution of 4-amino-3-(dec-1-ynyl)benzonitrile (6.51 g, 25.59 mmol) in acetonitrile (60 mL) was added palladium(II) chloride (0.318 g, 1.79 mmol). The reaction mixture was heated to reflux. After 1 hour, the reaction mixture was allowed to cool to room temperature and was concentrated to provide a brown solid. Flash chromatography using an Isco Combiflash unit (120 g SiO₂ column, 50% dichloromethane/hexanes to 100% dichloromethane) afforded 4.29 g (66%) of 2-octyl-1H-indole-5-carbonitrile (LXXXVI) as an off-white solid: ¹H NMR (CDCl₃) δ 8.27 (br s 1H), 7.85 (s, 1H), 7.37-7.31 (m, 2H), 6.30 (s, 1H), 2.77 (t, J=7.6 Hz, 2H), 1.78-1.68 (m, 2H), 1.45-1.20 (m, 10H), 0.88 (t, J=6.7 Hz, 3H) ppm.

### Step 3) Preparation of (2-octyl-1H-indol-5-yl)methanamine (LXXXVI)

Utilizing a procedure similar to that described in Step 2 in Example 36 (2-octyl-1H-indol-5-yl)methanamine (LXXXVI) was prepared from 2-octyl-1H-indole-5-carbonitrile (LXXXV), and obtained as a tan solid: ¹H NMR (CDCl₃) δ 7.86 (br s, 1H), 7.44 (s. 1H), 7.28-7.21 (m, 1H), 7.12-7.02 (m, 1H), 6.20 (s, 1H), 3.92 (s, 2H), 2.77-2.66 (m, 2H), 1.76-1.66 (m, 2H), 1.65-1.55 (m, 2H), 1.43-1.20 (m, 10H) 0.94-0.81 (m, 3H) ppm.

Utilizing a procedure similar to that described in Preparation A, the compound of Example 108 was prepared from (2-octyl-1H-indol-5-yl)methanamine and N-(tert-butoxycarbonyl)-trans-3-hydroxy-L-proline. The product was obtained as a white solid: ¹H NMR (CDCl₃) δ 7.96 (br s, 1H), 7.89-7.80 (m, 1H), 7.39 (s, 1H), 7.24 (d, J=8.3 Hz, 1H), 7.02-6.97 (m, 1H), 6.20 (s, 1H), 4.61-4.54 (m, 1H), 4.51-4.40 (m, 2H), 3.65 (d, J=2.3 Hz, 1H), 3.25-3.15 (m, 1H), 2.97-2.88 (m, 1H), 2.74 (t, J=7.6 Hz, 2H), 2.68-2.34 (m, 2H), 1.84-1.77 (m, 2H), 1.75-1.65 (m, 2H), 1.43-1.19 (m, 10H), 0.88 (t, J=6.8 Hz, 3H) ppm. MS (ESI) m/z 372 (M+H)⁺.

### Example 109

### (2S,3S)-3-Hydroxy-N-((1-methyl-2-octyl-1H-indol-5-yl)methyl)pyrrolidine-2-carboxamide

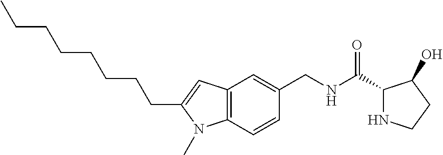

### Preparation of the intermediate (1-methyl-2-octyl-1H-indol-5-yl)methanamine

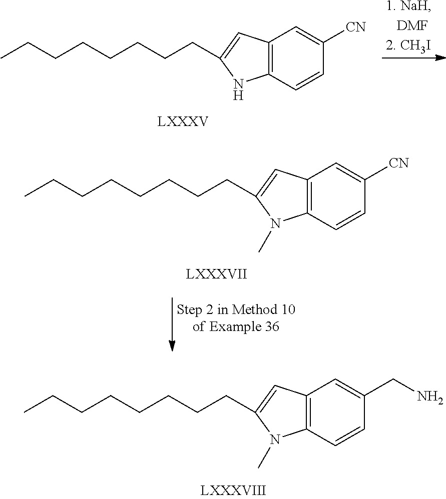

### Step 1) Preparation of 1-methyl-2-octyl-1H-indole-5-carbonitrile (LXXXVII)

To a stirred 0° C. solution of 2-octyl-1H-indole-5-carbonitrile (LXXXV) (1.50 g, 5.90 mmol) in N,N-dimethylformamide (15 mL) was added sodium hydride (95%, 0.170 g, 7.08 mmol. Gas evolution was noted. The reaction mixture was allowed to stir at 0° C. for 0.5 hour, and then iodomethane (0.921 g, 6.49 mmol) was added in one portion. The cooling bath was removed, and the reaction mixture allowed to warm to room temperature. After 1 hour, the reaction mixture was poured into saturated ammonium chloride solution. The mixture was extracted three times with diethyl ether. The combined organic phases were washed with brine, dried (magnesium sulfate), filtered, and concentrated to provide 1.47 g (93%) of 1-methyl-2-octyl-1H-indole-5-carbonitrile (LXXXVII) as a yellow solid: ¹H NMR (CDCl₃) δ 7.85 (s, 1H), 7.40-7.35 (m, 1H), 7.29 (d, J=8.5 Hz, 1H), 6.32 (s, 1H), 3.69 (s, 3H), 2.78-2.70 (m, 2H), 1.78-1.68 (m, 2H), 1.50-1.22 (m, 10H), 0.89 (t, J=6.9 Hz, 3H) ppm.

### Step 2) Preparation of (1-methyl-2-octyl-1H-indol-5-yl)methanamine (LXXXVIII)

Utilizing a procedure similar to that described in Step 2 in Example 36 (1-methyl-2-octyl-1H-indol-5-yl)methanamine (LXXXVIII) was obtained from 1-methyl-2-octyl-1H-indole-5-carbonitrile as a yellow oil: ¹H NMR (CDCl₃) δ 7.45 (s, 1H), 7.22 (d, J=8.3 Hz, 1H), 7.13-7.07 (m, 1H), 6.21 (s, 1H), 3.92 (s, 2H), 3.65 (s, 3H), 2.77-2.67 (m, 2H), 1.77-1.62 (m, 4H), 1.48-1.21 (m, 10), 0.89 (t, J=6.9 Hz, 3H) ppm.

Utilizing a procedure similar to that described in Preparation A, the compound of Example 109 was prepared from (1-methyl-2-octyl-1H-indol-5-yl)methanamine and N-(tert-butoxycarbonyl)-trans-3-hydroxy-L-proline. The product was obtained as a white solid: ¹H NMR (CDCl₃) δ 7.83 (s, 1H), 7.41 (s, 1H), 7.21 (d, J=8.4 Hz, 1H), 7.06-7.01 (m, 1H), 6.21 (s, 1H), 4.61-4.56 (m, 1H), 4.47 (d, J=5.7 Hz, 2H), 3.68-3.62 (m, 4H), 3.24-3.15 (m, 1H), 2.96-2.88 (m, 1H), 2.75-2.20 (m, 4H), 1.84-1.77 (m, 2H), 1.75-1.65 (m, 2H), 1.48-1.21 (m, 10H), 0.89 (t, J=6.8 Hz, 3H) ppm. MS (ESI) m/z 386 (M+H)⁺.

### Example 110

### (2S,3S)—N-((2-(3-(4-Fluorophenoxy)propyl)benzofuran-5-yl)methyl)-3-hydroxypyrrolidine-2-carboxamide

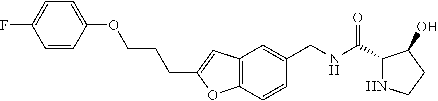

Utilizing a procedure similar to that described in Preparation A, the compound of Example 110 was prepared from (2-(3-(4-fluorophenoxy)propyl)benzofuran-5-yl)methanamine and N-(tert-butoxycarbonyl)-trans-3-hydroxy-L-proline. The product was obtained as a white ¹H NMR (CDCl₃) δ 7.93 (s, 1H), 7.40-7.32 (m, 2H), 7.12-7.07 (m, 1H), 7.00-6.91 (m, 2H), 6.85-6.78 (m, 2H), 6.38 (s, 1H), 4.60-4.56 (m, 1H), 4.52-4.41 (m, 2H), 3.98 (t, J=6.1 Hz, 2H), 3.68 (d, J=2.2 Hz, 1H), 3.27-3.18 (m, 1H), 3.00-2.90 (m, 3H), 2.88-2.42 (m, 2H), 2.25-2.16 (m, 2H), 1.85-1.78 (m, 2H) ppm. MS (ESI) m/z 413 (M+H)⁺.

The intermediate, (2-(3-(4-fluorophenoxy)propyl)benzofuran-5-yl)methanamine, was prepared using procedures similar to that described in Example 107 starting from 1-fluoro-4-(pent-4-ynyloxy)benzene (XVI, see Example 35). It was obtained as a white solid: ¹H NMR (CDCl₃) δ 7.42 (s, 1H), 7.36 (d, J=8.4 Hz, 1H), 7.16 (dd, J=1.7 Hz, 8.4 Hz, 1H), 7.00-6.92 (m, 2H), 6.86-6.79 (m, 2H), 6.39 (s, 1H), 3.99 (t, J=6.1 Hz, 2H), 3.93 (s, 2H), 2.97 (t, J=7.4 Hz, 2H), 2.26-2.17 (m, 2H), 1.43 (s, 2H) ppm.

### Example 111

### (2S,3S)—N-((2-(2-(4-Fluorophenoxy)ethyl)benzofuran-5-yl)methyl)-3-hydroxypyrrolidine-2-carboxamide

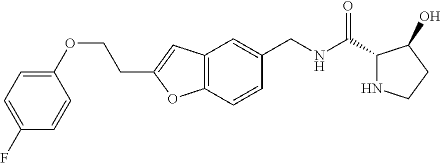

Utilizing a procedure similar to that described in Preparation A, the compound of Example 111 was prepared from (2-(2-(4-fluorophenoxy)ethyl)benzofuran-5-yl)methanamine and N-(tert-butoxycarbonyl)-trans-3-hydroxy-L-proline. The product was obtained as a white solid: ¹H NMR (CDCl₃) δ 7.97-7.88 (m, 1H), 7.40-7.33 (m 2H), 7.14-7.09 (m, 1H), 7.00-6.92 (m, 2H), 6.88-6.81 (m, 2H), 6.50 (s, 1H), 4.60-4.55 (m, 1H), 4.52-4.41 (m, 2H), 4.28 (t, J=6.6 Hz, 2H), 3.68 (d, J=2.3 Hz, 1H), 3.28-3.17 (m, 3H), 2.98-2.90 (m, 1H), 2.44 (br s, 2H), 1.86-1.78 (m, 2H) ppm. MS (ESI) m/z 399 (M+H)⁺.

The intermediate, (2-(2-(4-fluorophenoxy)ethyl)benzofuran-5-yl)methanamine, was prepared using procedures similar to that described in Example 107 starting from 1-(but-3-ynyloxy)-4-fluorobenzene prepared using similar procedures described in Step 1 in Example 28. (2-(2-(4-fluorophenoxy)ethyl)benzofuran-5-yl)methanamine was obtained as a yellow solid: ¹H NMR (CDCl₃) δ 7.44 (s, 1H), 7.39-7.35 (m, 1H), 7.20-7.15 (m, 1H), 7.00-6.93 (m, 2H), 6.88-6.81 (m, 2H), 6.50 (s, 1H), 4.28 (t, J=6.7 Hz, 2H), 3.93 (s, 2H), 3.24 (t, J=6.7 Hz, 2H), 1.43 (br s, 2H) ppm.

### Example 112

### (2S,3S)-3-Hydroxy-N-((2-octylbenzofuran-6-yl)methyl)pyrrolidine-2-carboxamide

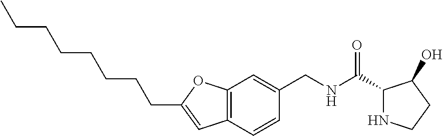

### Preparation of the intermediate (2-octylbenzofuran-6-yl)methanamine

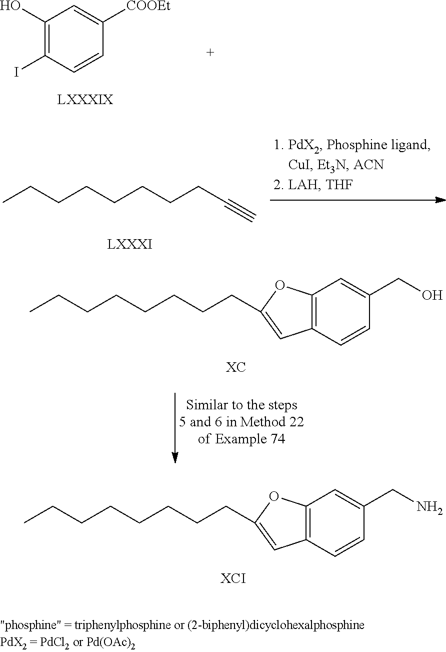

### Step 1) Preparation of ethyl 2-octylbenzofuran-6-carboxylate

To a stirred solution of ethyl 3-hydroxy-4-iodobenzoate (LXXXIX) (2.50 g, 8.56 mmol), 1-decyne (LXXXI) (1.18 g, 8.56 mmol), and diisopropylamine (2.60 g, 25.68 mmol) in tetrahydrofuran (50 mL) was added copper(I) iodide (0.163 g, 0.856 mmol) and bis(triphenylphosphine)palladium(II) chloride (0.300 g, 0.428 mmol). The reaction mixture was heated to reflux. After 17 hours, the reaction mixture was diluted with ethyl acetate and washed with 1N hydrochloric acid, 6N ammonium hydroxide, and brine. The organic phase was dried (magnesium sulfate), filtered, and concentrated to provide 3.01 g of an orange-brown oil. Flash chromatography using an Isco Combiflash unit (120 g SiO₂ column, 5-10% ethyl acetate/hexanes) afforded 1.15 g (44%) of ethyl 2-octylbenzofuran-6-carboxylate as an orange oil: ¹H NMR (CDCl₃) δ 8.10 (s, 1H), 7.93-7.87 (m, 1H), 7.49 (d, J=8.1 Hz, 1H), 6.42 (s, 1H), 4.39 (q, J=7.1 Hz, 2H), 2.83-2.73 (m, 2H), 1.81-1.69 (m, 2H), 1.45-1.21 (m, 13H), 0.92-0.82 (m, 3H) ppm.

### Step 2) Preparation of (2-octylbenzofuran-6-yl)methanol (XC)

To a stirred suspension of lithium aluminum hydride (0.216 g, 5.70 mmol) in tetrahydrofuran (10 mL) was added ethyl 2-octylbenzofuran-6-carboxylate (1.15 g, 3.80 mmol) in tetrahydrofuran (10 mL) over 5 min. The reaction mixture was allowed to stir at room temperature. After 0.5 hour, the reaction mixture treated with water (216 μL), 1N sodium hydroxide (216 μL), and water (648 μL). The resulting mixture was allowed to stir at room temperature for 0.5 hour, and then it was filtered through Celite with the aid of ethyl acetate. The filtrate was washed with saturated sodium potassium tartrate solution and brine, dried (magnesium sulfate), filtered, and concentrated to provide 0.953 g of (2-octylbenzofuran-6yl)methanol (XC) as a yellow solid: ¹H NMR (CDCl₃) δ 7.48-7.40 (m, 2H), 7.21-7.14 (m, 1H), 6.36 (s, 1H), 4.77 (s, 2H), 2.75 (t, J=7.5 Hz, 2H), 1.79-1.68 (m, 2H), 1.63 (br s, 1H), 1.44-1.20 (m, 10H), 0.91-0.84 (m, 3H) ppm.

The intermediate, (2-octylbenzofuran-6-yl)methanamine (XCI), was prepared horn (2-octylbenzofuran-6yl)methanol using procedures similar to that described in the steps 5 and 6 in Example 74. (2-octylbenzofuran-6-yl)methanamine (XCI) was obtained as an off-white solid; ¹H NMR (CDCl3) δ 7.42 (d, J=8.0 Hz, 1H), 7.36 (s, 1H), 7.15-7.10 (m, 1H), 6.34 (s, 1H), 3.95 (s, 2H), 2.75 (t, J=7.5 Hz, 2H), 1.78-1.68 (m, 2H), 1.50 (br s, 2), 1.43-1.22 (m, 10H), 0.88 (t, J=6.9 Hz, 3H) ppm.

Utilizing a procedure similar to that described in Preparation A, the compound of Example 112 was prepared from (2-octylbenzofuran-6-yl)methanamine and N-(tert-butoxycarbonyl)-trans-3-hydroxy-L-proline. The product was obtained as a white solid: ¹H NMR (CDCl₃) δ 7.99-7.89 (m, 1H), 7.41 (m, J=7.9 Hz, 1H), 7.29 (s, 1H), 7.09-7.04 (m, 1H), 6.34 (s, 1H), 4.61-4.56 (m, 1H), 4.59 (d, J=6.0 Hz, 2H), 3.68 (d, J=2.4 Hz, 1H), 3.27-3.18 (m, 1H), 2.99-2.92 (m, 1H), 2.74 (t, J=7.5 Hz, 2H), 2.69-2.19 (m, 2H), 1.86-1.79 (m, 2H), 1.77-1.67 (m, 2H), 1.43-1.20 (m, 10H), 0.91-0.84 (m, 3H) ppm. MS (ESI) m/z 373 (M+H)⁺.

### Example 113

### (2S,3S)—N-((2-Heptylbenzofuran-5-yl)methyl)-3-hydroxypyrrolidine-2-carboxamide

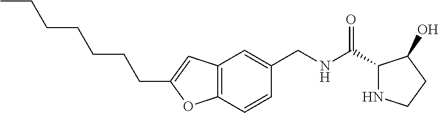

Utilizing a procedure similar to that described in Preparation A, the compound of Example 113 was prepared from (2-heptylbenzonfuran-5-yl)methanamine and N-(tert-butoxycarbonyl)-trans-3-hydroxy-L-proline. The product was obtained as a white solid: ¹H NMR (CDCl₃) δ 7.96-7.86 (m, 1H), 7.38-7.31 (m, 2H), 7.08 (d, J=8.4 Hz, 1H), 6.33 (s, 1H), 4.61-4.55 (m, 1H), 4.51-4.40 (m, 2H), 3.70-3.64 (m, 1H), 3.27-3.18 (m, 1H), 2.98-2.90 (m, 1H), 2.74 (t, J=7.5 Hz, 2H), 2.63-2.01 (m, 2H), 1.86-1.78 (m, 2H), 1.77-1.67 (m, 2H), 1.43-1.20 (m, 8H), 0.88 (t, J=6.6 Hz, 3H) ppm. MS (ESI) m/z 359 (M+H)⁺.

The intermediate, (2-heptylbenzonfuran-5-yl)methanamine, was prepared using procedures similar to that described in Example 107 starting from 4-hydroxy-3-iodobenzonitrile and 1-nonyne. (2-heptylbenzonfuran-5-yl)methanamine was obtained as a yellow oil: ¹H NMR (CDCl₃) δ 7.41 (S, 1H), 7.35 (d, J=8.3 Hz, 1H), 7.16-7.11 (m, 1H), 6.36-6.32 (m, 1H), 3.92 (s, 2H), 2.75 (t, J=7.4 Hz, 2H), 1.78-1.68 (m, 2H), 1.45 (br s, 2H), 1.41-1.22 (m, 8H), 0.88 (t, J=6.9 Hz, 3H) ppm.

### Example 114

### (2S,3S)—N-((2-Hexylbenzofuran-5-yl)methyl)-3-hydroxypyrrolidine-2-carboxamide

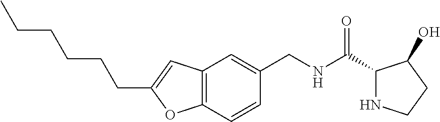

Utilizing, a procedure similar to that described in Preparation A, the compound of Example 114 was prepared from (2-hexylbenzonfuran-5-yl)methanamine and N-(tert-butoxcarbonyl)-trans-3-hydroxy-L-proline. The product was obtained as a white solid: ¹H NMR (CDCl₃) δ 7.94-7.85 (m, 1H), 7.38-7.31 (m, 2H), 7.11-7.05 (m, 1H), 6.33 (s, 1H), 4.50-4.55 (m, 1H), 4.52-4.40 (m, 2H), 3.67 (d, J=2.3 Hz, 1H), 3.27-3.17 (m, 1H), 2.98-2.90 (m, 1H), 2.74 (t, J=7.5 Hz, 2H), 2.67-2.04 (m, 2H), 1.87-1.78 (m, 2H), 1.77-1.67 (m, 2H), 1.44-1.25 (m, 6H), 0.89 (t, J=7.0 Hz, 3H) ppm. MS (ESI) m/z 345 (M+H)⁺.

The intermediate, (2-hexylbenzonfuran-5-yl)methanamine, was prepared using procedures similar to that described in Example 107 starting from 4-hydroxy-3-iodobenzonitrile and 1-octyne. (2-hexylbenzonfuran-5-yl)methanamine was obtained as a yellow oil: ¹H: NMR (CDCl₃) δ 7.41 (s, 1H), 7.35 (d, J=8.4 Hz, 1H), 7.16-7.11 (m, 1H), 6.34 (s, 1H), 3.92 (s, 2H), 2.75 (t, J=7.6 Hz, 2H), 1.78-1.67 (m, 2H), 1.48-1.25 (m, 8H), 0.89 (t, J=7.0 Hz, 3H) ppm.

### Example 115

### (2S,3S)—N-((2-Cyclohexylbenzofuran-5-yl)methyl)-3-hydroxypyrrolidine-2-carboxamide

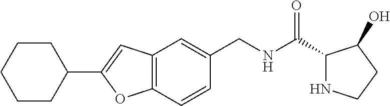

Utilizing, a procedure similar to that described in Preparation A, the compound of Example 115 was prepared from (2-cyclohexylbenzofuran-5-yl)methanamine and N-(tert-butoxycarbonyl)-trans-3-hydroxy-L-proline. The product was obtained as a white solid: ¹H NMR (CDCl₃) δ 7.94-7.85 (m, 1H), 7.39-7.31 (m, 2H), 7.11-7.05 (m, 1H), 6.30 (s, 1H), 4.60-4.55 (m, 1H), 4.51-4.40 (m, 2H), 3.67 (d, J=2.2 Hz, 1H), 3.26-3.17 (m, 1H), 2.98-2.89 (m, 1H), 2.79-2.69 (m, 1H), 2.67-2.28 (m, 2H), 2.14-2.06 (m, 2H), 1.87-1.69 (m, 5H), 1.54-1.20 (m, 5H), ppm, MS (ESI) m/z 343 (M+H)³⁰ .

The intermediate, (2-cyclohexylbenzofuran-5-yl)methanamine, was prepared using procedures similar to that described in Example 107 starting from 4-hydroxy-3-iodobenzonitrile and ethynylcyclohexane. (2-cyclohexylbenzofuran-5-yl)methanamine was obtained as a yellow oil: ¹H NMR (CDCl₃) δ 7.42 (s, 1H), 7.35 (d, J=8.4 Hz, 1H) 7.17-7.11 (m, 1H), 6.31 (s, 1H), 3.92 (s, 2H), 2.80-2.69 (m, 1H), 2.15-2.07 (m, 2H), 1.88-1.79 (m, 2H), 1.77-1.69 (m, 1H), 1.53-1.21 (m, 7H) ppm.

### Example 116

### (2S,3S)—N-((2-(Cyclohexylmethyl)benzofuran-5-yl)methyl)-3-hydroxypyrrolidine-2-carboxamide

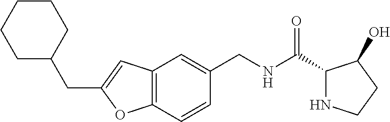

Utilizing a procedure similar to that described in Preparation A, the compound of Example 116 was prepared from (2-(cyclohexylmethyl)benzofuran-5-yl)methanamine and N-(tert-butoxycarbonyl)-trans-3-hydroxy-L-proline. The purified product was isolated as the hydrochloride salt by treatment of an ethereal solution of the free base with anhydrous hydrogen chloride: ¹H NMR (DMSO-d₆) δ 9.77 (br s, 1H), 9.24-9.15 (m, 1H), 8.65 (br s, 1H), 7.47-7.39 (m, 2H), 7.12 (d, J=8.5 Hz, 1H), 6.54 (s, 1H), 5.81 (m, 1H), 4.42-4.32 (m, 3H), 4.09-4.02 (m, 1H), 3.41-3.22 (m, 3H), 2.63 (d, J=6.6 Hz, 2H), 1.98-1.80 (m, 2H), 1.72-1.54 (m, 5H), 1.26-0.87 (m, 5H) ppm. MS (ESI) m/z 357 (M+H)⁺.

The intermediate, (2-(cyclobexylmethyl)benzofuran-5-yl)methanamine, was prepared using procedures similar to that described in Example 107 starting from 4-hydroxy-3-iodobenzonitrile and prop-2-ynylcyclohexane. (2-(cyclohexylmethyl)benzofuran-5-yl)methanamine was obtained as a yellow oil: ¹H NMR (CDCl₃) δ 7.41 (s, 1H), 7.36 (d, J=8.4 Hz, 1H), 7.16-7.12 (m, 1H), 6.33 (s, 1H), 3.92 (s, 2H), 2.63 (d, 6.6 Hz, 2H), 1.82-1.59 (m, 6H), 1.48-1.39 (m, 2H), 1.31-1.07 (m, 3H), 1.06-0.91 (m, 2H) ppm.

### Example 117

### (2S,3S)—N-((2-(2-Cyclohexylethyl)benzofuran-5-yl)-methyl)-3-hydroxypyrrolidine-2-carboxamide

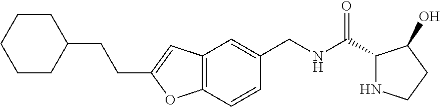

Utilizing a procedure similar to that described in Preparation A, the compound of Example 117 was prepared from (2(2-cyclohexylethyl)benzofuran-5-yl)methanamine and N-(tert-butoxycarbonyl)-trans-3-hydroxy-L-proline. The product was obtained as a white solid: ¹H NMR (CDCl₃) δ 7.99-7.90 (m, 1H), 7.37-7.30 (m, 2H), 7.07 (d, J=8.4 Hz, 1H), 6.32 (s, 1H), 4.60-4.54 (m, 1H), 4.51-4.40 (m, 2H), 3.72-3.67 (m, 1H), 3.27-3.18 (m, 1H), 2.99-2.90 (m, 1H), 2.84-2.62 (m, 4H), 1.85-1.57 (m, 9H), 1.36-1.12 (m, 4H), 1.00-0.87 (m, 2H) ppm. MS (ESI) m/z 371 (M+H)⁺.

The intermediate, (2-(2-cyclohexylethyl)benzofuran-5-yl)methanamine, was prepared using procedures similar to that described in Example 107 starting from 4-hydroxy-3-iodobenzonitrile and but-3-ynylcyclohexane. (2-(2-cyclohexylethyl)benzofuran-5-yl)methanamine was obtained as a yellow oil: ¹H NMR (CDCl₃) δ 7.41 (s, 1H), 7.35 (d, J=8.4 Hz, 1H), 7.16-7.12 (m, 1H), 6.33 (m, 1H), 3.92 (s, 2H), 2.80-2.73 (m, 2H), 1.82-1.59 (m, 7H), 1.50-1.41 (m, 2H), 1.37-1.12 (m, 4H), 1.01-0.88 (m, 2H) ppm.

### Example 118

### (2S,3S)—N-((7-Fluoro-2-octylbenzofuran-5-yl)methyl)-3-hydroxypyrrolidine-2-carboxamide

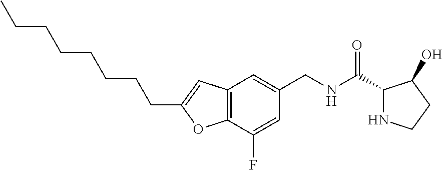

### Preparation of the intermediate (7-fluoro-2-octylbenzofuran-5-yl)methanamine (XCIV)

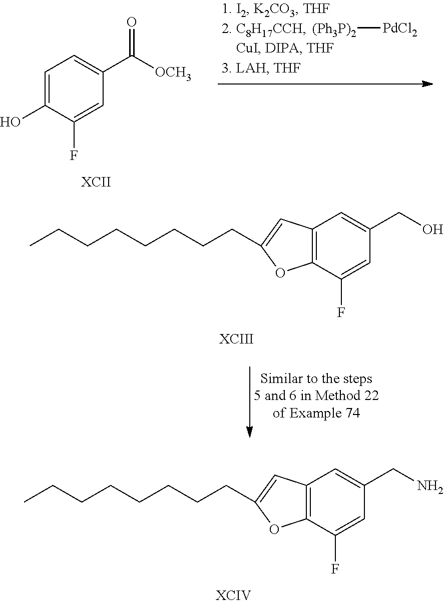

### Step 1) Preparation of methyl 3-fluoro-4-hydroxy-5-iodobenzoate

To a stirred suspension of methyl 3-fluoro-4-hydroxybenzoate (XCII) (4.99 g, 29.33 mmol) and potassium carbonate (4.86 g, 35.19 mmol) in tetrahydrofuran (80 mL) was added iodine (7.82 g, 30.80 mmol). The reaction mixture was allowed to stir at room temperature. After 18 hours, the reaction mixture was treated with additional potassium carbonate (0.600 g, 4.34 mmol) and iodine (1.34 g, 5.27 mmol), and the reaction was heated to reflux for 3 hours. The reaction mixture was then allowed to cool to room temperature and was diluted with ethyl acetate. The solution was washed with saturated sodium thiosulfate solution, 1N hydrochloric acid, and brine. The organic phase was dried (magnesium sulfate), filtered, and concentrated to provide 4.05 g (47%) of methyl 3-fluoro-4-hydroxy-5-iodobenzoate as a yellow solid (isolated with ˜7% of starting material): ¹H NMR (CDCl₃) δ 8.20 (t, J=1.7 Hz, 1H), 7.75 (dd, J=1.7, 10.5 Hz, 1H), 6.12 (br s, 1H), 3.90 (s, 3H) ppm.

### Step 2) Preparation of methyl 7-fluoro-2-octylbenzofuran-5-carboxylate

To a stirred solution of methyl 3-fluoro-4-hydroxy-5-iodobenzoate (3.00 g, 10.13 mmol), 1-decyne (1.40 g, 10.13 mmol), and diisopropylamine (3.08 g, 30.40 mmol) in tetrahydrofuran (50 mL) was added copper(I) iodide (0.193 g, 1.01 mmol) and bis(triphenylphosphine)palladium(II) chloride (0.356 g, 0.507 mmol). The reaction mixture was heated to reflux. After 2 hours, the reaction mixture was allowed to cool to room temperature and was diluted with ethyl acetate. The solution was washed with 1N hydrochloric acid, 6N ammonium hydroxide, and brine. The organic phase was dried (magnesium sulfate), filtered, and concentrated to provide 3.16 g of a brown oil. Flash chromatography using an Isco Combiflash unit (120 g SiO₂ column, 5-10% ethyl acetate/hexanes) afforded 1.34 g (43%) of methyl 7-fluoro-2-octylhenzofuran-5-carboxylate as an orange oil: ¹H NMR (CDCl₃) δ 8.00 (d, J=1.4 Hz, 1H), 7.66 (dd, J=1.4, 11.2 Hz, 1H), 6.47 (d, J=2.9 Hz, 1H), 3.93 (s, 3H), 2.79 (t, J=7.5 Hz, 2H), 1.81-1.70 (m, 2H), 1.44-1.22(m, 10H), 0.88 (t, J=6.9 Hz, 3H) ppm.

### Step 3) Preparation of (7-fluoro-2-octylbenzofuran-5-yl)methanol (XCIII)

To a stirred suspension of lithium aluminum hydride (0.232 g, 6.12 mmol) in tetrahydrofuran (15 mL) was added methyl 7-fluoro-2-octylbenzofuran-5-carboxylate (1.25 g, 4.08 mmol) in tetrahydrofuran (10 mL) over 10 min. The resulting green suspension was allowed to stir at room temperature. After 1 hour, the reaction mixture was cooled to 0° C. and treated with water (232 μL), 1N sodium hydroxide (232 μL), and water (696 μL). The resulting mixture was allowed to stir at room temperature for 0.5 hour, and then it was filtered through Celite with the aid of ethyl acetate. The filtrate was washed with saturated sodium potassium tartrate solution and brine, dried (magnesium sulfate), filtered, and concentrated to provide 1.13 g (99%) of (7-fluoro-2-octylbenzofuran-5-yl)methanol(XCIII) as a yellow oil: ¹H NMR (CDCl₃) δ 7.27-7.22. (m, 1H), 7.00-6.96 (m, 1H), 6.38 (d, J=3.0 Hz, 1H), 4.72 (s, 2H), 2.77 (t, J=7.5 Hz, 2H), 1.80-1.61 (m, 3H), 1.43-1.21 (m, 10H), 0.88 (t, J=6.9 Hz, 3H) ppm.

The intermediate, (7-fluoro-2-octylbenzofuran-5-yl)methanamine (XCIV), was prepared from (7-fluoro-2-octylbenzofuran-5-yl)methanol (XCIII) using procedures similar to that described in the steps 5 and 6 in Example 74. (7-fluoro-2-octylbenzofuran-5-yl)methanamine (XCIV) was obtained as a yellow oil: ¹H NMR (CDCl₃) δ 7.20-7.15 (m, 1H), 6.96-6.88 (m, 1H), 6.36 (d, J=3.0 Hz, 1H), 3.90 (s, 2H), 2.77 (t, J=7.6 Hz, 2H), 1.79-1.69 (m, 2H), 1.45 (br s, 2H), 1.41-1.21 (m, 10H), 0.88 (t, J=6.9 Hz, 3H) ppm.

Utilizing a procedure similar to that described in Preparation A, the compound of Example 118 was prepared from (7-fluoro-2-octylbenzofuran-5-yl)methanamine and N-(tert-butoxycarbonyl)-trans-3-hydroxy-L-proline. The product was obtained as a white solid: ¹H NMR (CDCl₃) δ 7.98-7.91 (m, 1H), 7.13 (m, 1H), 6.84 (m, 1H), 6.36 (d, J=2.9 Hz, 1H), 4.60-4.55 (m, 1H), 4.50-4.38 (m, 2H), 3.69-3.65 (m, 1H), 3.28-3.20 (m, 1H), 3.00-2.92 (m, 1H), 2.76 (t, J=7.5 Hz, 2H), 2.66-2.14 (m, 2H), 1.87-1.79 (m, 2H), 1.78-1.68 (m, 2H), 1.43-1.19 (m, 10H). 0.88 (t, J=6.8 Hz, 3H) ppm. MS (ESI) m/z 391 (M+H)⁺.

### Example 119

### (2S,3S)—N-((6-Fluoro-2-octylbenzofuran-5-yl)methyl)-3-hydroxypyrrolidine-2-carboxamide

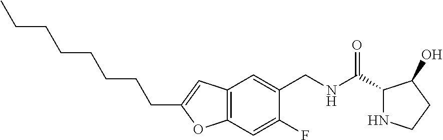

### Preparation of the intermediate (6-fluoro-2-octylbenzofuran-5-yl)methanamine (XCVII)

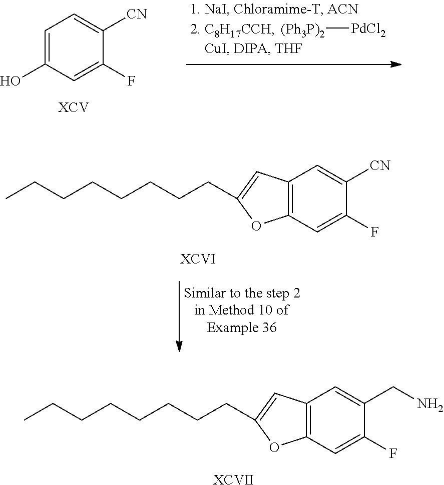

### Step 1) Preparation of 2-fluoro-4-hydroxy-5-iodobenzonitrile and 2-fluoro-4-hydroxy-3-iodobenzonitrile

To a stirred solution of 2-fluoro-4-hydroxybenzonitrile (XCV) (5.15 g, 37.56 mmol) and sodium iodide (6.19 g, 41.32 mmol) in acetonitrile (175 mL) was added Chloramine-T trihydrate (11.64 g, 41.32 mmol). The resulting red-brown mixture was allowed to stir at room temperature. After 1.5 hours, the reaction mixture was concentrated, and the residue partitioned between ethyl acetate and 1N hydrochloric acid. The phases were separated, and the organic phase washed with saturated sodium thiosulfate solution and brine. The organic phase was dried (magnesium sulfate), filtered, and concentrated to provide a white solid. Flash chromatography using an Isco Combiflash unit (330 g SiO₂ column, 25-50% ethyl acetatelhexanes) afforded 6.65 g (67%) of 2-fluoro-4-hydroxy-5-iodobenzonitrile as a white solid (contaminated with ˜16% 2-fluoro-4-hydroxybenzonitrile): ¹H NMR (CDCl₃) δ 7.92 (d, J=7.0 Hz, 1H), 6.86 (d, J=10.0 Hz, 1H), 6.54 (br s, 1H) ppm. Further elution provided 1.28 g (13%) of 2-fluoro-4-hydroxy-3-iodobenzonitrile as a white solid (contaminated with ˜12% p-toluenesulfonamide); ¹H NMR (CDCl₃) δ 7.51 (dd, J=7.3, 8.6 Hz, 1H), 6.88 (dd, J=1.1, 8.6 Hz, 1H), 6.26 (br s, 1H) ppm.

### Step 2) Preparation of 6-fluoro-2-octylbenzofuran-5-carbonitrile (XCVI)

To a stirred solution of 2-fluoro-4-hydroxy-5-iodobenzonitrile (2.00 g, 7.83 mmol), 1-decyne (1.08 g, 7.83 mmol), and diisopropylamine (2.38 g, 23.50 mmol) in tetrahydrofuran (40 mL) was added copper(I) iodide (0.149 g, 0.783 mmol) and bis(triphenylphosphine)palladium(II) chloride (0.274 g, 0.392 mmol). The reaction mixture was heated to reflux. After 17 hours, the reaction mixture was allowed to cool to room temperature and was diluted with ethyl acetate. The solution was washed with 1N hydrochloric acid, 6N ammonium hydroxide, and brine. The organic phase was dried (magnesium sulfate), filtered, and concentrated to provide 2.25 g of an orange oil. Flash chromatography using an Isco Combiflash unit (120 g SiO₂ column, 5-10% ethyl acetate/hexanes) afforded 1.18 g (55%) of 6-fluoro-2-octylbenzofuran-5-carbonitrile (XCVI) as a yellow oil: ¹H NMR (CDCl₃) δ 7.70 (d, J=6.1 Hz 1H) 7.25 (m, 1H (obscured by residual CHCl₃)), 6.40 (s, 1H), 2.76 (t, J=7.6 Hz, 2H), 1.78-1.67 (m, 2H), 1.43-1.20 (m, 10H), 0.88 (t, J=6.8 Hz, 3H) ppm.

The intermediate, (6-fluoro-2-octylbenzofuran-5-yl)methanamine (XCVII) was prepared from 6-fluoro-2-octylbenzofuran-5-carbonitrile (XCVI) using procedures similar to that described in the step 2 in Example 37. (6-fluoro-2-octylbenzofuran-5-yl)methanamine (XCVII) was obtained as a yellow oil: ¹H NMR (CDCl₃) δ 7.35 (d, J=7.4 Hz, 1H), 7.11 (d, J=10.0 Hz, 1H), 6.31 (s, 1H), 3.94 (s, 2H), 2.73 (t, J=7.5 Hz, 2H), 1.77-1.66 (m, 2H), 1.47 (br s, 2H), 1.42-1.20 (m, 10H), 0.88 (t, J=6.9 Hz, 3H) ppm.

Utilizing a procedure similar to that described in Preparation A, the compound of Example 119 was prepared from (6-fluoro-2-octylbenzofuran-5-yl)methanamine and N-(tert-butoxycarbonyl)-trans-3-hydroxy-L-proline. The product was obtained as a white solid: ¹H NMR (CDCl₃) δ 8.21-8.11 (m, 1H), 7.56 (d, J=7.3 Hz, 1H), 7.32 (d, J=10.0 Hz, 1H), 6.51 (s, 1H), 4.78-4.73 (m, 1H), 4.70 (d, J=6.0 Hz, 2H), 3.85 (d, J=2.1 Hz, 1H), 3.88-3.82 (m, 1H), 3.48-3.38 (m, 1H), 3.21-3.12 (m, 1H), 2.92 (t, J=7.5 Hz, 2H), 2.82-2.26 (m, 2H), 2.04-1.97 (m, 2H), 1.95-1.86 (m, 2H), 1.62-1.41 (m, 10H), 1.08 (t, J=6.7 Hz, 3H) ppm. MS (ESI) m/z 391

### Example 120

### (2S,3S)—N-((2-(Cyclopentylmethyl)-7-fluorobenzofuran-5-yl)methyl)-3-hydroxypyrrolidine-2-carboxamide

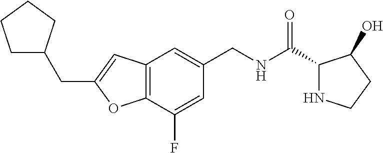

Utilizing a procedure similar to that described in Preparation A, the compound of Example 120 was prepared from (2-(cyclopentylmethyl)-7-fluorobenzofuran-5-yl)methanamine and N-(tert-butoxycarbonyl)-trans-3-hydroxy-L-proline. The purified product was isolated as the hydrochloride salt by treatment of an ethereal solution of the free base with anhydrous hydrogen chloride: ¹H NMR (DMSO-d₆)δ 9.88 (br s, 1H), 9.29 (t, J=5.8 Hz, 1H), 8.64 (br s, 1H), 7.28 (s, 1H), 7.06 (d, J=12.0 Hz, 1H), 6.67 (d, J=3.0 Hz, 1H), 5.86 (br s, 1H), 4.45-4.31 (m, 3H), 4.08 (m, 1H), 3.50-3.23 (m, 3H), 2.76 (d, J=7.3 Hz, 2H), 2.28-2.17 (m, 1H), 1.99-1.82 (m, 2H), 1.79-1.68 (m, 2H), 1.65-1.43 (m, 4H), 1.28-1.15 (m, 2H) ppm. MS (ESI) m/z 361 (M+H)⁺.

The intermediate, (2-(cyclopentylmethyl)-7-fluorobenzofuran-5-yl)methanamine, was prepared from 3-fluoro-4-hydroxy-5-iodobenzoate and prop-2-ynylcyclopentane using procedures similar to that described in Example 107. (2-(cyclopentylmethyl)-7-fluorobenzofuran-5-yl)methanamine was obtained as a colorless oil: ¹H NMR (CDCl₃) δ 7.27-7.16 (m, 1H (obscured by residual CHCl₃)), 6.94-6.89 (m, 1H), 6.37 (d, J=3.0 Hz, 1H), 3.90 (s, 2H), 2.76 (d, J=7.4 Hz, 2H), 2.38-2.25 (m, 1H), 1.87-1.76 (m, 2H), 1.71-1.51 (m, 4H), 1.46 (br s, 2H), 1.33-1.20 (m, 2H) ppm.

### Example 121

### (2S,3S)—N-((4-Fluoro-2-octylbenzofuran-5-yl)methyl)-3-hydroxypyrrolidine-2-carboxamide

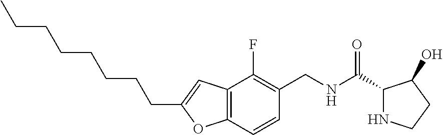

Utilizing a procedure similar to that described in Preparation A, the compound of Example 121 was prepared from (4-fluoro-2-octylbenzofuran-5-yl)methanamine and N-(tert-butoxycarbonyl)-trans-3-hydroxy-L-proline. The product was obtained as a white solid: ¹H NMR (CDCl₃) δ 7.97-7.86 (m, 1H), 7.18-7.06 (m, 2H), 6.44 (s, 1H), 4.59-4.53 (m, 1H), 4.51 (d, J=5.9 Hz, 2H), 3.69-3.62 (m, 1H), 3.27-3.16 (m, 1H), 2.99-2.90 (m, 1H), 2.85-2.27 (m, 4H), 1.83-1.68 (m, 4H), 1.43-1.21 (m, 10H), 0.88 (t, J=6.7 Hz, 3H) ppm. MS (ESI) m/z 391 (M+H)⁺.

The intermediate, (4-fluoro-2-octylbenzofuran-5-yl)methanamine, was prepared from 2-fluoro-4-hydroxy-3-iodobenzonitrile and 1-decyne using procedures similar to that described in Example 107. (4-fluoro-2-octylbenzofuran-5-yl)methanamine was obtained as a yellow oil: ¹H NMR (CDCl₃) δ 7.19-7.08 (m, 2H), 6.44 (d, J=0.8 Hz, 1H), 3.95 (s, 2H), 2.74 (t, J=7.5 Hz, 2H), 1.79-1.68 (m, 2H), 1.47 (br s, 2H), 1.43-1.22 (m, 10H), 0.88 (t, J=6.9 Hz, 3H) ppm.

### Example 122

### (2S,3S)—N-((2-Cyclohexyl-7-fluorobenzofuran-5-yl)methyl)-3-hydroxypyrrolidine-2-carboxamide

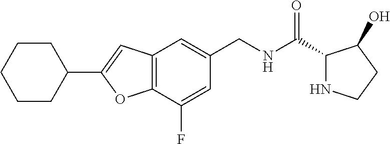

Utilizing a procedure similar to that described in Preparation A, the compound of Example 122 was prepared from (2-cyclohexyl-7-fluorobenzofuran-5-yl)methanamine and N-(tert-butoxycarbonyl)-trans-3-hydroxy-L-proline. The purified product was isolated as the hydrochloride salt by treatment of an ethereal solution of the tree base with anhydrous hydrogen chloride: ¹H NMR (DMSO-d₆) δ 9.88 (br s, 1H), 9.33-9.26 (m, 1H), 8.64 (s, 1H), 7.28 (s, 1H), 7.06 (d, J=11.9 Hz, 1H), 6.63 (d, J=2.8 Hz, 1H), 5.86 (br s, 1H), 4.45-4.31 (m, 3H), 4.08 (br s, 1H), 3.43-3.22 (m, 3H), 2.85-2.74 (m, 1H), 2.09-1.81 (m, 4H), 1.79-1.62 (m, 3H), 1.50-1.16 (m, 4H) ppm. MS (ESI) m/z 361 (M+H)⁺.

The intermediate, (4-fluoro-2-octylbenzofuran-5-yl)methanamine, was prepared from 3-fluoro-4-hydroxy-5-iodobenzonitrile and ethylnylcyclohexane using procedures similar to that described in Example 107. (2-cyclohexyl-7-fluorobenzofuran-5-yl)methanamine was obtained as a yellow oil: ¹H NMR (CDCl₃) δ 7.20-7.16 (m, 1H), 6.95-6.89 (m, 1H), 6.35-6.31 (m, 1H), 3.90 (s, 2H), 2.82-2.72 (m, 1H), 2.17-2.08 (m, 2H), 1.87-1.79 (m, 2H), 1.78-1.70 (m, 1H) 1.56-1.22 (m, 8H) ppm.

### Example 123

### (2R,4R)—N-(4-decylphenyl)-4-hydroxypiperidine-2-carboxamide

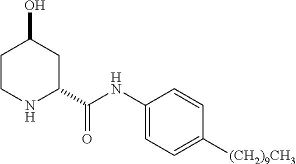

### Synthesis of (2R,4R)—N-(4-decylphenyl)-4-hydroxypiperidine-2-carboxamide

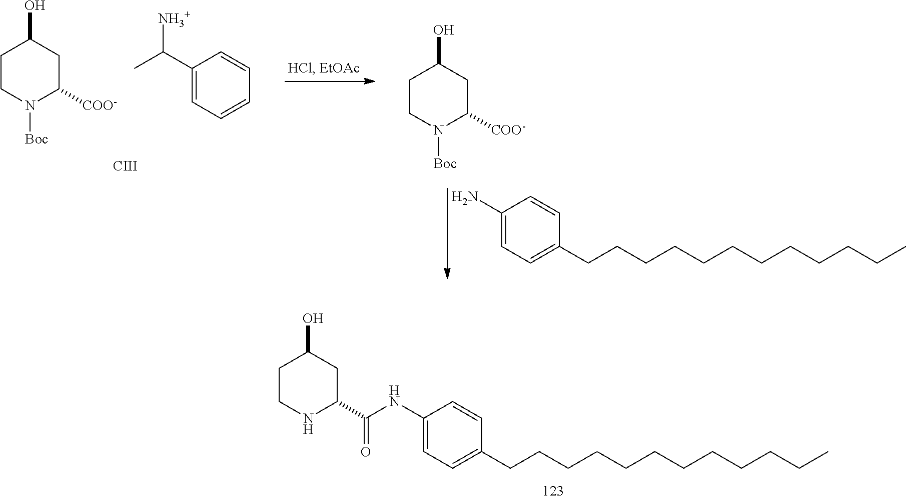

### Step 1) (2R,4R)-1-(tert-butoxycarbonyl)-4-hydroxypiperidine-2-carboxylic acid

The phenylmethanamine (2R,4R)-1-(tert-butoxycarbanyl)-4-hydroxypiperidine-2-carboxylate (CIII) (0.35 g, 1.0 mmol) was dissolved in cold EtOAc (100 mL) and washed with icy cold 1 M HCl (20 mL) twice, then with cold water to pH˜4, then saturated NACl. The organic layer was dried with MgSO4 and the solvent was evaporated, 0.2 g of colorless oil, (2R,4R)-1-(tert-butoxycarbonyl)-4-hydroxypiperidine-2-carboxylic acid, was obtained. ¹H NMR (CDCl₃): 5.00-4.80(m, 1H), 4.10-3.90(m, 1H), 3.72-3.60(m, 1H), 3.00-2.85(m, 1H), 2.45-2.15(m, 1H), 1.90-1.80(m, 1H), 1.62-1.50(m, 1H), 1.42-1.36(m, 10H).

Utilizing as procedure similar to that described in Preparation A, the compound of Example 123 was prepared from (2R,4R)-1-(tert-butoxycarbonyl)-4-hydroxypiperidine-2-carboxylic acid and 4-decylaniline. ¹HNMR (CDCl₃): 9.8(bs, 1H), 7.25-7.20(m, 2H), 7.00-6.90(m, 2H), 4.55-4.45(m, 1H), 4.20-4.10(m, 1H), 3.50-3.40(m, 1H), 3.20-3.10(m, 1H), 2.50-2.39(m, 2H), 2.30-2.20(m, 2H), 1.98-1.65(m, 4H), 1.50-1.40(m, 2H), 1.30-1.08(m, 12H), 0.82-0.72(m, 3H).

### Example 124

### (2S,4S)—N-(4-decylphenyl)-4-hydroxypiperidine-2-carboxamide

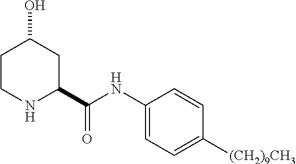

Utilizing a procedure similar to that described in Example 123, the compound of Example 124 was prepared from tert-butyl 4-(4-hydroxybutyl)phenylcarbamate and (2S,3R)-2-(tert-butoxycarbonylamino)-3-hydroxybutanoic acid (BOC protected L-threonine). Product was afforded as a pale yellow foam. ¹HNMR (CD₃OD): 7.55-7.50(m, 2H), 7.20-7.15(m, 2H), 4.10-4.02(m, 1H), 3.30-3.27(m, 1H), 2.61-2.57(m, 2H), 2.50-2.38(, 4H), 1.90-1.80(m, 2H), 1.40-1.20(m, 7H), 0.95-0.90(m, 3H).

### Reporter Assay

Sphingosine kinase is a 49 kDa enzyme that catalyzes the formation of sphingosine-1-phosphate (S-1-P) from sphingosine.

### Materials

Sphingosine kinase far the assay was obtained from BPS Bioscience (lot #01009). Fluorescein labeled sphingosine was obtained from from Echelon Biosciences (S-100F). Buffer reagents (1 M Hepes, 10 triton x-100, 100% glycerol, dithiothreitol 1 M MgCl₂, ATP, 500 mM EDTA and DMSO) were purchased from Sigma. Coating reagent (CR-3), was obtained from Caliper Life Sciences.

### Buffers

Reaction buffer included 100 mM Hepes (pH 7.5), 0,05% Triton X-100, 10% Glycerol, 4 mM DTT, 20 mM MgCl₂, and 25 μM ATP, Separation buffer included 100 mM Hepes (pH 7.5), 15.5 mM EDTA. 0.05% Triton X-100, 2.5% glycerol, 0.1% CR-3, and 0,6% DMSO. Termination butler included 100 mM Hepes (pH 7.5), 40 mM EDTA, 0.05% Triton X-100, 2.5% glycerol, 0.3% CR-3, and 0.6% DMSO.

### Assay

All assay components were made in reaction buffer. The assay included 10 ul of 4.2 uM Fl-sphingosine (final concentration of 2 μM), 1 ul of a compound of the invention in 100% DMSO (final concentration of 4.35% DMSO), and 10 ul of 630 ng/ml SKI (final concentration of 300 ng/ml). The assaylvas allowed to incubate at room temperature sealed and protected from light for 1 hour.

After 1 hour, 10 μl of termination buffer was added to terminate, the reaction, and the plate was read on a Lab Chip 3000. The plate was read for one cycle. The Lab Chip separated substrate and product based on charge. The upstream electrode was set at −500 V and the downstream electrode was set at −2400 Volts with a vacuum pressure of −2.1.

Results were calculated based on percent conversion of substrate to product using the following formula:

$$\% conversion = \frac{P}{P + S}$$

Percent inhibition and IC₅₀'s for compounds of the invention were determined using the percent conversion values obtained horn the Lab Chip. Table I summarizes the IC₅₀ values obtained for Examples 1-124 in accordance with the Reporter Assay method, where n represents the number of runs in the assay.

TABLE 1

| | | | | LC/MS | |
|---|---|---|---|---|---|
| | | | | (ret. | |
| Ex- | | | | Time | SPHK-1 |
| am- | | | | and | IC50 |
| ple | Structure | IUPAC name | MW | M + 1 | (n =) |
| 1 | 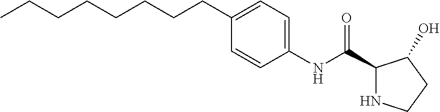 | (2S,3S)-3- Hydroxy-N- (4-octylphenyl) pyrrolidine-2- carboxamide | 318.454 | 7.95 min, 319 m/z | 0.062 μM (n = 3) |
| 2 | 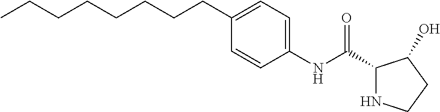 | (2S,3R)-3- Hydroxy-N- (4-octylphenyl) pyrrolidine-2- carboxamide | 318.454 | 7.56 min, 293 m/z | 3.4 μM (n = 1) |
| 3 | 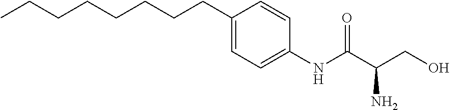 | (R)-2-Amino-3- hydroxy-N-(4- octylphenyl) propanamide | 292.416 | 7.78 min, 293 m/z | 5.0 μM (n = 1) |
| 4 | 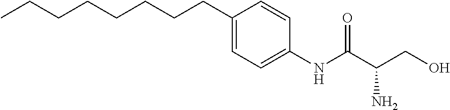 | (S)-2-Amino-3- hydroxy-N-(4- octylphenyl) propanamide | 292.416 | 7.77 min, 293 m/z | 4.3 μM (n = 1) |
| 5 | 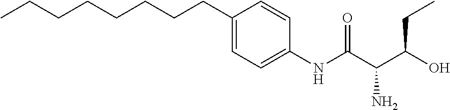 | (±)-erythro- DL-2- Amino-3- hydroxy- N-(4- octylphenyl) pentanamide | 320.47 | 8.38 min, 321 m/z | 3.2 μM (n = 2) |
| 6 | 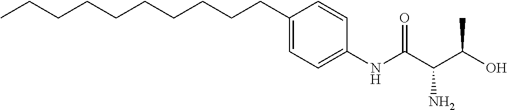 | (2S,3R)-2-Amino- N-(4- decylphenyl)-3- hydroxy- butanamide | 334.496 | 9.24 min, 335 m/z | 0.046 μM (n = 3) |
| 7 | 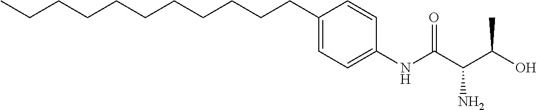 | (2S,3R)-2-Amino- 3-hydroxy-N-(4- undecylphenyl) butanamide | 348.523 | 9.82 min, 349 m/z | 0.11 μM (n = 6) |
| 8 | 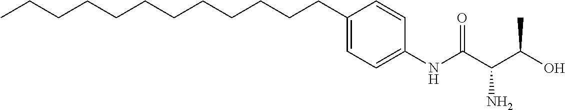 | (2S,3R)-2- Amino-N-(4- dodecylphenyl)- 3- hydroxy- butanamide | 362.549 | 10.50 min, 363 m/z | 0.19 μM (n = 3) |
| 9 | 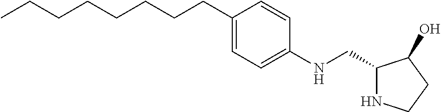 | (2R,3S)-2-((4- Octylphenyl- amino)methyl) pyrrolidin- 3-ol | 304.47 | 7.73 min, 305 m/z | 0.74 μM (n = 2) |
| 10 | 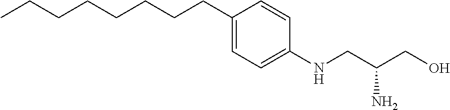 | (R)-2-Amino- 3-(4- octylphenyl- amino) propan-1-ol | 278.433 | 7.73 min, 279 m/z | 5.0 μM (n = 1) |
| 11 | 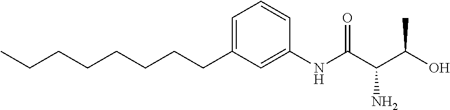 | (2S,3R)-2- Amino-3- hydroxy-N-(3- octylphenyl) butanamide | 306.477 | 7.94 min, 307 m/z | 2.9 μM (n = 1) |
| 12 | 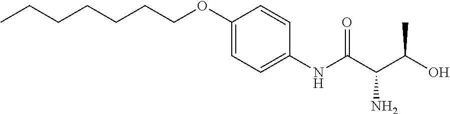 | (2S,3R)-2- Amino-N- (4-(heptyloxy) phenyl)-3- hydroxy- butanamide | 308.419 | 6.89 min, 309 m/z | 1.5 μM (n = 1) |
| 13 | 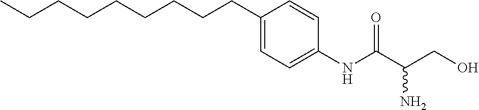 | (+/−)-2- Amino-3- hydroxy-N-(4- nonylphenyl) propanamide | 306.477 | 8.28 min, 307 m/z | 3.8 μM (n = 2) |
| 14 | 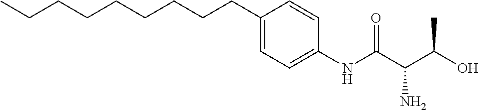 | (2S,3R)-2- Amino-3- hydroxy-N-(4- nonylphenyl) butanamide | 320.473 | 8.60 min, 321 m/z | 0.18 μM (n = 2) |
| 15 | 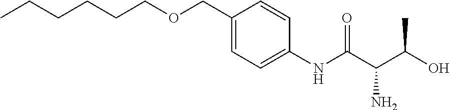 | (2S,3R)-2- Amino-N-(4- (hexyloxymethyl) phenyl)-3- hydroxy- butanamide | 308.419 | 6.09 min, 309 m/z | 8.2 μM (n = 2) |
| 16 | 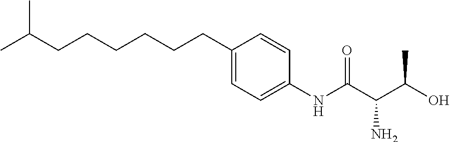 | (2S,3R)-2- Amino-3- hydroxy- N-(4-(7- methyloctyl) phenyl) butanamide | 320.473 | 8.56 min, 321 m/z | 0.15 μM (n = 2) |
| 17 | 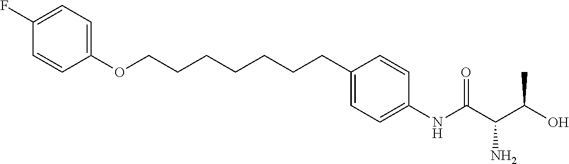 | (2S,3R)-2- Amino-N- (4-(7-(4- fluorophenoxy) heptyl)phenyl)-3- hydroxy- butanamide | 402.506 | 8.05 min, 403 m/z | 3.6 μM (n = 1) |
| 18 | 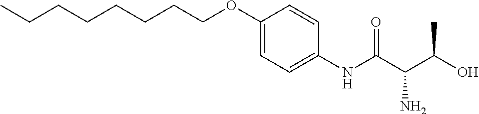 | (2S,3R)-2- Amino-3- hydroxy-N-(4- (octyloxy)phenyl) butanamide | 322.446 | 7.58 min, 323 m/z | 0.46 μM (n = 2) |
| 19 | 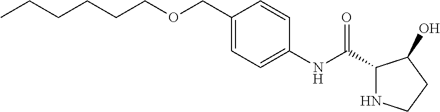 | (2S,3S)-N-(4- (Hexyloxy- methyl) phenyl)-3- hydroxy- pyrrolidine- 2-carboxamide | 320.43 | 6.17 min, 321 m/z | 0.99 μM (n = 2) |
| 20 | 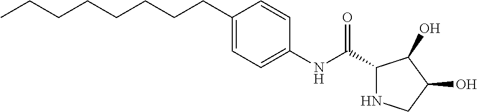 | (2S,3R,4S)-3,4- Dihydroxy-N-(4- octylphenyl) pyrrolidine-2- carboxamide | 334.457 | 7.75 min, 335 m/z | 8.3 μM (n = 1) |
| 21 | 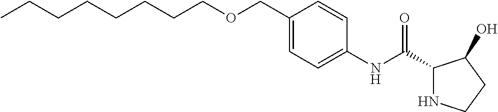 | (2S,3S)-3- Hydroxy- N-(4- (octyloxymethyl) phenyl) pyrrolidine- 2-carboxamide | 348.483 | 7.55 min, 349 m/z | 0.0049 μM (n = 1) |
| 22 | 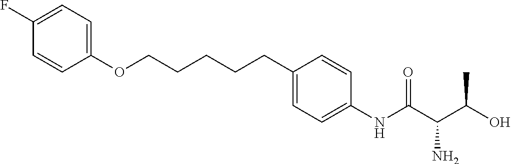 | (2S,3R)-2- Amino-N- (4-(5-(4- fluorophenoxy) pentyl)phenyl)-3- hydroxy- butanamide | 374.453 | 6.97 min, 375 m/z | 3.2 μM (n = 1) |
| 23 | 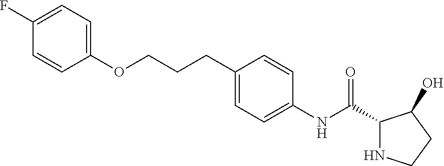 | (2S,3S)-N- (4-(3-(4- Fluorophenoxy) propyl)phenyl)- 3-hydroxy- pyrrolidine- 2-carboxamide | 358.411 | 6.05 min, 359 m/z | 1.5 μM (n = 2) |
| 24 | 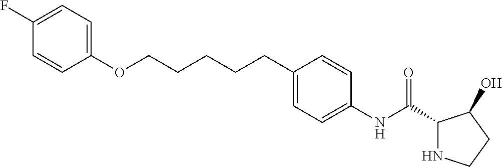 | (2S,3S)-N- (4-(5-(4- Fluorophenoxy) pentyl)phenyl)- 3-hydroxy- pyrrolidine- 2-carboxamide | 386.464 | 7.01 min, 387 m/z | 0.19 μM (n = 3) |
| 25 | 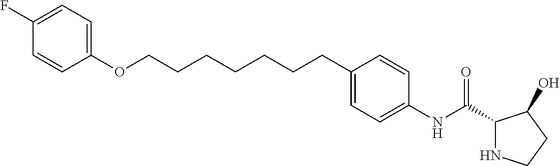 | (2S,3S)-N- (4-(7-(4- Fluorophenoxy) heptyl)phenyl)- 3-hydroxy- pyrrolidine- 2-carboxamide | 414.518 | 8.04 min, 415 m/z | 0.37 μM (n = 2) |
| 26 | 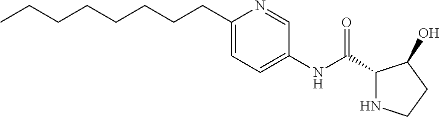 | (2S,3S)-3- Hydroxy- N-(6- octylpyridin- 3-yl) pyrrolidine-2- carboxamide | 319.446 | 6.44 min, 320 m/z | 3.0 μM (n = 2) |
| 27 | 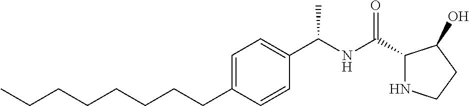 | (2S,3S)-3- Hydroxy- N-((S)-1-(4- octylphenyl)ethyl) pyrrolidine-2- carboxamide | 346.512 | 8.02 min, 347 m/z | 0.19 μM (n = 2) |
| 28 | 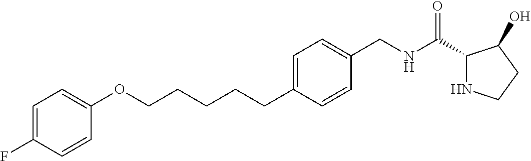 | (2S,3S)-N- (4-(5-(4- Fluorophenoxy) pentyl)benzyl)- 3-hydroxy- pyrrolidine- 2-carboxamide | 400.491 | 6.92 min, 401 m/z | 4.7 μM (n = 1) |
| 29 | 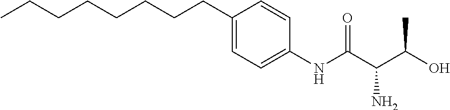 | (2S,3R)-2- Amino-3- hydroxy-N-(4- octylphenyl) butanamide | 306.447 | 7.89 min, 307 m/z | 0.87 μM (n = 18) |
| 30 | 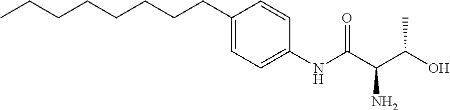 | (2R,3S)-2- Amino-3- hydroxy-N-(4- octylphenyl) butanamide | 306.447 | 7.89 min, 307 m/z | 2.1 μM (n = 1) |
| 31 | 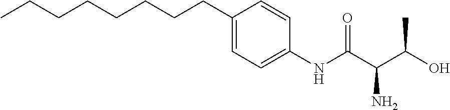 | (2R,3R)-2- Amino-3- hydroxy-N-(4- octylphenyl) butanamide | 306.477 | 7.92 min, 307 m/z | 1.6 μM (n = 1) |
| 32 | 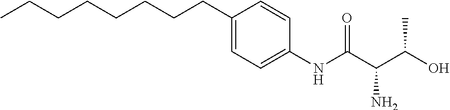 | (2S,3S)-2- Amino-3- hydroxy-N-(4- octylphenyl) butanamide | 306.447 | 7.91 min, 307 m/z | 1.1 μM (n = 1) |
| 33 | 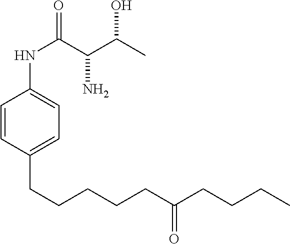 | (2S,3R)-2- amino-3- hydroxy-N- (4-(6- oxodecyl) phenyl) butanamide | 348.48 | 6.44 min, 349.15 m/z | 6.1 μM (n = 1) |
| 34 | 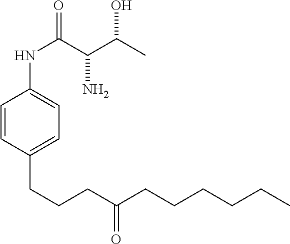 | (2S,3R)-2- amino-3- hydroxy- N-(4-(4- oxodecyl) phenyl) butanamide | 348.48 | 6.74 min, 349 m/z | 1.0 μM (n = 1) |
| 35 | 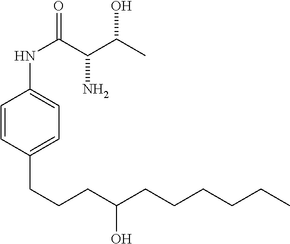 | (2S,3R)-2- amino-3- hydroxy-N- (4-(4- hydroxydecyl) phenyl) butanamide | 350.5 | 6.39 min, 351 m/z | 6.9 μM (n = 1) |
| 36 | 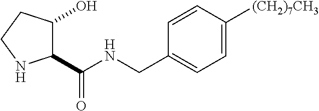 | (2S,3S)-3- hydroxy- N-(4- octylbenzyl) pyrrolidine-2- carboxamide | 332.48 | 8.84 min, 333 m/z | 2.1 μM (n = 2) |
| 37 | 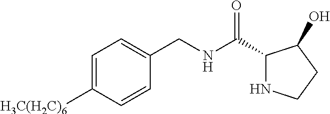 | (2S,3S)-N-(4- heptylbenzyl)-3- hydroxy- pyrrolidine- 2-carboxamide | 318.45 | 7.30 min, 319 m/z | 0.47 μM (n = 1) |
| 38 | 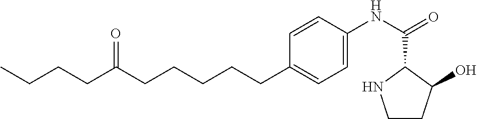 | (2S,3S)-3- hydroxy- N-(4- (6-oxodecyl) phenyl) pyrrolidine- 2-carboxamide | 360.49 | 6.43 min, 361 m/z | 0.086 μM (n = 4) |
| 39 | 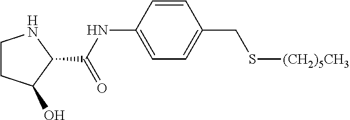 | (2S,3S)-N-(4- (hexylthiomethyl) phenyl)-3- hydroxy- pyrrolidine- 2-carboxamide | 336.49 | 6.98 min, 337 m/z | 0.11 μM (n = 2) |
| 40 | 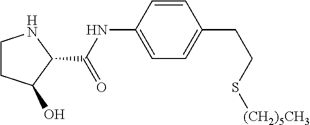 | (2S,3S)-N- (4-(2- (hexylthio)ethyl) phenyl)-3- hydroxy- pyrrolidine- 2-carboxamide | 350.52 | 7.38 min, 351 m/z | 0.007 μM (n = 2) |
| 41 | 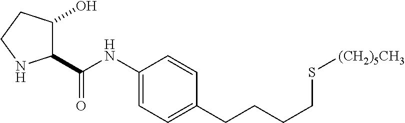 | (2S,3S)-N- (4-(4- (hexylthio) butyl) phenyl)-3- hydroxy- pyrrolidine- 2-carboxamide | 378.57 | 8.36 min, 379 m/z | 0.002 μM (n = 2) |
| 42 | 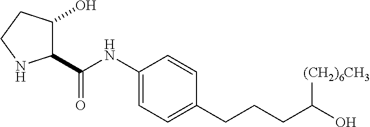 | (2S,3S)-3- hydroxy- N-(4-(4- hydroxy- undecyl) phenyl) pyrrolidine- 2-carboxamide | 376.53 | 7.05 min, 377 m/z | 0.12 μM (n = 1) |
| 43 | 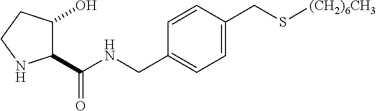 | (2S,3S)-N-(4- (heptylthio- methyl) benzyl)-3- hydroxy- pyrrolidine- 2-carboxamide | 364.55 | 8.44 min, 365 m/z | 0.18 μM (n = 3) |
| 44 | 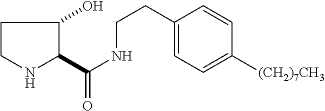 | (2S,3S)-3- hydroxy- N-(4- octylphenethyl) pyrrolidine- 2-carboxamide | 346.51 | 8.71 min, 347 m/z | 6.5 μM (n = 3) |
| 45 | 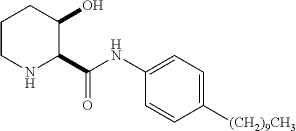 | cis-N-(4- decylphenyl)- 3-hydroxy- piperidine- 2-carboxamide | 360.53 | 8.79 min, 361 m/z | >10 μM (n = 3) |
| 46 | 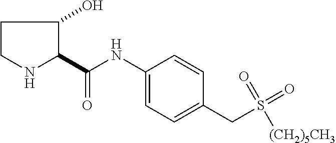 | (2S,3S)-N-(4- (hexylsulfonyl- methyl) phenyl)-3- hydroxy- pyrrolidine- 2-carboxamide | 368.49 | 4.94 min, 369 m/z | >10 μM (n = 3) |
| 47 | 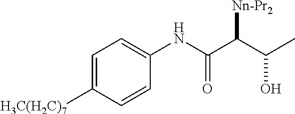 | (2S,3S)-2- (dipropyl- amino)-3- hydroxy-N-(4- octylphenyl) butanamide | 390.6 | N/A | >1 μM (n = 2) |
| 48 | 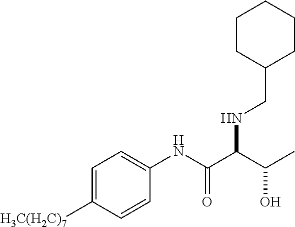 | (2S,3S)-2- (cyclohexyl- methyl- amino)-3- hydroxy- N-(4- octylphenyl) butanamide | 402.61 | 11.58 min, 403 m/z | >1 μM (n = 2) |
| 49 | 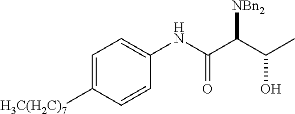 | (2S,3S)-2- (dibenzyl- amino)-3- hydroxy-N-(4- octylphenyl) butanamide | 486.69 | 12.45 min, 487 m/z | >1 μM (n = 2) |
| 50 | 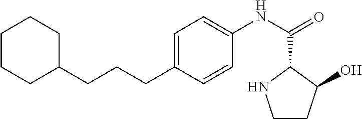 | (2S,3S)-N- (4-(3- cyclohexyl- propyl) phenyl)-3- hydroxy- pyrrolidine- 2-carboxamide | 330.46 | 7.86 min, 331 m/z | 0.95 μM (n = 2) |
| 51 | 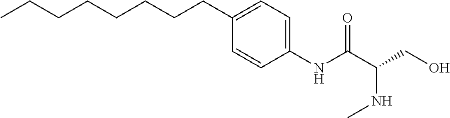 | (S)-3- Hydroxy-2- (methylamino)- N-(4- octylphenyl) propanamide | 306.231 | 7.78 min, 307 m/z | 2.8 μM (n = 1) |
| 52 | 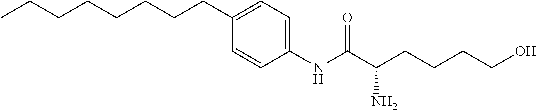 | (S)-2- Amino-6- hydroxy-N-(4- octylphenyl) hexanamide | 334.262 | 335 m/z | 4 μM (n = 3) |
| 53 | 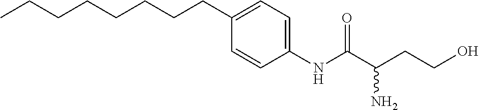 | (±)-2-Amino-4- hydroxy-N-(4- octylphenyl) butanamide | 306.231 | 7.54 min, 307 m/z | 0.175 μM (n = 2) |
| 54 | 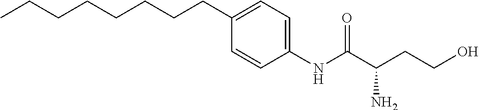 | (S)-2-Amino-4- hydroxy-N-(4- octylphenyl) butanamide | 306.231 | 7.67 min, 307 m/z | 0.027 μM (n = 2) |
| 55 | 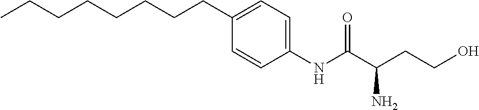 | (R)-2-Amino-4- hydroxy-N-(4- octylphenyl) butanamide | 306.231 | 7.67 min, 307 m/z | 2.2 μM (n = 2) |
| 56 | 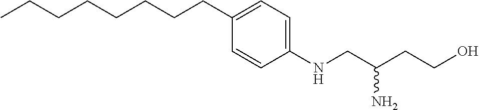 | (±)-3- Amino-4-(4- octylphenyl- amino) butan-1-ol | 292.252 | 7.79 min, 293 m/z | 10.0 μM (n = 2) |
| 57 | 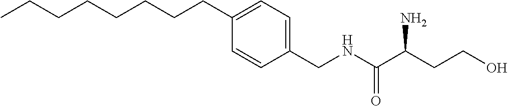 | (S)-2-Amino-4- hydroxy-N-(4- octylbenzyl) butanamide | 320.246 | 7.51 min, 321 m/z | 3.5 μM (n = 3) |
| 58 | 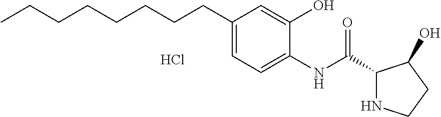 | (2S,3S)-3- hydroxy- N-(2- hydroxy-4- octylphenyl) pyrrolidine-2- carboxamide hydrochloride | 370.202 | 7.48 mim, 335 m/z | 3.6 μM (n = 3) |
| 59 | 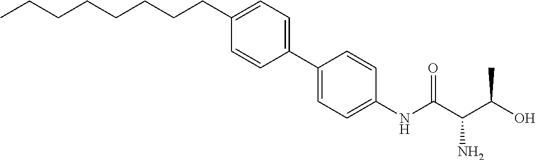 | (2S,3R)-2- Amino-3- hydroxy-N-(4′- octylbiphenyl- 4-yl) butanamide | 382.539 | 9.73 min, 383 m/z | 1.1 μM (n = 2) |
| 60 | 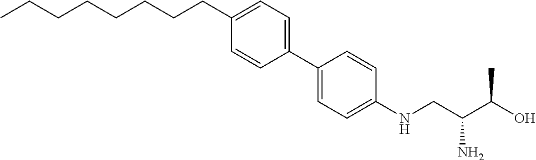 | (2R,3R)-3- Amino-4- (4′-octyl- biphenyl-4- ylamino) butan-2-ol | 368.555 | 9.37 min, 369 m/z | 2.3 μM (n = 2) |
| 61 | 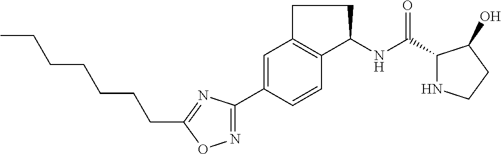 | (2S,3S)-N- ((R)-5-(5- Heptyl-1,2,4- oxadiazol-3- yl)-2,3- dihydro- 1H-inden-1- yl)-3-hydroxy- pyrrolidine-2- carboxamide | 412.531 | 7.47 min, 413 m/z | 6.9 μM (n = 1) |
| 62 | 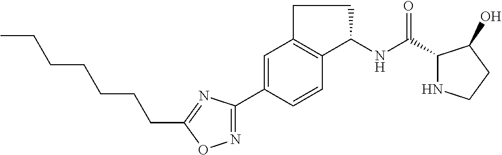 | (2S,3S)-N- ((S)-5-(5- heptyl-1,2,4- oxadiazol-3- yl)-2,3- dihydro-1H- inden-1- yl)-3-hydroxy- pyrrolidine-2- carboxamide | 412.531 | 7.47 min, 413 m/z | 0.060 μM (n = 4) |
| 63 | 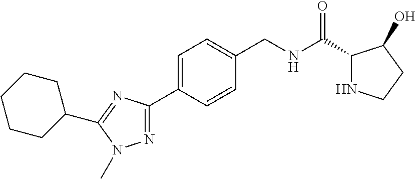 | (2S,3S)-N-(4-(5- Cyclohexyl-1- methyl- 1H-1,2,4- triazol-3- yl)benzyl)- 3-hydroxy- pyrrolidine-2- carboxamide | 383.487 | 4.53 min, 384 m/z | 2.1 μM (n = 3) |
| 64 | 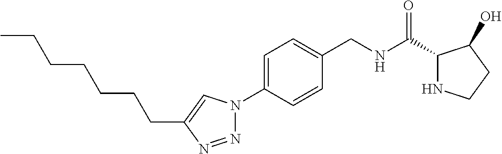 | (2S,3S)- N-(4-(4- Heptyl- 1H-1,2,3- triazol-1- yl)benzyl)- 3-hydroxy- pyrrolidine- 2-carboxamide | 385.509 | 6.42 min, 386 m/z | 1.1 μM (n = 4) |
| 65 | 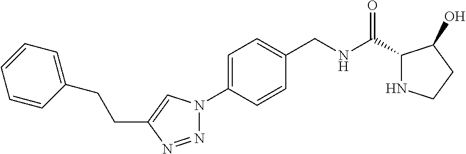 | (2S,3S)-3- Hydroxy- N-(4-(4- phenethyl- 1H-1,2,3- triazol-1- yl)benzyl) pyrrolidine-2- carboxamide | 391.473 | 5.02 min, 392 m/z | 1.6 μM (n = 2) |
| 66 | 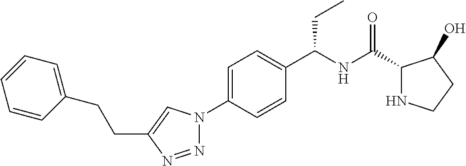 | (2S,3S)-3- Hydroxy- N-((S)-1- (4-(4-phenethyl- 1H-1,2,3- triazol-1- yl)phenyl) propyl) pyrrolidine-2- carboxamide | 419.526 | 5.39 min, 420 m/z | 0.14 μM (n = 2) |
| 67 | 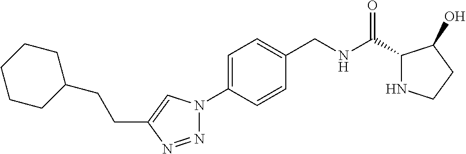 | (2S,3S)-N- (4-(4-(2- Cyclohexylethyl)- 1H-1,2,3- triazol-1- yl)benzyl)-3- hydroxy- pyrrolidine- 2-carboxamide | 397.519 | 6.05 min, 398 m/z | 0.29 μM (n = 2) |
| 68 | 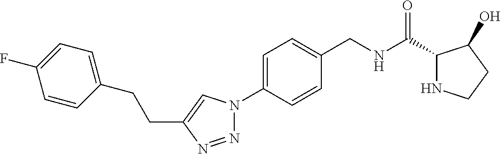 | (2S,3S)-N- (4-(4-(4- Fluoro- phenethyl)- 1H-1,2,3- triazol-1- yl)benzyl)-3- hydroxy- pyrrolidine- 2-carboxamide | 409.463 | 4.94 min, 410 m/z | 7.3 μM (n = 3) |
| 69 | 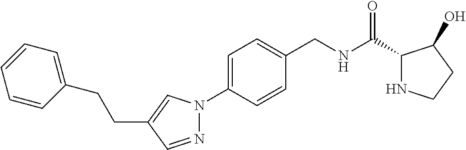 | (2S,3S)-3- Hydroxy- N-(4-(4- phenethyl- 1H-pyrazol- 1-yl)benzyl) pyrrolidine- 2-carboxamide | 390.484 | 5.71 min, 391 m/z | 0.065 μM (n = 5) |
| 70 | 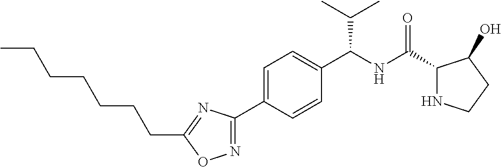 | (2S,3S)-N- ((S)-1-(4- (5-Heptyl- 1,2,4- oxadiazol-3-yl) phenyl)-2- methyl- propyl)-3- hydroxy- pyrrolidine- 2-carboxamide | 428.573 | 7.98 min, 429 m/z | 0.11 μM (n = 3) |
| 71 | 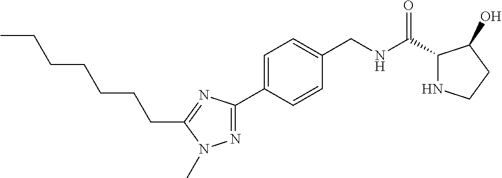 | (2S,3S)-N- (4-(5- Heptyl-1- methyl-1H- 1,2,4-triazol-3- yl)benzyl)-3- hydroxy- pyrrolidine- 2-carboxamide | 399.536 | 5.73 min, 400 m/z | 0.61 μM (n = 2) |
| 72 | 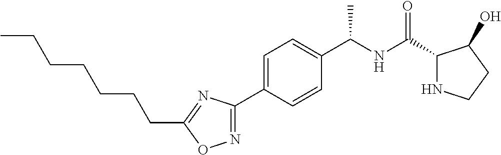 | (2S,3S)-N- ((S)-1-(4- (5-Heptyl-1,2,4- oxadiazol-3-yl) phenyl)ethyl)- 3-hydroxy- pyrrolidine- 2-carboxamide | 400.519 | 7.12 min, 401 m/z | 0.13 μM (n = 3) |
| 73 | 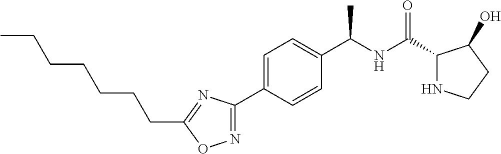 | (2S,3S)-N- ((R)-1-(4- (5-Heptyl-1,2,4- oxadiazol-3-yl) phenyl)ethyl)- 3-hydroxy- pyrrolidine- 2-carboxamide | 400.519 | 7.20 min, 401 m/z | 5.3 μM (n = 2) |
| 74 | 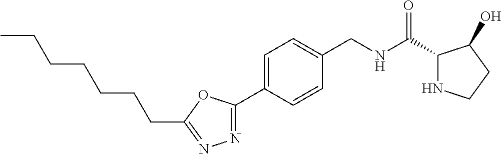 | (2S,3S)-N-(4-(5- Heptyl-1,3,4- oxadiazol-2-yl) benzyl)-3- hydroxy- pyrrolidine- 2-carboxamide | 386.493 | 6.09 min, 387 m/z | 2.2 μM (n = 3) |
| 75 | 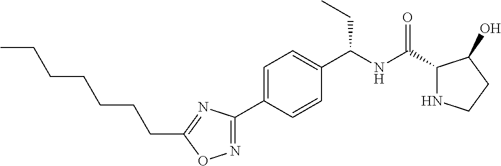 | (2S,3S)-N- ((S)-1-(4- (5-Heptyl-1,2,4- oxadiazol-3- yl)phenyl) propyl)-3- hydroxy- pyrrolidine- 2-carboxamide | 414.547 | 7.63 min, 415 m/z | 0.030 μM (n = 6) |
| 76 | 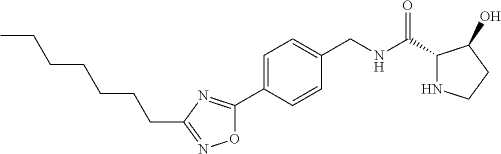 | (2S,3S)-N- (4-(3- Heptyl-1,2,4- oxadiazol-5-yl) benzyl)-3- hydroxy- pyrrolidine- 2-carboxamide | 386.493 | 6.97 min, 387 m/z | 0.061 μM (n = 8) |
| 77 | 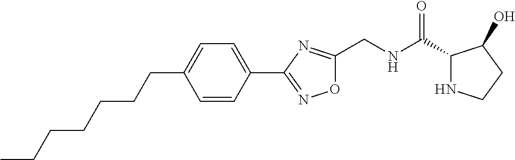 | (2S,3S)-N- ((3-(4- Heptylphenyl)- 1,2,4- oxadiazol-5- yl)methyl)-3- hydroxy- pyrrolidine- 2-carboxamide | 386.493 | 7.88 min, 387 m/z | 0.22 μM (n = 3) |
| 78 | 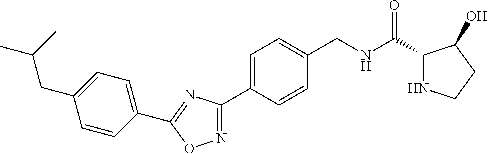 | (2S,3S)-3- Hydroxy- N-(4-(5-(4- isobutylphenyl)- 1,2,4- oxadiazol-3- yl)benzyl) pyrrolidine-2- carboxamide | 420.504 | 7.45 min, 421 m/z | 4.4 μM (n = 1) |
| 79 | 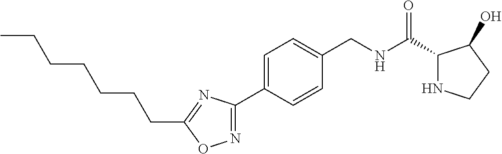 | (2S,3S)-N- (4-(5- Heptyl-1,2,4- oxadiazol-3-yl) benzyl)-3- hydroxy- pyrrolidine- 2-carboxamide | 386.493 | 6.97 min, 387 m/z | 0.068 μM (n = 45) |
| 80 | 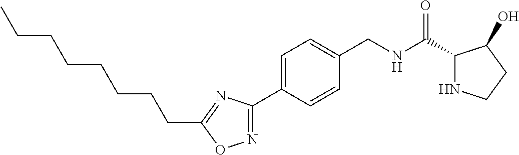 | (2S,3S)-3- Hydroxy- N-(4-(5- octyl-1,2,4- oxadiazol-3-yl) benzyl) pyrrolidine- 2-carboxamide | 400.52 | 7.62 min, 401 m/z | 0.086 μM (n = 3) |
| 81 | 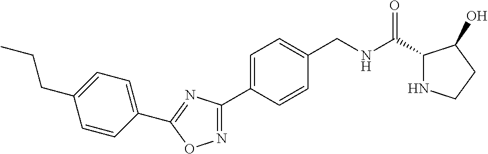 | (2S,3S)-3- Hydroxy- N-(4-(5-(4- propylphenyl)- 1,2,4- oxadiazol-3- yl)benzyl) pyrrolidine-2- carboxamide | 406.483 | 6.96 min, 407 m/z | 1.8 μM (n = 1) |
| 82 | 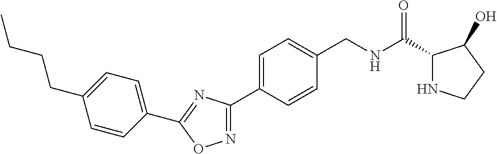 | (2S,3S)-N- (4-(5-(4- Butylphenyl)- 1,2,4- oxadiazol-3- yl)benzyl)-3- hydroxy- pyrrolidine- 2-carboxamide | 420.51 | 7.56 min, 421 m/z | 3.7 μM (n = 1) |
| 83 | 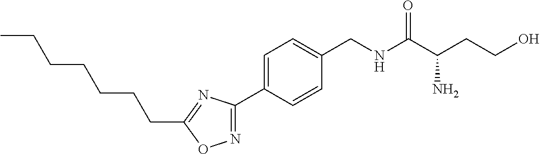 | (S)-2-Amino- N-(4- (5-heptyl-1,2,4- oxadiazol-3-yl) benzyl)-4- hydroxy- butanamide | 374.482 | 6.73 min, 375 m/z | 0.60 μM (n = 1) |
| 84 | 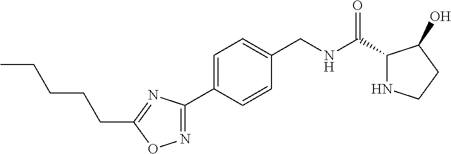 | (2S,3S)-3- Hydroxy- N-(4-(5-pentyl- 1,2,4- oxadiazol-3- yl)benzyl) pyrrolidine-2- carboxamide | 358.439 | 5.70 min, 359 m/z | 0.43 μM (n = 2) |
| 85 | 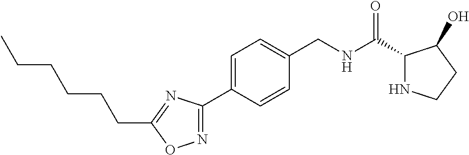 | (2S,3S)-N- (4-(5- Hexyl-1,2,4- oxadiazol-3-yl) benzyl)-3- hydroxy- pyrrolidine- 2-carboxamide | 372.466 | 6.38 min, 373 m/z | 0.048 μM (n = 2) |
| 86 | 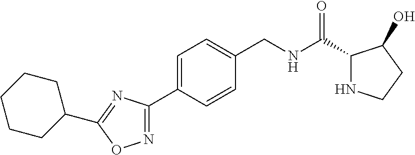 | (2S,3S)-N- (4-(5- Cyclohexyl- 1,2,4- oxadiazol-3-yl) benzyl)-3- hydroxy- pyrrolidine- 2-carboxamide | 370.45 | 5.72 min, 371 m/z | 0.37 μM (n = 24) |
| 87 | 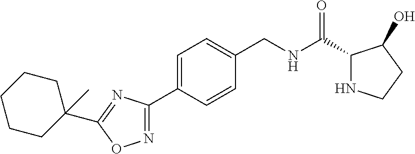 | (2S,3S)-3- Hydroxy- N-(4-(5- (1-methyl- cyclohexyl)- 1,2,4- oxadiazol-3-yl) benzyl) pyrrolidine- 2-carboxamide | 384.477 | 6.25 min, 385 m/z | 0.13 μM (n = 2) |
| 88 | 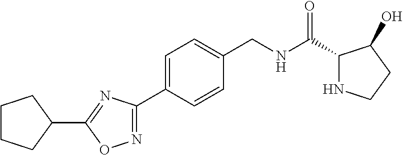 | (2S,3S)-N- (4-(5- Cyclopentyl- 1,2,4- oxadiazol-3-yl) benzyl)-3- hydroxy- pyrrolidine- 2-carboxamide | 356.424 | 5.09 min, 357 m/z | 4.5 μM (n = 3) |
| 89 | 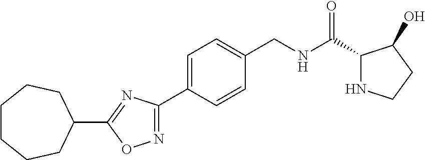 | (2S,3S)-N- (4-(5- Cycloheptyl- 1,2,4- oxadiazol-3-yl) benzyl)-3- hydroxy- pyrrolidine- 2-carboxamide | 384.477 | 6.20 min, 385 m/z | 0.61 μM (n = 3) |
| 90 | 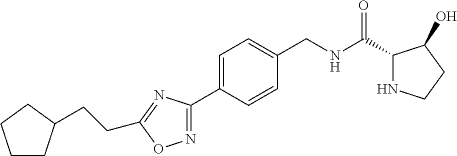 | (2S,3S)-N- (4-(5-(2- Cyclopentyl- ethyl)- 1,2,4- oxadiazol-3-yl) benzyl)-3- hydroxy- pyrrolidine- 2-carboxamide | 384.477 | 6.24 min, 385 m/z | 0.067 μM (n = 10) |
| 91 | 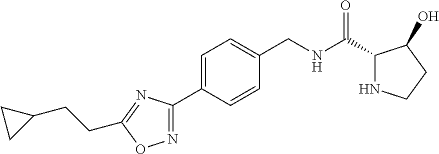 | (2S,3S)-N- (4-(5-(2- Cyclopropyl- ethyl)- 1,2,4- oxadiazol-3-yl) benzyl)-3- hydroxy- pyrrolidine- 2-carboxamide | 356.424 | 5.12 min, 357 m/z | 5.1 μM (n = 3) |
| 92 | 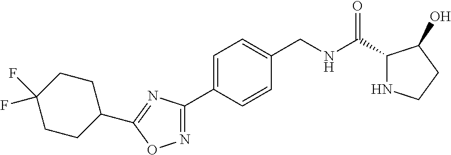 | (2S,3S)-N- (4-(5- (4,4-Difluoro- cyclohexyl)- 1,2,4- oxadiazol-3-yl) benzyl)-3- hydroxy- pyrrolidine-2- carboxamide | 406.431 | 5.15 min, 407 m/z | 6.4 μM (n = 5) |
| 93 | 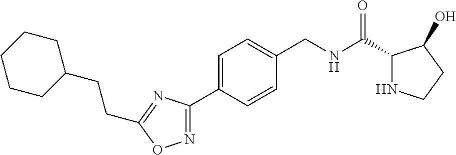 | (2S,3S)-N- (4-(5-(2- Cyclohexyl- ethyl)- 1,2,4- oxadiazol-3-yl) benzyl)-3- hydroxy- pyrrolidine- 2-carboxamide | 398.504 | 6.85 min, 399 m/z | 0.020 μM (n = 3) |
| 94 | 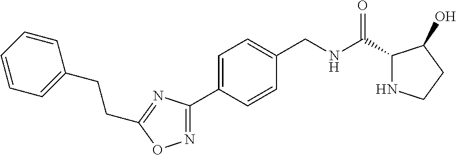 | (2S,3S)-3- Hydroxy- N-(4-(5- phenethyl- 1,2,4- oxadiazol-3- yl)benzyl) pyrrolidine-2- carboxamide | 392.457 | 5.45 min, 393 m/z | 0.73 μM (n = 2) |
| 95 | 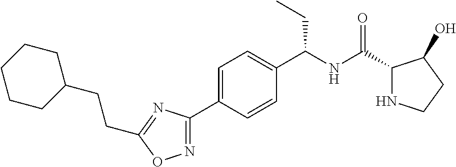 | (2S,3S)-N- ((S)-1-(4- (5-(2- Cyclohexyl- ethyl)- 1,2,4- oxadiazol-3-yl) phenyl) propyl)-3- hydroxy- pyrrolidine- 2-carboxamide | 426.558 | 7.52 min, 427 m/z | 0.010 μM (n = 6) |
| 96 | 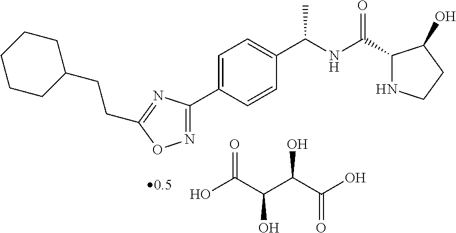 | (2S,3S)-N- ((S)-1-(4- (5-(2- Cyclohexyl- ethyl)- 1,2,4- oxadiazol-3-yl) phenyl) ethyl)-3- hydroxy- pyrrolidine- 2-carboxamide hemi-tartrate salt | 412.525 (free base) | 7.11 min, 413 m/z | 0.020 μM (n = 2) |
| 97 | 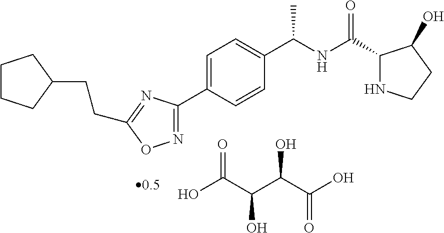 | (2S,3S)-N- ((S)-1-(4- (5-(2- Cyclopentyl- ethyl)- 1,2,4-oxadiazol- 3-yl) phenyl) ethyl)-3- hydroxy- pyrrolidine- 2-carboxamide hemi-tartrate salt | 398.499 (free base) | 6.69 min, 399 m/z | 0.058 μM (n = 2) |
| 98 | 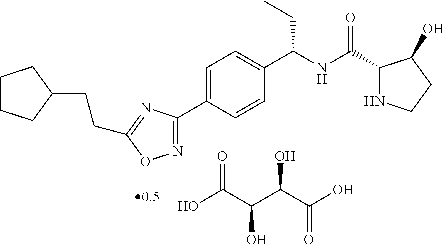 | (2S,3S)-N- ((S)-1-(4- (5-(2- Cyclopentyl- ethyl)- 1,2,4-oxadiazol- 3-yl) phenyl) propyl)-3- hydroxy- pyrrolidine- 2-carboxamide hemi-tartrate salt | 412.525 (free base) | 7.09 min, 413 m/z | 0.056 μM (n = 1) |
| 99 | 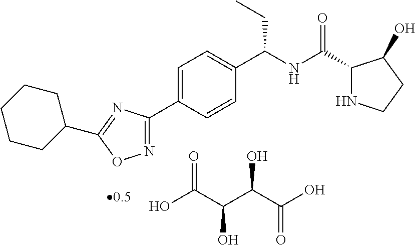 | (2S,3S)-N- ((S)-1-(4- (5-Cyclohexyl- 1,2,4-oxadiazol- 3-yl) phenyl) propyl)-3- hydroxy- pyrrolidine- 2-carboxamide hemi-tartrate salt | 398.499 (free base) | 1.01 min (UPLC), 399 m/z | 0.13 μM (n = 3) |
| 100 | 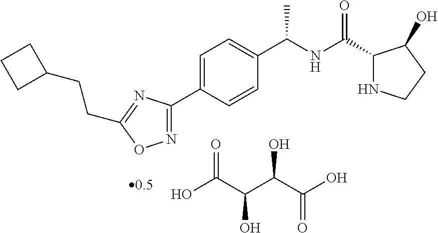 | (2S,3S)-N- ((S)-1-(4- (5-(2- Cyclobutyl- ethyl)- 1,2,4-oxadiazol- 3-yl) phenyl) ethyl)-3- hydroxy- pyrrolidine- 2-carboxamide hemi-tartrate salt | 384.472 (free base) | 1.08 min (UPLC), 385 m/z | 0.059 μM (n = 3) |
| 101 | 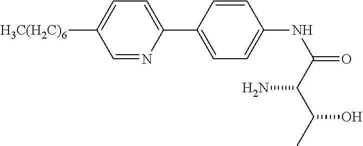 | (2S,3R)-2- amino-N- (4-(5-heptyl- pyridin-2- yl)phenyl)-3- hydroxy- butanamide | 369.5 | 7.67 min, 370 m/z | 0.9 μM (n = 2) |
| 102 | 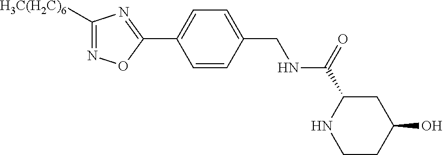 | (2S,4S)-N- (4-(3- heptyl-1,2,4- oxadiazol-5-yl) benzyl)-4- hydroxy- piperidine- 2-carboxamide | 400.51 | 6.82 min, 401 m/z | 2.7 μM (n = 1) |
| 103 | 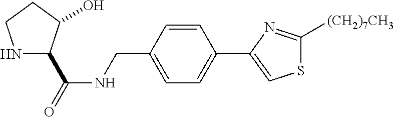 | (2S,3S)-3- hydroxy- N-(4-(2- octylthiazol- 4-yl)benzyl) pyrrolidine-2- carboxamide | 415.59 | 9.16 min, 416 m/z | 0.049 μM (n = 2) |
| 104 | 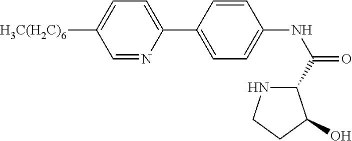 | (2S,3S)-N- (4-(5- heptylpyridin- 2-yl)phenyl)-3- hydroxy- pyrrolidine- 2-carboxamide | 381.51 | 9.01 min, 382 m/z | 1.6 μM (n = 2) |
| 105 | 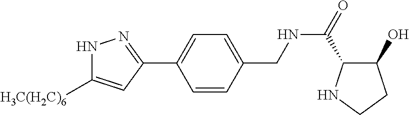 | (2S,3S)-N- (4-(5- heptyl-1H- pyrazol- 3-yl)benzyl)-3- hydroxy- pyrrolidine- 2-carboxamide | 384.52 | 7.36 min, 385 m/z | 1.1 μM (n = 2) |
| 106 | 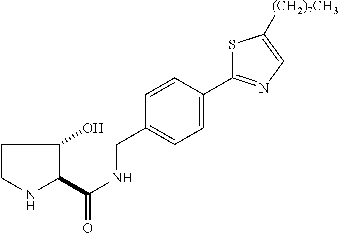 | (2S,3S)-3- hydroxy- N-(4-(5- octylthiazol- 2-yl)benzyl) pyrrolidine-2- carboxamide | 415.59 | 8.08 min, 416 m/z | 0.07 μM (n = 2) |
| 107 | 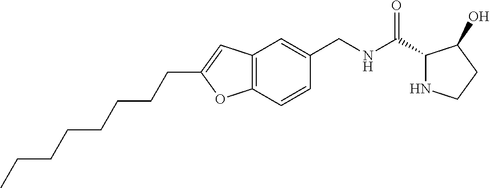 | (2S,3S)-3- Hydroxy- N-((2- octylbenzofuran- 5-yl)methyl) pyrrolidine-2- carboxamide | 372.506 | 8.31 min, 373 m/z | 0.031 μM (n = 2) |
| 108 | 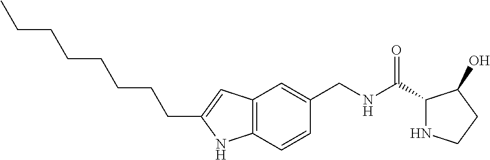 | (2S,3S)-3- Hydroxy- N-((2- octyl-1H-indol- 5-yl)methyl) pyrrolidine-2- carboxamide | 371.522 | 7.56 min, 372 m/z | 9.4 μM (n = 2) |
| 109 | 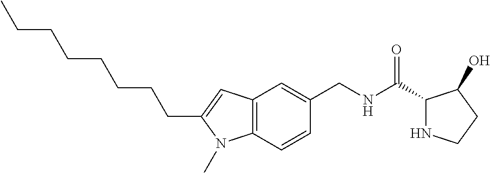 | (2S,3S)-3- Hydroxy- N-((1- methyl-2- octyl-1H- indol-5- yl)methyl) pyrrolidine-2- carboxamide | 385.549 | 8.06 min, 386 m/z | 0.30 μM (n = 2) |
| 110 | 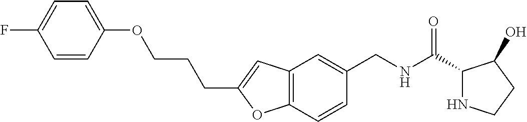 | (2S,3S)-N- ((2-(3-(4- Fluorophenoxy) propyl) benzofuran- 5-yl) methyl)-3- hydroxy- pyrrolidine- 2-carboxamide | 412.459 | 6.27 min, 413 m/z | 5.2 μM (n = 2) |
| 111 | 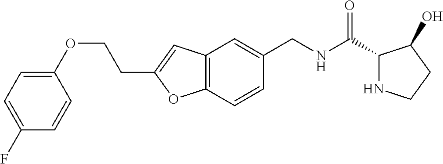 | (2S,3S)-N- ((2-(2-(4- Fluorophenoxy) ethyl) benzofuran- 5-yl)methyl)-3- hydroxy- pyrrolidine- 2-carboxamide | 398.432 | 6.88 min, 399 m/z | 5.0 μM (n = 2) |
| 112 | 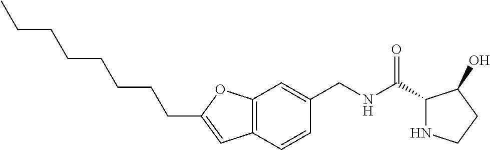 | (2S,3S)-3- Hydroxy- N-((2-octyl- benzofuran- 6-yl) methyl) pyrrolidine-2- carboxamide | 372.506 | 8.23 min, 373 m/z | 0.49 μM (n = 3) |
| 113 | 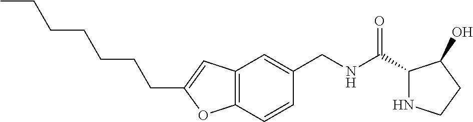 | (2S,3S)-N-((2- Heptyl- benzofuran- 5-yl)methyl)-3- hydroxy- pyrrolidine- 2-carboxamide | 358.479 | 7.67 min, 359 m/z | 0.16 μM (n = 7) |
| 114 | 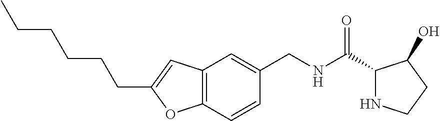 | (2S,3S)-N-((2- Hexyl- benzofuran- 5-yl) methyl)-3- hydroxy- pyrrolidine- 2-carboxamide | 344.452 | 7.07 min, 345 m/z | 0.53 μM (n = 6) |
| 115 | 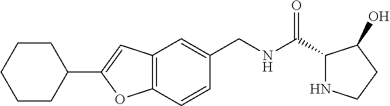 | (2S,3S)-N-((2- cyclohexyl- benzofuran- 5-yl)methyl)-3- hydroxy- pyrrolidine- 2-carboxamide | 342.436 | 6.51 min, 343 m/z | 1.2 μM (n = 3) |
| 116 | 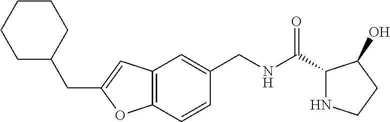 | (2S,3S)-N-((2- (Cyclohexyl- methyl) benzofuran- 5-yl) methyl)-3- hydroxy- pyrrolidine- 2-carboxamide | 356.459 | 7.05 min, 357 m/z | 0.11 μM (n = 3) |
| 117 | 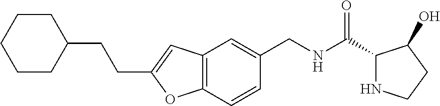 | (2S,3S)-N-((2- (2-Cyclohexyl- ethyl) benzofuran- 5-yl) methyl)-3- hydroxy- pyrrolidine- 2-carboxamide | 370.489 | 7.67 min, 371 m/z | 0.43 μM (n = 4) |
| 118 | 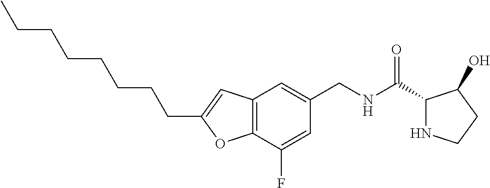 | (2S,3S)-N-((7- Fluoro-2- octylbenzo- furan-5- yl)methyl)-3- hydroxy- pyrrolidine- 2-carboxamide | 390.496 | 8.49 min, 391 m/z | 0.46 μM (n = 5) |
| 119 | 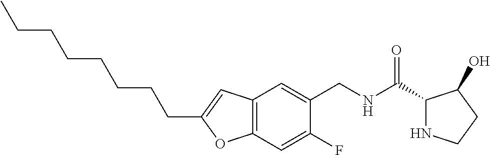 | (2S,3S)-N-((6- Fluoro-2- octylbenzo- furan-5- yl)methyl)-3- hydroxy- pyrrolidine- 2-carboxamide | 390.496 | 8.48 min, 391 m/z | 0.70 μM (n = 3) |
| 120 | 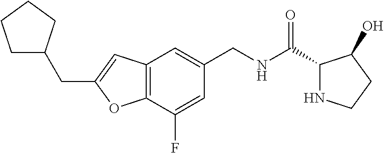 | (2S,3S)-N-((2- (Cyclopentyl- methyl)-7- fluorobenzo- furan-5- yl)methyl)-3- hydroxy- pyrrolidine- 2-carboxamide | 360.423 | 6.58 min, 361 m/z | 2.3 μM (n = 1) |
| 121 | 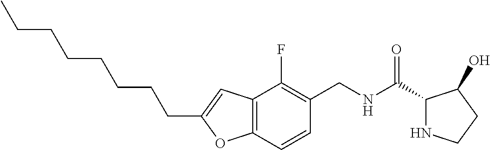 | (2S,3S)-N-((4- Fluoro-2- octylbenzo- furan-5- yl)methyl)-3- hydroxy- pyrrolidine- 2-carboxamide | 390.496 | 8.51 min, 391 m/z | 0.51 μM (n = 3) |
| 122 | 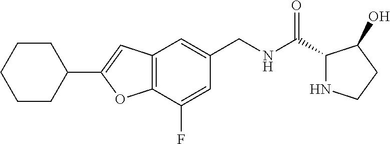 | (2S,3S)-N-((2- Cyclohexyl-7- fluorobenzo- furan-5- yl)methyl)-3- hydroxy- pyrrolidine- 2-carboxamide | 360.423 | 6.74 min, 361 m/z | 0.79 μM (n = 1) |
| 123 | 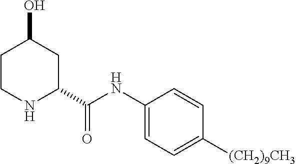 | (2R,4R)-N-(4- decylphenyl)- 4-hydroxy- piperidine- 2-carboxamide | 360.53 | 8.79 min, 361.16 m/z | 9.7 μM (n = 1) |
| 124 | 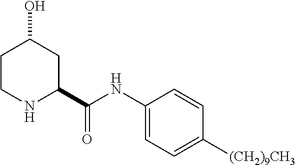 | (2S,4S)-N-(4- decylphenyl)- 4-hydroxy- piperidine- 2-carboxamide | 360.53 | 8.80 min, 361.16 m/z | 2.7 μM (n = 1) |

What is claimed is:

1. A compound of Formula I: 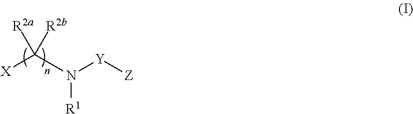
or a pharmaceutically acceptable salt, ester or prodrug the thereof, wherein
X is a (C₆-C₁₀)aryl or (C₂-C₉)heteroaryl group, wherein the (C₆-C₁₀)aryl or (C₂-C₉)heteroaryl groups are optionally independently substituted by one or more groups selected from:
(C₁-C₂₀)alkoxy, (C₁-C₂₀)alkoxy(C₁-C₂₀)alkyl,
(C₁-C₂₀)alkyl, (C₁-C₂₀)alkyl(C₃-C₁₀)cycloalkyl, (C₁-C₂₀)alkyl(C₆-C₁₀)aryl, (C₁-C₂₀)alkyl(C₆-C₁₀)aryl(C₂-C₉)heteroaryl, (C₁-C₂₀)alkyl(C₂-C₉)heteroaryl, (C₁-C₂₀)alkyloxo(C₁-C₂₀)alkyl, (C₁-C₂₀)alkylthio(C₁-C₂₀)alkyl, (C₁-C₂₀)alkyloxo(C₆-C₁₀)aryl, (C₁-C₂₀)alkylthio(C₆-C₁₀)aryl, (C₁-C₂₀)alkyl (C₂-C₉)heteroaryl(C₁-C₂₀)alkyl, (C₁-C₂₀)alkylsulfonyl(C₁-C₂₀)alkyl,
(C₃-C₁₀)cycloalkyl, (C₃-C₁₀)cycloalkyl(C₁-C₂₀)alkyl, (C₃-C₁₀)cycloalkyl(C₁-C₂₀)alkyl(C₂-C₉)heteroaryl, (C₃-C₁₀)cycloalkyl(C₂-C₉)heteroaryl,
(C₆-C₁₀)aryl, (C₆-C₁₀)aryl(C₁-C₂₀)alkyl, (C₆-C₁₀)aryl(C₁-C₂₀)alkyl(C₂-C₉)heteroaryl, (C₆-C₁₀)aryl(C₃-C₁₀)cycloalkyl, (C₆-C₁₀)aryloxy(C₁-C₂₀)alkyl (C₆-C₁₀)aryl(C₆-C₁₀)aryl, (C₆-C₁₀)aryl(C₁-C₂₀)alkyl(C₆-C₁₀)aryl, (C₆-C₁₀)aryl(C₃-C₁₀)cycloalkyl(C₆-C₁₀)aryl, (C₆-C₁₀)aryl(C₁-C₂₀)alkyl(C₃-C₁₀)cycloalkyl,
(C₂-C₉)heteroaryl, (C₂-C₉)heteroaryl(C₁-C₂₀)alkyl, (C₂-C₉)heteroaryl(C₃-C₁₀)cycloalkyl, (C₂-C₉)heteroaryl(C₆-C₁₀)aryl, (C₂-C₉)heteroaryl(C₆-C₁₀)aryl(C₁-C₂₀)alkyl, (C₂-C₉)heteroaryl(C₁-C₂₀)alkyl(C₆-C₁₀)aryl, (C₂-C₉)heteroaryl(C₃-C₁₀)cycloalkyl(C₆-C₁₀)aryl, (C₂-C₉)heteroaryl(C₁-C₂₀)alkyl(C₃-C₁₀)cycloalkyl,
hydroxyl,
halogen,
oxo(C₁-C₂₀)alkyl, and
thio(C₁-C₂₀)alkyl;
wherein the (C₁-C₂₀)alkyl, (C₃-C₁₀)cycloalkyl, (C₁-C₂₀)alkoxy, (C₆-C₁₀)aryl, (C₂-C₉)heteroaryl, oxo(C₁-C₂₀)alkyl, and thio(C₁-C₂₀)alkyl groups are optionally independently substituted by one or more groups selected from: (C₁-C₂₀)alkyl, amino, hydroxyl, carbonyl, and halogen;
n is 0, 1, 2, or 3;
Y is carbonyl or —CH₂—;
Z is (C₁-C₂₀)alkyl, (C₃-C₁₀)cycloalkyl, or (C₂-C₉)heterocycloalkyl, wherein the (C₁-C₂₀)alkyl or (C₂-C₉)heterocycloalkyl groups are optionally independently substituted by one or more groups selected from: amino, hydroxyl, carbonyl, —N[(C₁-C₂₀)alkyl]₂, —NH[(C₁-C₂₀)alkyl], —N[(C₃-C₁₀)cycloalkyl]₂, —NH[(C₃-C₁₀)cycloalkyl, —N[(C₃-C₁₀)cycloalkyl(C₁-C₂₀)alkyl]₂, —NH[(C₃-C₁₀)cycloalkyl(C₁-C₂₀)alkyl], and halogen;
R¹ is hydrogen, (C₁-C₁₂)alkyl, (C₃-C₁₀)cycloalkyl, or (C₂-C₉)heterocycloalkyl, and
R^{2a}, R^{2b}
are each independently hydrogen, halogen, hydroxyl, (C₁-C₁₂)alkyl, (C₃-C₁₀)cycloalkyl, or (C₂0C₉)heterocycloalkyl wherein each R^{2a} and R^{2b} is not halogen or hydroxyl at the position α to the nitrogen atom in Formula I, and wherein optionally R^{2a} and R^{2b} may together form a 3- to 7-membered ring, and
wherein optionally R¹ and one of R^{2a} and R^{2b} may together form a 3- to 7-membered ring.

2. The compound of claim 1, wherein Y is carbonyl.

3. The compound of claim 2, wherein n is 0.

4. The compound of claim 2, wherein n is 1.

5. The compound of claim 2, wherein n is 2.

6. The compound of claim 2, wherein R¹ is hydrogen, (C₁-C₆)alkyl or (C₃-C₆)cycloalkyl.

7. The compound of claim 6, wherein R¹ is hydrogen or (C₁-C₃)alkyl.

8. The compound of claim 7, wherein R¹ is hydrogen.

9. The compound of claim 2, wherein X is (C₆)aryl, (C₄-C₅)heteroaryl, or (C₇-C₈)heteroaryl.

10. The compound of claim 9, wherein X is (C₆)aryl and n is 0 or 1.

11. The compound of claim 9, wherein X is (C₄-C₅)heteroaryl and n is 0 or 1.

12. The compound of claim 9, wherein X is (C₇-C₈)heteroaryl and n is 0 or 1.

13. The compound of claim 11, wherein X is: 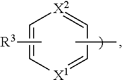
wherein
X¹, X²
are each independently N or —CH—, wherein at least one of X¹ and X² is nitrogen;
R³ is one or more groups selected from:
(C₁-C₂₀)alkoxy, (C₁-C₂₀)alkoxyalkyl,
(C₁-C₂₀)alkyl, (C₁-C₂₀)alkylcycloalkyl, (C₁-C₂₀)alkylaryl, (C₁-C₂₀)alkylarylheteroaryl, (C₁-C₂₀)alkylheteroaryl, (C₁-C₂₀)alkyloxoalkyl, (C₁-C₂₀)alkylthioalkyl, (C₁-C₂₀)alkyloxoaryl, (C₁-C₂₀)alkylthioaryl, (C₁-C₂₀)alkylheteroarylalkyl,
(C₃-C₁₀)cycloalkyl, (C₃-C₁₀)cycloalkylalkyl, (C₃-C₁₀)cycloalkylalkylheteroaryl, (C₃-C₁₀)cycloalkylheteroaryl,
(C₆-C₁₀)aryl, (C₆-C₁₀)arylalkyl, (C₆-C₁₀)arylcycloalkyl, (C₆-C₁₀)arylaryl, (C₆-C₁₀)arylalkylaryl, (C₆-C₁₀)arylcycloalkylaryl, (C₆-C₁₀)arylalkylcycloalkyl,
(C₂-C₉)heteroaryl, (C₂-C₉)heteroarylalkyl, (C₂-C₉)heteroarylcycloalkyl, (C₂-C₉)heteroarylaryl, (C₂-C₉)heteroarylarylalkyl, (C₂-C₉)heteroarylalkylaryl, (C₂-C₉)heteroarylcycloalkylaryl, (C₂-C₉)heteroarylalkylcycloalkyl,
hydroxyl,
halogen,
oxoalkyl, and
thioalkyl;
wherein the alkyl, cycloalkyl, alkoxy, aryl, heteroaryl, oxoalkyl, and thioalkyl groups are optionally independently substituted by one or more groups selected from: amino, hydroxyl, carbonyl, and halogen.

14. The compound of claim 12, wherein X is: 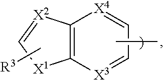
wherein
X¹ is N or O;
X², X³, X⁴
are each N or —CH—, wherein at least one of X², X³, and X⁴ is nitrogen;
R³ is one or more groups selected from:
(C₁-C₂₀)alkoxy, (C₁-C₂₀)alkoxyalkyl,
(C₁-C₂₀)alkyl, (C₁-C₂₀)alkylcycloalkyl, (C₁-C₂₀)alkylaryl, (C₁-C₂₀) alkylarylheteroaryl, (C₁-C₂₀)alkylheteroaryl, (C₁-C₂₀)akyloxoalkyl, (C₁-C₂₀)alkylthioalkyl, (C₁-C₂₀)alkyloxoaryl, (C₁-C₂₀)alkylthioaryl, (C₁-C₂₀)alkylheteroarylalkyl, (C₁-C₂₀)alkylsulfonyl(C₁-C₂₀)alkyl,
(C₃-C₁₀)cycloalkyl, (C₃-C₁₀)cycloalkylalkyl, (C₃-C₁₀)cycloalkylalkylheteroaryl, (C₃-C₁₀)cycloalkylheteroaryl,
(C₆-C₁₀)aryl, (C₆-C₁₀)arylalkyl, (C₆-C₁₀)arylcycloalkyl, (C₆-C₁₀)arylaryl, (C₆-C₁₀)arylalkylaryl, (C₆-C₁₀)arylcycloalkylaryl, (C₆-C₁₀)arylalkylcycloalkyl,
(C₂-C₉)heteroaryl, (C₂-C₉)heteroarylalkyl, (C₂-C₉)heteroarylcycloalkyl, (C₂-C₉)heteroarylaryl, (C₂-C₉)heteroarylarylalkyl, (C₂-C₉)heteroarylalkylaryl, (C₂-C₉)heteroarylcycloalkylaryl, (C₂-C₉)heteroarylalkylcycloalkyl,
hydroxyl,
halogen,
oxoalkyl, and
thioalkyl,
wherein the alkyl, cycloalkyl, alkoxy, aryl, heteroaryl, oxoalkyl, and thioalkyl groups are optionally independently substituted by one or more groups selected from: amino, hydroxyl, carbonyl, and halogen.

15. The compound of claim 2, wherien X is substituted with a halogen.

16. The compound of claim 15, wherein the halogen if F or Cl.

17. The compound of claim 2, wherein X is substituted with
R⁵-A-R⁴—),
wherein
A is (C₆)aryl or (C₂-C₅)heteroaryl;
R⁴ is a single bond or (C₁-C₄)alkylene or (C₁-C₃)heteroalkylene; and
R⁵ is a group selected from:
(C₂-C₂₀)alkyl, (C₂-C₂₀)alkylcycloalkyl, (C₂-C₂₀)alkylaryl,
(C₁-C₁₉)heteroalkyl, and (C₁-C₁₉)heteroalkylcycloalkyl.

18. The compound of claim 17, wherein R⁴ is a single bond.

19. The compound of claim 17, wherein R⁵ is (C₂-C₁₂)alkyl or (C₁-C₁₁)heteroalkyl.

20. The compound of claim 17, wherein A is a (C₂-C₃)heteroaryl.

21. The compound of claim 20, wherein A is selected from: 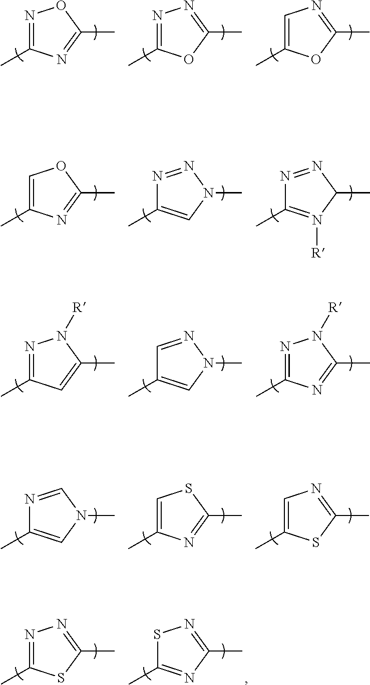
and all regioisomers thereof,
wherein
R′ is a group selected from the group of:
(C₂-C₂₀)alkyl, (C₂-C₂₀)alkylcycloalkyl, (C₂-C₂₀)alkylaryl,
(C₁-C₁₉)heteroalkyl, and (C₁-C₁₉)heteroalkylcycloalkyl.

22. The compound of claim 2, wherien Z is (C₁-C₁₂)alkyl, optionally substituted by one or more groups selected from: amino, hydroxyl, carbonyl, and halogen.

23. The compound of claim 2, wherein Z is (C₂-C₉)heterocycloalkyl, optionally substituted by one or more groups selected from: amino, hydroxyl, carbonyl, and halogen.

24. The compound of claim 2, wherien Z is: 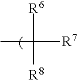
wherein,
R⁶, R⁷
are each independently a hydrogen, halogen, hydroxyl,
(C₁-C₁₀)alkyl, (C₁-C₁₀)heteroalkyl, or
(C₃-C₈)cycloalkyl or (C₂-C₇)heterocycloalkyl;
R⁸ is —B—(R^{8a})ₘ, wherein
B is N, O, or S;
m is 1 when B is O or S, or 2 when B is N;
each R^{8a} is independently a hydrogen, (C₁-C₁₀)alkyl; (C₁-C₁₀)heteroalkyl, or (C₃-C₈)cycloalkyl or (C₂-C₇)heterocycloalkyl;
wherein, when B is N, a R^{8a} may together form a ring with the other R^{8a}, R⁶, or R⁷; and
when B is O or S, R^{8a} may together form a ring with R⁶ or R⁷.

25. The compound of claim 24, wherein B is N.

26. The compound of claim 25, wherein a R^{8a} together with R⁷ form a ring Q, 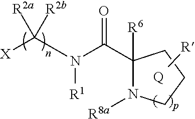
wherein
R′ is absent, one or more amino, halogen, hydroxyl, carbonyl, and (C₁-C₁₀)alkyl; (C₁-C₁₀)heteroalkyl, or (C₃-C₈)cycloalkyl or (C₂-C₇)heterocycloalkyl; and
P is 0, 1, 2, or 3.

27. The compound of claim 26, wherein n is 1 or 2, and the R^{2b} at α-position to the nitrogen atom in the formula is hydrogen:

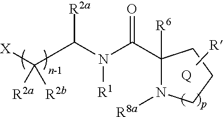

28. The compound of claim 27, wherein X is (C₆)aryl, (C₄-C₅)heteroaryl, or (C₇-C₈)heteroaryl.

29. The compound of claim 27, wherein X is substituted with
R⁵-A-R⁴—),
wherein
A is (C₆)aryl or (C₂-C₅)heteroaryl;
R⁴ is a single bond or (C₁-C₄)alkylene or (C₁-C₃)heteroalkylene; and
R⁵ is a group selected from:
(C₂-C₂₀)alkyl, (C₂-C₂₀)alkylcycloalkyl, (C₂-C₂₀)alkylaryl,
(C₁-C₁₉)heteroalkyl, and (C₁-C₁₉)heteroalkylcycloalkyl.

30. The compound of claim 29, wherein
R⁴ is a single bond; and
A is (C₂-C₃)heteroaryl.

31. The compound of claim 30, wherein A is selected from: 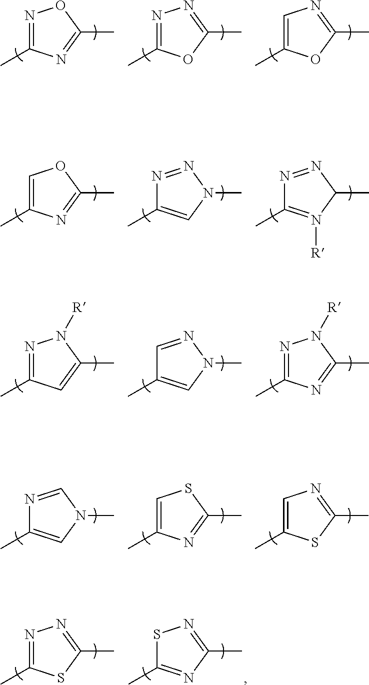
and all regioisomers thereof,
wherein
R′ is a group selected from the group of:
(C₂-C₂₀)alkyl, (C₂-C₂₀)alkylcycloalkyl, (C₂-C₂₀)alkylaryl,
(C₁-C₁₉)heteroalkyl, and (C₁-C₁₉)heteroalkylcycloalkyl.

32. The compound of claim 29, wherein X is (C₆)aryl.

33. The compound of claim 25, wherein X is substituted with
R⁵-A-R⁴—),
wherein
A is (C₆)aryl or (C₂-C₅)heteroaryl;
R⁴ is a single bond or (C₁-C₄)alkylene or (C₁-C₃)heteroalkylene; and
R⁵ is a group selected from:
(C₂-C₂₀)alkyl, (C₂-C₂₀)alkylcycloalkyl, (C₂-C₂₀)alkylaryl,
(C₁-C₁₉)heteroalkyl, and (C₁-C₁₉)heteroalkylcycloalkyl.

34. The compound of claim 33, wherein
R⁴ is a single bond; and
A is (C₃-C₃)heteroaryl.

35. The compound of claim 34, wherein A is selected from: 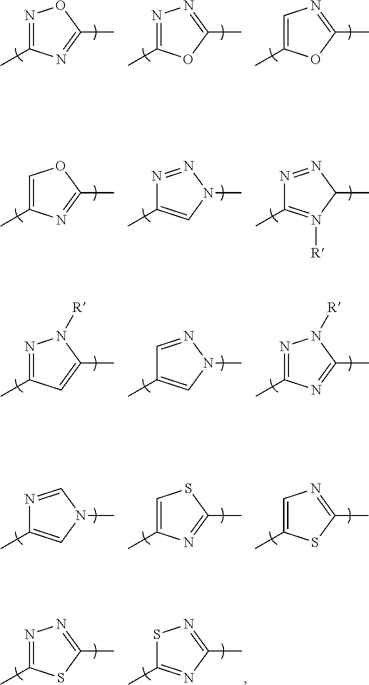
and all regioisomers thereof,
wherein
R′ is a group selected from the group of:
(C₂-C₂₀)alkyl, (C₂-C₂₀)alkylcycloalkyl, (C₂-C₂₀)alkylaryl,
(C₁-C₁₉)heteroalkyl, and (C₁-C₁₉)heteroalkylcycloalkyl.

36. The compound of claim 33, wherein X is (C₆)aryl.

37. The compound of claim 33, wherien X is (C₄-C₅)heteroaryl.

38. The compound of claim 33, wherien X is (C₇-C₈)heteroaryl.

39. The compound of claim 36, wherein R⁶ is 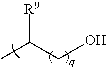
wherein
q is 0, 1, 2, 3, or 4;
R⁹ is hydrogen, one or more halogen, hydroxy, amino, and (C₁-C₁₂)alkyl, (C₁-C₁₂)heteroalkyl, (C₃-C₈)cycloalkyl or (C₂-C₇)heterocycloalkyl.

40. The compound of claim 2, wherein X is aryl.

41. The compound of claim 2, wherein X is heteroaryl.

42. A pharmaceutical composition comprising an amount of a compound of Formula I 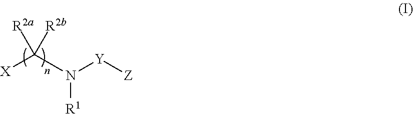
or a pharmaceutically acceptable salt, ester or pro-drug thereof, effective in the treatment or prevention of disorder or condition selected from the group consisting of inflammation and immune-mediated disease, cancer, diabetes, inflammatory bowel disease, fibrosis, polycystic kidney disease, arteriosclerosis, pulmonary diseases, and viral infections, or a related disorder or condition thereof in a mammal, including a human, and a pharmaceutically effective carrier, wherein
X is a (C₆-C₁₀)aryl or (C₂-C₉)heteroaryl group, wherein the aryl or heteroaryl groups are optionally independently substituted by one or more groups selected from:
(C₁-C_{20l )alkoxy, (C}₁-C₂₀)alkoxyalkyl,
(C₁-C₂₀)alkyl, (C₁-C₂₀)alkylcycloalkyl, (C₁-C₂₀)alkylaryl, (C₁-C₂₀)alkylarylheteroaryl, (C₁-C₂₀)alkylheteroaryl, (C₁-C₂₀)alkyloxoalkyl, (C₁-C₂₀)alkylthioalkyl, (C₁-C₂₀)alkyloxoaryl, (C₁-C₂₀)alkylthioaryl, (C₁-C₂₀)alkylheteroarylalkyl, (C₁-C₂₀)alkylsulfonyl(C₁-C₂₀)alkyl,
(C₃-C₁₀)cycloalkyl, (C₃-C₁₀)cycloalkylalkyl, (C₃-C₁₀)cycloalkylkylheteroaryl, (C₃-C₁₀)cycloalkylheteroaryl,
(C₆-C₁₀)aryl, (C₆-C₁₀)arylalkyl, (C₆-C₁₀)arylcycloalkyl, (C₆-C₁₀)arylaryl, (C₆-C₁₀)arylalkylaryl, (C₆-C₁₀)arylcycloalkylaryl, (C₆-C₁₀)arylalkylcycloalkyl,
(C₂-C₉)heteroaryl, (C₂-C₉)heteroarylalkyl, (C₂-C₉)heteroarylcycloalkyl, (C₂-C₉)heteroarylaryl, (C₂-C₉)heteroarylarylalkyl, (C₂-C₉)heteroarylalkylaryl, (C₂-C₉)heteroarylcycloalkylaryl, (C₂-C₉)heteroarylalkylcycloalkyl,
hydroxyl,
halogen,
oxoalkyl, and
thioalkyl;
wherein the alkyl, cycloalkyl, alkoxy, aryl, heteroaryl, oxoalkyl, and thioalkyl groups are optionally independently substituted by one or more groups selected from: amino, hydroxyl, carbonyl, and halogen;
n is 0, 1, 2, or 3;
Y is carbonyl or —CH₂—;
Z is (C₁-C₂₀)alkyl, (C₃-C₁₀)cycloalkyl, or (C₂-C₉)heterocycloalkyl, wherien the (C₁-C₂₀)alkyl or (C₂-C₉)heterocycloalkyl groups are optionally independently substituted by one or more groups selected from: amino, hydroxyl, carbonyl, —N[(C₁-C₂₀)alkyl]₂, —NH[(C₁-C₂₀)alkyl], —N[(C₃-C₁₀)cycloalkyl]₂, —NH[(C₃-C₁₀)cycloalkyl], —N[(C₃-C₁₀)cycloalkyl(C₁-C₂₀)alkyl]₂, —NH[(C₃-C₁₀)cycloalkyl(C₁-C₂₀)alkyl], and halogen;
R¹ is hydrogen, (C₁-C₁₂)alkyl, (C₃-C₁₀)cycloalkyl, or (C₂-C₉)heterocycloalkyl; and
R^{2a}, R^{2b}
each independently is hydrogen, halogen, hydroxyl, (C₁-C₁₂)alkyl, (C₃-C₁₀)cycloalkyl, or (C₂-C₉)heterocycloalkyl wherein each R^{2a} and R^{2b} is not halogen or hydroxyl at the position α to the nitrogen atom in Formula I, and wherein optionally R^{2a} and R^{2b} may together form a 3- to 7-membered ring, and
wherein optionally R¹ and one of R^{2a} and R^{2b} may together form a 3- to 7-membered ring.

43. The pharmaceutical composition of claim 42, wherein the disorder or condition is an immune-mediated disease.

44. The pharmaceutical composition of claim 42, wherein the disorder or condition is cancer.

45. The pharmaceutical composition of claim 42, wherein the disorder or condition is an type of diabetes.

46. The pharmaceutical composition of any of claim 42, wherein the mammal is a human.

47. A method of treating or preventing a disorder or condition in a mammal, including a human, comprising administering to a subject in need thereof an therapeutically effective amount of a pharmaceutical composition comprising a compound of Formula I 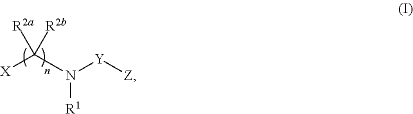
or a pharmaceutically acceptable salt, ester or pro-drug thereof,
wherein the disorder or condition is selected from the group consisting of inflammation and immune-mediated disease, cancer, diabetes, inflammatory bowel disease, fibrosis, polycystic kidney disease, arteriosclerosis, pulmonary diseases, and viral infections or a related disorder or condition thereof,
wherein
X is a (C₆-C₁₀)aryl or (C₂-C₉)heteroaryl group, wherein the aryl or heteroaryl groups are optionally independently substituted by one or more groups selected from
(C₁-C₂₀)alkoxy, (C₁-C₂₀)alkoxyalkyl,
(C₁-C₂₀)alkyl, (C₁-C₂₀)alkylcycloalkyl, (C₁-C₂₀)alkylaryl, (C₁-C₂₀)alkylarylheteroaryl, (C₁-C₂₀)alkylheteroaryl, (C₁-C₂₀)alkyloxoalkyl, (C₁-C₂₀)alkylthioalkyl, (C₁-C₂₀)alkyloxoaryl, (C₁-C₂₀)alkylthioaryl, (C₁-C₂₀)alkylheteroarylalkyl, (C₁-C₂₀)alkylsulfonyl(C₁-C₂₀)alkyl,
(C₃-C₁₀)cycloalkyl, (C₃-C₁₀)cycloalkylalkyl, (C₃-C₁₀)cycloalkylalkylheteroaryl, (C₃-C₁₀)cycloalkylheteroaryl,
(C₆-C₁₀)aryl, (C₆-C₁₀)arylalkyl, (C₆-C₁₀)arylcycloalkyl, (C₆-C₁₀)arylaryl, (C₆-C₁₀)arylalkylaryl, (C₆-C₁₀)arylcycloalkylaryl, (C₆-C₁₀)arylkylcycloalkyl,
(C₂-C₉)heteroaryl, (C₂-C₉)heteroarylalkyl, (C₂-C₉)heteroarylcycloalkyl, (C₂-C₉)heteroarylaryl, (C₂-C₉)heteroarylarylalkyl, (C₂-C₉)heteroarylalkylaryl, (C₂-C₉)heteroarylcycloalkylaryl, (C₂-C₉)heteroarylalkylcycloalkyl,
hydroxyl,
halogen,
oxoalkyl, and
thioalkyl;
wherein the alkyl, cycloalkyl, alkoxy, aryl, heteroaryl, oxoalkyl, and thioalkyl groups are optionally independently substituted by one or more groups selected from: amino, hydroxyl, carbonyl, and halogen;
n is 1, 2, or 3;
Y is carbonyl or —CH₂—;
Z is (C₁-C₂₀)alkyl, (C₃-C₁₀)cycloalkyl, or (C₂-C₉)heterocycloalkyl, wherein the (C₁-C₂₀)alkyl or (C₂-C₉)heterocycloalkyl groups are optionally independently substituted by one or more groups selected from: amino, hydroxyl, carbonyl, —N[(C₁-C₂₀)alkyl]₂, —NH[(C₁-C₂₀)alkyl], —N[(C₃-C₁₀)cycloalkyl]₂, —NH[(C₃-C₁₀)cycloalkyl], —N[(C₃-C₁₀)cycloalkyl(C₁-C₂₀)alkyl]₂, —NH[(C₃-C₁₀)cycloalkyl(C₁-C₂₀)alkyl], and halogen;
R¹ is hydrogen, (C₁-C₁₂)alkyl, (C₃-C₁₀)cycloalkyl, or (C₂-C₉)heterocycloalkyl; and
R^{2a}, R^{2b}
each independently is hydrogen, halogen, hydroxyl, (C₁-C₁₂)alkyl, (C₃-C₁₀)cycloalkyl, or (C₂-C₉)heterocycloalkyl wherien each R^{2a} and R^{2b} is not halogen or hydroxyl at the position α to the nitrogen atom in Formula I, and wherein optionally R^{2a} and R^{2b} may together form a 3- to 7-membered ring, and
wherein optionally R¹ and one of R^{2a} and R^{2b} may together form a 3- to 7-membered ring.

48. The method of claim 47, wherein the disorder or condition is an immune-mediated disease.

49. The method of claim 47, wherein the disorder or condition is cancer.

50. The method of claim 47, wherein the disorder or condition is an type of diabetes.

51. The method of claim 47, wherein the disorder or conditions is a HIV or HCV viral infection.

52. The method of claim 47, wherein the mammal is a human.

53. A method for treating a disorder or condition mediated by sphingosine kinase-1, the method comprising administering to a subject in need thereof a therapeutically effective amount of a pharmaceutical composition comprising a compound of Formula I 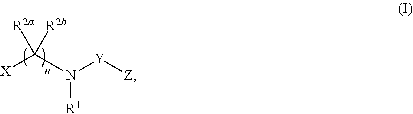
or a pharmaceutically acceptable salt, ester or pro-drug thereof,
wherein
wherein
X is a (C₆-C₁₀)aryl or (C₂-C₉)heteroaryl group, wherein the aryl or heteroaryl groups are optionally independently substituted by one or more groups selected from:
(C₁-C₂₀)alkoxy, (C₁-C₂₀)alkoxyalkyl,
(C₁-C₂₀)alkyl, (C₁-C₂₀)alkylcycloalkyl, (C₁-C₂₀)alkylaryl, (C₁-C₂₀)alkylarylheteroaryl, (C₁-C₂₀)alkylheteroaryl, (C₁-C₂₀)alkyloxoalkyl, (C₁-C₂₀)alkylthioalkyl, (C₁-C₂₀)alkyloxoaryl, (C₁-C₂₀)alkylthioaryl, (C₁-C₂₀)alkylheteroarylalkyl, (C₁-C₂₀)alkylsulfonyl(C₁-C₂₀)alkyl,
(C₃-C₁₀)cycloalkyl, (C₅-C₁₀)cycloalkylalkyl, (C₃-C₁₀)cycloalkylkylheteroaryl, (C₃-C₁₀)cycloalkylheteroaryl,
(C₆-C₁₀)aryl, (C₆-C₁₀)arylalkyl, (C₆-C₁₀)arylcycloalkyl, (C₆-C₁₀)arylaryl, (C₆-C₁₀)arylalkylaryl, (C₆-C₁₀)arylcycloalkylaryl, (C₆-C₁₀)arylalkylcycloalkyl,
(C₂-C₉)heteroaryoaryl, (C₂-C₉)heteroarylalkyl, (C₂-C₉)heteroarylcycloalkyl, (C₂-C₉)heteroarylaryl, (C₂-C₉)heteroarylarylalkyl, (C₂-C₉)heteroarylalkylaryl, (C₂-C₉heteroarylcycloalkylaryl, (C₂-C₉)heteroarylalkylcycloalkyl,
hydroxyl,
halogen,
oxoalkyl, and
thioalkyl;
wherein the alkyl, cycloalkyl, alkoxy, aryl, heteroaryl, oxoalkyl, and thioalkyl groups are optionally independently substituted by one or more groups selected from: amino, hydroxyl, carbonyl, and halogen;
n is 0, 1, 2, or 3;
Y is carbonyl or —-CH₂—;
Z is (C₁-C₂₀)alkyl, (C₃-C₁₀)cycloalkyl, (C₂-C₉)heterocycloalkyl, wherein the (C₁-C₂₀)alkyl or (C₂-C₉ )heterocycloalkyl groups are optionally independently substituted by one or more groups selected from: amino, hydroxyl, carbonyl, —N[(C₁-C₂₀)alkyl]₂, —NH[(C₁-C₂₀)alkyl], —N[(C₃-C₁₀)cycloalkyl]₂, —NH[(C₃-C₁₀)cycloalkyl], —N[(C₃-C₁₀)cycloalkyl(C₁-C₂₀)alkyl]₂, —NH[(C₃-C₁₀)cycloalkyl(C₁-C₂₀)alkyl], and halogen;
R¹ is hydrogen, (C₁-C₁₂)alkyl, (C₃-C₁₀)cycloalkyl, or (C₂-C₉)heterocycloalkyl; and
R^{2a}, R^{2b}
each independently is hydrogen, halogen, hydroxyl, (C₁-C₁₂)alkyl, (C₃-C₁₀)cycloalkyl, or (C₂-C₉)heterocycloalkyl wherein each R^{2a} and R^{2b} is not halogen or hydroxyl at the position α to the nitrogen atom in Formula I, and wherein optionally R^{2a} and R^{2b} may together form a 3- to 7-membered ring, and
wherein optionally R¹ and one of R^{2a} and R^{2b} may together form a 3- or 7-membered ring.

54. A compound selected from:
(2S,3S)-3-Hydroxy-N-(4-octylphenyl)pyrrolidine-2-carboxamide
(2S,3R)-3-Hydroxy-N-(4-octylphenyl)pyrrolidine-2-carboxamide
(R)-2-Amino-3-hydroxy-N-(4-octylphenyl)propanamide
(S)-2-Amino-3-hydroxy-N-(4-octylphenyl)propanamide
(±)-erythro-DL-2-Amino-3-hydroxy-N-(4-octylphenyl)pentanamide
(2S,3R)-2-Amino-N-(4-decylphenyl)-3-hydroxybutanamide
(2S,3R)-2-Amino-3-hydroxy-N-(4-undecylphenyl)butanamide
(2S,3R)-2-Amino-N-(4-dodecylphenyl)-3-hydroxybutanamide
(2R,3S)-2-((4-Octylphenylamino)methyl)pyrrolidin-3-ol
(R)-2-Amino-3-(4-octylphenylamino)propan-1-ol
(2S,3R)-2-Amino-3-hydroxy-N-(3-octylphenyl)butanamide
(2S,3R)-2-Amino-N-(4-(heptyloxy)phenyl)-3-hydroxybutanamide
(+/−)-2-Amino-3-hydroxy-N-(4-nonylphenyl)propanamide
(2S,3R)-2-Amino-3-hydroxy-N-(4-nonylphenyl)butanamide
(2S,3R)-2-Amino-N-(4-(hexyloxymethyl)phenyl)-3-hydroxybutanamide
(2S,3R)-2-Amino-3-hydroxy-N-(4-(7-methyloctyl)phenyl)butanamide
(2S,3R)-2-Amino-N-(4-(7-(4-fluorophenoxy)heptyl)phenyl)-3-hydroxybutanamide
(2S,3R)-2-Amino-3-hydroxy-N-(4-(octyloxy)phenyl)butanamide
(2S,3S)-N-(4-(Hexyloxymethyl)phenyl)-3-hydroxyprrolidine-2-carboxamide
(2S,3R,4S)-3,4-Dihydroxy-N-(4-octylphenyl)pyrrolidine-2-carboxamide
(2S,3S)-3-Hydroxy-N-(4-(octyloxymethyl)phenyl)pyrrolidine-2-carboxamide
(2S,3R)-2-Amino-N-(4-(5-(4-fluorophenoxy)pentyl)phenyl)-3-hydroxybutanamide
(2S,3S)-N-(4-(3-(4-Fluorophenoxy)propyl)phenyl)-3-hydroxypyrrolidine-2-carboxamide
(2S,3S)-N-(4-(5-(4-Fluorophenoxy)pentyl)phenyl)-3-hydroxypyrrolidine-2-carboxamide
(2S,3S)-N-(4-(7-(4-Fluorophenoxy)heptyl)phenyl)-3-hydroxypyrrolidine-2-carboxamide
(2S,3S)-3-Hydroxy-N-(6-octylpyridin-3-yl)pyrrolidine-2-carboxamide
(2S,3S)-3-Hydroxy-N-((S)-1-(4-octylphenyl)ethyl)pyrrolidine-2-carboxamide
(2S,3S)-N-(4-(5-(4-Fluorophenoxy)pentyl)benzyl)-3-hydroxypyrrolidine-2-carboxamide
(2S,3R)-2-Amino-3-hydroxy-N-(4-octylphenyl)butanamide
(2R,3S)-2-Amino-3-hydroxy-N-(4-octylphenyl)butanamide
(2R,3R)-2-Amino-3-hydroxy-N-(4-octylphenyl)butanamide
(2S,3S)-2-Amino-3-hydroxy-N-(4-octylphenyl)butanamide
(2S,3R)-2-amino-3-hydroxy-N-(4-(6-oxodecyl)phenyl)butanamide
(2S,3R)-2-amino-3-hydroxy-N-(4-(4-oxodecyl)phenyl)butanamide
(2S,3R)-2-amino-3-hydroxy-N-(4-(4-hydroxydecyl)phenyl)butanamide
(2S,3S)-3-hydroxy-N-(4-octylbenzyl)pyrrolidine-2-carboxamide
(2S,3S)-N-(4-heptylbenzyl)-3-hydroxypyrrolidine-2-carboxamide
(2S,3S)-3-hydroxy-N-(4-(6-oxodecyl)phenyl)pyrrolidine-2-carboxamide
(2S,3S)-N-(4-(hexylthiomethyl)phenyl)-3-hydroxypyrrolidine-2-carboxamide
(2S,3S)-N-(4-(2-(hexylthio)ethyl)phenyl)-3-hydroxypyrrolidine-2-carboxamide
(2S,3S)-N-(4-(4-(hexylthio)butyl)phenyl)-3-hydroxypyrrolidine-2-carboxamide
(2S,3S)-3-hydroxy-N-(4-(4-hydroxyundecyl)phenyl)pyrrolidine-2-carboxamide
(2S,3S)-N-(4-heptylthiomethyl)benzyl)-3-hydroxypyrrolidine-2-carboxamide
(2S,3S)-3-hydroxy-N-(4-octylphenethyl)pyrrolidine-2-carboxamide
cis-N-(4-decylphenyl)-3-hydroxypiperidine-2-carboxamide
(2S,3S)-N-4-(hexylsulfonylmethyl)phenyl)-3-hydroxypyrrolidine-2-carboxamide
(2S,3S)-2-(dipropylamino)-3-hydroxy-N-(4-octylphenyl)butanamide
(2S,3S)-2-(cyclohexylmethylamino)-3-hydroxy-N-(4-octylphenyl)butanamide
(2S,3S)-2-(dibenzylamino)-3-hydroxy-N-(4-octylphenyl)butanamide
(2S,3S)-N-(4-(3-cyclohexylpropyl)phenyl)-3-hydroxypyrrolidine-2-carboxamide
(S)-3-Hydroxy-2-(methylamino)-N-(4-octylphenyl)propanamide
(S)-2-Amino-6-hydroxy-N-(4-octylphenyl)hexanamide
(±)-2-Amino-4-hydroxy-N-(4-octylphenyl)butanamide
(S)-2-Amino-4-hydroxy-N-(4-octylphenyl)butanamide
(R)-2-Amino-4-hydroxy-N-(4-octylphenyl)butanamide
(±)-3-Amino-4-(4-octylphenylamino)butan-1-ol
(S)-2-Amino-4-hydroxy-N-(4-octylbenzyl)butanamide
(2S,3S)-3-hydroxy-N-(2-hydroxy-4-octylphenyl)pyrrolidine-2-
carboxamide hydrochloride
(2S,3R)-2-Amino-3-hydroxy-N-(4′-octylbiphenyl-4-yl)butanamide
(2R,3R)-3-Amino-4-(4′-octylbiphenyl-4-ylamino)butan-2-ol
(2S,3S)-N-((R)-5-(5-Heptyl-1,2,4-oxadiazol-3-yl)-2,3-dihydro-1H-inden-1-yl)-3-
hydroxypyrrolidine-2-carboxamide
(2S,3S)-N-((S)-5-(5-heptyl-1,2,4-oxadiazol-3-yl)-2,3-dihydro-1H-inden-1-yl)-3-
hydroxypyrrolidine-2-carboxamide
(2S,3S)-N-(4-(5-Cyclohexyl-1-methyl-1H-1,2,4-triazol-3-yl)benzyl)-3-
hydroxypyrrolidine-2-carboxamide
(2S,3S)-N-(4-(4-Heptyl-1H-1,2,3-triazol-1-yl)benzyl)-3-hydroxypyrrolidine-2-
carboxamide
(2S,3S)-3-Hydroxy-N-(4-(4-phenethyl-1H-1,2,3-triazol-1-yl)benzyl)pyrrolidine-2-
carboxamide
(2S,3S)-3-Hydroxy-N-((S)-1-(4-(4-phenethyl-1H-1,2,3-triazol-1-
yl)phenyl)propyl)pyrrolidine-2-carboxamide
(2S,3S)-N-(4-(4-(2-Cyclohexylethyl)-1H-1,2,3-triazol-1-yl)benzyl)-3-hydroxypyrrolidine-
2-carboxamide
(2S,3S)-N-(4-(4-(4-Fluorophenethyl)-1H-1,2,3-triazol-1-yl)benzyl)-3-hydroxypyrrolidine-
2-carboxamide
(2S,3S)-3-Hydroxy-N-(4-(4-phenethyl-1H-pyrazol-1-yl)benzyl)pyrrolidine-2-
carboxamide
(2S,3S)-N-((S)-1-(4-(5-Heptyl-1,2,4-oxadiazol-3-yl)phenyl)-2-methylpropyl)-3-
hydroxypyrrolidine-2-carboxamide
(2S,3S)-N-(4-(5-Heptyl-1-methyl-1H-1,2,4-triazol-3-yl)benzyl)-3-hydroxypyrrolidine-2-
carboxamide
(2S,3S)-N-((S)-1-(4-(5-Heptyl-1,2,4-oxadiazol-3-yl)phenyl)ethyl)-3-hydroxypyrrolidine-
2-carboxamide
(2S,3S)-N-((R)-1-(4-(5-Heptyl-1,2,4-oxadiazol-3-yl)phenyl)ethyl)-3-hydroxypyrrolidine-
2-carboxamide
(2S,3S)-N-(4-(5-Heptyl-1,3,4-oxadiazol-2-yl)benzyl)-3-hydroxypyrrolidine-2-
carboxamide
(2S,3S)-N-((S)-1-(4-(5-Heptyl-1,2,4-oxadiazol-3-yl)phenyl)propyl)-3-
hydroxypyrrolidine-2-carboxamide
(2S,3S)-N-(4-(3-Heptyl-1,2,4-oxadiazol-5-yl)benzyl)-3-hydroxypyrrolidine-2-
carboxamide
(2S,3S)-N-((3-(4-Heptylphenyl)-1,2,4-oxadiazol-5-yl)methyl)-3-hydroxypyrrolidine-2-
carboxamide
(2S,3S)-3-Hydroxy-N-(4-(5-(4-isobutylphenyl)-1,2,4-oxadiazol-3-yl)benzyl)pyrrolidine-
2-carboxamide
(2S,3S)-N-4-(5-Heptyl-1,2,4-oxadiazol-3-yl)benzyl)-3-hydroxypyrrolidine-2-
carboxamide
(2S,3S)-3-Hydroxy-N-(4-(5-octyl-1,2,4-oxadiazol-3-yl)benzyl)pyrrolidine-2-carboxamide
(2S,3S)-3-Hydroxy-N-(4-(5-(4-propylphenyl)-1,2,4-oxadiazol-3-yl)pyrrolidine-2-
carboxamide
(2S,3S)-N-(4-5-(4-Butylphenyl)-1,2,4-oxadiazol-3-yl)benzyl)-3-hydroxypyrrolidine-2-
carboxamide
(S)-2-Amino-N-(4-(5-heptyl-1,2,4-oxadiazol-3-yl)benzyl)-4-hydroxybutanamide
(2S,3S)-3-Hydroxy-N-(4-(5-pentyl-1,2,4-oxadiazol-3-yl)benzyl)pyrrolidine-2-
carboxamide
(2S,3S)-N-(4-(5-Hexyl-1,2,4-oxadiazol-3-yl)benzyl)-3-hydroxypyrrolidine-2-
carboxamide
(2S,3S)-N-(4-(5-Cyclohexyl-1,2,4-oxadiazol-3-yl)benzyl)-3-hydroxypyrrolidine-2-
carboxamide
(2S,3S)-3-Hydroxy-N-(4-(5-(1-methylcyclohexyl)-1,2,4-oxadiazol-3-
yl)benzyl)pyrrolidine-2-carboxamide
(2S,3S)-N-(4-(5-Cyclopentyl-1,2,4-oxadiazol-3-yl)benzyl)-3-hydroxypyrrolidine-2-
carboxamide
(2S,3S)-N-(4-(5-Cycloheptyl-1,2,4-oxadiazol-3-yl)benzyl)-3-hydroxypyrrolidine-2-
carboxamide
(2S,3S)-N-(4-(5-(2-Cyclopentylethyl-1,2,4-oxadiazol-3-yl)benzyl)-3-hydroxypyrrolidine-
2-carboxamide
(2S,3S)-N-(4-(5-(2-Cyclopropylethyl-1,2,4-oxadiazol-3-yl)benzyl)-3-
hydroxypyrrolidine-2-carboxamide
(2S,3S)-N-(4-(5-(4,4-Difluorocyclohexyl)-1,2,4-oxadiazol-3-yl)benzyl)-3-
hydroxypyrrolidine-2-carboxamide
(2S,3S)-N-(4-(5-(2-Cyclohexylethyl)-1,2,4-oxadiazol-3-yl)benzyl)-3-hydroxypyrrolidine-
2-carboxamide
(2S,3S)-3-Hydroxy-N-(4-(5-phenethyl-1,2,4-oxadiazol-3-yl)benzyl)pyrrolidine-2-
carboxamide
(2S,3S)-N-((S)-1-(4-(5-(2-Cyclohexylethyl)-1,2,4-oxadiazol-3-yl)phenyl)propyl)-3-
hydroxypyrrolidine-2-carboxamide
(2S,3S)-N-((S)-1-(4-(5-(2-Cyclohexylethyl)-1,2,4-oxadiazol-3-yl)phenyl)ethyl)-3-
hydroxypyrrolidine-2-carboxamide hemi-tartrate salt
(2S,3S)-N-((S)-1-(4-(5-(2-Cyclopentylethyl)-1,2,4-oxadiazol-3-yl)phenyl)ethyl)-3-
hydroxypyrrolidine-2-carboxamide hemi-tartrate salt
(2S,3S)-N-((S)-1-(4-(5-(2-Cyclopentylethyl)-1,2,4-oxadiazol-3-yl)phenyl)propyl)-3-
hydroxypyrrolidine-2-carboxamide hemi-tartrate salt
(2S,3S)-N-((S)-1-(4-(5-(2-Cyclohexyl-1,2,4-oxadiazol-3-yl)phenyl)propyl)-3-
hydroxypyrrolidine-2-carboxamide hemi-tartrate salt
(2S,3S)-N-((S)-1-(4-(5-(2-Cyclobutylethyl)-1,2,4-oxadiazol-3-yl)phenyl)ethyl)-3-
hydroxypyrrolidine-2-carboxamide hemi-tartrate salt
(2S,3R)-2-amino-N-(4-(5-heptylpyridin-2-yl)phenyl)-3-hydroxybutanamide
(2S,4S)-N-(4-(3-heptyl-1,2,4-oxadiazol-5-yl)benzyl)-4-hydroxypiperidine-2-carboxamide
(2S,3S)-3-hydroxy-N-(4-(2-octylthiazol-4-yl)benzyl)pyrrolidine-2-carboxamide
(2S,3S)-N-(4-(5-heptylpyridin-2-yl)phenyl)-3-hydroxypyrrolidine-2-carboxamide
(2S,3S)-N-(4-(5-heptyl-1H-pyrazol-3-yl)benzyl)-3-hydroxypyrrolidine-2-carboxamide
(2S,3S)-3-hydroxy-N-(4-(5-octylthiazol-2-yl)benzyl)pyrrolidine-2-carboxamide
(2S,3S)-3-Hydroxy-N-((2-octylbenzofuran-5-yl)methyl)pyrrolidine-2-carboxamide
(2S,3S)-3-Hydroxy-N-((2-octyl-1H-indol-5-yl)methyl)pyrrolidine-2-carboxamide
(2S,3S)-3-Hydroxy-N-((1-methyl-2-octyl-1H-indol-5-yl)methyl)pyrrolidine-2-
carboxamide
(2S,3S)-N-((2-(3-(4-Fluorophenoxy)propyl)benzofuran-5-yl)methyl)-3-
hydroxypyrrolidine-2-carboxamide
(2S,3S)-N-((2-(2-(4-Fluorophenoxy)ethyl)benzofuran-5-yl)methyl)-hydroxypyrrolidine-2-
carboxamide
(2S,3S)-3-Hydroxy-N-(2-octylbenzofuran-6-yl)methyl)pyrrolidine-2-carboxamide
(2S,3S)-N-((2-Heptylbenzofuran-5-yl)methyl)-3-hydroxypyrrolidine-2-carboxamide
(2S,3S)-N-((2-Hexylbenzofuran-5-yl)methyl)-3-hydroxypyrrolidine-2-carboxamide
(2S,3S)-N-((2-cyclohexylbenzofuran-5-yl)methyl)-3-hydroxypyrrolidine-2-carboxamide
(2S,3S)-N-((2-(Cyclohexylmethyl)benzofuran-5-yl)methyl)-3-hydroxypyrrolidine-2-
carboxamide
(2S,3S)-N-((2-(2-Cyclohexylethyl)benzofuran-5-yl)methyl)-3-hydroxypyrrolidine-2-
carboxamide
(2S,3S)-N-((7-Fluoro-2-octylbenzofuran-5-yl)methyl)-3-hydroxypyrrolidine-2-
carboxamide
(2S,3S)-N-((6-Fluoro-2-octylbenzofuran-5-yl)methyl)-3-hydroxypyrrolidine-2-
carboxamide
(2S,3S)-N-((2-(Cyclopentylmethyl)-7-fluorobenzofuran-5-yl)methyl)-3-
hydroxypyrrolidine-2-carboxamide
(2S,3S)-N-((4-Fluoro-2-octylbenzofuran-5-yl)methyl)-3-hydroxypyrrolidine-2-
carboxamide
(2S,3S)-N-((2-Cyclohexyl-7-fluorobenzofuran-5-yl)methyl)-3-hydroxypyrrolidine-2-
carboxamide
(2R,3S)-2-(6-octyl-1H-benzo[d]imidazol-2-yl)pyrrolidin-3-ol
(3-(hydroxymethyl)piperazin-1-yl)(4-octylphenyl)methanone hydrochloride
(2R,4R)-N-(4-decylphenyl)-4-hydroxypiperidine-2-carboxamide
(2S,4S)-N-(4-decylphenyl)-4-hydroxypiperidine-2-carboxamide
2-amino-2-ethyl-4-(4-nonylphenyl)butan-1-ol
2-amino-2-benzyl-4-(4-nonylphenyl)butan-1-ol
or a pharmaceutically acceptable salt, ester or prodrug thereof.
